(19) United States
(12) Patent Application Publication
(10) Pub. No.: US 2025/0034576 A1
(43) Pub. Date: Jan. 30, 2025

(54) Double Stranded Oligonucleotide For Modulating JAK1 Expression

(71) Applicant: Hoffmann-La Roche Inc., Little Falls, NJ (US)

(72) Inventors: Philipp Friedrich BERNINGER, Basel BS (CH); Konrad BLEICHER, Basel BS (CH); Erik FUNDER, Basel BS (CH); Helle JACOBSEN, Basel BS (CH); Dennis Jul HANSEN, Basel BS (CH); Michael KELLER, Basel BS (CH); Inna Appeldorff LARSEN, Basel BS (CH); Meiling LI, Basel BS (CH); Disa Elisabet TEHLER, Basel BS (CH); Lotte WINTHER, Basel BS (CH); Jesper WORM, Basel BS (CH); Lena WYSS, Basel BS (CH); Tiago Francisco SANTOS FERREIRA, Basel BS (CH)

(21) Appl. No.: 18/743,170

(22) Filed: Jun. 14, 2024

(30) Foreign Application Priority Data
Jun. 16, 2023 (EP) 23179895.0

(51) Int. Cl. C12N 15/113

(52) U.S. Cl. CPC C12N 15/1137; C12Y 207/10002; C12N 2310/14; C12N 2310/315; C12N 2310/321; C12N 2310/322; C12N 2310/351

(57) ABSTRACT

The present invention relates to double stranded oligonucleotides that are complementary to JAK1, leading to modulation of the expression of JAK1. Modulation of JAK1 expression is beneficial for a range of medical disorders including inflammatory bowel disease, organ transplant rejection, graft-versus-host disease, multiple sclerosis, rheumatoid arthritis (RA), juvenile idiopathic arthritis, psoriasis, dermatitis, diabetic nephropathy, systemic lupus erythematosus (SLE), dry eye disease, cancer, myelofibrosis, and asthma. Also included are compositions comprising the double stranded oligonucleotide and methods of treatment using the double stranded oligonucleotide.

### REFERENCE TO AN ELECTRONIC SEQUENCE LISTING

This application contains a sequence listing which is submitted electronically and is hereby incorporated by reference in its entirety. The sequence listing submitted herewith is contained in the XML filed created Jun. 13, 2024 entitled “P127679PCT_ST26.xml” and is 5,469 kilobytes in size.

### FIELD OF INVENTION

The present invention relates to double stranded oligonucleotides that are complementary to JAK1, leading to modulation of the expression of JAK1. Modulation of JAK1 expression is beneficial for a range of medical disorders including inflammatory bowel disease, organ transplant rejection, graft-versus-host disease, multiple sclerosis, rheumatoid arthritis (RA), juvenile idiopathic arthritis, psoriasis, dermatitis, diabetic nephropathy, systemic lupus erythematosus (SLE), dry eye disease, cancer, myelofibrosis, and asthma.

### BACKGROUND

The Janus kinase (JAK) family of human tyrosine kinases are non-receptor tyrosine kinases that transduce cytokine-mediated signals via the JAK-STAT pathway. JAKSs possess two highly similar phosphate-transferring domains, with one exhibiting kinase activity, and the other negatively regulating kinase activity of the first.

JAKs are involved in providing catalytic kinase activity for the type I and type II cytokine receptor signal transduction pathways. As members of these receptor families lack catalytic kinase activity, JAKs are required to phosphorylate and activate downstream proteins involved in the signal transduction pathways. Modulation of JAKs is connected with atopic dermatitis, rheumatoid arthritis, psoriasis, polycythemia vera, alopecia, essential thrombocythemia, ulcerative colitis, myeloid metaplasia with myelofibrosis and vitiligo.

JAK kinases are implicated in initiating responses to multiple major cytokine receptor families. Modulation of JAK1 expression is connected with a range of medical disorders including dry eye disease, as well as inflammatory bowel disease, organ transplant rejection, graft-versus-host disease, multiple sclerosis, rheumatoid arthritis (RA), juvenile idiopathic arthritis, psoriasis, dermatitis, diabetic nephropathy, systemic lupus erythematosus (SLE), cancer, myelofibrosis, and asthma.

Dry eye disease is a disease of the tears and ocular surface that is accompanied by increased osmolarity of the tear film and inflammation of the cornea and conjunctiva. It can result in discomfort, visual disturbance, tear film instability, and possible damage to the ocular surface. Dry eye disease generally results from a disturbance or lack of function in the lacrimal glands, ocular surface and lids, as well as in in the nerves connecting them. The inflammation can be initiated either by chronic irritative stress (e.g. wearing contact lens) or a systemic inflammatory autoimmune disease like rheumatoid arthritis.

Dry eye disease is typically more prevalent in women than men, and prevalence also increases with age. Currently it is often treated by artificial lubricants called “artificial tears” comprising hypotonic or isotonic buffered solutions containing electrolytes, surfactants and various types of viscosity agents. Alternatively, tear retention devices can be implanted, or moisture chamber glasses worn. Anti-inflammatory drugs can also be used to treat any inflammation caused by the disease, such as topical corticosteroid drops. Corticosteroids can rapidly and effectively relieve the symptoms of dry eye disease.

The type I and type II cytokine receptor signal transduction pathways that JAK kinases are involved in have roles in the immune response, being linked to defences against extracellular infections. They can also contribute to pathogenesis of some autoimmune inflammatory diseases including dry eye disease. Given this role in the pathogenesis of dry eye disease, cytokines, via JAK kinases, are attractive targets for treatment and use as anti-inflammatory agents.

### OBJECTIVE OF THE INVENTION

The present invention identifies regions of the JAK1 transcript (JAK1) for antisense inhibition in vitro or in vivo, and provides for dsRNAs which target these regions of the JAK1 pre-mRNA or mature mRNA. The present invention identifies dsRNAs which inhibit human JAK1 which are useful in the treatment of a range of medical disorders including inflammatory bowel disease, organ transplant rejection, graft-versus-host disease, multiple sclerosis, rheumatoid arthritis (RA), juvenile idiopathic arthritis, psoriasis, dermatitis, diabetic nephropathy, systemic lupus erythematosus (SLE), dry eye disease, cancer, myelofibrosis, and asthma.

### SUMMARY OF THE INVENTION

The present invention provides double stranded ribonucleic acid (dsRNA) molecules which target JAK1.

Double stranded RNA molecules, such as siRNA molecules, can modulate the expression of a target nucleic acid, in particular by binding to complementary mRNA sequences after transcription, typically leading to degradation and loss of translation of the target mRNA, and decrease in the level of expression of the target nucleic acid.

siRNA molecules are capable of inducing RNA-dependent gene silencing via the RNA-induced silencing complex (RISC) in a cell's cytoplasm, where they interact with the catalytic RISC component Argonaute.

In a first aspect the invention provides a compound comprising a double stranded ribonucleic acid (dsRNA) for reducing the expression of Janus kinase 1 (JAK1), the dsRNA comprising a sense strand and an antisense strand,
wherein the sense strand comprises a first contiguous nucleotide sequence of at least 15 nucleotides in length,
wherein the antisense strand comprises a second contiguous nucleotide sequence of at least 15 nucleotides in length which is complementary to a JAK1 nucleic acid sequence which comprises or consists of SEQ ID NO: 1 or a naturally occurring variant thereof, and
wherein the first contiguous nucleotide sequence and the second contiguous nucleotide sequence form a double stranded region of complementarity.

The double stranded region of complementarity may be 15-21 nucleotides in length.

The second contiguous nucleotide sequence may be 15-24 nucleotides in length.

The second contiguous nucleotide sequence may be complementary to a target sequence within the JAK1 nucleic acid sequence, wherein the target sequence is any one of the sequences of SEQ ID NOs 385-575.

The second contiguous nucleotide sequence may comprise a sequence having at least 80% identity to any one of the sequences of SEQ ID NOs 194-384.

The first contiguous nucleotide sequence may be 15-24 nucleotides in length.

The first contiguous nucleotide sequence may comprises a sequence having at least 80% identity to any one of the sequences of SEQ ID NOs 3-193.

In another aspect of the invention the antisense strand and the sense strand may form a duplex selected from the group consisting of Duplex ID NOs: 1-191 of Table 1.

The dsRNA may be an siRNA.

Within the compound of the invention, the dsRNA may be covalently attached to at least one conjugate moiety.

The invention also provides a compound comprising or consisting of a compound selected from the group of compounds 614, 673, 724, 728, 753, 756, 818, 874, 875, 876, 877, 878, 883, 884, 1069, 1075, 1085, 1107, 1108, 1138, 1182, 1189, 1190, 1304, 1306, 1311, 1367, 1368, 1372, 1412, 1413, 1432, 1579, 1580, 1581, 1583, 1584, 1586, 1587, 1588, 1595, 1596, 1601, 1602, 1603, 1608, 1609, 1611, 1640, 1642, 1671, 1672, 1673, 1674, 1677, 1678, 1690, 1692, 1698, 1699, 1723, 1769, 1770, 1780, 1798, 1876, 1927, 1928, 1929, 1936, 1952, 1954, 1956, 1958, 1978, 2066, 2068, 2102, 2111, 2138, 2146, 2148, 2205, 2206, 2218, 2229, 2230, 2237, 2238, 2239, 2269, 2308, 2317, 2318, 2319, 2320, 2321, 2322, 2323, 2520, 2527, 2647, 2761, 2762, 2763, 2764, 2811, 2962, 2975, 2977, 3028, 3032, 3081, 3131, 3134, 3141, 3144, 3146, 3147, 3159, 3160, 3229, 3247, 3250, 3251, 3252, 3254, 3255, 3258, 3259, 3260, 3261, 3265, 3268, 3272, 3275, 3276, 3278, 3279, 3281, 3282, 3283, 3284, 3285, 3286, 3313, 3314, 3323, 3353, 3365, 3367, 3368, 3371, 3372, 3376, 3409, 3505, 3556, 3557, 3558, 3559, 3654, 3662, 3663, 3683, 3689, 3694, 3695, 3698, 3702, 3719, 3781, 3894, 4099, 4169, 4239, 4305, 4374, 4411, 4475, 4612, 4671, 4672, 4679, 4682, 4683, 4684, 4690, 4794, 4803, and 4807 as shown in Table 3, preferably a compound selected from compounds 614, 673, 1182, 1770, 1954, 2319, 3131, 3255, 3265, and 3313 as shown in Table 3.

The invention also provides a compound comprising or consisting of a compound selected from the group of compound 614, 673, 1182, 1770, 1954, 2319, 3131, 3255, 3265, and 3313 as shown in Table 4.

The invention also provides a compound comprising or consisting of a compound selected from the group of compounds 614_C22, 673_C22, 1182_C22, 1770_C22, 1954_C22, 2319_C22, 3131_C22, 3255_C22, 3265_C22, and 3313_C22 as shown in Table 4.

The invention also provides a compound comprising or consisting of a compound selected from the group of compounds 614_C16, 673_C16, 1182_C16, 1770_C16, 1954_C16, 2319_C16, 3131_C16, 3255_C16, 3265_C16, and 3313_C16.

The invention also provides a compound comprising or consisting of a structure selected from the structures as shown in any of FIGS. 7 to 76.

The compound of the invention may be capable of decreasing the expression of JAK1 mRNA by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%, compared to a control.

The compound of the invention may be capable of decreasing the expression of JAK1 protein by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%, compared to a control.

In another aspect the invention provides a pharmaceutical composition comprising the compound of the invention and a pharmaceutically acceptable diluent, solvent, carrier, salt and/or adjuvant.

The pharmaceutical composition may comprise one or more additional therapeutic agents, such as a JAK1 inhibitor, a JAK1 antagonist therapeutic, or an anti-JAK1 antibody.

In another aspect the invention provides an in vivo or in vitro method for suppressing JAK1 expression in a target cell by administering the compound or the pharmaceutical composition of the invention, in an effective amount, to the cell.

In another aspect the invention provides a method for treating or preventing a disease comprising administering a therapeutically or prophylactically effective amount of the compound or pharmaceutical composition of the invention to a subject suffering from or susceptible to a disease.

The invention also provides the compound or the pharmaceutical composition of the invention for use in a method for treating or preventing a disease.

The invention also provides use of the compound or the pharmaceutical composition of the invention for the preparation of a medicament for treatment or prevention of a disease in a subject.

The disease may be associated with signalling through JAK1, such as inflammatory bowel disease, organ transplant rejection, graft-versus-host disease, multiple sclerosis, rheumatoid arthritis (RA), juvenile idiopathic arthritis, psoriasis, dermatitis, diabetic nephropathy, systemic lupus erythematosus (SLE), dry eye disease, cancer, myelofibrosis, and asthma.

In another aspect the invention provides a kit comprising the compound of the invention and instructions for use. The kit may also comprise one or more additional therapeutic agents.

### BRIEF DESCRIPTION OF THE FIGURES

FIGS. 1a and 1b show qPCR of JAK1 mRNA expression after transfection of HCEC cells with the siRNAs targeting JAK1 (Compound #614, 673, 1182, 1770, 1954, 2319, 3131, 3255, 3265, and 3313).

FIGS. 2a and 2b show qPCR of JAK1 mRNA expression after Gymnosis for 5 days of SIRC1 cells with the C22 conjugated siRNAs targeting JAK1 (compound #614_C22, 673_C22, 1182_C22, 1770_C22, 1954_C22, 2319_C22, 3131_C22, 3255_C22, 3265_C22, and 3313_C22).

FIGS. 3a and 3b show qPCR of JAK1 mRNA expression after Gymnosis for 5 days of HCEC cells with the C22 conjugated siRNAs targeting JAK1 (compound #614_C22, 673_C22, 1182_C22, 1770_C22, 1954_C22, 2319_C22, 3131_C22, 3255_C22, 3265_C22, and 3313_C22).

FIG. 4 show JAK1 mRNA expression normalized to GAPDH in EYEPRIM samples measured by ddPCR showing knockdown in vivo in rabbits after topical administration of JAK1 C22 conjugated siRNA (compound #614_C22, 673_C22, 1182_C22, 1770_C22, 1954_C22, 2319_C22, 3131_C22, 3255_C22, 3265_C22, and 3313_C22).

FIG. 5 show JAK1 mRNA expression normalized to GAPDH in EYEPRIM samples measured by ddPCR showing knockdown in vivo in rabbits after topical administration of JAK1 C22 conjugated siRNA (Compound #614_C22, and 1182_C22).

FIG. 6 show CXCL10 mRNA expression normalized to GAPDH in EYEPRIM samples measured by ddPCR showing upregulation by IFNg and reduction of CXCL10 in vivo in rabbits after topical administration of JAK1 C22 conjugated siRNA (C22-conjugated compound #614_C22 and 1182_C22).

FIG. 7 shows compound 614.

FIG. 8 shows compound 614_C16.

FIG. 9 shows compound 614_C22.

FIG. 10 shows the antisense strand of compounds 614, 614_C16 and 614_C22.

FIG. 11 shows the sense strand of compound 614.

FIG. 12 shows the sense strand of compound 614_C16.

FIG. 13 shows the sense strand of compound 614_C22.

FIG. 14 shows compound 673.

FIG. 15 shows compound 673_C16.

FIG. 16 shows compound 673_C22.

FIG. 17 shows the antisense strand of compounds 673, 673_C16 and 673_C22.

FIG. 18 shows the sense strand of compound 673.

FIG. 19 shows the sense strand of compound 673_C16.

FIG. 20 shows the sense strand of compound 673_C22.

FIG. 21 shows compound 1182.

FIG. 22 shows compound 1182_C16.

FIG. 23 shows compound 1182_C22.

FIG. 24 shows the antisense strand of compounds 1182, 1182_C16 and 1182_C22.

FIG. 25 shows the sense strand of compound 1182.

FIG. 26 shows the sense strand of compound 1182_C16.

FIG. 27 shows the sense strand of compound 1182_C22.

FIG. 28 shows compound 1770.

FIG. 29 shows compound 1770_C16.

FIG. 30 shows compound 1770_C22.

FIG. 31 shows the antisense strand of compounds 1770, 1770_C16 and 1770_C22.

FIG. 32 shows the sense strand of compound 1770.

FIG. 33 shows the sense strand of compound 1770_C16.

FIG. 34 shows the sense strand of compound 1770_C22.

FIG. 35 shows compound 1954.

FIG. 36 shows compound 1954_C16.

FIG. 37 shows compound 1954_C22.

FIG. 38 shows the antisense strand of compounds 1954, 1954_C16 and 1954_C22.

FIG. 39 shows the sense strand of compound 1954.

FIG. 40 shows the sense strand of compound 1954_C16.

FIG. 41 shows the sense strand of compound 1954_C22.

FIG. 42 shows compound 2319.

FIG. 43 shows compound 2319_C16.

FIG. 44 shows compound 2319_C22.

FIG. 45 shows the antisense strand of compounds 2319, 2319_C16 and 2319_C22.

FIG. 46 shows the sense strand of compound 2319.

FIG. 47 shows the sense strand of compound 2319_C16.

FIG. 48 shows the sense strand of compound 2319_C22.

FIG. 49 shows compound 3131.

FIG. 50 shows compound 3131_C16.

FIG. 51 shows compound 3131_C22.

FIG. 52 shows the antisense strand of compounds 3131, 3131_C16 and 3131_C22.

FIG. 53 shows the sense strand of compound 3131.

FIG. 54 shows the sense strand of compound 3131_C16.

FIG. 55 shows the sense strand of compound 3131_C22.

FIG. 56 shows compound 3255.

FIG. 57 shows compound 3255_C16.

FIG. 58 shows compound 3255_C22.

FIG. 59 shows the antisense strand of compounds 3255, 3255_C16 and 3255_C22.

FIG. 60 shows the sense strand of compound 3255.

FIG. 61 shows the sense strand of compound 3255_C16.

FIG. 62 shows the sense strand of compound 3255_C22.

FIG. 63 shows compound 3265.

FIG. 64 shows compound 3265_C16.

FIG. 65 shows compound 3265_C22.

FIG. 66 shows the antisense strand of compounds 3265, 3265_C16 and 3265_C22.

FIG. 67 shows the sense strand of compound 3265.

FIG. 68 shows the sense strand of compound 3265_C16.

FIG. 69 shows the sense strand of compound 3265_C22.

FIG. 70 shows compound 3313.

FIG. 71 shows compound 3313_C16.

FIG. 72 shows compound 3313_C22.

FIG. 73 shows the antisense strand of compounds 3313, 3313_C16 and 3313_C22.

FIG. 74 shows the sense strand of compound 3313.

FIG. 75 shows the sense strand of compound 3313_C16.

FIG. 76 shows the sense strand of compound 3313_C22.

FIG. 77 shows JAK1 expression in rabbit eyes (from samples removed by the EYEPRIM™ medical device to perform reliable conjunctival impressions) treated with different C16 siRNAs (as well as compounds 614 and 614_C22 aa controls) and normalized to a combined set of housekeeping genes (“HKG”), namely HPRT1 (Hypoxanthin-Guanin-Phosphoribosyltransferase), PPIA (peptidylprolyl isomerase A) and GAPDH (glyceraldehyde-3-phosphate dehydrogenase); PBS: phosphate buffered saline.

### DETAILED DESCRIPTION OF THE INVENTION

The invention provides a compound comprising a double stranded ribonucleic acid (dsRNA) for reducing the expression of Janus kinase 1 (JAK1), the dsRNA comprising a sense strand and an antisense strand,
wherein the sense strand comprises a first contiguous nucleotide sequence of at least 15 nucleotides in length,
wherein the antisense strand comprises a second contiguous nucleotide sequence of at least 15 nucleotides in length which is complementary to a JAK1 nucleic acid sequence which comprises or consists of SEQ ID NO: 1 or a naturally occurring variant thereof, and
wherein the first contiguous nucleotide sequence and the second contiguous nucleotide sequence form a double stranded region of complementarity.

### Compound

A “compound” is a physical entity which may have any features as defined further herein. The term “compound” merely denotes a physical entity and does not in itself imply any additional features.

Herein the terms “compound of the invention”, “compound”, “antisense compound of the invention”, “antisense compound”, “nucleic acid molecule of the invention”, “nucleic acid molecule”, “ribonucleic acid of the invention” and “ribonucleic acid” are used interchangeably.

The term “compound” encompasses conjugated compounds (i.e. compounds which comprise a conjugate moiety) and non-conjugated compounds (i.e. compounds which do not comprise a conjugate moiety).

### Ribonucleic Acid

A “ribonucleic acid” as described herein is a type of nucleic acid molecule comprising predominantly ribonucleotides, i.e. nucleotides comprising a ribose sugar.

A ribonucleic acid comprises at least about 50% ribonucleotides, such as at least about 60%, at least about 70%, at least about 80%, at least about 90%, at least about 95%, at least about 99% or about 100% ribonucleotides. The ribonucleic acid may also comprises one or more deoxyribose containing nucleotides (i.e. DNA nucleotides), such as one, two, three, four, five, six, seven, eight, nine, ten or more deoxyribose containing nucleotides.

### Nucleoside, Nucleotide, Oligonucleotide and Nucleic Acid

Nucleotides and nucleosides are the building blocks of nucleic acid molecules, oligonucleotides and polynucleotides, and for the purposes of the present invention include both naturally occurring and non-naturally occurring nucleotides and nucleosides.

Nucleotides, such as DNA and RNA nucleotides, comprise a deoxyribose or ribose sugar moiety, a nucleobase moiety and one or more phosphate groups.

Nucleosides comprise a deoxyribose or ribose sugar moiety and a nucleobase moiety.

Nucleosides and nucleotides may also interchangeably be referred to as “units”, “monomers”, “bases” or “nucleobases”.

The term “oligonucleotide” as used herein is defined, as is generally understood by the skilled person, as a molecule comprising two or more covalently linked nucleosides. Such covalently bound nucleosides may also be referred to as nucleic acid molecules or oligomers.

Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. Oligonucleotides as described herein are man-made, chemically synthesized, and are typically purified or isolated. Oligonucleotides may comprise one or more modified nucleosides or nucleotides.

The term “nucleic acid”, “nucleic acid molecule” or “therapeutic nucleic acid molecule” as used herein is defined as it is generally understood by the skilled person as a molecule comprising two or more covalently linked nucleosides (i.e. a nucleotide sequence).

A “nucleic acid molecule” may be a deoxyribose nucleic acid or a ribonucleic acid.

Nucleic acid molecules, such as for siRNAs, shRNAs and antisense oligonucleotides, are typically for inhibiting the expression of a target nucleic acid(s).

As used herein, the terms “oligonucleotide”, “polynucleotide”, “nucleic acid”, “nucleic acid molecule” and “nucleic acid sequence” are intended to be synonymous with each other.

Nucleic acid molecules are commonly made in the laboratory by solid-phase chemical synthesis followed by purification and isolation. When referring to a sequence of the nucleic acid molecule, reference is made to the sequence or order of nucleobase moieties, or modifications thereof, of the covalently linked nucleotides or nucleosides. Nucleic acid molecules referred to herein are man-made, are chemically synthesized, and are typically purified or isolated. A nucleic acid molecule may comprise one or more modified nucleosides or nucleotides as described further herein.

A “portion” means a defined number of contiguous (i.e. linked) nucleobases of a nucleic acid. In certain embodiments, a portion is a defined number of contiguous nucleobases of a target nucleic acid.

### Single Stranded, Double Stranded and Hybridization

The term “single stranded” is generally understood by the skilled person in the art as a nucleic acid having only one strand. Especially it is understood that a single stranded nucleic acid can form hairpins or intermolecular duplex structures (duplex between two molecules of the same oligonucleotide), as long as the degree of intra or inter self-complementarity is less than 50% across of the full length of the oligonucleotide.

The ribonucleic acids of the invention as described as being “double stranded”. The term double stranded is generally understood by the skilled person in the art and requires the ribonucleic acid to contain two strands, which hybridise along a proportion of the two strands. The two strands may hybridise along at least about 50% of the length of the shortest strand, such as at least about 60%, at least about 70%, at least about 80%, at least about 90%, at least about 95%, at least about 99% or about 100% of the length of the shortest strand. The term “duplex” is also used herein to refer to a double-stranded region.

There is no requirement for the two strands to be the same length or to hybridise along the entire length of either strand.

The terms “hybridizing” or “hybridizes” as used herein are to be understood as two nucleic acid strands (e.g. an a sense strand and an antisense strand) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537, which is hereby incorporated by reference in its entirety).

The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the sense strand and the antisense strand. ΔG° is the energy associated with a reaction where aqueous concentrations are 1M, the pH is 7, and the temperature is 37° C. The hybridization of nucleic acid strands is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today, which are incorporated by reference in their entirety. The skilled person will know that commercial equipment is available for ΔG° measurements. ΔG° can also be estimated numerically by using the nearest neighbour model as described by SantaLucia, 1998, Proc Natl Acad Sci USA. 95:1460-1465 (incorporated by reference in its entirety) using appropriately derived thermodynamic parameters described by Sugimoto et al., 1995, Biochemistry 34:11211-11216 and McTigue et al., 2004, Biochemistry 43:5388-5405 (incorporated by reference in their entirety).

In some embodiments the degree or strength of hybridization is measured by the standard state Gibbs free energy ΔG°. The nucleic acid strands may hybridize with estimated ΔG° values below the range of −10 kcal, such as below −15 kcal, such as below −20 kcal and such as below −25 kcal. In some embodiments the nucleic acid strands may hybridize with an estimated ΔG° value of −10 to −60 kcal, such as −12 to −40, such as from −15 to −30 kcal, or −16 to −27 kcal such as −18 to −25 kcal.

RNAi Molecule, siRNA and shRNA

The compound of the invention is an RNAi molecule.

Herein, the term “RNA interference (RNAi) molecule”, “RNAi molecule” or “RNAi” refers to a short, typically double stranded, RNA molecule capable of inducing RNA-dependent gene silencing via the RNA-induced silencing complex (RISC) in a cell's cytoplasm, where they interact with the catalytic RISC component argonaute.

In some embodiments, the compound of the invention is an siRNA. In some embodiments, the dsRNA is a siRNA.

A small interfering RNA (siRNA) is a typically double stranded RNA molecule that, by binding to a complementary mRNA after transcription, typically leads to degradation of the mRNA and loss in translation. In other words, the term “siRNA” or “siRNA molecule” as used herein is defined as a nucleic acid molecule capable of modulating expression of a target by binding to a target nucleic acid, in particular to a contiguous sequence on a target nucleic acid.

siRNA molecules are typically 20-24 base pairs in length and usually have phosphorylated 5′ ends and hydroxylated 3′ ends with two overhanging nucleotides.

A small interfering RNA (siRNA) may also be known as a short interfering RNA or a silencing RNA.

Another type of RNAi molecule is a small hairpin RNA (shRNA) which is an artificial RNA molecule with a hairpin structure which, upon expression, is able to reduce the level of a target mRNA via the DICER and RNA reducing silencing complex (RISC). A small hairpin RNA (shRNA) may also be known as a short hairpin RNA.

RNAi molecules can be designed on the basis of the RNA sequence of the gene of interest. Corresponding RNAi molecules can then be synthesized chemically or by in vitro transcription, or expressed from a vector or PCR product.

siRNA and shRNA molecules are generally between 20 and 50 nucleotides in length, such as between 25 and 35 nucleotides in length, and may interact with the endonuclease known as Dicer which is believed to process double stranded RNA into 19-23 base pair short interfering RNAs (siRNAs) with characteristic two base 3′ overhangs which are then incorporated into an RNA-induced silencing complex (RISC).

Effective extended forms of Dicer substrates have been described in U.S. Pat. Nos. 8,349,809 and 8,513,207, hereby incorporated by reference. Upon binding to the appropriate target mRNA, one or more endonucleases within the RISC cleave the target to induce silencing. RNAi agents may be chemically modified using modified internucleotide linkages and high affinity nucleosides, such as 2′-4′ bicyclic ribose modified nucleosides, including LNA and cET, as described further below.

### JAK1 Nucleic Acid Sequence

The second contiguous nucleotide sequence is complementary to a JAK1 nucleic acid sequence which comprises or consists of SEQ ID NO: 1 or a naturally occurring variant thereof.

SEQ ID NO: 1 is the JAK1 mRNA sequence set forth in GENBANK Accession No. NM_002227.4 dated 22 Jan. 2023, and is as follows:

GCGTCGCTGAGCGCAGGCCGCGGCGGCCGCGGAGTATCCTGGAGCTGCAGACAGTG
CGGGCCTGCGCCCAGTCCCGGCTGTCCTCGCCGCGACCCCTCCTCAGCCCTGGGCG
CGCGCACGCTGGGGCCCCGCGGGGCTGGCCGCCTAGCGAGCCTGCCGGTCGACCCC
AGCCAGCGCAGCGACGGGGCGCTGCCTGGCCCAGGCGCACACGGAAGTGCGCTTCT
CTGAAGTAGCTTTGGAAAGTAGAGAAGAAAATCCAGTTTGCTTCTTGGAGAACACTGGA
CAGCTGAATAAATGCAGTATCTAAATATAAAAGAGGACTGCAATGCCATGGCTTTCTGT
GCTAAAATGAGGAGCTCCAAGAAGACTGAGGTGAACCTGGAGGCCCCTGAGCCAGGG
GTGGAAGTGATCTTCTATCTGTCGGACAGGGAGCCCCTCCGGCTGGGCAGTGGAGAG
TACACAGCAGAGGAACTGTGCATCAGGGCTGCACAGGCATGCCGTATCTCTCCTCTTT
GTCACAACCTCTTTGCCCTGTATGACGAGAACACCAAGCTCTGGTATGCTCCAAATCGC
ACCATCACCGTTGATGACAAGATGTCCCTCCGGCTCCACTACCGGATGAGGTTCTATTT
CACCAATTGGCATGGAACCAACGACAATGAGCAGTCAGTGTGGCGTCATTCTCCAAAG
AAGCAGAAAAATGGCTACGAGAAAAAAAAGATTCCAGATGCAACCCCTCTCCTTGATGC
CAGCTCACTGGAGTATCTGTTTGCTCAGGGACAGTATGATTTGGTGAAATGCCTGGCTC
CTATTCGAGACCCCAAGACCGAGCAGGATGGACATGATATTGAGAACGAGTGTCTAGG
GATGGCTGTCCTGGCCATCTCACACTATGCCATGATGAAGAAGATGCAGTTGCCAGAA
CTGCCCAAGGACATCAGCTACAAGCGATATATTCCAGAAACATTGAATAAGTCCATCAG
ACAGAGGAACCTTCTCACCAGGATGCGGATAAATAATGTTTTCAAGGATTTCCTAAAGG
AATTTAACAACAAGACCATTTGTGACAGCAGCGTGTCCACGCATGACCTGAAGGTGAAA
TACTTGGCTACCTTGGAAACTTTGACAAAACATTACGGTGCTGAAATATTTGAGACTTCC
ATGTTACTGATTTCATCAGAAAATGAGATGAATTGGTTTCATTCGAATGACGGTGGAAAC
GTTCTCTACTACGAAGTGATGGTGACTGGGAATCTTGGAATCCAGTGGAGGCATAAACC
AAATGTTGTTTCTGTTGAAAAGGAAAAAAATAAACTGAAGCGGAAAAAACTGGAAAATAA
ACACAAGAAGGATGAGGAGAAAAACAAGATCCGGGAAGAGTGGAACAATTTTTCTTACT
TCCCTGAAATCACTCACATTGTAATAAAGGAGTCTGTGGTCAGCATTAACAAGCAGGAC
AACAAGAAAATGGAACTGAAGCTCTCTTCCCACGAGGAGGCCTTGTCCTTTGTGTCCCT
GGTAGATGGCTACTTCCGGCTCACAGCAGATGCCCATCATTACCTCTGCACCGACGTG
GCCCCCCCGTTGATCGTCCACAACATACAGAATGGCTGTCATGGTCCAATCTGTACAGA
ATACGCCATCAATAAATTGCGGCAAGAAGGAAGCGAGGAGGGGATGTACGTGCTGAGG
TGGAGCTGCACCGACTTTGACAACATCCTCATGACCGTCACCTGCTTTGAGAAGTCTGA
GCAGGTGCAGGGTGCCCAGAAGCAGTTCAAGAACTTTCAGATCGAGGTGCAGAAGGG
CCGCTACAGTCTGCACGGTTCGGACCGCAGCTTCCCCAGCTTGGGAGACCTCATGAGC
CACCTCAAGAAGCAGATCCTGCGCACGGATAACATCAGCTTCATGCTAAAACGCTGCT
GCCAGCCCAAGCCCCGAGAAATCTCCAACCTGCTGGTGGCTACTAAGAAAGCCCAGGA
GTGGCAGCCCGTCTACCCCATGAGCCAGCTGAGTTTCGATCGGATCCTCAAGAAGGAT
CTGGTGCAGGGCGAGCACCTTGGGAGAGGCACGAGAACACACATCTATTCTGGGACC
CTGATGGATTACAAGGATGACGAAGGAACTTCTGAAGAGAAGAAGATAAAAGTGATCCT
CAAAGTCTTAGACCCCAGCCACAGGGATATTTCCCTGGCCTTCTTCGAGGCAGCCAGC
ATGATGAGACAGGTCTCCCACAAACACATCGTGTACCTCTATGGCGTCTGTGTCCGCG
ACGTGGAGAATATCATGGTGGAAGAGTTTGTGGAAGGGGGTCCTCTGGATCTCTTCAT
GCACCGGAAAAGCGATGTCCTTACCACACCATGGAAATTCAAAGTTGCCAAACAGCTG
GCCAGTGCCCTGAGCTACTTGGAGGATAAAGACCTGGTCCATGGAAATGTGTGTACTA
AAAACCTCCTCCTGGCCCGTGAGGGCATCGACAGTGAGTGTGGCCCATTCATCAAGCT
CAGTGACCCCGGCATCCCCATTACGGTGCTGTCTAGGCAAGAATGCATTGAACGAATC
CCATGGATTGCTCCTGAGTGTGTTGAGGACTCCAAGAACCTGAGTGTGGCTGCTGACA
AGTGGAGCTTTGGAACCACGCTCTGGGAAATCTGCTACAATGGCGAGATCCCCTTGAA
AGACAAGACGCTGATTGAGAAAGAGAGATTCTATGAAAGCCGGTGCAGGCCAGTGACA
CCATCATGTAAGGAGCTGGCTGACCTCATGACCCGCTGCATGAACTATGACCCCAATC
AGAGGCCTTTCTTCCGAGCCATCATGAGAGACATTAATAAGCTTGAAGAGCAGAATCCA
GATATTGTTTCAGAAAAAAAACCAGCAACTGAAGTGGACCCCACACATTTTGAAAAGCG
CTTCCTAAAGAGGATCCGTGACTTGGGAGAGGGCCACTTTGGGAAGGTTGAGCTCTGC
AGGTATGACCCCGAAGGGGACAATACAGGGGAGCAGGTGGCTGTTAAATCTCTGAAGC
CTGAGAGTGGAGGTAACCACATAGCTGATCTGAAAAAGGAAATCGAGATCTTAAGGAAC
CTCTATCATGAGAACATTGTGAAGTACAAAGGAATCTGCACAGAAGACGGAGGAAATGG
TATTAAGCTCATCATGGAATTTCTGCCTTCGGGAAGCCTTAAGGAATATCTTCCAAAGAA
TAAGAACAAAATAAACCTCAAACAGCAGCTAAAATATGCCGTTCAGATTTGTAAGGGGA
TGGACTATTTGGGTTCTCGGCAATACGTTCACCGGGACTTGGCAGCAAGAAATGTCCTT
GTTGAGAGTGAACACCAAGTGAAAATTGGAGACTTCGGTTTAACCAAAGCAATTGAAAC
CGATAAGGAGTATTACACCGTCAAGGATGACCGGGACAGCCCTGTGTTTTGGTATGCT
CCAGAATGTTTAATGCAATCTAAATTTTATATTGCCTCTGACGTCTGGTCTTTTGGAGTC
ACTCTGCATGAGCTGCTGACTTACTGTGATTCAGATTCTAGTCCCATGGCTTTGTTCCT
GAAAATGATAGGCCCAACCCATGGCCAGATGACAGTCACAAGACTTGTGAATACGTTAA
AAGAAGGAAAACGCCTGCCGTGCCCACCTAACTGTCCAGATGAGGTTTATCAACTTATG
AGGAAATGCTGGGAATTCCAACCATCCAATCGGACAAGCTTTCAGAACCTTATTGAAGG
ATTTGAAGCACTTTTAAAATAAGAAGCATGAATAACATTTAAATTCCACAGATTATCAAGT
CCTTCTCCTGCAACAAATGCCCAAGTCATTTTTTAAAAATTTCTAATGAAAGAAGTTTGT
GTTCTGTCCAAAAAGTCACTGAACTCATACTTCAGTACATATACATGTATAAGGCACACT
GTAGTGCTTAATATGTGTAAGGACTTCCTCTTTAAATTTGGTACCAGTAACTTAGTGACA
CATAATGACAACCAAAATATTTGAAAGCACTTAAGCACTCCTCCTTGTGGAAAGAATATA
CCACCATTTCATCTGGCTAGTTCACCATCACAACTGCATTACCAAAAGGGGATTTTTGAA
AACGAGGAGTTGACCAAAATAATATCTGAAGATGATTGCTTTTCCCTGCTGCCAGCTGA
TCTGAAATGTTTTGCTGGCACATTAATCATAGATAAAGAAAGATTGATGGACTTAGCCCT
CAAATTTCAGTATCTATACAGTACTAGACCATGCATTCTTAAAATATTAGATACCAGGTA
GTATATATTGTTTCTGTACAAAAATGACTGTATTCTCTCACCAGTAGGACTTAAACTTTGT
TTCTCCAGTGGCTTAGCTCCTGTTCCTTTGGGTGATCACTAGCACCCATTTTTGAGAAA
GCTGGTTCTACATGGGGGGATAGCTGTGGAATAGATAATTTGCTGCATGTTAATTCTCA
AGAACTAAGCCTGTGCCAGTGCTTTCCTAAGCAGTATACCTTTAATCAGAACTCATTCCC
AGAACCTGGATGCTATTACACATGCTTTTAAGAAACGTCAATGTATATCCTTTTATAACT
CTACCACTTTGGGGCAAGCTATTCCAGCACTGGTTTTGAATGCTGTATGCAACCAGTCT
GAATACCACATACGCTGCACTGTTCTTAGAGGGTTTCCATACTTACCACCGATCTACAA
GGGTTGATCCCTGTTTTTACCATCAATCATCACCCTGTGGTGCAACACTTGAAAGACCC
GGCTAGAGGCACTATGGACTTCAGGATCCACTAGACAGTTTTCAGTTTGCTTGGAGGTA
GCTGGGTAATCAAAAATGTTTAGTCATTGATTCAATGTGAACGATTACGGTCTTTATGAC
CAAGAGTCTGAAAATCTTTTTGTTATGCTGTTTAGTATTCGTTTGATATTGTTACTTTTCA
CCTGTTGAGCCCAAATTCAGGATTGGTTCAGTGGCAGCAATGAAGTTGCCATTTAAATT
TGTTCATAGCCTACATCACCAAGGTCTCTGTGTCAAACCTGTGGCCACTCTATATGCAC
TTTGTTTACTCTTTATACAAATAAATATACTAAAGACTTTA

The term “naturally occurring variant” refers to variants of the JAK1 nucleic acid sequence which originate from the same genetic locus as the JAK1 nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms, and allelic variants. The compound of the invention may target the JAK1 nucleic acid and naturally occurring variants thereof.

In some embodiments, the JAK1 nucleic acid sequence comprises SEQ ID NO: 1.

In some embodiments, the JAK1 nucleic acid sequence consists of SEQ ID NO: 1.

Unless otherwise indicated or contradicted by context, in the present disclosure, thymine (T) nucleobases within RNA sequences disclosed herein (siRNAs or mRNA target sequences) are to be interpreted as uracil (U) nucleobases.

### Complementarity

In the present invention, the second contiguous nucleotide sequence is “complementary” to the JAK1 nucleic acid sequence or to a target sequence within the JAK1 nucleic acid sequence, and the first and second contiguous nucleotide sequences form a double-stranded region of “complementarity”.

The terms “complementary” and “complementarity” describe the capacity for Watson-Crick base-pairing of nucleosides/nucleotides. Watson-Crick base pairs are guanine (G)-cytosine (C) and adenine (A)-thymine (T)/uracil (U). For example, in DNA, adenine (A) is complementary to thymine (T) and guanine (G) is complementary to cytosine (C). For example, in RNA, adenine (A) is complementary to uracil (U) and guanine (G) is complementary to cytosine (C). In some embodiments, complementary nucleotides can base pair in the Watson-Crick manner or in any other manner that allows for the formation of stable duplexes.

It will be understood that oligonucleotides may comprise nucleosides with modified nucleobases, for example 5-methyl cytosine is often used in place of cytosine, and as such the term complementarity encompasses Watson-Crick base-paring between non-modified and modified nucleobases (see for example Hirao et al., 2012, Accounts of Chemical Research, 45, 2055 and Bergstrom, 2009, Curr. Protoc. Nucleic Acid Chem., 37, 1.4.1, which are incorporated by reference in their entirety).

The term “complementary” (such as in the phrase “the second contiguous nucleotide sequence is complementary to a JAK1 nucleic acid sequence”) does not require 100% complementarity. Rather, within the present invention, the term “complementary” requires that the two sequences are sufficiently complementary (i.e. form a sufficient number of Watson-Crick base-pairs) to hybridise to one another and form a double-stranded structure (i.e. duplex).

In some embodiments, a “complementary” sequence is at least about 70% complementary to another sequence, such as at least about 75% complementary, at least about 80% complementary, at least about 85% complementary, at least about 90% complementary, at least about 95% complementary, or at least about 99% complementary.

The term “% complementary” as used herein, refers to the proportion of nucleotides (in percent) within a double-stranded region of complementarity which across the length of the double-stranded region of complementarity, are complementary (i.e. form Watson-Crick base-pairs).

The term “% complementary” is also used herein to refer to the proportion of nucleotides (in percent) within a query sequence (such as a second contiguous nucleotide sequence or an antisense strand, as described herein) which are complementary to a reference sequence (such as a first contiguous nucleotide sequence, a sense strand, a JAK1 nucleic acid sequence or a target sequence within the JAK1 nucleic acid sequence, as described herein).

To calculate the percentage of complementarity, the query sequence and reference sequence are first aligned, with the query sequence running 5′-3′ and the reference sequence 3′-5′. The sequences are aligned to maximise the number of complementary base pairs (i.e. Watson-Crick base pairs) between the two sequences. The percentage complementarity is calculated by counting the number of aligned nucleobases that are complementary (form Watson-Crick base pairs) between the two aligned sequences, dividing that number by the total number of nucleotides in the portion of the query sequence aligned with the reference sequence (i.e. any nucleotides at the 5′ and 3′ ends of the query sequence that are not complementary to the reference sequence, and any nucleotides at the 5′ and 3′ ends of the reference sequence that are not complementary to the query sequence, do not count towards the length of the sequence) and multiplying by 100. The resulting number is the percentage complementarity of the query sequence to the reference sequence.

In such a comparison a nucleobase/nucleotide which does not align (form a base pair) is termed a mismatch. Insertions and deletions are not allowed in the calculation of % complementarity of a contiguous nucleotide sequence.

It will be understood that in determining complementarity, chemical modifications of the nucleobases are disregarded as long as the functional capacity of the nucleobase to form Watson-Crick base pairing is retained (e.g. 5′-methyl cytosine is considered identical to a cytosine for the purpose of calculating % complementarity).

Target Sequence within the JAK1 Nucleic Acid Sequence

In some embodiments, the second contiguous nucleotide sequence is complementary to a target sequence within the JAK1 nucleic acid sequence, wherein the target sequence is any one of the sequences of SEQ ID NOs 385-575 (as shown in Table 1 herein).

In some embodiments, the second contiguous nucleotide sequence is complementary to a target sequence within the JAK1 nucleic acid sequence, wherein the target sequence is any one of the sequences of SEQ ID NOs 385, 386, 405, 447, 456, 479, 498, 512, 517 and 530 (as shown in Table 1 herein). These are the target sequences for the preferred compounds depicted in Table 4 herein.

In some embodiments, the second contiguous nucleotide sequence is at least 80% complementary to the target sequence. In other words, the second contiguous nucleotide sequence is at least 80% complementary to any one of the sequences of SEQ ID NOs 385-575, preferably any one of the sequences of SEQ ID NOs 385, 386, 405, 447, 456, 479, 498, 512, 517 and 530.

In some embodiments, the second contiguous nucleotide sequence is at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% complementary to the target sequence. In some embodiments, the second contiguous nucleotide sequence is fully (i.e. 100%) complementary to the target sequence.

In one embodiment, the second contiguous nucleotide sequence is complementary, such as at least 80% complementary, such as fully complementary, to SEQ ID NO: 385 (target sequence of compound 614).

In another embodiment, the second contiguous nucleotide sequence is complementary, such as at least 80% complementary, such as fully complementary, to SEQ ID NO: 386 (target sequence of compound 673).

In another embodiment the second contiguous nucleotide sequence is complementary, such as at least 80% complementary, such as fully complementary, to SEQ ID NO: 405 (target sequence of compound 1182).

In another embodiment the second contiguous nucleotide sequence is complementary, such as at least 80% complementary, such as fully complementary, to SEQ ID NO: 447 (target sequence of compound 1770).

In another embodiment the second contiguous nucleotide sequence is complementary, such as at least 80% complementary, such as fully complementary, to SEQ ID NO: 456 (target sequence of compound 1954).

In another embodiment the second contiguous nucleotide sequence is complementary, such as at least 80% complementary, such as fully complementary, to SEQ ID NO: 479 (target sequence of compound 2319).

In another embodiment the second contiguous nucleotide sequence is complementary, such as at least 80% complementary, such as fully complementary, to SEQ ID NO: 498 (target sequence of compound 3131).

In another embodiment the second contiguous nucleotide sequence is complementary, such as at least 80% complementary, such as fully complementary, to SEQ ID NO: 512 (target sequence of compound 3255).

In another embodiment the second contiguous nucleotide sequence is complementary, such as at least 80% complementary, such as fully complementary, to SEQ ID NO: 517 (target sequence of compound 3265).

In another embodiment the second contiguous nucleotide sequence is complementary, such as at least 80% complementary, such as fully complementary, to SEQ ID NO: 530 (target sequence of compound 3313).

In view of the definition of complementarity herein, the expression “wherein the second contiguous nucleotide sequence is at least 80% complementary to the target sequence” will be understood to mean that when the second contiguous nucleotide sequence and target sequence (i.e. any one of the sequences of SEQ ID NOs 385-575) are aligned to maximise complementarity, and any non-complementary (i.e. overhanging nucleotides) at the 5′ and 3′ ends of each contiguous nucleotide sequence are disregarded, the percentage of nucleotides in either sequence which form complementary base pairs with the other sequence is at least 80%.

A corresponding meaning will be understood to apply to all other percentage complementarity values between the second contiguous nucleotide sequence and the target sequence described herein, such as 81%, 82%, 83%, 84%, 85%, 86%, 87%, 88%, 89%, 90%, 91%, 92%, 93%, 94%, 95%, 96%, 97%, 98%, 99% and 100%.

Thus, the expression “wherein the second contiguous nucleotide sequence is at least 90% complementary to the target sequence” will be understood to mean that when the second contiguous nucleotide sequence and target sequence (i.e. any one of the sequences of SEQ ID NOs 385-575) are aligned to maximise complementarity, and any non-complementary (i.e. overhanging nucleotides) at the 5′ and 3′ ends of each contiguous nucleotide sequence are disregarded, the percentage of nucleotides in either sequence which form complementary base pairs with the other sequence is at least 90%.

The expression “wherein the second contiguous nucleotide sequence is at least 95% complementary to the target sequence” will be understood to mean that when the second contiguous nucleotide sequence and target sequence (i.e. any one of the sequences of SEQ ID NOs 385-575) are aligned to maximise complementarity, and any non-complementary (i.e. overhanging nucleotides) at the 5′ and 3′ ends of each contiguous nucleotide sequence are disregarded, the percentage of nucleotides in either sequence which form complementary base pairs with the other sequence is at least 95%.

The expression “the second contiguous nucleotide sequence is fully complementary to a target sequence” will be understood to mean that when the second contiguous nucleotide sequence and target sequence are aligned to maximise complementarity, and any non-complementary (i.e. overhanging nucleotides) at the 5′ and 3′ ends of each sequence are disregarded, all nucleotides in each sequence form complementary base pairs with the other sequence (i.e. 100% complementarity).

Likewise, the expression “the antisense strand is fully complementary to a target sequence” will be understood to mean that when the antisense strand sequence and target sequence are aligned to maximise complementarity, and any non-complementary (i.e. overhanging nucleotides) at the 5′ and 3′ ends of each sequence are disregarded, all nucleotides in each sequence form complementary base pairs with the other sequence (i.e. 100% complementarity).

### Double Stranded Region of Complementarity

Within the double stranded ribonucleic acids of the invention there is a double stranded region of complementarity.

A “double stranded region of complementarity” is the region of the ribonucleic acid containing multiple nucleotides that are complementary with each other.

In some embodiments, the first contiguous nucleotide sequence and the second contiguous nucleotide sequence within the double stranded region of complementarity are at least about 80% complementary.

In some embodiments the first contiguous nucleotide sequence and the second contiguous nucleotide sequence within the double stranded region of complementarity are at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% complementary. In some embodiments the first contiguous nucleotide sequence and the second contiguous nucleotide sequence within the double stranded region of complementarity are fully complementary (i.e. about 100% complementary, or 100% complementary).

In view of the definition of complementarity herein, the expression “wherein the first contiguous nucleotide sequence and the second contiguous nucleotide sequence within the double stranded region of complementarity are at least 80% complementary” will be understood to mean that when the first contiguous nucleotide sequence and second contiguous nucleotide sequence are aligned to maximise complementarity, and any non-complementary (i.e. overhanging nucleotides) at the 5′ and 3′ ends of each contiguous nucleotide sequence are disregarded, the percentage of nucleotides in either sequence which form complementary base pairs with the other sequence is at least 80%.

A corresponding meaning will be understood to apply to all other percentage complementarity values between the first contiguous nucleotide sequence and the second contiguous nucleotide sequence within the double stranded region of complementarity described herein, such as 81%, 82%, 83%, 84%, 85%, 86%, 87%, 88%, 89%, 90%, 91%, 92%, 93%, 94%, 95%, 96%, 97%, 98%, 99% and 100%.

Thus, the expression “wherein the first contiguous nucleotide sequence and the second contiguous nucleotide sequence within the double stranded region of complementarity are at least 90% complementary” will be understood to mean that when the first contiguous nucleotide sequence and second contiguous nucleotide sequence are aligned to maximise complementarity, and any non-complementary (i.e. overhanging nucleotides) at the 5′ and 3′ ends of each contiguous nucleotide sequence are disregarded, the percentage of nucleotides in either sequence which form complementary base pairs with the other sequence is at least 90%.

The expression “wherein the first contiguous nucleotide sequence and the second contiguous nucleotide sequence within the double stranded region of complementarity are at least 95% complementary” will be understood to mean that when the first contiguous nucleotide sequence and second contiguous nucleotide sequence are aligned to maximise complementarity, and any non-complementary (i.e. overhanging nucleotides) at the 5′ and 3′ ends of each contiguous nucleotide sequence are disregarded, the percentage of nucleotides in either sequence which form complementary base pairs with the other sequence is at least 95%.

The expression “wherein the first contiguous nucleotide sequence and the second contiguous nucleotide sequence within the double stranded region of complementarity are fully complementary” will be understood to mean that when the first contiguous nucleotide sequence and second contiguous nucleotide sequence are aligned to maximise complementarity, and any non-complementary (i.e. overhanging nucleotides) at the 5′ and 3′ ends of each contiguous nucleotide sequence are disregarded, all nucleotides in each sequence form complementary base pairs with the other sequence (i.e. 100% complementarity).

In some embodiments, the double-stranded region of complementarity comprises one or more mismatches. In some embodiments, the double-stranded region of complementarity comprises one, two, three, four or five mismatches. In some embodiments, the double stranded region of complementarity comprises one mismatch. In some embodiments, the double stranded region of complementarity comprises two mismatches. In some embodiments, the double stranded region of complementarity comprises three mismatches.

In some embodiments, the double stranded region of complementarity is 15-21 nucleotides long. In some embodiments, the double stranded region of complementarity is 15, 16, 17, 18, 19, 20 or 21 nucleotides long. In some embodiments, the double stranded region of complementarity is 19 nucleotides long

### Seed Region

A “seed region” (or “seed”) refers to a region between nucleotides 2 to 8 of the antisense strand (counting from the 5′ end of the antisense strand). For example, the seed region may be from position 2 to position 8 of the antisense strand or from position 2 to position 7 of the antisense strand.

In some embodiments, the antisense strand comprises a seed region. In some embodiments the second contiguous nucleotide sequence comprises the seed region. In some embodiments, the seed region comprises or consists of a sequence of at least 6 contiguous nucleotides of any one of the sequence of SEQ ID NOs 576-766. In some embodiments, the seed region comprises any one of the sequence of SEQ ID NOs 576-766. In some embodiments, the seed region consists of any one of the sequence of SEQ ID NOs 576-766.

In preferred embodiments the seed region comprises the sequence of SEQ ID NO: 576 (seed region of compound 614).

In preferred embodiments the seed region comprises the sequence of SEQ ID NO: 577 (seed region of compound 673).

In preferred embodiments the seed region comprises the sequence of SEQ ID NO: 596 (seed region of compound 1182).

In preferred embodiments the seed region comprises the sequence of SEQ ID NO: 638 (seed region of compound 1770).

In preferred embodiments the seed region comprises the sequence of SEQ ID NO: 647 (seed region of compound 1954).

In preferred embodiments the seed region comprises the sequence of SEQ ID NO: 670 (seed region of compound 2319).

In preferred embodiments the seed region comprises the sequence of SEQ ID NO: 689 (seed region of compound 3131).

In preferred embodiments the seed region comprises the sequence of SEQ ID NO: 703 (seed region of compound 3255).

In preferred embodiments the seed region comprises the sequence of SEQ ID NO: 708 (seed region of compound 3265).

In preferred embodiments the seed region comprises the sequence of SEQ ID NO: 721 (seed region of compound 3313).

In preferred embodiments the seed region consists of at least 6 contiguous nucleotides of SEQ ID NO: 576.

In preferred embodiments the seed region consists of at least 6 contiguous nucleotides of SEQ ID NO: 577.

In preferred embodiments the seed region consists of at least 6 contiguous nucleotides of SEQ ID NO: 596.

In preferred embodiments the seed region consists of at least 6 contiguous nucleotides of SEQ ID NO: 638.

In preferred embodiments the seed region consists of at least 6 contiguous nucleotides of SEQ ID NO: 647.

In preferred embodiments the seed region consists of at least 6 contiguous nucleotides of SEQ ID NO: 670.

In preferred embodiments the seed region consists of at least 6 contiguous nucleotides of SEQ ID NO: 689.

In preferred embodiments the seed region consists of at least 6 contiguous nucleotides of SEQ ID NO: 703.

In preferred embodiments the seed region consists of at least 6 contiguous nucleotides of SEQ ID NO: 708.

In preferred embodiments the seed region consists of at least 6 contiguous nucleotides of SEQ ID NO: 721.

In preferred embodiments the seed region consists of the sequence of SEQ ID NO: 576.

In preferred embodiments the seed region consists of the sequence of SEQ ID NO: 577.

In preferred embodiments the seed region consists of the sequence of SEQ ID NO: 596.

In preferred embodiments the seed region consists of the sequence of SEQ ID NO: 638.

In preferred embodiments the seed region consists of the sequence of SEQ ID NO: 647.

In preferred embodiments the seed region consists of the sequence of SEQ ID NO: 670.

In preferred embodiments the seed region consists of the sequence of SEQ ID NO: 689.

In preferred embodiments the seed region consists of the sequence of SEQ ID NO: 703.

In preferred embodiments the seed region consists of the sequence of SEQ ID NO: 708.

In preferred embodiments the seed region consists of the sequence of SEQ ID NO: 721.

### Identity

The term “identity” as used herein, refers to the proportion of nucleotides (expressed in percent) of a contiguous nucleotide sequence in a nucleic acid molecule which at a given position, are identical to (i.e. in their ability to form Watson-Crick base pairs with the complementary nucleoside) a contiguous nucleotide sequence, at a given position of a separate nucleic acid molecule.

The percentage identity is thus calculated by counting the number of aligned nucleobases that are identical (a Match) between two sequences, dividing that number by the total number of nucleotides in the oligonucleotide and multiplying by 100. Therefore, percentage identity=(matches×100)/length of aligned region (e.g. the contiguous nucleotide sequence). Preferably, insertions and deletions are not allowed in the calculation of the percentage identity of a contiguous nucleotide sequence. It will be understood that in determining identity, chemical modifications of the nucleobases are disregarded as long as the functional capacity of the nucleobase to form Watson-Crick base pairing is retained (e.g. 5-methyl cytosine is considered identical to a cytosine for the purpose of calculating % identity).

### Second Contiguous Nucleotide Sequence

The term “contiguous nucleotide sequence” refers to a region of the antisense strand of the ribonucleic acid which is complementary to a region of the sense strand of the ribonucleic acid, and vice versa.

The term “contiguous nucleotide sequence” is used interchangeably herein with the term “contiguous nucleobase sequence”. In some embodiments, all the nucleotides of the sense strand and/or the antisense strand constitute the contiguous nucleotide sequence. In some embodiments, the sense strand and/or the antisense strand comprises the contiguous nucleotide sequence and may optionally comprise further nucleotide(s), for example a nucleotide linker region which may be used to attach a functional group to the first contiguous nucleotide sequence or the second contiguous nucleotide sequence. The nucleotide linker region may or may not be complementary to the target nucleic acid.

The antisense strand comprises a second contiguous nucleotide sequence of at least 15 nucleotides in length.

As described elsewhere herein, the second contiguous nucleotide sequence is complementary to the JAK1 nucleic acid sequence and to the target sequence within the JAK1 nucleic acid sequence. Also as described elsewhere herein, the second contiguous nucleotide sequence forms a double-stranded region of complementarity with the first contiguous nucleotide sequence.

In some embodiments, the second contiguous nucleotide sequence is 15, 16, 17, 18, 19, 20 or 21 nucleotides long. In some embodiments, the second contiguous nucleotide sequence is 20 or 21 nucleotides long.

In some embodiments, the second contiguous nucleotide sequence is 15 nucleotides long.

In some embodiments, the second contiguous nucleotide sequence is 16 nucleotides long.

In some embodiments, the second contiguous nucleotide sequence is 17 nucleotides long.

In some embodiments, the second contiguous nucleotide sequence is 18 nucleotides long.

In some embodiments, the second contiguous nucleotide sequence is 19 nucleotides long.

In some embodiments, the second contiguous nucleotide sequence is 20 nucleotides long.

In some embodiments, the second contiguous nucleotide sequence is 21 nucleotides long.

In some embodiments the second contiguous nucleotide sequence corresponds to a portion of SEQ ID NO: 2 (which is JAK1 cDNA sequence) in which thymine (T) nucleobases are replaced with uracil (U) nucleobases.

SEQ ID NO: 2 is as follows:

TAAAGTCTTTAGTATATTTATTTGTATAAAGAGTAAACAAAGTGCATATAGAGTGGCCAC
AGGTTTGACACAGAGACCTTGGTGATGTAGGCTATGAACAAATTTAAATGGCAACTTCA
TTGCTGCCACTGAACCAATCCTGAATTTGGGCTCAACAGGTGAAAAGTAACAATATCAA
ACGAATACTAAACAGCATAACAAAAAGATTTTCAGACTCTTGGTCATAAAGACCGTAATC
GTTCACATTGAATCAATGACTAAACATTTTTGATTACCCAGCTACCTCCAAGCAAACTGA
AAACTGTCTAGTGGATCCTGAAGTCCATAGTGCCTCTAGCCGGGTCTTTCAAGTGTTGC
ACCACAGGGTGATGATTGATGGTAAAAACAGGGATCAACCCTTGTAGATCGGTGGTAA
GTATGGAAACCCTCTAAGAACAGTGCAGCGTATGTGGTATTCAGACTGGTTGCATACAG
CATTCAAAACCAGTGCTGGAATAGCTTGCCCCAAAGTGGTAGAGTTATAAAAGGATATA
CATTGACGTTTCTTAAAAGCATGTGTAATAGCATCCAGGTTCTGGGAATGAGTTCTGATT
AAAGGTATACTGCTTAGGAAAGCACTGGCACAGGCTTAGTTCTTGAGAATTAACATGCA
GCAAATTATCTATTCCACAGCTATCCCCCCATGTAGAACCAGCTTTCTCAAAAATGGGT
GCTAGTGATCACCCAAAGGAACAGGAGCTAAGCCACTGGAGAAACAAAGTTTAAGTCC
TACTGGTGAGAGAATACAGTCATTTTTGTACAGAAACAATATATACTACCTGGTATCTAA
TATTTTAAGAATGCATGGTCTAGTACTGTATAGATACTGAAATTTGAGGGCTAAGTCCAT
CAATCTTTCTTTATCTATGATTAATGTGCCAGCAAAACATTTCAGATCAGCTGGCAGCAG
GGAAAAGCAATCATCTTCAGATATTATTTTGGTCAACTCCTCGTTTTCAAAAATCCCCTTT
TGGTAATGCAGTTGTGATGGTGAACTAGCCAGATGAAATGGTGGTATATTCTTTCCACA
AGGAGGAGTGCTTAAGTGCTTTCAAATATTTTGGTTGTCATTATGTGTCACTAAGTTACT
GGTACCAAATTTAAAGAGGAAGTCCTTACACATATTAAGCACTACAGTGTGCCTTATACA
TGTATATGTACTGAAGTATGAGTTCAGTGACTTTTTGGACAGAACACAAACTTCTTTCAT
TAGAAATTTTTAAAAAATGACTTGGGCATTTGTTGCAGGAGAAGGACTTGATAATCTGTG
GAATTTAAATGTTATTCATGCTTCTTATTTTAAAAGTGCTTCAAATCCTTCAATAAGGTTC
TGAAAGCTTGTCCGATTGGATGGTTGGAATTCCCAGCATTTCCTCATAAGTTGATAAAC
CTCATCTGGACAGTTAGGTGGGCACGGCAGGCGTTTTCCTTCTTTTAACGTATTCACAA
GTCTTGTGACTGTCATCTGGCCATGGGTTGGGCCTATCATTTTCAGGAACAAAGCCATG
GGACTAGAATCTGAATCACAGTAAGTCAGCAGCTCATGCAGAGTGACTCCAAAAGACCA
GACGTCAGAGGCAATATAAAATTTAGATTGCATTAAACATTCTGGAGCATACCAAAACAC
AGGGCTGTCCCGGTCATCCTTGACGGTGTAATACTCCTTATCGGTTTCAATTGCTTTGG
TTAAACCGAAGTCTCCAATTTTCACTTGGTGTTCACTCTCAACAAGGACATTTCTTGCTG
CCAAGTCCCGGTGAACGTATTGCCGAGAACCCAAATAGTCCATCCCCTTACAAATCTGA
ACGGCATATTTTAGCTGCTGTTTGAGGTTTATTTTGTTCTTATTCTTTGGAAGATATTCCT
TAAGGCTTCCCGAAGGCAGAAATTCCATGATGAGCTTAATACCATTTCCTCCGTCTTCT
GTGCAGATTCCTTTGTACTTCACAATGTTCTCATGATAGAGGTTCCTTAAGATCTCGATT
TCCTTTTTCAGATCAGCTATGTGGTTACCTCCACTCTCAGGCTTCAGAGATTTAACAGCC
ACCTGCTCCCCTGTATTGTCCCCTTCGGGGTCATACCTGCAGAGCTCAACCTTCCCAAA
GTGGCCCTCTCCCAAGTCACGGATCCTCTTTAGGAAGCGCTTTTCAAAATGTGTGGGGT
CCACTTCAGTTGCTGGTTTTTTTTCTGAAACAATATCTGGATTCTGCTCTTCAAGCTTATT
AATGTCTCTCATGATGGCTCGGAAGAAAGGCCTCTGATTGGGGTCATAGTTCATGCAGC
GGGTCATGAGGTCAGCCAGCTCCTTACATGATGGTGTCACTGGCCTGCACCGGCTTTC
ATAGAATCTCTCTTTCTCAATCAGCGTCTTGTCTTTCAAGGGGATCTCGCCATTGTAGCA
GATTTCCCAGAGCGTGGTTCCAAAGCTCCACTTGTCAGCAGCCACACTCAGGTTCTTG
GAGTCCTCAACACACTCAGGAGCAATCCATGGGATTCGTTCAATGCATTCTTGCCTAGA
CAGCACCGTAATGGGGATGCCGGGGTCACTGAGCTTGATGAATGGGCCACACTCACTG
TCGATGCCCTCACGGGCCAGGAGGAGGTTTTTAGTACACACATTTCCATGGACCAGGT
CTTTATCCTCCAAGTAGCTCAGGGCACTGGCCAGCTGTTTGGCAACTTTGAATTTCCAT
GGTGTGGTAAGGACATCGCTTTTCCGGTGCATGAAGAGATCCAGAGGACCCCCTTCCA
CAAACTCTTCCACCATGATATTCTCCACGTCGCGGACACAGACGCCATAGAGGTACAC
GATGTGTTTGTGGGAGACCTGTCTCATCATGCTGGCTGCCTCGAAGAAGGCCAGGGAA
ATATCCCTGTGGCTGGGGTCTAAGACTTTGAGGATCACTTTTATCTTCTTCTCTTCAGAA
GTTCCTTCGTCATCCTTGTAATCCATCAGGGTCCCAGAATAGATGTGTGTTCTCGTGCC
TCTCCCAAGGTGCTCGCCCTGCACCAGATCCTTCTTGAGGATCCGATCGAAACTCAGC
TGGCTCATGGGGTAGACGGGCTGCCACTCCTGGGCTTTCTTAGTAGCCACCAGCAGGT
TGGAGATTTCTCGGGGCTTGGGCTGGCAGCAGCGTTTTAGCATGAAGCTGATGTTATC
CGTGCGCAGGATCTGCTTCTTGAGGTGGCTCATGAGGTCTCCCAAGCTGGGGAAGCTG
CGGTCCGAACCGTGCAGACTGTAGCGGCCCTTCTGCACCTCGATCTGAAAGTTCTTGA
ACTGCTTCTGGGCACCCTGCACCTGCTCAGACTTCTCAAAGCAGGTGACGGTCATGAG
GATGTTGTCAAAGTCGGTGCAGCTCCACCTCAGCACGTACATCCCCTCCTCGCTTCCTT
CTTGCCGCAATTTATTGATGGCGTATTCTGTACAGATTGGACCATGACAGCCATTCTGT
ATGTTGTGGACGATCAACGGGGGGGCCACGTCGGTGCAGAGGTAATGATGGGCATCT
GCTGTGAGCCGGAAGTAGCCATCTACCAGGGACACAAAGGACAAGGCCTCCTCGTGG
GAAGAGAGCTTCAGTTCCATTTTCTTGTTGTCCTGCTTGTTAATGCTGACCACAGACTCC
TTTATTACAATGTGAGTGATTTCAGGGAAGTAAGAAAAATTGTTCCACTCTTCCCGGATC
TTGTTTTTCTCCTCATCCTTCTTGTGTTTATTTTCCAGTTTTTTCCGCTTCAGTTTATTTTT
TTCCTTTTCAACAGAAACAACATTTGGTTTATGCCTCCACTGGATTCCAAGATTCCCAGT
CACCATCACTTCGTAGTAGAGAACGTTTCCACCGTCATTCGAATGAAACCAATTCATCTC
ATTTTCTGATGAAATCAGTAACATGGAAGTCTCAAATATTTCAGCACCGTAATGTTTTGT
CAAAGTTTCCAAGGTAGCCAAGTATTTCACCTTCAGGTCATGCGTGGACACGCTGCTGT
CACAAATGGTCTTGTTGTTAAATTCCTTTAGGAAATCCTTGAAAACATTATTTATCCGCAT
CCTGGTGAGAAGGTTCCTCTGTCTGATGGACTTATTCAATGTTTCTGGAATATATCGCTT
GTAGCTGATGTCCTTGGGCAGTTCTGGCAACTGCATCTTCTTCATCATGGCATAGTGTG
AGATGGCCAGGACAGCCATCCCTAGACACTCGTTCTCAATATCATGTCCATCCTGCTCG
GTCTTGGGGTCTCGAATAGGAGCCAGGCATTTCACCAAATCATACTGTCCCTGAGCAAA
CAGATACTCCAGTGAGCTGGCATCAAGGAGAGGGGTTGCATCTGGAATCTTTTTTTTCT
CGTAGCCATTTTTCTGCTTCTTTGGAGAATGACGCCACACTGACTGCTCATTGTCGTTG
GTTCCATGCCAATTGGTGAAATAGAACCTCATCCGGTAGTGGAGCCGGAGGGACATCT
TGTCATCAACGGTGATGGTGCGATTTGGAGCATACCAGAGCTTGGTGTTCTCGTCATAC
AGGGCAAAGAGGTTGTGACAAAGAGGAGAGATACGGCATGCCTGTGCAGCCCTGATG
CACAGTTCCTCTGCTGTGTACTCTCCACTGCCCAGCCGGAGGGGCTCCCTGTCCGACA
GATAGAAGATCACTTCCACCCCTGGCTCAGGGGCCTCCAGGTTCACCTCAGTCTTCTT
GGAGCTCCTCATTTTAGCACAGAAAGCCATGGCATTGCAGTCCTCTTTTATATTTAGATA
CTGCATTTATTCAGCTGTCCAGTGTTCTCCAAGAAGCAAACTGGATTTTCTTCTCTACTT
TCCAAAGCTACTTCAGAGAAGCGCACTTCCGTGTGCGCCTGGGCCAGGCAGCGCCCC
GTCGCTGCGCTGGCTGGGGTCGACCGGCAGGCTCGCTAGGCGGCCAGCCCCGCGGG
GCCCCAGCGTGCGCGCGCCCAGGGCTGAGGAGGGGTCGCGGCGAGGACAGCCGGG
ACTGGGCGCAGGCCCGCACTGTCTGCAGCTCCAGGATACTCCGCGGCCGCCGCGGC
CTGCGCTCAGCGACGC

In some embodiments, the second contiguous nucleotide sequence comprises any one of the sequences of SEQ ID NOs 194-384 (as shown in Table 1 herein). In some embodiments, the second contiguous nucleotide sequence comprises any one of the sequences of SEQ ID NOs 194, 195, 214, 256, 265, 288, 307, 321, 326 and 339. These are the sequences of the second contiguous nucleotide sequence of the preferred compounds depicted in Table 4 herein.

In some embodiments, the second contiguous nucleotide sequence consists of any one of the sequences of SEQ ID NOs 194-384. In some embodiments, the second contiguous nucleotide sequence consist of any one of the sequences of SEQ ID NOs 194, 195, 214, 256, 265, 288, 307, 321, 326 and 339.

In preferred embodiments, the second contiguous nucleotide sequence comprises the sequence of SEQ ID NO: 194.

In preferred embodiments, the second contiguous nucleotide sequence comprises the sequence of SEQ ID NO: 195.

In preferred embodiments, the second contiguous nucleotide sequence comprises the sequence of SEQ ID NO: 214.

In preferred embodiments, the second contiguous nucleotide sequence comprises the sequence of SEQ ID NO: 256.

In preferred embodiments, the second contiguous nucleotide sequence comprises the sequence of SEQ ID NO: 265.

In preferred embodiments, the second contiguous nucleotide sequence comprises the sequence of SEQ ID NO: 288.

In preferred embodiments, the second contiguous nucleotide sequence comprises the sequence of SEQ ID NO: 307.

In preferred embodiments, the second contiguous nucleotide sequence comprises the sequence of SEQ ID NO: 321.

In preferred embodiments, the second contiguous nucleotide sequence comprises the sequence of SEQ ID NO: 326.

In preferred embodiments, the second contiguous nucleotide sequence comprises the sequence of SEQ ID NO: 339.

In preferred embodiments, the second contiguous nucleotide sequence consists of the sequence of SEQ ID NO: 194.

In preferred embodiments, the second contiguous nucleotide sequence consists of the sequence of SEQ ID NO: 195.

In preferred embodiments, the second contiguous nucleotide sequence consists of the sequence of SEQ ID NO: 214.

In preferred embodiments, the second contiguous nucleotide sequence consists of the sequence of SEQ ID NO: 256.

In preferred embodiments, the second contiguous nucleotide sequence consists of the sequence of SEQ ID NO: 265.

In preferred embodiments, the second contiguous nucleotide sequence consists of the sequence of SEQ ID NO: 288.

In preferred embodiments, the second contiguous nucleotide sequence consists of the sequence of SEQ ID NO: 307.

In preferred embodiments, the second contiguous nucleotide sequence consists of the sequence of SEQ ID NO: 321.

In preferred embodiments, the second contiguous nucleotide sequence consists of the sequence of SEQ ID NO: 326.

In preferred embodiments, the second contiguous nucleotide sequence consists of the sequence of SEQ ID NO: 339.

In some embodiments, the second contiguous nucleotide sequence comprises a sequence having at least 80% identity to any one of the sequences of SEQ ID NOs 194-384. In some embodiments, the second contiguous nucleotide sequence comprises a sequence having at least 85%, 90%, 91%, 92%, 93%, 94%, 95%, 96%, 97%, 98% or 99% identity to any one of the sequences of SEQ ID NOs 194-384. In some embodiments, the second contiguous nucleotide sequence comprises a sequence having at least 80% identity to any one of the sequences of SEQ ID NOs 194, 195, 214, 256, 265, 288, 307, 321, 326 and 339. In some embodiments, the second contiguous nucleotide sequence comprises a sequence having at least 85%, 90%, 91%, 92%, 93%, 94%, 95%, 96%, 97%, 98% or 99% identity to any one of the sequences of SEQ ID NOs 194, 195, 214, 256, 265, 288, 307, 321, 326 and 339.

In some embodiments, the second contiguous nucleotide sequence consists of a sequence having at least 80% identity to any one of the sequences of SEQ ID NOs 194-384. In some embodiments, the second contiguous nucleotide sequence consists of a sequence having at least 85%, 90%, 91%, 92%, 93%, 94%, 95%, 96%, 97%, 98% or 99% identity to any one of the sequences of SEQ ID NOs 194-384. In some embodiments, the second contiguous nucleotide sequence consists of a sequence having at least 80% identity to any one of the sequences of SEQ ID NOs 194, 195, 214, 256, 265, 288, 307, 321, 326 and 339. In some embodiments, the second contiguous nucleotide sequence consists of a sequence having at least 85%, 90%, 91%, 92%, 93%, 94%, 95%, 96%, 97%, 98% or 99% identity to any one of the sequences of SEQ ID NOs 194, 195, 214, 256, 265, 288, 307, 321, 326 and 339.

### Antisense Strand

An “antisense strand” or “guide strand” refers to the strand of a nucleic acid molecule that includes a region substantially complementary to a target sequence, e.g. a JAK1 mRNA. The antisense strand is substantially complementary to the sense strand.

In some embodiments the antisense strand consists of the second contiguous nucleotide sequence. Therein, the antisense strand may possess the features described hereinabove for the second contiguous nucleotide sequence.

Thus, in some embodiments, the antisense strand is complementary to a target sequence within the JAK1 nucleic acid sequence, wherein the target sequence is any one of the sequences of SEQ ID NOs 385-575 (as shown in Table 1 herein). In some embodiments, the antisense strand is complementary to a target sequence within the JAK1 nucleic acid sequence, wherein the target sequence is any one of the sequences of SEQ ID NOs 385, 386, 405, 447, 456, 479, 498, 512, 517 and 530. These are the target sequences for the preferred compounds depicted in Table 4 herein.

In some embodiments, the antisense strand is at least 80% complementary to the target sequence. In other words, the antisense strand is at least 80% complementary to any one of the sequences of SEQ ID NOs 385-575, preferably any one of the sequences of SEQ ID NOs 385, 386, 405, 447, 456, 479, 498, 512, 517 and 530.

In some embodiments, the antisense strand is at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% complementary to the target sequence. In some embodiments, the antisense strand is fully (i.e. 100%) complementary to the target sequence.

In one embodiment, the antisense strand is complementary, such as at least 80% complementary, such as fully complementary, to SEQ ID NO: 385 (target sequence of compound 614).

In another embodiment, the antisense strand is complementary, such as at least 80% complementary, such as fully complementary, to SEQ ID NO: 386 (target sequence of compound 673).

In another embodiment the antisense strand is complementary, such as at least 80% complementary, such as fully complementary, to SEQ ID NO: 405 (target sequence of compound 1182).

In another embodiment the antisense strand is complementary, such as at least 80% complementary, such as fully complementary, to SEQ ID NO: 447 (target sequence of compound 1770).

In another embodiment the antisense strand is complementary, such as at least 80% complementary, such as fully complementary, to SEQ ID NO: 456 (target sequence of compound 1954).

In another embodiment the antisense strand is complementary, such as at least 80% complementary, such as fully complementary, to SEQ ID NO: 479 (target sequence of compound 2319).

In another embodiment the antisense strand is complementary, such as at least 80% complementary, such as fully complementary, to SEQ ID NO: 498 (target sequence of compound 3131).

In another embodiment the antisense strand is complementary, such as at least 80% complementary, such as fully complementary, to SEQ ID NO: 512 (target sequence of compound 3255).

In another embodiment the antisense strand is complementary, such as at least 80% complementary, such as fully complementary, to SEQ ID NO: 517 (target sequence of compound 3265).

In another embodiment the antisense strand is complementary, such as at least 80% complementary, such as fully complementary, to SEQ ID NO: 530 (target sequence of compound 3313).

In some embodiments, the antisense strand comprises any one of the sequences of SEQ ID NOs 194-384 (as shown in Table 1 herein). In some embodiments, the antisense strand comprises any one of the sequences of SEQ ID NOs 194, 195, 214, 256, 265, 288, 307, 321, 326 and 339. These are the sequences of the antisense strand of the preferred compounds depicted in Table 4 herein.

In some embodiments, the antisense strand consists of any one of the sequences of SEQ ID NOs 194-384. In some embodiments, the antisense strand consist of any one of the sequences of SEQ ID NOs 194, 195, 214, 256, 265, 288, 307, 321, 326 and 339.

In preferred embodiments, the antisense strand comprises the sequence of SEQ ID NO: 194.

In preferred embodiments, the antisense strand comprises the sequence of SEQ ID NO: 195.

In preferred embodiments, the antisense strand comprises the sequence of SEQ ID NO: 214.

In preferred embodiments, the antisense strand comprises the sequence of SEQ ID NO: 256.

In preferred embodiments, the antisense strand comprises the sequence of SEQ ID NO: 265.

In preferred embodiments, the antisense strand comprises the sequence of SEQ ID NO: 288.

In preferred embodiments, the antisense strand comprises the sequence of SEQ ID NO: 307.

In preferred embodiments, the antisense strand comprises the sequence of SEQ ID NO: 321.

In preferred embodiments, the antisense strand comprises the sequence of SEQ ID NO: 326.

In preferred embodiments, the antisense strand comprises the sequence of SEQ ID NO: 339.

In preferred embodiments, the antisense strand consists of the sequence of SEQ ID NO: 194.

In preferred embodiments, the antisense strand consists of the sequence of SEQ ID NO: 195.

In preferred embodiments, the antisense strand consists of the sequence of SEQ ID NO: 214.

In preferred embodiments, the antisense strand consists of the sequence of SEQ ID NO: 256.

In preferred embodiments, the antisense strand consists of the sequence of SEQ ID NO: 265.

In preferred embodiments, the antisense strand consists of the sequence of SEQ ID NO: 288.

In preferred embodiments, the antisense strand consists of the sequence of SEQ ID NO: 307.

In preferred embodiments, the antisense strand consists of the sequence of SEQ ID NO: 321.

In preferred embodiments, the antisense strand consists of the sequence of SEQ ID NO: 326.

In preferred embodiments, the antisense strand consists of the sequence of SEQ ID NO: 339.

In some embodiments, the antisense strand comprises a sequence having at least 80% identity to any one of the sequences of SEQ ID NOs 194-384. In some embodiments, the antisense strand comprises a sequence having at least 85%, 90%, 91%, 92%, 93%, 94%, 95%, 96%, 97%, 98% or 99% identity to any one of the sequences of SEQ ID NOs 194-384. In some embodiments, the antisense strand comprises a sequence having at least 80% identity to any one of the sequences of SEQ ID NOs 194, 195, 214, 256, 265, 288, 307, 321, 326 and 339. In some embodiments, the antisense strand comprises a sequence having at least 85%, 90%, 91%, 92%, 93%, 94%, 95%, 96%, 97%, 98% or 99% identity to any one of the sequences of SEQ ID NOs 194, 195, 214, 256, 265, 288, 307, 321, 326 and 339.

In some embodiments, the antisense strand consists of a sequence having at least 80% identity to any one of the sequences of SEQ ID NOs 194-384. In some embodiments, the antisense strand consists of a sequence having at least 85%, 90%, 91%, 92%, 93%, 94%, 95%, 96%, 97%, 98% or 99% identity to any one of the sequences of SEQ ID NOs 194-384. In some embodiments, the antisense strand consists of a sequence having at least 80% identity to any one of the sequences of SEQ ID NOs 194, 195, 214, 256, 265, 288, 307, 321, 326 and 339. In some embodiments, the antisense strand consists of a sequence having at least 85%, 90%, 91%, 92%, 93%, 94%, 95%, 96%, 97%, 98% or 99% identity to any one of the sequences of SEQ ID NOs 194, 195, 214, 256, 265, 288, 307, 321, 326 and 339.

### Vinyl Phosphonate

In some embodiments, the antisense strand comprises a vinyl phosphonate at the 5′ end of the antisense strand (i.e. the 5′-most nucleotide of the antisense strand has a phosphonate group instead of a phosphodiester group at the 5′ position of the sugar moiety). In some embodiments, the most 5′ end nucleotide is a 5′-vinyl phosphonate nucleotide.

The structure of vinyl phosphonic acid (i.e. vinyl phosphonate group in isolation) is as follows:

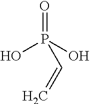

Vinyl phosphonic acid has the same structure as phosphoric acid (i.e. phosphate group), except that one of the oxygen atoms bonded to the central phosphorous atom is replaced with a vinyl group.

Vinyl phosphonic acid forms a covalent bond with the 5′ position of the sugar moiety of a nucleotide via the vinyl group. Thus, the general structure of a nucleotide comprising a 5′ vinyl phosphonate group is as follows:

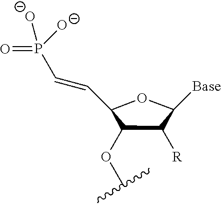

In some embodiments, the most 5′ end nucleotide is a 5′-vinyl phosphonate 2′OMe nucleotide (i.e. the 5′-most nucleotide is a 2′-O-Methyl nucleotide).

In some embodiments, the most 5′ end nucleotide is a 5′-vinyl phosphonate 2′F nucleotide (i.e. the 5′-most nucleotide is a 2′-fluoro nucleotide).

In some embodiments, the most 5′ end nucleotide is a 5′-vinyl phosphonate 2′MOE nucleotide (i.e. the 5′-most nucleotide is a 2′MOE nucleotide).

In some embodiments, the antisense strand comprises a uridine nucleotide located at the 5′ end of the antisense strand (i.e. a 5′ uridine nucleotide). In some embodiments, the 5′ uridine nucleotide is comprises an O-Methyl group at the 2′position of its sugar moiety (i.e. the 5′ uridine nucleotide is a 2′-O-Methyl uridine nucleotide).

In some embodiments, the antisense strand comprises a Vinyl phosphonate 2′-O-Methyl uridine nucleotide located at the 5′ end of the antisense strand (i.e. 5′-Vinyl phosphonate 2′-O-Methyl uridine). The structure of 5′-Vinyl phosphonate 2′-O-Methyl uridine is as follows (wherein U is the nucleobase uracil):

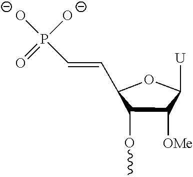

### Antisense Strand Length

In some embodiments, the antisense strand is 19 to 27 nucleotides long. In some embodiments, the antisense strand is 19, 20, 21, 22, 23, 24, 25, 26 or 27 nucleotides long.

In some embodiments, the antisense strand is 19 nucleotides long. In some embodiments, the antisense strand is 20 nucleotides long. In some embodiments, the antisense strand is 21 nucleotides long. In some embodiments, the antisense strand is 22 nucleotides long. In some embodiments, the antisense strand is 23 nucleotides long. In some embodiments, the antisense strand is 24 nucleotides long. In some embodiments, the antisense strand is 25 nucleotides long. In some embodiments, the antisense strand is 26 nucleotides long. In some embodiments, the antisense strand is 27 nucleotides long.

### First Contiguous Nucleotide Sequence

The antisense strand comprises a first contiguous nucleotide sequence of at least 15 nucleotides in length.

As described elsewhere herein, the first contiguous nucleotide sequence forms a double-stranded region of complementarity with the first contiguous nucleotide sequence.

In some embodiment, the first contiguous nucleotide sequence is 15-24 nucleotides long.

In some embodiments, the first contiguous nucleotide sequence is 15, 16, 17, 18, 19, 20 or 21 nucleotides long.

In some embodiments, the first contiguous nucleotide sequence is 15 nucleotides long. In some embodiments, the first contiguous nucleotide sequence is 16 nucleotides long. In some embodiments, the first contiguous nucleotide sequence is 17 nucleotides long. In some embodiments, the first contiguous nucleotide sequence is 18 nucleotides long. In some embodiments, the first contiguous nucleotide sequence is 19 nucleotides long. In some embodiments, the first contiguous nucleotide sequence is 20 nucleotides long. In some embodiments, the first contiguous nucleotide sequence is 21 nucleotides long.

In some embodiments, the second contiguous nucleotide sequence is 21 nucleotides long and the first contiguous nucleotide sequence is 19 nucleotides long such that the double stranded region of complementarity is 19 nucleotides long.

In some embodiments, the first contiguous nucleotide sequence comprises any one of the sequences of SEQ ID NOs 3-193 (as shown in Table 1 herein). In some embodiments, the second contiguous nucleotide sequence comprises any one of the sequences of SEQ ID NOs 3, 4, 23, 65, 74, 97, 116, 130, 135 and 148. These are the sequences of the first contiguous nucleotide sequence of the preferred compounds depicted in Table 4 herein.

In some embodiments, the first contiguous nucleotide sequence consists of any one of the sequences of SEQ ID NOs 3-193. In some embodiments, the first contiguous nucleotide sequence consist of any one of the sequences of SEQ ID NOs 3, 4, 23, 65, 74, 97, 116, 130, 135 and 148.

In preferred embodiments, the first contiguous nucleotide sequence comprises the sequence of SEQ ID NO: 3.

In preferred embodiments, the first contiguous nucleotide sequence comprises the sequence of SEQ ID NO: 4.

In preferred embodiments, the first contiguous nucleotide sequence comprises the sequence of SEQ ID NO: 23.

In preferred embodiments, the first contiguous nucleotide sequence comprises the sequence of SEQ ID NO: 65.

In preferred embodiments, the first contiguous nucleotide sequence comprises the sequence of SEQ ID NO: 74.

In preferred embodiments, the first contiguous nucleotide sequence comprises the sequence of SEQ ID NO: 97.

In preferred embodiments, the first contiguous nucleotide sequence comprises the sequence of SEQ ID NO: 116.

In preferred embodiments, the first contiguous nucleotide sequence comprises the sequence of SEQ ID NO: 130.

In preferred embodiments, the first contiguous nucleotide sequence comprises the sequence of SEQ ID NO: 135.

In preferred embodiments, the first contiguous nucleotide sequence comprises the sequence of SEQ ID NO: 148.

In preferred embodiments, the first contiguous nucleotide sequence consists of the sequence of SEQ ID NO: 3.

In preferred embodiments, the first contiguous nucleotide sequence consists of the sequence of SEQ ID NO: 4.

In preferred embodiments, the first contiguous nucleotide sequence consists of the sequence of SEQ ID NO: 23.

In preferred embodiments, the first contiguous nucleotide sequence consists of the sequence of SEQ ID NO: 65.

In preferred embodiments, the first contiguous nucleotide sequence consists of the sequence of SEQ ID NO: 74.

In preferred embodiments, the first contiguous nucleotide sequence consists of the sequence of SEQ ID NO: 97.

In preferred embodiments, the first contiguous nucleotide sequence consists of the sequence of SEQ ID NO: 116.

In preferred embodiments, the first contiguous nucleotide sequence consists of the sequence of SEQ ID NO: 130.

In preferred embodiments, the first contiguous nucleotide sequence consists of the sequence of SEQ ID NO: 135.

In preferred embodiments, the first contiguous nucleotide sequence consists of the sequence of SEQ ID NO: 148.

In some embodiments, the first contiguous nucleotide sequence comprises a sequence having at least 80% identity to any one of the sequences of SEQ ID NOs 3-193. In some embodiments, the first contiguous nucleotide sequence comprises a sequence having at least 85%, 90%, 91%, 92%, 93%, 94%, 95%, 96%, 97%, 98% or 99% identity to any one of the sequences of SEQ ID NOs 3-193. In some embodiments, the first contiguous nucleotide sequence comprises a sequence having at least 80% identity to any one of the sequences of SEQ ID NOs 3, 4, 23, 65, 74, 97, 116, 130, 135 and 148. In some embodiments, the first contiguous nucleotide sequence comprises a sequence having at least 85%, 90%, 91%, 92%, 93%, 94%, 95%, 96%, 97%, 98% or 99% identity to any one of the sequences of SEQ ID NOs 3, 4, 23, 65, 74, 97, 116, 130, 135 and 148.

In some embodiments, the first contiguous nucleotide sequence consists of a sequence having at least 80% identity to any one of the sequences of SEQ ID NOs 3-193. In some embodiments, the first contiguous nucleotide sequence consists of a sequence having at least 85%, 90%, 91%, 92%, 93%, 94%, 95%, 96%, 97%, 98% or 99% identity to any one of the sequences of SEQ ID NOs 3-193. In some embodiments, the first contiguous nucleotide sequence consists of a sequence having at least 80% identity to any one of the sequences of SEQ ID NOs 3, 4, 23, 65, 74, 97, 116, 130, 135 and 148. In some embodiments, the first contiguous nucleotide sequence consists of a sequence having at least 85%, 90%, 91%, 92%, 93%, 94%, 95%, 96%, 97%, 98% or 99% identity to any one of the sequences of SEQ ID NOs 3, 4, 23, 65, 74, 97, 116, 130, 135 and 148.

### Sense Strand

A “sense strand” or “passenger strand” refers to the strand of a nucleic acid that includes a region encoding a target sequence or portion thereof, e g. a nucleic acid encoding a portion of JAK1. The sense strand is substantially complementary to the antisense strand.

In some embodiments the sense strand consists of the first contiguous nucleotide sequence. Therein, the sense strand may possess the features described above for the first contiguous nucleotide sequence.

In some embodiments, the sense strand comprises any one of the sequences of SEQ ID NOs 3-193 (as shown in Table 1 herein). In some embodiments, the sense strand comprises any one of the sequences of SEQ ID NOs 3, 4, 23, 65, 74, 97, 116, 130, 135 and 148. These are the sequences of the sense strand of the preferred compounds depicted in Table 4 herein.

In some embodiments, the antisense strand consists of any one of the sequences of SEQ ID NOs 3-193. In some embodiments, the antisense strand consist of any one of the sequences of SEQ ID NOs 3, 4, 23, 65, 74, 97, 116, 130, 135 and 148.

In preferred embodiments, the antisense strand comprises the sequence of SEQ ID NO: 3.

In preferred embodiments, the antisense strand comprises the sequence of SEQ ID NO: 4.

In preferred embodiments, the antisense strand comprises the sequence of SEQ ID NO: 23.

In preferred embodiments, the antisense strand comprises the sequence of SEQ ID NO: 65.

In preferred embodiments, the antisense strand comprises the sequence of SEQ ID NO: 74.

In preferred embodiments, the antisense strand comprises the sequence of SEQ ID NO: 97.

In preferred embodiments, the antisense strand comprises the sequence of SEQ ID NO: 116.

In preferred embodiments, the antisense strand comprises the sequence of SEQ ID NO: 130.

In preferred embodiments, the antisense strand comprises the sequence of SEQ ID NO: 135.

In preferred embodiments, the antisense strand comprises the sequence of SEQ ID NO: 148.

In preferred embodiments, the antisense strand consists of the sequence of SEQ ID NO: 3.

In preferred embodiments, the antisense strand consists of the sequence of SEQ ID NO: 4.

In preferred embodiments, the antisense strand consists of the sequence of SEQ ID NO: 23.

In preferred embodiments, the antisense strand consists of the sequence of SEQ ID NO: 65.

In preferred embodiments, the antisense strand consists of the sequence of SEQ ID NO: 74.

In preferred embodiments, the antisense strand consists of the sequence of SEQ ID NO: 97.

In preferred embodiments, the antisense strand consists of the sequence of SEQ ID NO: 116.

In preferred embodiments, the antisense strand consists of the sequence of SEQ ID NO: 130.

In preferred embodiments, the antisense strand consists of the sequence of SEQ ID NO: 135.

In preferred embodiments, the antisense strand consists of the sequence of SEQ ID NO: 148.

In some embodiments, the sense strand comprises a sequence having at least 80% identity to any one of the sequences of SEQ ID NOs 3-193. In some embodiments, the sense strand comprises a sequence having at least 85%, 90%, 91%, 92%, 93%, 94%, 95%, 96%, 97%, 98% or 99% identity to any one of the sequences of SEQ ID NOs 3-193. In some embodiments, the sense strand comprises a sequence having at least 80% identity to any one of the sequences of SEQ ID NOs 3, 4, 23, 65, 74, 97, 116, 130, 135 and 148. In some embodiments, the sense strand comprises a sequence having at least 85%, 90%, 91%, 92%, 93%, 94%, 95%, 96%, 97%, 98% or 99% identity to any one of the sequences of SEQ ID NOs 3, 4, 23, 65, 74, 97, 116, 130, 135 and 148.

In some embodiments, the sense strand consists of a sequence having at least 80% identity to any one of the sequences of SEQ ID NOs 3-193. In some embodiments, the sense strand consists of a sequence having at least 85%, 90%, 91%, 92%, 93%, 94%, 95%, 96%, 97%, 98% or 99% identity to any one of the sequences of SEQ ID NOs 3-193. In some embodiments, the sense strand consists of a sequence having at least 80% identity to any one of the sequences of SEQ ID NOs 3, 4, 23, 65, 74, 97, 116, 130, 135 and 148. In some embodiments, the sense strand consists of a sequence having at least 85%, 90%, 91%, 92%, 93%, 94%, 95%, 96%, 97%, 98% or 99% identity to any one of the sequences of SEQ ID NOs 3, 4, 23, 65, 74, 97, 116, 130, 135 and 148.

### Sense Strand Length

In some embodiments, the sense strand is 19 to 27 nucleotides long. In some embodiments, the sense strand is 19, 20, 21, 22, 23, 24, 25, 26 or 27 nucleotides long.

In some embodiments, the sense strand is 19 nucleotides long. In some embodiments, the sense strand is 20 nucleotides long. In some embodiments, the sense strand is 21 nucleotides long. In some embodiments, the sense strand is 22 nucleotides long. In some embodiments, the sense strand is 23 nucleotides long. In some embodiments, the sense strand is 24 nucleotides long. In some embodiments, the sense strand is 25 nucleotides long. In some embodiments, the sense strand is 26 nucleotides long. In some embodiments, the sense strand is 27 nucleotides long.

In some embodiments the antisense strand is 21 nucleotides in length and sense strand is 19 nucleotides in length.

Additional 5′ and/or 3′ Nucleosides

The sense strand and/or antisense may comprise additional nucleotides at their 5′-end and/or 3′-end.

The additional 5′ and/or 3′ nucleotides may for joining the dsRNA of the invention to a conjugate moiety or another functional group (i.e. the additional 5′ and/or 3′ nucleosides may for a biocleavable linker as described elsewhere herein).

Alternatively, the further 5′ and/or 3′ nucleosides may be used to provide exonucleoase protection or for ease of synthesis or manufacture of the dsRNA.

When part of the antisense strand, the further 5′ and/or 3′ nucleosides may or may not be fully complementary to the target nucleic acid. Regardless of whether the 5′ and/or 3′ nucleosides are complementary to the target sequence or not, the additional 5′ and/or 3′ nucleosides are not considered to be part of the sequence of the antisense strand or sense strand.

### Duplexes

The invention provides duplexes comprising a sense strand and an antisense strand as described herein.

In some embodiments the sequence of the sense strand is any one of the sequences of SEQ ID NOs 3-193 and the sequence of the antisense strand is any one of the sequences of SEQ ID NOs 194-384 as shown in Table 1 herein.

In preferred embodiments, the sequence of the sense strand is the sequence of SEQ ID NO 3 and the sequence of the antisense strand is the sequence of SEQ ID NO 194 (i.e. the sequences of compound 614).

In preferred embodiments, the sequence of the sense strand is the sequence of SEQ ID NO 4 and the sequence of the antisense strand is the sequence of SEQ ID NO 195 (i.e. the sequences of compound 673).

In preferred embodiments, the sequence of the sense strand is the sequence of SEQ ID NO 23 and the sequence of the antisense strand is the sequence of SEQ ID NO 214 (i.e. the sequences of compound 1182).

In preferred embodiments, the sequence of the sense strand is the sequence of SEQ ID NO 65 and the sequence of the antisense strand is the sequence of SEQ ID NO 256 (i.e. the sequences of compound 1770).

In preferred embodiments, the sequence of the sense strand is the sequence of SEQ ID NO 74 and the sequence of the antisense strand is the sequence of SEQ ID NO 265 (i.e. the sequences of compound 1954).

In preferred embodiments, the sequence of the sense strand is the sequence of SEQ ID NO 97 and the sequence of the antisense strand is the sequence of SEQ ID NO 288 (i.e. the sequences of compound 2319).

In preferred embodiments, the sequence of the sense strand is the sequence of SEQ ID NO 116 and the sequence of the antisense strand is the sequence of SEQ ID NO 307 (i.e. the sequences of compound 3131).

In preferred embodiments, the sequence of the sense strand is the sequence of SEQ ID NO 130 and the sequence of the antisense strand is the sequence of SEQ ID NO 321 (i.e. the sequences of compound 3255).

In preferred embodiments, the sequence of the sense strand is the sequence of SEQ ID NO 135 and the sequence of the antisense strand is the sequence of SEQ ID NO 326 (i.e. the sequences of compound 3265).

In preferred embodiments, the sequence of the sense strand is the sequence of SEQ ID NO 148 and the sequence of the antisense strand is the sequence of SEQ ID NO 339 (i.e. the sequences of compound 3313).

In some embodiments the antisense strand and the sense strand form a duplex selected from the group consisting of duplex numbers 1-191 of Table 1.

In preferred embodiments the antisense strand and the sense strand form duplex 1 of Table 1.

In preferred embodiments the antisense strand and the sense strand form duplex 2 of Table 1.

In preferred embodiments the antisense strand and the sense strand form duplex 21 of Table 1.

In preferred embodiments the antisense strand and the sense strand form duplex 63 of Table 1.

In preferred embodiments the antisense strand and the sense strand form duplex 72 of Table 1.

In preferred embodiments the antisense strand and the sense strand form duplex 95 of Table 1.

In preferred embodiments the antisense strand and the sense strand form duplex 114 of Table 1.

In preferred embodiments the antisense strand and the sense strand form duplex 128 of Table 1.

In preferred embodiments the antisense strand and the sense strand form duplex 133 of Table 1.

In preferred embodiments the antisense strand and the sense strand form duplex 146 of Table 1.

### Modified Nucleotide

In some embodiments, the compound of the invention comprises at least one modified nucleotide.

The term “modified nucleotide” or “nucleotide modification” as used herein refers to nucleosides modified as compared to the equivalent DNA or RNA nucleoside by the introduction of one or more modifications of the sugar moiety or the (nucleo) base moiety.

The terms “modified nucleotide”, “modified nucleoside”, “nucleoside analogue”, “modified units” and “modified monomers” are used interchangeably herein.

A “DNA nucleotide” is a nucleotide comprising an unmodified DNA sugar moiety. An “RNA nucleotide” is a nucleotide comprising a RNA sugar moiety. Nucleotides with modifications in the base region of the DNA or RNA nucleoside are still termed DNA or RNA if they allow Watson Crick base pairing.

The pattern in which the modified nucleotides (such as high affinity modified nucleosides) are incorporated into the oligonucleotide sequence is generally termed “oligonucleotide design”.

A high affinity modified nucleotide is a modified nucleotide which, when incorporated into a nucleic acid, enhances the affinity of the nucleic acid for its complementary target, for example as measured by the melting temperature (Tₘ). A high affinity modified nucleotide of the present invention preferably results in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleotides as well as locked nucleic acids (LNA) (see e.g. Freier & Altmann, Nucl. Acid Res., 1997, 25, 4429-4443 and Uhlmann; Curr. Opinion in Drug Development, 2000, 3 (2), 203-213).

Exemplary modified nucleotides include LNA, 2′-O-MOE, 2′OMe and morpholino nucleotide analogues. These are discussed further below.

The antisense strand may comprise at least one modified nucleotide. The antisense strand may comprise 1, 2, 3, 4, 5, 6, 7, 8, 9, 10, 11, 12, 13, 14, 15, 16, 17, 18, 19, 20, 21, 22, 23, 24, 25, 26, 27 or more modified nucleotides.

The sense strand may comprise at least one modified nucleotide. The sense strand may comprise 1, 2, 3, 4, 5, 6, 7, 8, 9, 10, 11, 12, 13, 14, 15, 16, 17, 18, 19, 20, 21, 22, 23, 24, 25, 26, 27 or more modified nucleotides.

In some embodiments, both the sense strand and the antisense strand comprise at least one modified nucleotide.

### Modified Sugar Moiety

In some embodiments, the at least one modified nucleotide comprises a modified sugar moiety. In other words, in some embodiments, the compounds of the invention comprise at least one nucleotide comprising a modified sugar moiety.

Numerous nucleotides with modification of the ribose sugar moiety are known in the art, primarily with the function of improving certain properties of nucleic acid, such as affinity and/or nuclease resistance.

A modified sugar moiety is a sugar moiety that is modified when compared to the ribose sugar moiety found in DNA and RNA.

Each modified sugar moiety may be independently selected from a bicyclic sugar moiety or a non-bicyclic sugar moiety. In some embodiments, the modified sugar moiety is a bicyclic sugar moiety. In some embodiments, the modified sugar moiety is a non-bicyclic sugar moiety.

Modified nucleotides also include nucleotides where the sugar moiety is replaced with a non-sugar moiety, for example in the case of peptide nucleic acids (PNA), or morpholino nucleic acids.

Sugar modifications also include modifications made via altering the substituent groups on the ribose ring to groups other than hydrogen, or the 2′-OH group naturally found in DNA and RNA nucleosides. Substituents may, for example be introduced at the 2′, 3′, 4′ or 5′ positions.

Sugar modifications include those where the ribose ring structure is modified, e.g. by replacement with a hexose ring (HNA), or a bicyclic ring.

Other sugar modified nucleosides include, for example, bicyclohexose nucleic acids (see WO 2011/017521) or tricyclic nucleic acids (see WO 2013/154798).

In some embodiments, each non-bicyclic sugar moiety is independently selected from 2′-O-alkyl-RNA, 2′-O-methyl-RNA (2′OMe modified sugar), 2′-alkoxy-RNA, 2′-O-methoxyethyl-RNA, 2′-amino-DNA, 2′-fluoro-DNA (2′F modified sugar), arabino nucleic acid (ANA), 2′-fluoro-ANA, Glycol nucleic acid (GNA), and unlocked nucleic acid (UNA). UNA lacks a bond between the C2 and C3 carbons.

### 2′ Sugar Modified Nucleotides

2′ sugar modified nucleotides are particularly preferred in the compounds of the invention.

A 2′ sugar modified nucleotide is a nucleotide which has a substituent other than —H or —OH at the 2′ position (2′ substituted nucleotide) or comprises a 2′ linked biradical capable of forming a bridge between the 2′ carbon and a second carbon in the ribose ring, such as LNA (2′-4′ biradical bridged) nucleosides. In other words, a 2′ sugar modified nucleotide is a nucleotide comprising a modified sugar moiety comprising a group other than —H or —OH at the 2′ position of the ribose ring.

Numerous 2′ substituted nucleosides have been found to have beneficial properties when incorporated into nucleic acids. For example, the 2′ modified sugar may provide enhanced binding affinity and/or increased nuclease resistance to the nucleic acid. Examples of 2′ substituted modified nucleosides are 2′-O-alkyl-RNA, 2′-O-methyl-RNA (2′OMe). 2′-alkoxy-RNA, 2′-O-methoxyethyl-RNA (2′MOE), 2′-amino-DNA, 2′-Fluoro-RNA (2′F), and 2′-F-ANA nucleoside. For further examples, see for example Freier & Altmann; Nucl. Acid Res., 1997, 25, 4429-4443 and Uhlmann; Curr. Opinion in Drug Development 2000, 3 (2), 203-213, and Deleavey and Damha, Chemistry and Biology 2012, 19, 937.

Below are illustrations of some 2′ substituted modified nucleosides.

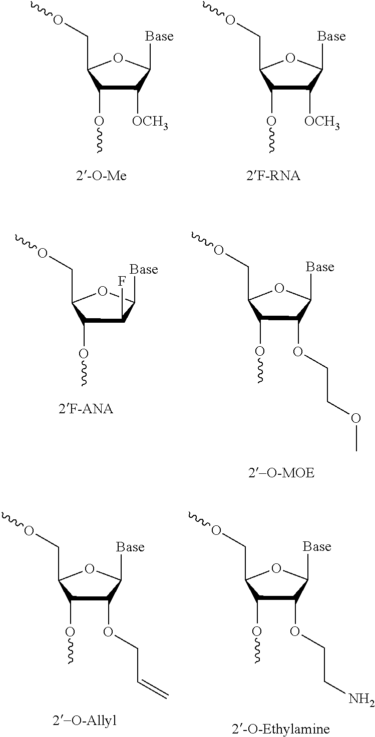

“2′-O-methoxyethyl” (also 2′-MOE and 2′-OCH₂CH₂—OCH₃ and MOE) refers to an O-methoxyethyl modification of the 2′ position of a furanose ring. A 2′-O-methoxyethyl modified sugar is a modified sugar.

“2′-MOE nucleoside” (also 2′-O-methoxyethyl nucleoside) means a nucleoside comprising a 2′MOE modified sugar moiety.

In some embodiments, each non-bicyclic sugar moiety is independently selected from a 2′F modified sugar, a 2′OMe modified sugar and a 2′MOE modified sugar moiety. In preferred embodiments, each non-bicyclic sugar moiety is independently selected from a 2′OMe modified sugar and a 2′F modified sugar.

In some embodiments, the compound comprises one or more 2′OMe modified sugar moiety. In some embodiments, the compound comprises one or more 2′F modified sugar. In some embodiments, the compound comprises one or more 2′OMe modified sugar moiety and one or more 2′F modified sugar.

In some embodiments at least about 50%, such as 55%, 60%, 70%, 75%, 80%, 95%, 90%, 95%, 96%, 97%, 98%, 99% or all of the sugar moieties within the compound of the invention are 2′OMe modified sugar moieties.

In some embodiments 50-85% of the sugar moieties are 2′OMe modified sugar moieties. In some embodiments 68-85%, such as 68%, 69%, 70%, 71%, 72%, 73%, 74%, 75%, 76%, 77%, 78%, 79%, 80%, 81%, 82%, 83%, 84% or 85%, of the sugar moieties are 2′OMe modified sugar moieties.

In some embodiments, each sugar moiety in the sense strand is independently selected from a 2′OMe modified sugar and a 2′F modified sugar (i.e. every sugar moiety in the sense strand is either a 2′OMe modified sugar or a 2′F modified sugar). In some embodiments, each sugar moiety in the antisense strand is independently selected from a 2′OMe modified sugar and a 2′F modified sugar (i.e. every sugar moiety in the antisense strand is either a 2′OMe modified sugar or a 2′F modified sugar). In some embodiments, each sugar moiety in both the sense strand and antisense strand is independently selected from a 2′OMe modified sugar and a 2′F modified sugar (i.e. every sugar moiety in the dsRNA is either a 2′OMe modified sugar or a 2′F modified sugar).

Locked Nucleic Acid Nucleosides (LNA nucleoside)

In some embodiments the bicyclic sugar moiety may be a sugar moiety comprising a bridge connecting two carbon atoms of the sugar ring, thereby forming a bicyclic ring system.

In some embodiments the bridge may connect the 4′-carbon and the 2′-carbon of the ribosyl ring. In some embodiments the modified sugar moiety may be independently selected from a locked nucleic acid (LNA) and a constrained ethyl nucleic acid (cEt).

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring.

These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into a nucleic acid or a complementary RNA or DNA molecule. This can be routinely determined by measuring the melting temperature of the nucleic acid.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett., 12, 73-76, Seth et al., J. Org. Chem., 2010, Vol 75 (5) pp. 1569-81, Mitsuoka et al., Nucleic Acids Research, 2009, 37 (4), 1225-1238, and Wan and Seth, J. Medical Chemistry, 2016, 59, 9645-9667.

Further non limiting, exemplary LNA nucleosides are disclosed below:

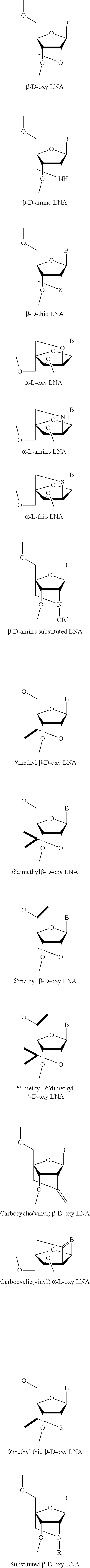

Particular LNA nucleosides are beta-D-oxy-LNA, 6′-methyl-beta-D-oxy LNA such as(S)-6′-methyl-beta-D-oxy-LNA (ScET) and ENA.

A particularly advantageous LNA is beta-D-oxy-LNA.

### Totalmers

In some embodiments, the antisense strand is a totalmer. In some embodiments, the sense strand is a totalmer. In some embodiments, both the antisense strand and the sense strand are totalmers.

A “totalmer” is nucleic acid which does not comprise DNA or RNA nucleosides. In some embodiments, every nucleotide of the antisense strand is independently selected from a 2′F nucleotide and a 2′OMe nucleotide. In some embodiments, every nucleotide of the sense strand is independently selected from a 2′F nucleotide and a 2′OMe nucleotide. In some embodiments, every nucleotide of both the antisense strand and the sense strand is independently selected from a 2′F nucleotide and a 2′OMe nucleotide.

### Modified Internucleotide Linkage

In some embodiments, the compound of the invention (i.e. the dsRNA) comprises least one modified internucleotide linkage.

The term “modified internucleotide linkage” is defined as generally understood by the skilled person as linkages, other than phosphodiester (PO) linkages, which covalently couple two nucleosides together. Nucleotides with a modified internucleotide linkage may also be referred to as “modified nucleotides” herein.

For naturally occurring oligonucleotides, the internucleotide linkage includes phosphate groups creating a phosphodiester bond between adjacent nucleosides. The modified internucleotide linkage may increase the nuclease resistance of the nucleic acid molecules of the invention compared to a phosphodiester linkage. Modified internucleotide linkages are particularly useful in stabilizing nucleic acids for in vivo use, and may serve to protect against nuclease cleavage at regions of DNA or RNA nucleosides in the nucleic acid of the invention.

A phosphorothioate internucleotide linkage is particularly useful due to nuclease resistance, beneficial pharmacokinetics and ease of manufacture.

The dsRNA may however comprise internucleotide linkages other than phosphorothioate, such as phosphodiester linkages, in particular in regions where modified nucleosides, such as LNA, protect the linkage against nuclease degradation. Inclusion of phosphodiester linkages, such as one or two linkages, particularly between or adjacent to modified nucleoside units (typically in the non-nuclease recruiting regions) can modify the bioavailability and/or bio-distribution of an oligonucleotide (see for example WO2008/113832).

The terms “modified internucleotide linkage” and “modified internucleoside linkage” are used interchangeably herein, and will both be understood to mean the chemical structure linking the sugar moieties of adjacent nucleosides.

In some embodiments, the antisense strand comprises at least one modified internucleotide linkage. In some embodiments, the sense strand comprises at least one modified internucleotide linkage. In some embodiments, both the sense strand and the antisense strand comprise at least one modified internucleotide linkage.

In some embodiments, the modified internucleotide linkages are independently selected from a phosphorothioate internucleotide linkage (PS), a diphosphorothioate internucleotide linkage and a boranophosphate internucleotide linkage.

In some embodiments each internucleotide linkage within the antisense strand is either a phosphodiester internucleotide linkage (PO) or a phosphorothioate internucleotide linkage (PS). In some embodiments each internucleotide linkage within the sense strand is either a phosphodiester internucleotide linkage or a phosphorothioate internucleotide linkage. In some embodiments each internucleotide linkage within the antisense strand and the sense strand is either a phosphodiester internucleotide linkage or a phosphorothioate internucleotide linkage.

### Modified Nucleobase

In some embodiments, the dsRNA comprises at least one modified nucleobase.

The term nucleobase includes the purine (e.g. adenine and guanine) and pyrimidine (e.g. uracil, thymine and cytosine) moiety present in nucleosides and nucleotides which form hydrogen bonds in nucleic acid hybridization.

Modified nucleobases differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al., 2012, Accounts of Chemical Research, 45, 2055-2065 and Bergstrom, 2009, Curr. Protoc. Nucleic Acid Chem., 37, 1.4.1-1.4.32.

In some embodiments, a nucleobase moiety is modified by changing the purine or pyrimidine into a modified purine or pyrimidine, such as substituted purine or substituted pyrimidine, such as a nucleobase selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified nucleic acids, the nucleobase moieties are selected from A, U, G, C, and 5-methyl cytosine. Optionally, for LNA gapmers, 5-methyl cytosine LNA nucleosides may be used. 5-methyl cytosine may be denoted as “E”.

Unless otherwise indicated or contradicted by context, in the present disclosure, thymine (T) nucleobases within RNA sequences disclosed herein (e.g. siRNAs or mRNA target sequences) are to be interpreted as uracil (U) nucleobases.

In some embodiments, the modified nucleobase is 5-methyl cytosine.

“5-methylcytosine” or “5-me-C” means a methylated form of the DNA base cytosine (C) in which a methyl group is attached to the fifth carbon of the 6 atoms ring. 5-methyl cytosine may be used in place of cytosine, and forms the same Watson-Crick base-pairs as cytosine.

In some embodiments, the modified nucleobase is inosine.

### Conjugate Moiety

In some embodiments, the dsRNA is covalently attached to at least one conjugate moiety.

The term “conjugate moiety” as used herein refers to a non-nucleotide moiety which is covalently attached to the dsRNA (i.e. the ribonucleic acid) of the compound of the invention. The noun “conjugate” may be used to refer to a compound of the invention comprising a conjugate moiety and a dsRNA.

A “compound of the invention” may thus be an isolated dsRNA (i.e. a dsRNA with no further moieties attached, also referred to as “naked dsRNA” or “naked siRNA”) or a dsRNA with conjugate moiety attached.

In some embodiments, the conjugate moiety is covalently attached directly to the dsRNA. In other words, an atom of the dsRNA forms a covalent bond with an atom of the conjugate moiety.

In some embodiments, the conjugate moiety is covalently attached to the dsRNA via a linker (i.e. the conjugate moiety is indirectly covalently attached to the dsRNA). In other words, an atom of the dsRNA forms a covalent bond with an atom of the linker, and an atom of the linker forms a covalent bond with an atom of the conjugate moiety.

Thus, the term “covalently attached” encompasses direct attachment and indirect attachment (i.e. attachment via a linker). The terms “attached”, “positioned”, “linked” and “conjugated” are interchangeable in reference to the dsRNA and the conjugate moiety.

Attachment of a conjugate moiety to a dsRNA of the invention is termed “conjugation” herein. Conjugation of a dsRNA of the invention to one or more conjugate moieties may improve the pharmacology of the compound, e.g. by affecting the activity, cellular distribution, cellular uptake or stability of the compound. In some embodiments the conjugate moiety may modify or enhance the pharmacokinetic properties of the compound by improving cellular distribution, bioavailability, metabolism, excretion, permeability, and/or cellular uptake. In particular the conjugate moiety may target the compound to a specific organ, tissue or cell type and thereby enhance the effectiveness of the compound in that organ, tissue or cell type. At the same time the conjugate moiety may serve to reduce activity of the compound in non-target cell types, tissues or organs, e.g. off target activity or activity in non-target cell types, tissues or organs.

Nucleic acid conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103.

In some embodiments, the dsRNA is covalently attached to one or more conjugate moiety. In some embodiments, the dsRNA is covalently attached to two or more conjugate moieties, such as two, three, four or five conjugate moieties.

In some embodiments, the conjugate moiety is covalently attached to the sense strand. In some embodiments, the conjugate moiety is covalently attached at the 3′-end of the sense strand. In some embodiments, the conjugate moiety is covalently attached at the 5′-end of the sense strand. Preferably, the conjugate moiety is covalently attached at the 3′-end of the sense strand.

In some embodiments, the conjugate moiety is covalently attached to the antisense strand. In some embodiments, the conjugate moiety is covalently attached at the 3′-end of the antisense strand. In some embodiments, the conjugate moiety is covalently attached at the 5′-end of the antisense strand.

In some embodiments a conjugate moiety is attached to both the sense and antisense strands.

Herein the terms “5′-end” and “3′-end” refer to the direction of the nucleic acid strand and have their generally recognised meaning in the art.

Within the context of the invention, a conjugate moiety which is described as being attached “at the 3′-end” of a nucleic acid strand is preferably attached to 3′ terminal nucleotide. Alternatively, the conjugate moiety may be attached within one, two or three nucleotides of the 3′-end of the nucleic acid strand.

Similarly, a conjugate moiety which is described as being attached “at the 5′-end” of a nucleic acid strand is preferably attached to 5′ terminal nucleotide. Alternatively, the conjugate moiety may be attached within one, two or three nucleotides of the 5′-end of the nucleic acid strand.

In some embodiments, the conjugate moiety is not positioned at an end terminal position of either strand (i.e. the conjugate moiety is not positioned at the 5′ end or the 3′ end of either strand). For example, the conjugate moiety may be attached to a position in the middle or centre region of the contiguous nucleotide sequence. Herein the terms “middle” and “centre” are intended to indicate that the conjugate moiety is not located at either end of the strand, and not that the conjugate moiety is position equidistant from each end.

In some embodiments, the conjugate moiety is positioned at any position of the contiguous nucleotide sequence. In some embodiments, the conjugate moiety is positioned at any position on the dsRNA.

In some embodiments, the conjugate moiety is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) and combinations thereof.

### Fatty Acid Conjugate Moieties

In some embodiments, the conjugate moiety is a fatty acid.

A fatty acid is a carboxylic acid with an aliphatic chain. The general chemical formula of a carboxylic acid is as follows:

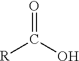

In a fatty acid, the R group of the carboxylic acid formula above is an aliphatic chain (i.e. a chain of carbon atoms). The aliphatic chain may be saturated (i.e. all carbon-carbon bonds in the chain are single) or unsaturated (i.e. not all carbon-carbon bonds in the chain are single, so the chain may, for example, contain one or more carbon-carbon double bonds).

A fatty acid may be defined by its total number of carbon (C) atoms (i.e. the C atom of the carboxyl group plus the C atoms of the aliphatic chain). A fatty acid with X number of C atoms may be referred to as a “CX fatty acid”. For example, a C16 fatty acid is any fatty acid having exactly 16 C atoms.

In some embodiments, the fatty acid molecule may be a molecule with 3-40 carbon atoms (i.e. a C3-C40 fatty acid).

In some embodiments, the fatty acid molecule is selected from a C3, C4, C5, C6, C7, C8, C9, C10, C11, C12, C13, C14, C15, C16, C17, C18, C19, C20, C21, C22, C23, C24, C25, C26, C27, C28, C29, C30, C31, C32, C33, C34, C35, C36, C37, C38, C39 and C40 fatty acid.

In some embodiments, the fatty acid molecule may be a molecule with 12-24 carbon atoms (e.g. C12-C24).

In some embodiments, the fatty acid molecule is selected from a C12, C13, C14, C15, C16, C17, C18, C19, C20, C21, C22, C23 and C24 fatty acid.

In some embodiments, the fatty acid molecule is branched or unbranched.

In some embodiments, the fatty acid molecule is a partially saturated fatty acid. In some embodiments, the fatty acid molecule is a fully saturated fatty acid. In some embodiments, the fatty acid molecule is an unsaturated fatty acid.

In some embodiments, the fatty acid molecule is a C22 fatty acid. In some embodiments, the conjugate moiety is behenic acid (which is a C22 fatty acid wherein the aliphatic chain is fully saturated). The systematic name for behenic acid is docosanoic acid. When conjugated to a dsRNA of the invention, behenic acid has the following chemical structure (wherein the wavy line indicates the covalent bond is formed with an atom of a linker or of a dsRNA):

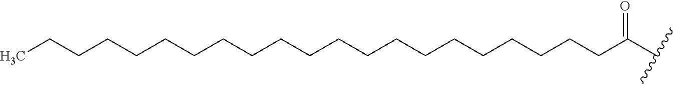 [SMILES: *C(=O)CCCCCCCCCCCCCCCCCCCCC]

An siRNA compound of the invention comprising or consisting of an siRNA duplex conjugated to behenic acid may be referred to herein as a “C22 siRNA”.

In some embodiments, the conjugate moiety is a C16 fatty acid (i.e. a fatty acid comprising sixteen carbons). In some embodiments, the conjugate moiety is palmitic acid (which is a C16 fatty acid wherein the aliphatic chain is fully saturated). The systematic name for palmitic acid is hexdecanoic acid. When conjugated to a dsRNA of the invention, palmitic acid has the following chemical structure (wherein the wavy line indicates the covalent bond is formed with an atom of a linker or of a dsRNA):

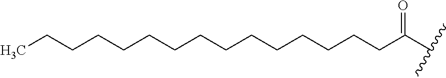 [SMILES: *C(=O)CCCCCCCCCCCCCCC]

An siRNA compound of the invention comprising or consisting of an siRNA duplex conjugated to palmitic acid may be referred to herein as a “C16 siRNA”.

### Linkers

In some embodiments, the dsRNA of the invention is covalently attached to the conjugate moiety via a linker. In other words, the compound of the invention may comprise a linker which is positioned between the dsRNA and the conjugate moiety. The linker may be attached to the contiguous nucleotide sequence of a strand of the dsRNA and the conjugate moiety.

A linker (also referred to herein as a “conjugate linker”, “linkage”, “linker moiety”, “linker group”, “linker region” or “spacer”) is a connection between two atoms that links one chemical group or segment of interest to another chemical group or segment of interest via one or more covalent bonds. Conjugate moieties can be attached to the dsRNA directly or through a linker. A linker serves to covalently connect a conjugate moiety to a dsRNA.

In some embodiments, the linker is a cleavable linker. In some embodiments, the linker is a biocleavable linker.

### Cleavable Linkers—Including Linker Nucleotides

Biocleavable linkers comprise or consist of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In some embodiments, the biocleavable linker is susceptible to S1 nuclease cleavage. In some embodiments the nuclease susceptible linker comprises between 1 and 5 nucleosides, such as DNA nucleoside(s) comprising at least two consecutive phosphodiester linkages. Phosphodiester containing biocleavable linkers are described in more detail in WO 2014/076195.

Accordingly, the biocleavable linker may be one or more nucleotides, referred to as “linker nucleotides”. Thus, in some embodiments, the linker comprises 1 to 3 linker nucleotides. In some embodiments, the linker consists of 1 to 3 linker nucleotides.

In some embodiments, the linker comprises 1 linker nucleotide. In some embodiments, the linker comprises 2 linker nucleotides (i.e. a dinucleotide). In some embodiments, the linker comprises a CA dinucleotide. In some embodiments, the linker comprises 3 linker nucleotides (i.e. a trinucleotide).

In some embodiments, the linker consists of 1 linker nucleotide. In some embodiments, the linker consists of 2 linker nucleotides (i.e. a dinucleotide). In some embodiments, the linker consist of a CA dinucleotide. In some embodiments, the linker consists of 3 linker nucleotides (i.e. a trinucleotide).

In embodiments wherein the conjugate moiety is attached at the 5′-end or at the 3′end of the sense strand or antisense strand, the linker nucleotides are positioned at the 5′-end or 3′-end of the sense strand or antisense strand and are thus contiguous with the sense strand or antisense strand. Such linker nucleotides may be referred to herein as “further 5′ and/or 3′ nucleotides” or “additional 5′ and/or 3′ nucleotides”.

In some embodiments the additional 5′ and/or 3′ end nucleotides are linked with phosphodiester linkages, and are DNA or RNA. Nucleotide based biocleavable linkers suitable for such use are disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs within a single oligonucleotide.

### Non-Cleavable Linkers—Including Alkyl Linkers

In some embodiments, the linker is not a biocleavable linker.

Linkers that are not biocleavable but primarily serve to covalently connect a conjugate moiety to an oligonucleotide are known. These linkers may comprise a chain structure or an oligomer of repeating units such as ethylene glycol, amino acid units or amino alkyl groups, or a combination thereof.

In some embodiments, the linker is an amino alkyl (i.e. an amino alkyl group or an amino alkyl linker).

An amino alkyl is a molecule consisting of an amino group (—NH₂) and an alkyl group (—CₙH₂ₙ₊₁). An amino alkyl linker may be described by the number of carbon (C) atoms in the alkyl group (i.e. the number of C atoms in the carbon chain). For example, a C6 amino alkyl linker is a 6-carbon alkyl group with an amino group on the sixth carbon.

In some embodiments, the linker comprises a C2 to C36 amino alkyl linker (i.e. the carbon chain consists of from 2 to 36 carbon atoms). In some embodiments, the linker comprises a C6 to C12 amino alkyl linker (i.e. the carbon chain consists of from 2 to 36 carbon atoms). In some embodiments, the linker comprises a C6 amino alkyl linker (i.e. the carbon chain consists of 6 carbon atoms).

In some embodiments, the linker consists of a C2 to C36 amino alkyl linker. In some embodiments, the linker consists of a C6 to C12 amino alkyl linker. In some embodiments, the linker consists of a C6 amino alkyl linker.

A “C6 amino alkyl linker” is a six-carbon chain with an amino group covalently attached to carbon 6. A “C6 amino alkyl linker” has the following structure (wherein the wavy lines indicate covalent bonds to the conjugate moiety or the compound of the invention):

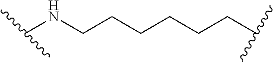 [SMILES: *CCCCCCN*]

In some embodiments, the compound of the invention is attached to the conjugate moiety palmitic acid via a C6 amino alkyl linker.

In some embodiments, the compound of the invention is attached to the conjugate moiety behenic acid via a C6 amino alkyl linker at the 3′ end of the sense strand. In preferred embodiments, the compound of the invention is attached to the conjugate moiety palmitic acid via a C6 amino alkyl linker at the 3′ end of the sense strand. In such embodiments, the amino group of the C6 amino alkyl linker forms an amide bond with the carboxylic acid group on palmitic acid or behenic acid, and the carbon at the opposite end of the linker to the amino group links to a phosphate group at the 3′ end of the sense strand.

The structure below shows behenic acid (C22) attached via a C6 amino alkyl linker to the 3′ end of the sense strand:

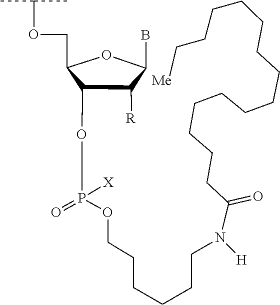

R=OMe, F, H, OH or O(CH₂)₂OCH₃
X=S or O
B=adenine, uridine, thymidine, guanine or cytosine

The structure below shows palmitic acid (C16) attached via a C6 amino alkyl linker to the 3′ end of the sense strand:

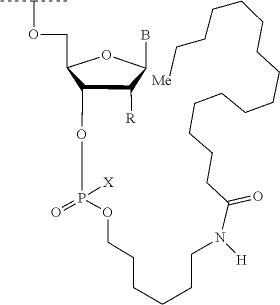

R=OMe, F, H, OH or O(CH₂)₂OCH₃
X=S or O
B=adenine, uridine, thymidine, guanine or cytosine

In some embodiments, the linker is a C6 alkyl linker (i.e. a 6-carbon chain).

In some embodiments, the linker is polyethylene glycol (PEG). In some embodiments, the linker is triethylene gycol (TEG).

### Compound Activity

The compound of the invention is for reducing the expression of Janus kinase 1 (JAK1). The skilled person will understand that within the context of the invention any reduction in expression JAK1 is contemplated.

The term “modulation of expression” as used herein is to be understood as an overall term for a nucleic acid molecules ability to alter the amount of a target when compared to the amount of the target before administration of the nucleic acid molecule. Alternatively, modulation of expression may be determined by reference to a control experiment. It is generally understood that the control is an individual or target cell treated with a saline composition or an individual or target cell treated with a non-targeting or nucleic acid molecule (mock). It may however also be an individual treated with the standard of care.

“Reducing the expression” or “decreasing the expression” of a target nucleic acid is one type of modulating the expression of the target nucleic acid.

The compounds of the invention have the ability to inhibit, down-regulate, reduce, decrease, remove, stop, prevent, lessen, lower, avoid or terminate expression of JAK1, e.g. by degradation of mRNA or blockage of transcription.

In some embodiments the compound of the invention may decrease expression of JAK1 mRNA.

In other embodiments the compound of the invention may decrease expression of JAK1 protein.

In still further embodiments the compound of the invention may decrease expression of JAK1 mRNA and JAK1 protein.

In some embodiments the compound of the invention is capable of decreasing the expression of JAK1 mRNA by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%, compared to a control.

In some embodiments the compound of the invention is capable of decreasing the expression of JAK1 protein by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%, compared to a control.

In some embodiments the compound of the invention is capable of decreasing the expression of JAK1 mRNA and JAK1 protein by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%, compared to a control.

Within the context of the invention a control may be a cell that has not been exposed to the compound, such as a cell which has been exposed to an equal volume of placebo, such as phosphate buffered saline.

### Pharmaceutically Acceptable Salts

In some embodiments the compound of the invention may be in the form of a pharmaceutically acceptable salt.

The term “salts” as used herein conforms to its generally known meaning, i.e. an ionic assembly of anions and cations. For example, the salt may comprise a metal cation, such as a sodium salt or a potassium salt.

As used herein the term “pharmaceutically acceptable salt” is any salt of a compound of the invention which is generally considered to be safe and non-toxic to humans and/or animals.

The pharmaceutically acceptable salt may be a sodium salt or a potassium salt.

### Composition

The compound of the invention may be in the form of a “composition”.

A nucleic acid molecule composition has less than 20% impurities, preferably less than 15% or 10% impurities, more preferably less than 9%, 8%, 7% or 6% impurities, most preferably less than 5% impurities. The impurities are typically nucleic acid molecules which are one or two nucleotides shorter (n−1 or n−2) than the primary nucleic acid molecule component.

The invention provides a pharmaceutical composition comprising the compound of the invention and a pharmaceutically acceptable diluent, solvent, carrier, salt and/or adjuvant.

Within the context of the invention the pharmaceutical composition may comprise an aqueous diluent or solvent, such as phosphate buffered saline, such as a sterile phosphate buffered saline solution.

The pharmaceutical composition of the invention may comprise one or more additional therapeutic agents.

In some embodiments the additional therapeutic agent may be a JAK1 inhibitor, such as a JAK1 antagonist therapeutic or an anti-JAK1 antibody.

The term “antibody” refers to a molecule characterized by reacting specifically with an antigen (JAK1, in the context of the present invention) in some way, where the antibody and the antigen are each defined in terms of the other. Herein the term “antibody” may refer to a complete antibody molecule or any fragment or region thereof, such as the heavy chain, the light chain, Fab region, and Fc region.

In some embodiments, the compound of the invention may be is encapsulated in a lipid-based delivery vehicle, covalently linked to or encapsulated in a dendrimer, or conjugated to an aptamer.

### Method of Suppression

The invention provides an in vivo or in vitro method for suppressing JAK1 expression in a target cell. The method comprises administering the compound of the invention or the pharmaceutical composition of the invention, in an effective amount, to the cell.

The term “suppress” (or suppressing or “suppression”) is synonymous with “down-regulating”, “decreasing” and “inhibiting”.

In some embodiments the method is an in vivo method.

In other embodiments the method is an in vitro method

In some embodiments the cell is a human cell or a mammalian cell.

In some embodiments the method decreases expression of JAK1 mRNA.

In other embodiments the method decreases expression of JAK1 protein.

In still further embodiments the method decreases expression of JAK1 mRNA and JAK1 protein.

In some embodiments the method of the invention decrease the expression of JAK1 mRNA by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%, compared to a control.

In other embodiments the method of the invention decrease the expression of JAK1 protein by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%, compared to a control.

In still further embodiments the method of the invention decrease the expression of JAK1 mRNA and JAK1 protein by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%, compared to a control.

Within the context of the invention a control may be a cell that has not been exposed to the compound, such as a cell which has been exposed to an equal volume of placebo, such as phosphate buffered saline.

In some embodiments the method of the invention comprise administering one or more additional therapeutic agents.

In some embodiments the additional therapeutic agent is a JAK1 inhibitor, such as a JAK1 antagonist therapeutic or an anti-JAK1 antibody.

### Method of Treatment and Medical Use

The invention provides a method for treating or preventing a disease comprising administering a therapeutically or prophylactically effective amount of the compound of the invention or the pharmaceutical composition of the invention, to a subject suffering from or susceptible to a disease.

The invention also provides the compound of the invention or the pharmaceutical composition of the invention for use in a method for treating or preventing a disease.

The invention also provides use of the compound of the invention or the pharmaceutical composition of the invention for the preparation of a medicament for a method of treatment or prevention of a disease in a subject.

In some embodiments the method comprises administering one or more additional therapeutic agents.

In some embodiments the additional therapeutic agent is a JAK1 inhibitor, such as a JAK1 antagonist therapeutic or an anti-JAK1 antibody.

The compounds and compositions of the invention may be used for the treatment of a disease associated with increased expression of JAK1.

In some embodiments the disease to be treated is selected from the group consisting of inflammatory bowel disease, organ transplant rejection, graft-versus-host disease, multiple sclerosis, rheumatoid arthritis (RA), juvenile idiopathic arthritis, psoriasis, dermatitis, diabetic nephropathy, systemic lupus erythematosus (SLE), dry eye disease, cancer, myelofibrosis, and asthma.

In some embodiments, the disease is dry eye disease.

### Treatment and Prevention

The terms “treatment”, “treating”, “treats” and the like are used herein generally mean obtaining a desired pharmacological and/or physiological effect. This effect is therapeutic in terms of partially or completely curing a disease and/or adverse effect attributed to the disease.

The term “treatment” as used herein covers any treatment of a disease in a subject and includes: (a) inhibiting the disease, i.e. arresting its development; (b) ameliorating (i.e. relieving) the disease, i.e. causing regression of the disease; and (c) preventing the disease, i.e. stopping the disease starting or progressing. Thus, a compound that ameliorates and/or inhibits an infection is a compound that treats an infection.

Preferably, the term “treatment” as used herein relates to medical intervention of an already manifested disorder, like the treatment of an already defined and manifested infection.

“Amelioration” refers to a lessening, slowing, stopping, or reversing of at least one indicator of the severity of a syndrome or condition. The severity of indicators may be determined by subjective or objective measures, which are known to those skilled in the art.

Herein the term “preventing”, “prevention” or “prevents” relates to a prophylactic treatment, i.e. to a measure or procedure the purpose of which is to prevent, rather than to cure a disease. Prevention means that a desired pharmacological and/or physiological effect is obtained that is prophylactic in terms of completely or partially preventing a disease or symptom thereof.

### Subject

For the purposes of the present invention the “subject” (or “patient”) may be a vertebrate. In context of the present invention, the term “subject” includes both humans and other animals, particularly mammals, and other organisms. Thus, the herein provided means and methods are applicable to both human therapy and veterinary applications. Accordingly, herein the subject may be an animal such as a mouse, rat, hamster, rabbit, guinea pig, ferret, cat, dog, chicken, sheep, bovine species, horse, camel, or primate. Preferably, the subject is a mammal. More preferably the subject is human.

### Administering

“Administering” or “administration” means providing a pharmaceutical agent (e.g. a double stranded RNA or a composition of the invention) to a subject in a manner that is pharmacologically useful (e.g. to treat a condition in a subject) and includes, but is not limited to, administering by a medical professional and self-administering.

Methods of administration may include parenteral, intravenous, intramuscular, subcutaneous, oral, rectal, vaginal or inhaled. It is understood that the skilled person will be able to determine an appropriate method of administration.

The administration can be a “concomitant administration” in which two pharmaceutical agents are administered to a subject at the same time (i.e. “administered concomitantly”). Concomitant administration does not require that both pharmaceutical agents are administered in a single pharmaceutical composition, in the same dosage form, or by the same route of administration. Concomitant administration can be either simultaneous, sequential or separate administration of the two pharmaceutical agents. The effects of both pharmaceutical agents do not need to manifest themselves at the same time or location. Preferably, the effects only need to overlap for a period of time.

### Kits

The invention provides a kit comprising the compound of the invention or the pharmaceutical composition of the invention and instructions for use. In some embodiments the kit also comprises one or more additional therapeutic agents. In some embodiments the additional therapeutic agent is a JAK1 inhibitor, such as a JAK1 antagonist therapeutic or an anti-JAK1 antibody.

### Definitions

Unless specific definitions are provided, it should be understood that the nomenclature utilized in connection with analytical chemistry, synthetic organic chemistry, and medicinal and pharmaceutical chemistry are those well known and commonly used in the art. Standard techniques may be used for chemical synthesis, and chemical analysis.

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) than the stated reference value unless otherwise stated or otherwise evident from the context (except where such number would exceed 100% of a possible value and it is apparent that this was not intended).

The term “independently selected” means that each time a selection is made it is made independently of any other selection. For example, if there are twenty nucleotides within a nucleic acid molecule and each is independently selected from adenine (A), cytosine (C), guanine (G) and uracil (U), the selection of any of the four nucleotides at any position is permitted.

### Compounds of the Invention

Compounds of the invention used in the Examples herein are presented below in Tables 1 to 4.

In the context of a compound of the invention, the term “sequence” refers to the order of bases of an oligonucleotide (e.g. of a sense strand or antisense strand). The chemistry of a sequence is not limited, other than the order of bases. For example, the nucleotides of a given sequence may have any type of 2′ sugar modifications or any type of internucleotide linkages.

Accordingly, the terms “sense strand sequence” or “sequence of the sense strand” refer solely to the order of bases in the sense strand. The term “sense strand” refers to the sense strand molecule per se, and thus incorporates the order of bases as well as other chemistry of the molecule such as internucleoside linkages.

Likewise, the terms “antisense strand sequence” or “sequence of the antisense strand” refer solely to the order of bases in the antisense strand. The term “antisense strand” refers to the antisense strand molecule per se, and thus incorporates the order of bases as well as other chemistry of the molecule such as internucleoside linkages.

Sequence identifier numbers (SEQ ID NOs) are assigned to both “sequences” (i.e. order of bases) and to “strands” (i.e. molecules per se) herein to meet formal requirements. Thus, a given oligonucleotide (e.g. sense strand or antisense strand) is assigned a first SEQ ID NO for its sequence (i.e. order of bases) and a second SEQ ID NO for the molecule per se.

“Duplex number” refers to the duplex (i.e. double-stranded molecule) formed by the corresponding sense strand sequence and antisense strand sequence shown in Table 1 below. For example, “duplex 1” refers to the duplex formed by SEQ ID NO 3 (sense strand sequence) and SEQ ID NO 194 (antisense strand sequence). The chemistry of the oligonucleotides of the duplex, other than the base sequences, is not limited by the duplex number. For example, the nucleotides of a given duplex number may have any type of 2′ sugar modifications or any type of base modifications or any type of internucleotide linkages.

“Compound number” refers to the i.e. double-stranded molecule formed by the corresponding sense strand and antisense strand as shown in Table 3 below, which include the chemical modifications of the strands (e.g. 2′ sugar modifications and/or base modifications and/or internucleotide linkages). Compound numbers are not assigned consecutively (i.e. 1, 2, 3, 4 . . . etc.) but rather each compound number is the number of the start position nucleotide in JAK1 mRNA (NM_002227.4) of the target sequence for that compound.

Compounds with the suffix “_C16” (e.g. 614_C16) comprise a palmitic acid conjugate moiety attached to the sense strand, such as attached to a terminal position of the sense strand (e.g. attached to the 3′ end of the sense strand) or in a middle or centre region position of the sense strand (i.e. not at a terminal position of the sense strand).

Compounds with the suffix “_C22” (e.g. 614_C22) comprise a C22 conjugate moiety attached to the sense strand, such as attached to a terminal position of the sense strand (e.g. attached to the 3′ end of the sense strand) or in a middle or centre region position of the sense strand (i.e. not at a terminal position of the sense strand).

### Table 1: Sense and Antisense Strand Sequences and Duplex Numbers

Table 1 below presents the sequences of the sense strand and antisense strand of each of the compounds of the invention used in the Examples herein. As noted hereinabove, the term “sequence” refers solely to the order of bases in the strand; the chemistry of the strand (such as the group at the 2′ position of each nucleoside and the chemistry of each internucleotide linkage) is not otherwise limited by these sequences.

* The term “20nt+U” means that the antisense strand consists of a sequence of 21 nucleotides in which the 20 3′-end-most contiguous nucleotides are 100% complementary to a same-length portion of JAK1 mRNA, and the U nucleotide at the 5′-end does not match with a complementary nucleobase at the corresponding position on the JAK1 mRNA. For example, for Duplex #2, the antisense strand of SEQ ID NO: 195 (which has a 20nt+U design) is 100% complementary to SEQ ID NO: 386 (see Table 2) over 20nt (from position 673 to 692 of NM_002227.4) but the U nucleotide at the 5′-end is not complementary to the nucleobase located on position 693 of NM_002227.4.

The term “21nt” means that the antisense strand consists of a sequence of 21 nucleotides which is 100% complementary to a same-length portion of JAK1 mRNA. For example, for Duplex #1, the antisense strand of SEQ ID NO: 194 (which has a 21nt design) is 100% complementary to SEQ ID NO: 385 (see Table 2) over its entire length of 21nt, from position 614 to 634 of NM_002227.4.

TABLE 1

| | Sense | | | | | |
|---|---|---|---|---|---|---|
| Start pos. in | strand | | Antisense | | | |
| NM_002227.4 | sequence | | strand | | | Antisense |
| (corresponds to | SEQ ID | Sense strand | sequence | Antisense strand | | strand |
| Compound #) | NO | sequence (5′-3′) | SEQ ID NO | sequence (5′-3′) | Duplex # | design* |
| 614 | 3 | CGGAUGAGGUUCUAUUUCA | 194 | UGAAAUAGAACCUCAUCCGGU | 1 | 21 nt |
| 673 | 4 | GCGUCAUUCUCCAAAGAAA | 195 | UUUCUUUGGAGAAUGACGCCA | 2 | 20 nt + U |
| 724 | 5 | AGAUGCAACCCCUCUCCUA | 196 | UAGGAGAGGGGUUGCAUCUGG | 3 | 20 nt + U |
| 728 | 6 | GCAACCCCUCUCCUUGAUA | 197 | UAUCAAGGAGAGGGGUUGCAU | 4 | 20 nt + U |
| 753 | 7 | CACUGGAGUAUCUGUUUGA | 198 | UCAAACAGAUACUCCAGUGAG | 5 | 20 nt + U |
| 756 | 8 | UGGAGUAUCUGUUUGCUCA | 199 | UGAGCAAACAGAUACUCCAGU | 6 | 21 nt |
| 818 | 9 | CCCAAGACCGAGCAGGAUA | 200 | UAUCCUGCUCGGUCUUGGGGU | 7 | 20 nt + U |
| 874 | 10 | CCUGGCCAUCUCACACUAA | 201 | UUAGUGUGAGAUGGCCAGGAC | 8 | 20 nt + U |
| 875 | 11 | CUGGCCAUCUCACACUAUA | 202 | UAUAGUGUGAGAUGGCCAGGA | 9 | 20 nt + U |
| 876 | 12 | UGGCCAUCUCACACUAUGA | 203 | UCAUAGUGUGAGAUGGCCAGG | 10 | 20 nt + U |
| 877 | 13 | GGCCAUCUCACACUAUGCA | 204 | UGCAUAGUGUGAGAUGGCCAG | 11 | 20 nt + U |
| 878 | 14 | GCCAUCUCACACUAUGCCA | 205 | UGGCAUAGUGUGAGAUGGCCA | 12 | 21 nt |
| 883 | 15 | CUCACACUAUGCCAUGAUA | 206 | UAUCAUGGCAUAGUGUGAGAU | 13 | 20 nt + U |
| 884 | 16 | UCACACUAUGCCAUGAUGA | 207 | UCAUCAUGGCAUAGUGUGAGA | 14 | 21 nt |
| 1069 | 17 | CAGCGUGUCCACGCAUGAA | 208 | UUCAUGCGUGGACACGCUGCU | 15 | 20 nt + U |
| 1075 | 18 | GUCCACGCAUGACCUGAAA | 209 | UUUCAGGUCAUGCGUGGACAC | 16 | 20 nt + U |
| 1085 | 19 | GACCUGAAGGUGAAAUACA | 210 | UGUAUUUCACCUUCAGGUCAU | 17 | 20 nt + U |
| 1107 | 20 | CUACCUUGGAAACUUUGAA | 211 | UUCAAAGUUUCCAAGGUAGCC | 18 | 20 nt + U |
| 1108 | 21 | UACCUUGGAAACUUUGACA | 212 | UGUCAAAGUUUCCAAGGUAGC | 19 | 21 nt |
| 1138 | 22 | UGCUGAAAUAUUUGAGACA | 213 | UGUCUCAAAUAUUUCAGCACC | 20 | 20 nt + U |
| 1182 | 23 | AUGAGAUGAAUUGGUUUCA | 214 | UGAAACCAAUUCAUCUCAUUU | 21 | 21 nt |
| 1189 | 24 | GAAUUGGUUUCAUUCGAAA | 215 | UUUCGAAUGAAACCAAUUCAU | 22 | 20 nt + U |
| 1190 | 25 | AAUUGGUUUCAUUCGAAUA | 216 | UAUUCGAAUGAAACCAAUUCA | 23 | 20 nt + U |
| 1304 | 26 | AAAAAUAAACUGAAGCGGA | 217 | UCCGCUUCAGUUUAUUUUUUU | 24 | 21 nt |
| 1306 | 27 | AAAUAAACUGAAGCGGAAA | 218 | UUUCCGCUUCAGUUUAUUUUU | 25 | 21 nt |
| 1311 | 28 | AACUGAAGCGGAAAAAACA | 219 | UGUUUUUUCCGCUUCAGUUUA | 26 | 20 nt + U |
| 1367 | 29 | AUCCGGGAAGAGUGGAACA | 220 | UGUUCCACUCUUCCCGGAUCU | 27 | 21 nt |
| 1368 | 30 | UCCGGGAAGAGUGGAACAA | 221 | UUGUUCCACUCUUCCCGGAUC | 28 | 21 nt |
| 1372 | 31 | GGAAGAGUGGAACAAUUUA | 222 | UAAAUUGUUCCACUCUUCCCG | 29 | 20 nt + U |
| 1412 | 32 | CACAUUGUAAUAAAGGAGA | 223 | UCUCCUUUAUUACAAUGUGAG | 30 | 20 nt + U |
| 1413 | 33 | ACAUUGUAAUAAAGGAGUA | 224 | UACUCCUUUAUUACAAUGUGA | 31 | 20 nt + U |
| 1432 | 34 | UGUGGUCAGCAUUAACAAA | 225 | UUUGUUAAUGCUGACCACAGA | 32 | 20 nt + U |
| 1579 | 35 | CCCGUUGAUCGUCCACAAA | 226 | UUUGUGGACGAUCAACGGGGG | 33 | 20 nt + U |
| 1580 | 36 | CCGUUGAUCGUCCACAACA | 227 | UGUUGUGGACGAUCAACGGGG | 34 | 21 nt |
| 1581 | 37 | CGUUGAUCGUCCACAACAA | 228 | UUGUUGUGGACGAUCAACGGG | 35 | 20 nt + U |
| 1583 | 38 | UUGAUCGUCCACAACAUAA | 229 | UUAUGUUGUGGACGAUCAACG | 36 | 20 nt + U |
| 1584 | 39 | UGAUCGUCCACAACAUACA | 230 | UGUAUGUUGUGGACGAUCAAC | 37 | 21 nt |
| 1586 | 40 | AUCGUCCACAACAUACAGA | 231 | UCUGUAUGUUGUGGACGAUCA | 38 | 21 nt |
| 1587 | 41 | UCGUCCACAACAUACAGAA | 232 | UUCUGUAUGUUGUGGACGAUC | 39 | 21 nt |
| 1588 | 42 | CGUCCACAACAUACAGAAA | 233 | UUUCUGUAUGUUGUGGACGAU | 40 | 20 nt + U |
| 1595 | 43 | AACAUACAGAAUGGCUGUA | 234 | UACAGCCAUUCUGUAUGUUGU | 41 | 20 nt + U |
| 1596 | 44 | ACAUACAGAAUGGCUGUCA | 235 | UGACAGCCAUUCUGUAUGUUG | 42 | 21 nt |
| 1601 | 45 | CAGAAUGGCUGUCAUGGUA | 236 | UACCAUGACAGCCAUUCUGUA | 43 | 20 nt + U |
| 1602 | 46 | AGAAUGGCUGUCAUGGUCA | 237 | UGACCAUGACAGCCAUUCUGU | 44 | 20 nt + L |
| 1603 | 47 | GAAUGGCUGUCAUGGUCCA | 238 | UGGACCAUGACAGCCAUUCUG | 45 | 21 nt |
| 1608 | 48 | GCUGUCAUGGUCCAAUCUA | 239 | UAGAUUGGACCAUGACAGCCA | 46 | 20 nt + U |
| 1609 | 49 | CUGUCAUGGUCCAAUCUGA | 240 | UCAGAUUGGACCAUGACAGCC | 47 | 20 nt + U |
| 1611 | 50 | GUCAUGGUCCAAUCUGUAA | 241 | UUACAGAUUGGACCAUGACAG | 48 | 20 nt + U |
| 1640 | 51 | AUCAAUAAAUUGCGGCAAA | 242 | UUUGCCGCAAUUUAUUGAUGG | 49 | 20 nt + U |
| 1642 | 52 | CAAUAAAUUGCGGCAAGAA | 243 | UUCUUGCCGCAAUUUAUUGAU | 50 | 21 nt |
| 1671 | 53 | AGGGGAUGUACGUGCUGAA | 244 | UUCAGCACGUACAUCCCCUCC | 51 | 20 nt + U |
| 1672 | 54 | GGGGAUGUACGUGCUGAGA | 245 | UCUCAGCACGUACAUCCCCUC | 52 | 20 nt + U |
| 1673 | 55 | GGGAUGUACGUGCUGAGGA | 246 | UCCUCAGCACGUACAUCCCCU | 53 | 20 nt + U |
| 1674 | 56 | GGAUGUACGUGCUGAGGUA | 247 | UACCUCAGCACGUACAUCCCC | 54 | 20 nt + U |
| 1677 | 57 | UGUACGUGCUGAGGUGGAA | 248 | UUCCACCUCAGCACGUACAUC | 55 | 20 nt + U |
| 1678 | 58 | GUACGUGCUGAGGUGGAGA | 249 | UCUCCACCUCAGCACGUACAU | 56 | 20 nt + U |
| 1690 | 59 | GUGGAGCUGCACCGACUUA | 250 | UAAGUCGGUGCAGCUCCACCU | 57 | 20 nt + U |
| 1692 | 60 | GGAGCUGCACCGACUUUGA | 251 | UCAAAGUCGGUGCAGCUCCAC | 58 | 21 nt |
| 1698 | 61 | GCACCGACUUUGACAACAA | 252 | UUGUUGUCAAAGUCGGUGCAG | 59 | 20 nt + U |
| 1699 | 62 | CACCGACUUUGACAACAUA | 253 | UAUGUUGUCAAAGUCGGUGCA | 60 | 20 nt + U |
| 1723 | 63 | GACCGUCACCUGCUUUGAA | 254 | UUCAAAGCAGGUGACGGUCAU | 61 | 20 nt + U |
| 1769 | 64 | AAGCAGUUCAAGAACUUUA | 255 | UAAAGUUCUUGAACUGCUUCU | 62 | 20 nt + U |
| 1770 | 65 | AGCAGUUCAAGAACUUUCA | 256 | UGAAAGUUCUUGAACUGCUUC | 63 | 21 nt |
| 1780 | 66 | GAACUUUCAGAUCGAGGUA | 257 | UACCUCGAUCUGAAAGUUCUU | 64 | 20 nt + U |
| 1798 | 67 | GCAGAAGGGCCGCUACAGA | 258 | UCUGUAGCGGCCCUUCUGCAC | 65 | 20 nt + U |
| 1876 | 68 | GCAGAUCCUGCGCACGGAA | 259 | UUCCGUGCGCAGGAUCUGCUU | 66 | 20 nt + U |
| 1927 | 69 | GCCCAAGCCCCGAGAAAUA | 260 | UAUUUCUCGGGGCUUGGGCUG | 67 | 20 nt + U |
| 1928 | 70 | CCCAAGCCCCGAGAAAUCA | 261 | UGAUUUCUCGGGGCUUGGGCU | 68 | 20 nt + U |
| 1929 | 71 | CCAAGCCCCGAGAAAUCUA | 262 | UAGAUUUCUCGGGGCUUGGGC | 69 | 20 nt + U |
| 1936 | 72 | CCGAGAAAUCUCCAACCUA | 263 | UAGGUUGGAGAUUUCUCGGGG | 70 | 20 nt + U |
| 1952 | 73 | CUGCUGGUGGCUACUAAGA | 264 | UCUUAGUAGCCACCAGCAGGU | 71 | 21 nt |
| 1954 | 74 | GCUGGUGGCUACUAAGAAA | 265 | UUUCUUAGUAGCCACCAGCAG | 72 | 21 nt |
| 1956 | 75 | UGGUGGCUACUAAGAAAGA | 266 | UCUUUCUUAGUAGCCACCAGC | 73 | 20 nt + U |
| 1958 | 76 | GUGGCUACUAAGAAAGCCA | 267 | UGGCUUUCUUAGUAGCCACCA | 74 | 20 nt + U |
| 1978 | 77 | GGAGUGGCAGCCCGUCUAA | 268 | UUAGACGGGCUGCCACUCCUG | 75 | 20 nt + U |
| 2066 | 78 | GGCACGAGAACACACAUCA | 269 | UGAUGUGUGUUCUCGUGCCUC | 76 | 20 nt + U |
| 2068 | 79 | CACGAGAACACACAUCUAA | 270 | UUAGAUGUGUGUUCUCGUGCC | 77 | 20 nt + U |
| 2102 | 80 | GAUUACAAGGAUGACGAAA | 271 | UUUCGUCAUCCUUGUAAUCCA | 78 | 20 nt + U |
| 2111 | 81 | GAUGACGAAGGAACUUCUA | 272 | UAGAAGUUCCUUCGUCAUCCU | 79 | 20 nt + U |
| 2138 | 82 | AAGAUAAAAGUGAUCCUCA | 273 | UGAGGAUCACUUUUAUCUUCU | 80 | 21 nt |
| 2146 | 83 | AGUGAUCCUCAAAGUCUUA | 274 | UAAGACUUUGAGGAUCACUUU | 81 | 21 nt |
| 2148 | 84 | UGAUCCUCAAAGUCUUAGA | 275 | UCUAAGACUUUGAGGAUCACU | 82 | 21 nt |
| 2205 | 85 | CAGCCAGCAUGAUGAGACA | 276 | UGUCUCAUCAUGCUGGCUGCC | 83 | 21 nt |
| 2206 | 86 | AGCCAGCAUGAUGAGACAA | 277 | UUGUCUCAUCAUGCUGGCUGC | 84 | 20 nt + U |
| 2218 | 87 | GAGACAGGUCUCCCACAAA | 278 | UUUGUGGGAGACCUGUCUCAU | 85 | 21 nt |
| 2229 | 88 | CCCACAAACACAUCGUGUA | 279 | UACACGAUGUGUUUGUGGGAG | 86 | 21 nt |
| 2230 | 89 | CCACAAACACAUCGUGUAA | 280 | UUACACGAUGUGUUUGUGGGA | 87 | 20 nt + U |
| 2237 | 90 | CACAUCGUGUACCUCUAUA | 281 | UAUAGAGGUACACGAUGUGUU | 88 | 20 nt + U |
| 2238 | 91 | ACAUCGUGUACCUCUAUGA | 282 | UCAUAGAGGUACACGAUGUGU | 89 | 20 nt + U |
| 2239 | 92 | CAUCGUGUACCUCUAUGGA | 283 | UCCAUAGAGGUACACGAUGUG | 90 | 20 nt + U |
| 2269 | 93 | CGACGUGGAGAAUAUCAUA | 284 | UAUGAUAUUCUCCACGUCGCG | 91 | 20 nt + U |
| 2308 | 94 | GGGUCCUCUGGAUCUCUUA | 285 | UAAGAGAUCCAGAGGACCCCC | 92 | 20 nt + U |
| 2317 | 95 | GGAUCUCUUCAUGCACCGA | 286 | UCGGUGCAUGAAGAGAUCCAG | 93 | 20 nt + U |
| 2318 | 96 | GAUCUCUUCAUGCACCGGA | 287 | UCCGGUGCAUGAAGAGAUCCA | 94 | 21 nt |
| 2319 | 97 | AUCUCUUCAUGCACCGGAA | 288 | UUCCGGUGCAUGAAGAGAUCC | 95 | 21 nt |
| 2320 | 98 | UCUCUUCAUGCACCGGAAA | 289 | UUUCCGGUGCAUGAAGAGAUC | 96 | 21 nt |
| 2321 | 99 | CUCUUCAUGCACCGGAAAA | 290 | UUUUCCGGUGCAUGAAGAGAU | 97 | 21 nt |
| 2322 | 100 | UCUUCAUGCACCGGAAAAA | 291 | UUUUUCCGGUGCAUGAAGAGA | 98 | 20 nt + U |
| 2323 | 101 | CUUCAUGCACCGGAAAAGA | 292 | UCUUUUCCGGUGCAUGAAGAG | 99 | 20 nt + U |
| 2520 | 102 | CCAUUACGGUGCUGUCUAA | 293 | UUAGACAGCACCGUAAUGGGG | 100 | 20 nt + U |
| 2527 | 103 | GGUGCUGUCUAGGCAAGAA | 294 | UUCUUGCCUAGACAGCACCGU | 101 | 21 nt |
| 2647 | 104 | AAUCUGCUACAAUGGCGAA | 295 | UUCGCCAUUGUAGCAGAUUUC | 102 | 20 nt + U |
| 2761 | 105 | CAUGACCCGCUGCAUGAAA | 296 | UUUCAUGCAGCGGGUCAUGAG | 103 | 20 nt + U |
| 2762 | 106 | AUGACCCGCUGCAUGAACA | 297 | UGUUCAUGCAGCGGGUCAUGA | 104 | 20 nt + U |
| 2763 | 107 | UGACCCGCUGCAUGAACUA | 298 | UAGUUCAUGCAGCGGGUCAUG | 105 | 21 nt |
| 2764 | 108 | GACCCGCUGCAUGAACUAA | 299 | UUAGUUCAUGCAGCGGGUCAU | 106 | 20 nt + U |
| 2811 | 109 | CCAUCAUGAGAGACAUUAA | 300 | UUAAUGUCUCUCAUGAUGGCU | 107 | 21 nt |
| 2962 | 110 | GCUCUGCAGGUAUGACCCA | 301 | UGGGUCAUACCUGCAGAGCUC | 108 | 20 nt + U |
| 2975 | 111 | GACCCCGAAGGGGACAAUA | 302 | UAUUGUCCCCUUCGGGGUCAU | 109 | 21 nt |
| 2977 | 112 | CCCCGAAGGGGACAAUACA | 303 | UGUAUUGUCCCCUUCGGGGUC | 110 | 21 nt |
| 3028 | 113 | UGAGAGUGGAGGUAACCAA | 304 | UUGGUUACCUCCACUCUCAGG | 111 | 20 nt + U |
| 3032 | 114 | AGUGGAGGUAACCACAUAA | 305 | UUAUGUGGUUACCUCCACUCU | 112 | 20 nt + U |
| 3081 | 115 | GGAACCUCUAUCAUGAGAA | 306 | UUCUCAUGAUAGAGGUUCCUU | 113 | 21 nt |
| 3131 | 116 | GACGGAGGAAAUGGUAUUA | 307 | UAAUACCAUUUCCUCCGUCUU | 114 | 21 nt |
| 3134 | 117 | GGAGGAAAUGGUAUUAAGA | 308 | UCUUAAUACCAUUUCCUCCGU | 115 | 20 nt + U |
| 3141 | 118 | AUGGUAUUAAGCUCAUCAA | 309 | UUGAUGAGCUUAAUACCAUUU | 116 | 20 nt + U |
| 3144 | 119 | GUAUUAAGCUCAUCAUGGA | 310 | UCCAUGAUGAGCUUAAUACCA | 117 | 21 nt |
| 3146 | 120 | AUUAAGCUCAUCAUGGAAA | 311 | UUUCCAUGAUGAGCUUAAUAC | 118 | 20 nt + U |
| 3147 | 121 | UUAAGCUCAUCAUGGAAUA | 312 | UAUUCCAUGAUGAGCUUAAUA | 119 | 20 nt + U |
| 3159 | 122 | UGGAAUUUCUGCCUUCGGA | 313 | UCCGAAGGCAGAAAUUCCAUG | 120 | 20 nt + U |
| 3160 | 123 | GGAAUUUCUGCCUUCGGGA | 314 | UCCCGAAGGCAGAAAUUCCAU | 121 | 21 nt |
| 3229 | 124 | GCAGCUAAAAUAUGCCGUA | 315 | UACGGCAUAUUUUAGCUGCUG | 122 | 20 nt + U |
| 3247 | 125 | UCAGAUUUGUAAGGGGAUA | 316 | UAUCCCCUUACAAAUCUGAAC | 123 | 20 nt + U |
| 3250 | 126 | GAUUUGUAAGGGGAUGGAA | 317 | UUCCAUCCCCUUACAAAUCUG | 124 | 20 nt + U |
| 3251 | 127 | AUUUGUAAGGGGAUGGACA | 318 | UGUCCAUCCCCUUACAAAUCU | 125 | 20 nt + U |
| 3252 | 128 | UUUGUAAGGGGAUGGACUA | 319 | UAGUCCAUCCCCUUACAAAUC | 126 | 21 nt |
| 3254 | 129 | UGUAAGGGGAUGGACUAUA | 320 | UAUAGUCCAUCCCCUUACAAA | 127 | 20 nt + U |
| 3255 | 130 | GUAAGGGGAUGGACUAUUA | 321 | UAAUAGUCCAUCCCCUUACAA | 128 | 20 nt + U |
| 3258 | 131 | AGGGGAUGGACUAUUUGGA | 322 | UCCAAAUAGUCCAUCCCCUUA | 129 | 20 nt + U |
| 3259 | 132 | GGGGAUGGACUAUUUGGGA | 323 | UCCCAAAUAGUCCAUCCCCUU | 130 | 20 nt + U |
| 3260 | 133 | GGGAUGGACUAUUUGGGUA | 324 | UACCCAAAUAGUCCAUCCCCU | 131 | 20 nt + U |
| 3261 | 134 | GGAUGGACUAUUUGGGUUA | 325 | UAACCCAAAUAGUCCAUCCCC | 132 | 20 nt + U |
| 3265 | 135 | GGACUAUUUGGGUUCUCGA | 326 | UCGAGAACCCAAAUAGUCCAU | 133 | 20 nt + U |
| 3268 | 136 | CUAUUUGGGUUCUCGGCAA | 327 | UUGCCGAGAACCCAAAUAGUC | 134 | 21 nt |
| 3272 | 137 | UUGGGUUCUCGGCAAUACA | 328 | UGUAUUGCCGAGAACCCAAAU | 135 | 20 nt + U |
| 3275 | 138 | GGUUCUCGGCAAUACGUUA | 329 | UAACGUAUUGCCGAGAACCCA | 136 | 20 nt + U |
| 3276 | 139 | GUUCUCGGCAAUACGUUCA | 330 | UGAACGUAUUGCCGAGAACCC | 137 | 21 nt |
| 3278 | 140 | UCUCGGCAAUACGUUCACA | 331 | UGUGAACGUAUUGCCGAGAAC | 138 | 20 nt + U |
| 3279 | 141 | CUCGGCAAUACGUUCACCA | 332 | UGGUGAACGUAUUGCCGAGAA | 139 | 20 nt + U |
| 3281 | 142 | CGGCAAUACGUUCACCGGA | 333 | UCCGGUGAACGUAUUGCCGAG | 140 | 20 nt + U |
| 3282 | 143 | GGCAAUACGUUCACCGGGA | 334 | UCCCGGUGAACGUAUUGCCGA | 141 | 21 nt |
| 3283 | 144 | GCAAUACGUUCACCGGGAA | 335 | UUCCCGGUGAACGUAUUGCCG | 142 | 20 nt + U |
| 3284 | 145 | CAAUACGUUCACCGGGACA | 336 | UGUCCCGGUGAACGUAUUGCC | 143 | 20 nt + U |
| 3285 | 146 | AAUACGUUCACCGGGACUA | 337 | UAGUCCCGGUGAACGUAUUGC | 144 | 20 nt + U |
| 3286 | 147 | AUACGUUCACCGGGACUUA | 338 | UAAGUCCCGGUGAACGUAUUG | 145 | 20 nt + U |
| 3313 | 148 | AAAUGUCCUUGUUGAGAGA | 339 | UCUCUCAACAAGGACAUUUCU | 146 | 20 nt + U |
| 3314 | 149 | AAUGUCCUUGUUGAGAGUA | 340 | UACUCUCAACAAGGACAUUUC | 147 | 20 nt + U |
| 3323 | 150 | GUUGAGAGUGAACACCAAA | 341 | UUUGGUGUUCACUCUCAACAA | 148 | 20 nt + U |
| 3353 | 151 | GACUUCGGUUUAACCAAAA | 342 | UUUUGGUUAAACCGAAGUCUC | 149 | 20 nt + U |
| 3365 | 152 | ACCAAAGCAAUUGAAACCA | 343 | UGGUUUCAAUUGCUUUGGUUA | 150 | 20 nt + U |
| 3367 | 153 | CAAAGCAAUUGAAACCGAA | 344 | UUCGGUUUCAAUUGCUUUGGU | 151 | 20 nt + U |
| 3368 | 154 | AAAGCAAUUGAAACCGAUA | 345 | UAUCGGUUUCAAUUGCUUUGG | 152 | 21 nt |
| 3371 | 155 | GCAAUUGAAACCGAUAAGA | 346 | UCUUAUCGGUUUCAAUUGCUU | 153 | 20 nt + U |
| 3372 | 156 | CAAUUGAAACCGAUAAGGA | 347 | UCCUUAUCGGUUUCAAUUGCU | 154 | 21 nt |
| 3376 | 157 | UGAAACCGAUAAGGAGUAA | 348 | UUACUCCUUAUCGGUUUCAAU | 155 | 20 nt + U |
| 3409 | 158 | UGACCGGGACAGCCCUGUA | 349 | UACAGGGCUGUCCCGGUCAUC | 156 | 20 nt + U |
| 3505 | 159 | GCAUGAGCUGCUGACUUAA | 350 | UUAAGUCAGCAGCUCAUGCAG | 157 | 20 nt + U |
| 3556 | 160 | CCUGAAAAUGAUAGGCCCA | 351 | UGGGCCUAUCAUUUUCAGGAA | 158 | 21 nt |
| 3557 | 161 | CUGAAAAUGAUAGGCCCAA | 352 | UUGGGCCUAUCAUUUUCAGGA | 159 | 21 nt |
| 3558 | 162 | UGAAAAUGAUAGGCCCAAA | 353 | UUUGGGCCUAUCAUUUUCAGG | 160 | 20 nt + U |
| 3559 | 163 | GAAAAUGAUAGGCCCAACA | 354 | UGUUGGGCCUAUCAUUUUCAG | 161 | 20 nt + U |
| 3654 | 164 | CAGAUGAGGUUUAUCAACA | 355 | UGUUGAUAAACCUCAUCUGGA | 162 | 20 nt + U |
| 3662 | 165 | GUUUAUCAACUUAUGAGGA | 356 | UCCUCAUAAGUUGAUAAACCU | 163 | 21 nt |
| 3663 | 166 | UUUAUCAACUUAUGAGGAA | 357 | UUCCUCAUAAGUUGAUAAACC | 164 | 21 nt |
| 3683 | 167 | UGCUGGGAAUUCCAACCAA | 358 | UUGGUUGGAAUUCCCAGCAUU | 165 | 20 nt + U |
| 3689 | 168 | GAAUUCCAACCAUCCAAUA | 359 | UAUUGGAUGGUUGGAAUUCCC | 166 | 20 nt + U |
| 3694 | 169 | CCAACCAUCCAAUCGGACA | 360 | UGUCCGAUUGGAUGGUUGGAA | 167 | 21 nt |
| 3695 | 170 | CAACCAUCCAAUCGGACAA | 361 | UUGUCCGAUUGGAUGGUUGGA | 168 | 21 nt |
| 3698 | 171 | CCAUCCAAUCGGACAAGCA | 362 | UGCUUGUCCGAUUGGAUGGUU | 169 | 20 nt + U |
| 3702 | 172 | CCAAUCGGACAAGCUUUCA | 363 | UGAAAGCUUGUCCGAUUGGAU | 170 | 21 nt |
| 3719 | 173 | CAGAACCUUAUUGAAGGAA | 364 | UUCCUUCAAUAAGGUUCUGAA | 171 | 20 nt + U |
| 3781 | 174 | CCACAGAUUAUCAAGUCCA | 365 | UGGACUUGAUAAUCUGUGGAA | 172 | 20 nt + U |
| 3894 | 175 | CAUAUACAUGUAUAAGGCA | 366 | UGCCUUAUACAUGUAUAUGUA | 173 | 21 nt |
| 4099 | 176 | CGAGGAGUUGACCAAAAUA | 367 | UAUUUUGGUCAACUCCUCGUU | 174 | 21 nt |
| 4169 | 177 | GCUGGCACAUUAAUCAUAA | 368 | UUAUGAUUAAUGUGCCAGCAA | 175 | 20 nt + U |
| 4239 | 178 | CUAGACCAUGCAUUCUUAA | 369 | UUAAGAAUGCAUGGUCUAGUA | 176 | 21 nt |
| 4305 | 179 | GUAUUCUCUCACCAGUAGA | 370 | UCUACUGGUGAGAGAAUACAG | 177 | 20 nt + U |
| 4374 | 180 | ACUAGCACCCAUUUUUGAA | 371 | UUCAAAAAUGGGUGCUAGUGA | 178 | 20 nt + U |
| 4411 | 181 | GGGGAUAGCUGUGGAAUAA | 372 | UUAUUCCACAGCUAUCCCCCC | 179 | 20 nt + U |
| 4475 | 182 | GCUUUCCUAAGCAGUAUAA | 373 | UUAUACUGCUUAGGAAAGCAC | 180 | 20 nt + U |
| 4612 | 183 | GAAUGCUGUAUGCAACCAA | 374 | UUGGUUGCAUACAGCAUUCAA | 181 | 20 nt + U |
| 4671 | 184 | CAUACUUACCACCGAUCUA | 375 | UAGAUCGGUGGUAAGUAUGGA | 182 | 21 nt |
| 4672 | 185 | AUACUUACCACCGAUCUAA | 376 | UUAGAUCGGUGGUAAGUAUGG | 183 | 20 nt + U |
| 4679 | 186 | CCACCGAUCUACAAGGGUA | 377 | UACCCUUGUAGAUCGGUGGUA | 184 | 20 nt + U |
| 4682 | 187 | CCGAUCUACAAGGGUUGAA | 378 | UUCAACCCUUGUAGAUCGGUG | 185 | 20 nt + U |
| 4683 | 188 | CGAUCUACAAGGGUUGAUA | 379 | UAUCAACCCUUGUAGAUCGGU | 186 | 20 nt + U |
| 4684 | 189 | GAUCUACAAGGGUUGAUCA | 380 | UGAUCAACCCUUGUAGAUCGG | 187 | 20 nt + U |
| 4690 | 190 | CAAGGGUUGAUCCCUGUUA | 381 | UAACAGGGAUCAACCCUUGUA | 188 | 20 nt + U |
| 4794 | 191 | CAGUUUGCUUGGAGGUAGA | 382 | UCUACCUCCAAGCAAACUGAA | 189 | 20 nt + U |
| 4803 | 192 | UGGAGGUAGCUGGGUAAUA | 383 | UAUUACCCAGCUACCUCCAAG | 190 | 20 nt + U |
| 4807 | 193 | GGUAGCUGGGUAAUCAAAA | 384 | UUUUGAUUACCCAGCUACCUC | 191 | 21 nt |

### Table 2: Target Sequences and Seed Regions

Table 2 below presents the target sequence, the seed sequence and the seed target sequence of each of the compounds of the invention used in the Examples herein.

TABLE 2

| | | | | Target | | siRNA | Seed | Target |
|---|---|---|---|---|---|---|---|---|
| | Target | Target start | | sequence | Seed | seed | target | sequence |
| Duplex | SEQ | pos. in | Target sequence of | length | SEQ | region | SEQ ID | complementary |
| # | ID NO | NM_002227.4 | antisense strand | (nt) | ID NO | (nt 2-8) | NO | to seed |
| 1 | 385 | 614 | ACCGGAUGAGGUUCUAUUUCA | 21 | 576 | GAAAUAG | 767 | CUAUUUC |
| 2 | 386 | 673 | UGGCGUCAUUCUCCAAAGAA | 20 | 577 | UUCUUUG | 768 | CAAAGAA |
| 3 | 387 | 724 | CCAGAUGCAACCCCUCUCCU | 20 | 578 | AGGAGAG | 769 | CUCUCCU |
| 4 | 388 | 728 | AUGCAACCCCUCUCCUUGAU | 20 | 579 | AUCAAGG | 770 | CCUUGAU |
| 5 | 389 | 753 | CUCACUGGAGUAUCUGUUUG | 20 | 580 | CAAACAG | 771 | CUGUUUG |
| 6 | 390 | 756 | ACUGGAGUAUCUGUUUGCUCA | 21 | 581 | GAGCAAA | 772 | UUUGCUC |
| 7 | 391 | 818 | ACCCCAAGACCGAGCAGGAU | 20 | 582 | AUCCUGC | 773 | GCAGGAU |
| 8 | 392 | 874 | GUCCUGGCCAUCUCACACUA | 20 | 583 | UAGUGUG | 774 | CACACUA |
| 9 | 393 | 875 | UCCUGGCCAUCUCACACUAU | 20 | 584 | AUAGUGU | 775 | ACACUAU |
| 10 | 394 | 876 | CCUGGCCAUCUCACACUAUG | 20 | 585 | CAUAGUG | 776 | CACUAUG |
| 11 | 395 | 877 | CUGGCCAUCUCACACUAUGC | 20 | 586 | GCAUAGU | 777 | ACUAUGC |
| 12 | 396 | 878 | UGGCCAUCUCACACUAUGCCA | 21 | 587 | GGCAUAG | 778 | CUAUGCC |
| 13 | 397 | 883 | AUCUCACACUAUGCCAUGAU | 20 | 588 | AUCAUGG | 779 | CCAUGAU |
| 14 | 398 | 884 | UCUCACACUAUGCCAUGAUGA | 21 | 589 | CAUCAUG | 780 | CAUGAUG |
| 15 | 399 | 1069 | AGCAGCGUGUCCACGCAUGA | 20 | 590 | UCAUGCG | 781 | CGCAUGA |
| 16 | 400 | 1075 | GUGUCCACGCAUGACCUGAA | 20 | 591 | UUCAGGU | 782 | ACCUGAA |
| 17 | 401 | 1085 | AUGACCUGAAGGUGAAAUAC | 20 | 592 | GUAUUUC | 783 | GAAAUAC |
| 18 | 402 | 1107 | GGCUACCUUGGAAACUUUGA | 20 | 593 | UCAAAGU | 784 | ACUUUGA |
| 19 | 403 | 1108 | GCUACCUUGGAAACUUUGACA | 21 | 594 | GUCAAAG | 785 | CUUUGAC |
| 20 | 404 | 1138 | GGUGCUGAAAUAUUUGAGAC | 20 | 595 | GUCUCAA | 786 | UUGAGAC |
| 21 | 405 | 1182 | AAAUGAGAUGAAUUGGUUUCA | 21 | 596 | GAAACCA | 787 | UGGUUUC |
| 22 | 406 | 1189 | AUGAAUUGGUUUCAUUCGAA | 20 | 597 | UUCGAAU | 788 | AUUCGAA |
| 23 | 407 | 1190 | UGAAUUGGUUUCAUUCGAAU | 20 | 598 | AUUCGAA | 789 | UUCGAAU |
| 24 | 408 | 1304 | AAAAAAAUAAACUGAAGCGGA | 21 | 599 | CCGCUUC | 790 | GAAGCGG |
| 25 | 409 | 1306 | AAAAAUAAACUGAAGCGGAAA | 21 | 600 | UUCCGCU | 791 | AGCGGAA |
| 26 | 410 | 1311 | UAAACUGAAGCGGAAAAAAC | 20 | 601 | GUUUUUU | 792 | AAAAAAC |
| 27 | 411 | 1367 | AGAUCCGGGAAGAGUGGAACA | 21 | 602 | GUUCCAC | 793 | GUGGAAC |
| 28 | 412 | 1368 | GAUCCGGGAAGAGUGGAACAA | 21 | 603 | UGUUCCA | 794 | UGGAACA |
| 29 | 413 | 1372 | CGGGAAGAGUGGAACAAUUU | 20 | 604 | AAAUUGU | 795 | ACAAUUU |
| 30 | 414 | 1412 | CUCACAUUGUAAUAAAGGAG | 20 | 605 | CUCCUUU | 796 | AAAGGAG |
| 31 | 415 | 1413 | UCACAUUGUAAUAAAGGAGU | 20 | 606 | ACUCCUU | 797 | AAGGAGU |
| 32 | 416 | 1432 | UCUGUGGUCAGCAUUAACAA | 20 | 607 | UUGUUAA | 798 | UUAACAA |
| 33 | 417 | 1579 | CCCCCGUUGAUCGUCCACAA | 20 | 608 | UUGUGGA | 799 | UCCACAA |
| 34 | 418 | 1580 | CCCCGUUGAUCGUCCACAACA | 21 | 609 | GUUGUGG | 800 | CCACAAC |
| 35 | 419 | 1581 | CCCGUUGAUCGUCCACAACA | 20 | 610 | UGUUGUG | 801 | CACAACA |
| 36 | 420 | 1583 | CGUUGAUCGUCCACAACAUA | 20 | 611 | UAUGUUG | 802 | CAACAUA |
| 37 | 421 | 1584 | GUUGAUCGUCCACAACAUACA | 21 | 612 | GUAUGUU | 803 | AACAUAC |
| 38 | 422 | 1586 | UGAUCGUCCACAACAUACAGA | 21 | 613 | CUGUAUG | 804 | CAUACAG |
| 39 | 423 | 1587 | GAUCGUCCACAACAUACAGAA | 21 | 614 | UCUGUAU | 805 | AUACAGA |
| 40 | 424 | 1588 | AUCGUCCACAACAUACAGAA | 20 | 615 | UUCUGUA | 806 | UACAGAA |
| 41 | 425 | 1595 | ACAACAUACAGAAUGGCUGU | 20 | 616 | ACAGCCA | 807 | UGGCUGU |
| 42 | 426 | 1596 | CAACAUACAGAAUGGCUGUCA | 21 | 617 | GACAGCC | 808 | GGCUGUC |
| 43 | 427 | 1601 | UACAGAAUGGCUGUCAUGGU | 20 | 618 | ACCAUGA | 809 | UCAUGGU |
| 44 | 428 | 1602 | ACAGAAUGGCUGUCAUGGUC | 20 | 619 | GACCAUG | 810 | CAUGGUC |
| 45 | 429 | 1603 | CAGAAUGGCUGUCAUGGUCCA | 21 | 620 | GGACCAU | 811 | AUGGUCC |
| 46 | 430 | 1608 | UGGCUGUCAUGGUCCAAUCU | 20 | 621 | AGAUUGG | 812 | CCAAUCU |
| 47 | 431 | 1609 | GGCUGUCAUGGUCCAAUCUG | 20 | 622 | CAGAUUG | 813 | CAAUCUG |
| 48 | 432 | 1611 | CUGUCAUGGUCCAAUCUGUA | 20 | 623 | UACAGAU | 814 | AUCUGUA |
| 49 | 433 | 1640 | CCAUCAAUAAAUUGCGGCAA | 20 | 624 | UUGCCGC | 815 | GCGGCAA |
| 50 | 434 | 1642 | AUCAAUAAAUUGCGGCAAGAA | 21 | 625 | UCUUGCC | 816 | GGCAAGA |
| 51 | 435 | 1671 | GGAGGGGAUGUACGUGCUGA | 20 | 626 | UCAGCAC | 817 | GUGCUGA |
| 52 | 436 | 1672 | GAGGGGAUGUACGUGCUGAG | 20 | 627 | CUCAGCA | 818 | UGCUGAG |
| 53 | 437 | 1673 | AGGGGAUGUACGUGCUGAGG | 20 | 628 | CCUCAGC | 819 | GCUGAGG |
| 54 | 438 | 1674 | GGGGAUGUACGUGCUGAGGU | 20 | 629 | ACCUCAG | 820 | CUGAGGU |
| 55 | 439 | 1677 | GAUGUACGUGCUGAGGUGGA | 20 | 630 | UCCACCU | 821 | AGGUGGA |
| 56 | 440 | 1678 | AUGUACGUGCUGAGGUGGAG | 20 | 631 | CUCCACC | 822 | GGUGGAG |
| 57 | 441 | 1690 | AGGUGGAGCUGCACCGACUU | 20 | 632 | AAGUCGG | 823 | CCGACUU |
| 58 | 442 | 1692 | GUGGAGCUGCACCGACUUUGA | 21 | 633 | CAAAGUC | 824 | GACUUUG |
| 59 | 443 | 1698 | CUGCACCGACUUUGACAACA | 20 | 634 | UGUUGUC | 825 | GACAACA |
| 60 | 444 | 1699 | UGCACCGACUUUGACAACAU | 20 | 635 | AUGUUGU | 826 | ACAACAU |
| 61 | 445 | 1723 | AUGACCGUCACCUGCUUUGA | 20 | 636 | UCAAAGC | 827 | GCUUUGA |
| 62 | 446 | 1769 | AGAAGCAGUUCAAGAACUUU | 20 | 637 | AAAGUUC | 828 | GAACUUU |
| 63 | 447 | 1770 | GAAGCAGUUCAAGAACUUUCA | 21 | 638 | GAAAGUU | 829 | AACUUUC |
| 64 | 448 | 1780 | AAGAACUUUCAGAUCGAGGU | 20 | 639 | ACCUCGA | 830 | UCGAGGU |
| 65 | 449 | 1798 | GUGCAGAAGGGCCGCUACAG | 20 | 640 | CUGUAGC | 831 | GCUACAG |
| 66 | 450 | 1876 | AAGCAGAUCCUGCGCACGGA | 20 | 641 | UCCGUGC | 832 | GCACGGA |
| 67 | 451 | 1927 | CAGCCCAAGCCCCGAGAAAU | 20 | 642 | AUUUCUC | 833 | GAGAAAU |
| 68 | 452 | 1928 | AGCCCAAGCCCCGAGAAAUC | 20 | 643 | GAUUUCU | 834 | AGAAAUC |
| 69 | 453 | 1929 | GCCCAAGCCCCGAGAAAUCU | 20 | 644 | AGAUUUC | 835 | GAAAUCU |
| 70 | 454 | 1936 | CCCCGAGAAAUCUCCAACCU | 20 | 645 | AGGUUGG | 836 | CCAACCU |
| 71 | 455 | 1952 | ACCUGCUGGUGGCUACUAAGA | 21 | 646 | CUUAGUA | 837 | UACUAAG |
| 72 | 456 | 1954 | CUGCUGGUGGCUACUAAGAAA | 21 | 647 | UUCUUAG | 838 | CUAAGAA |
| 73 | 457 | 1956 | GCUGGUGGCUACUAAGAAAG | 20 | 648 | CUUUCUU | 839 | AAGAAAG |
| 74 | 458 | 1958 | UGGUGGCUACUAAGAAAGCC | 20 | 649 | GGCUUUC | 840 | GAAAGCC |
| 75 | 459 | 1978 | CAGGAGUGGCAGCCCGUCUA | 20 | 650 | UAGACGG | 841 | CCGUCUA |
| 76 | 460 | 2066 | GAGGCACGAGAACACACAUC | 20 | 651 | GAUGUGU | 842 | ACACAUC |
| 77 | 461 | 2068 | GGCACGAGAACACACAUCUA | 20 | 652 | UAGAUGU | 843 | ACAUCUA |
| 78 | 462 | 2102 | UGGAUUACAAGGAUGACGAA | 20 | 653 | UUCGUCA | 844 | UGACGAA |
| 79 | 463 | 2111 | AGGAUGACGAAGGAACUUCU | 20 | 654 | AGAAGUU | 845 | AACUUCU |
| 80 | 464 | 2138 | AGAAGAUAAAAGUGAUCCUCA | 21 | 655 | GAGGAUC | 846 | GAUCCUC |
| 81 | 465 | 2146 | AAAGUGAUCCUCAAAGUCUUA | 21 | 656 | AAGACUU | 847 | AAGUCUU |
| 82 | 466 | 2148 | AGUGAUCCUCAAAGUCUUAGA | 21 | 657 | CUAAGAC | 848 | GUCUUAG |
| 83 | 467 | 2205 | GGCAGCCAGCAUGAUGAGACA | 21 | 658 | GUCUCAU | 849 | AUGAGAC |
| 84 | 468 | 2206 | GCAGCCAGCAUGAUGAGACA | 20 | 659 | UGUCUCA | 850 | UGAGACA |
| 85 | 469 | 2218 | AUGAGACAGGUCUCCCACAAA | 21 | 660 | UUGUGGG | 851 | CCCACAA |
| 86 | 470 | 2229 | CUCCCACAAACACAUCGUGUA | 21 | 661 | ACACGAU | 852 | AUCGUGU |
| 87 | 471 | 2230 | UCCCACAAACACAUCGUGUA | 20 | 662 | UACACGA | 853 | UCGUGUA |
| 88 | 472 | 2237 | AACACAUCGUGUACCUCUAU | 20 | 663 | AUAGAGG | 854 | CCUCUAU |
| 89 | 473 | 2238 | ACACAUCGUGUACCUCUAUG | 20 | 664 | CAUAGAG | 855 | CUCUAUG |
| 90 | 474 | 2239 | CACAUCGUGUACCUCUAUGG | 20 | 665 | CCAUAGA | 856 | UCUAUGG |
| 91 | 475 | 2269 | CGCGACGUGGAGAAUAUCAU | 20 | 666 | AUGAUAU | 857 | AUAUCAU |
| 92 | 476 | 2308 | GGGGGUCCUCUGGAUCUCUU | 20 | 667 | AAGAGAU | 858 | AUCUCUU |
| 93 | 477 | 2317 | CUGGAUCUCUUCAUGCACCG | 20 | 668 | CGGUGCA | 859 | UGCACCG |
| 94 | 478 | 2318 | UGGAUCUCUUCAUGCACCGGA | 21 | 669 | CCGGUGC | 860 | GCACCGG |
| 95 | 479 | 2319 | GGAUCUCUUCAUGCACCGGAA | 21 | 670 | UCCGGUG | 861 | CACCGGA |
| 96 | 480 | 2320 | GAUCUCUUCAUGCACCGGAAA | 21 | 671 | UUCCGGU | 862 | ACCGGAA |
| 97 | 481 | 2321 | AUCUCUUCAUGCACCGGAAAA | 21 | 672 | UUUCCGG | 863 | CCGGAAA |
| 98 | 482 | 2322 | UCUCUUCAUGCACCGGAAAA | 20 | 673 | UUUUCCG | 864 | CGGAAAA |
| 99 | 483 | 2323 | CUCUUCAUGCACCGGAAAAG | 20 | 674 | CUUUUCC | 865 | GGAAAAG |
| 100 | 484 | 2520 | CCCCAUUACGGUGCUGUCUA | 20 | 675 | UAGACAG | 866 | CUGUCUA |
| 101 | 485 | 2527 | ACGGUGCUGUCUAGGCAAGAA | 21 | 676 | UCUUGCC | 867 | GGCAAGA |
| 102 | 486 | 2647 | GAAAUCUGCUACAAUGGCGA | 20 | 677 | UCGCCAU | 868 | AUGGCGA |
| 103 | 487 | 2761 | CUCAUGACCCGCUGCAUGAA | 20 | 678 | UUCAUGC | 869 | GCAUGAA |
| 104 | 488 | 2762 | UCAUGACCCGCUGCAUGAAC | 20 | 679 | GUUCAUG | 870 | CAUGAAC |
| 105 | 489 | 2763 | CAUGACCCGCUGCAUGAACUA | 21 | 680 | AGUUCAU | 871 | AUGAACU |
| 106 | 490 | 2764 | AUGACCCGCUGCAUGAACUA | 20 | 681 | UAGUUCA | 872 | UGAACUA |
| 107 | 491 | 2811 | AGCCAUCAUGAGAGACAUUAA | 21 | 682 | UAAUGUC | 873 | GACAUUA |
| 108 | 492 | 2962 | GAGCUCUGCAGGUAUGACCC | 20 | 683 | GGGUCAU | 874 | AUGACCC |
| 109 | 493 | 2975 | AUGACCCCGAAGGGGACAAUA | 21 | 684 | AUUGUCC | 875 | GGACAAU |
| 110 | 494 | 2977 | GACCCCGAAGGGGACAAUACA | 21 | 685 | GUAUUGU | 876 | ACAAUAC |
| 111 | 495 | 3028 | CCUGAGAGUGGAGGUAACCA | 20 | 686 | UGGUUAC | 877 | GUAACCA |
| 112 | 496 | 3032 | AGAGUGGAGGUAACCACAUA | 20 | 687 | UAUGUGG | 878 | CCACAUA |
| 113 | 497 | 3081 | AAGGAACCUCUAUCAUGAGAA | 21 | 688 | UCUCAUG | 879 | CAUGAGA |
| 114 | 498 | 3131 | AAGACGGAGGAAAUGGUAUUA | 21 | 689 | AAUACCA | 880 | UGGUAUU |
| 115 | 499 | 3134 | ACGGAGGAAAUGGUAUUAAG | 20 | 690 | CUUAAUA | 881 | UAUUAAG |
| 116 | 500 | 3141 | AAAUGGUAUUAAGCUCAUCA | 20 | 691 | UGAUGAG | 882 | CUCAUCA |
| 117 | 501 | 3144 | UGGUAUUAAGCUCAUCAUGGA | 21 | 692 | CCAUGAU | 883 | AUCAUGG |
| 118 | 502 | 3146 | GUAUUAAGCUCAUCAUGGAA | 20 | 693 | UUCCAUG | 884 | CAUGGAA |
| 119 | 503 | 3147 | UAUUAAGCUCAUCAUGGAAU | 20 | 694 | AUUCCAU | 885 | AUGGAAU |
| 120 | 504 | 3159 | CAUGGAAUUUCUGCCUUCGG | 20 | 695 | CCGAAGG | 886 | CCUUCGG |
| 121 | 505 | 3160 | AUGGAAUUUCUGCCUUCGGGA | 21 | 696 | CCCGAAG | 887 | CUUCGGG |
| 122 | 506 | 3229 | CAGCAGCUAAAAUAUGCCGU | 20 | 697 | ACGGCAU | 888 | AUGCCGU |
| 123 | 507 | 3247 | GUUCAGAUUUGUAAGGGGAU | 20 | 698 | AUCCCCU | 889 | AGGGGAU |
| 124 | 508 | 3250 | CAGAUUUGUAAGGGGAUGGA | 20 | 699 | UCCAUCC | 890 | GGAUGGA |
| 125 | 509 | 3251 | AGAUUUGUAAGGGGAUGGAC | 20 | 700 | GUCCAUC | 891 | GAUGGAC |
| 126 | 510 | 3252 | GAUUUGUAAGGGGAUGGACUA | 21 | 701 | AGUCCAU | 892 | AUGGACU |
| 127 | 511 | 3254 | UUUGUAAGGGGAUGGACUAU | 20 | 702 | AUAGUCC | 893 | GGACUAU |
| 128 | 512 | 3255 | UUGUAAGGGGAUGGACUAUU | 20 | 703 | AAUAGUC | 894 | GACUAUU |
| 129 | 513 | 3258 | UAAGGGGAUGGACUAUUUGG | 20 | 704 | CCAAAUA | 895 | UAUUUGG |
| 130 | 514 | 3259 | AAGGGGAUGGACUAUUUGGG | 20 | 705 | CCCAAAU | 896 | AUUUGGG |
| 131 | 515 | 3260 | AGGGGAUGGACUAUUUGGGU | 20 | 706 | ACCCAAA | 897 | UUUGGGU |
| 132 | 516 | 3261 | GGGGAUGGACUAUUUGGGUU | 20 | 707 | AACCCAA | 898 | UUGGGUU |
| 133 | 517 | 3265 | AUGGACUAUUUGGGUUCUCG | 20 | 708 | CGAGAAC | 899 | GUUCUCG |
| 134 | 518 | 3268 | GACUAUUUGGGUUCUCGGCAA | 21 | 709 | UGCCGAG | 900 | CUCGGCA |
| 135 | 519 | 3272 | AUUUGGGUUCUCGGCAAUAC | 20 | 710 | GUAUUGC | 901 | GCAAUAC |
| 136 | 520 | 3275 | UGGGUUCUCGGCAAUACGUU | 20 | 711 | AACGUAU | 902 | AUACGUU |
| 137 | 521 | 3276 | GGGUUCUCGGCAAUACGUUCA | 21 | 712 | GAACGUA | 903 | UACGUUC |
| 138 | 522 | 3278 | GUUCUCGGCAAUACGUUCAC | 20 | 713 | GUGAACG | 904 | CGUUCAC |
| 139 | 523 | 3279 | UUCUCGGCAAUACGUUCACC | 20 | 714 | GGUGAAC | 905 | GUUCACC |
| 140 | 524 | 3281 | CUCGGCAAUACGUUCACCGG | 20 | 715 | CCGGUGA | 906 | UCACCGG |
| 141 | 525 | 3282 | UCGGCAAUACGUUCACCGGGA | 21 | 716 | CCCGGUG | 907 | CACCGGG |
| 142 | 526 | 3283 | CGGCAAUACGUUCACCGGGA | 20 | 717 | UCCCGGU | 908 | ACCGGGA |
| 143 | 527 | 3284 | GGCAAUACGUUCACCGGGAC | 20 | 718 | GUCCCGG | 909 | CCGGGAC |
| 144 | 528 | 3285 | GCAAUACGUUCACCGGGACU | 20 | 719 | AGUCCCG | 910 | CGGGACU |
| 145 | 529 | 3286 | CAAUACGUUCACCGGGACUU | 20 | 720 | AAGUCCC | 911 | GGGACUU |
| 146 | 530 | 3313 | AGAAAUGUCCUUGUUGAGAG | 20 | 721 | CUCUCAA | 912 | UUGAGAG |
| 147 | 531 | 3314 | GAAAUGUCCUUGUUGAGAGU | 20 | 722 | ACUCUCA | 913 | UGAGAGU |
| 148 | 532 | 3323 | UUGUUGAGAGUGAACACCAA | 20 | 723 | UUGGUGU | 914 | ACACCAA |
| 149 | 533 | 3353 | GAGACUUCGGUUUAACCAAA | 20 | 724 | UUUGGUU | 915 | AACCAAA |
| 150 | 534 | 3365 | UAACCAAAGCAAUUGAAACC | 20 | 725 | GGUUUCA | 916 | UGAAACC |
| 151 | 535 | 3367 | ACCAAAGCAAUUGAAACCGA | 20 | 726 | UCGGUUU | 917 | AAACCGA |
| 152 | 536 | 3368 | CCAAAGCAAUUGAAACCGAUA | 21 | 727 | AUCGGUU | 918 | AACCGAU |
| 153 | 537 | 3371 | AAGCAAUUGAAACCGAUAAG | 20 | 728 | CUUAUCG | 919 | CGAUAAG |
| 154 | 538 | 3372 | AGCAAUUGAAACCGAUAAGGA | 21 | 729 | CCUUAUC | 920 | GAUAAGG |
| 155 | 539 | 3376 | AUUGAAACCGAUAAGGAGUA | 20 | 730 | UACUCCU | 921 | AGGAGUA |
| 156 | 540 | 3409 | GAUGACCGGGACAGCCCUGU | 20 | 731 | ACAGGGC | 922 | GCCCUGU |
| 157 | 541 | 3505 | CUGCAUGAGCUGCUGACUUA | 20 | 732 | UAAGUCA | 923 | UGACUUA |
| 158 | 542 | 3556 | UUCCUGAAAAUGAUAGGCCCA | 21 | 733 | GGGCCUA | 924 | UAGGCCC |
| 159 | 543 | 3557 | UCCUGAAAAUGAUAGGCCCAA | 21 | 734 | UGGGCCU | 925 | AGGCCCA |
| 160 | 544 | 3558 | CCUGAAAAUGAUAGGCCCAA | 20 | 735 | UUGGGCC | 926 | GGCCCAA |
| 161 | 545 | 3559 | CUGAAAAUGAUAGGCCCAAC | 20 | 736 | GUUGGGC | 927 | GCCCAAC |
| 162 | 546 | 3654 | UCCAGAUGAGGUUUAUCAAC | 20 | 737 | GUUGAUA | 928 | UAUCAAC |
| 163 | 547 | 3662 | AGGUUUAUCAACUUAUGAGGA | 21 | 738 | CCUCAUA | 929 | UAUGAGG |
| 164 | 548 | 3663 | GGUUUAUCAACUUAUGAGGAA | 21 | 739 | UCCUCAU | 930 | AUGAGGA |
| 165 | 549 | 3683 | AAUGCUGGGAAUUCCAACCA | 20 | 740 | UGGUUGG | 931 | CCAACCA |
| 166 | 550 | 3689 | GGGAAUUCCAACCAUCCAAU | 20 | 741 | AUUGGAU | 932 | AUCCAAU |
| 167 | 551 | 3694 | UUCCAACCAUCCAAUCGGACA | 21 | 742 | GUCCGAU | 933 | AUCGGAC |
| 168 | 552 | 3695 | UCCAACCAUCCAAUCGGACAA | 21 | 743 | UGUCCGA | 934 | UCGGACA |
| 169 | 553 | 3698 | AACCAUCCAAUCGGACAAGC | 20 | 744 | GCUUGUC | 935 | GACAAGC |
| 170 | 554 | 3702 | AUCCAAUCGGACAAGCUUUCA | 21 | 745 | GAAAGCU | 936 | AGCUUUC |
| 171 | 555 | 3719 | UUCAGAACCUUAUUGAAGGA | 20 | 746 | UCCUUCA | 937 | UGAAGGA |
| 172 | 556 | 3781 | UUCCACAGAUUAUCAAGUCC | 20 | 747 | GGACUUG | 938 | CAAGUCC |
| 173 | 557 | 3894 | UACAUAUACAUGUAUAAGGCA | 21 | 748 | GCCUUAU | 939 | AUAAGGC |
| 174 | 558 | 4099 | AACGAGGAGUUGACCAAAAUA | 21 | 749 | AUUUUGG | 940 | CCAAAAU |
| 175 | 559 | 4169 | UUGCUGGCACAUUAAUCAUA | 20 | 750 | UAUGAUU | 941 | AAUCAUA |
| 176 | 560 | 4239 | UACUAGACCAUGCAUUCUUAA | 21 | 751 | UAAGAAU | 942 | AUUCUUA |
| 177 | 561 | 4305 | CUGUAUUCUCUCACCAGUAG | 20 | 752 | CUACUGG | 943 | CCAGUAG |
| 178 | 562 | 4374 | UCACUAGCACCCAUUUUUGA | 20 | 753 | UCAAAAA | 944 | UUUUUGA |
| 179 | 563 | 4411 | GGGGGGAUAGCUGUGGAAUA | 20 | 754 | UAUUCCA | 945 | UGGAAUA |
| 180 | 564 | 4475 | GUGCUUUCCUAAGCAGUAUA | 20 | 755 | UAUACUG | 946 | CAGUAUA |
| 181 | 565 | 4612 | UUGAAUGCUGUAUGCAACCA | 20 | 756 | UGGUUGC | 947 | GCAACCA |
| 182 | 566 | 4671 | UCCAUACUUACCACCGAUCUA | 21 | 757 | AGAUCGG | 948 | CCGAUCU |
| 183 | 567 | 4672 | CCAUACUUACCACCGAUCUA | 20 | 758 | UAGAUCG | 949 | CGAUCUA |
| 184 | 568 | 4679 | UACCACCGAUCUACAAGGGU | 20 | 759 | ACCCUUG | 950 | CAAGGGU |
| 185 | 569 | 4682 | CACCGAUCUACAAGGGUUGA | 20 | 760 | UCAACCC | 951 | GGGUUGA |
| 186 | 570 | 4683 | ACCGAUCUACAAGGGUUGAU | 20 | 761 | AUCAACC | 952 | GGUUGAU |
| 187 | 571 | 4684 | CCGAUCUACAAGGGUUGAUC | 20 | 762 | GAUCAAC | 953 | GUUGAUC |
| 188 | 572 | 4690 | UACAAGGGUUGAUCCCUGUU | 20 | 763 | AACAGGG | 954 | CCCUGUU |
| 189 | 573 | 4794 | UUCAGUUUGCUUGGAGGUAG | 20 | 764 | CUACCUC | 955 | GAGGUAG |
| 190 | 574 | 4803 | CUUGGAGGUAGCUGGGUAAU | 20 | 765 | AUUACCC | 956 | GGGUAAU |
| 191 | 575 | 4807 | GAGGUAGCUGGGUAAUCAAAA | 21 | 766 | UUUGAUU | 957 | AAUCAAA |

### Table 3: Chemistry of Sense and Antisense Strands

Table 3 below presents the sense strand and antisense strand of each of the compounds of the invention used in the Examples herein. The chemistry of the 2′ position of each nucleoside and of each internucleotide linkage are shown according to the following code.

Lower case a, c, g or u indicates 2′-O-methyl modified nucleotide.

Upper case A, C, G or U followed by f (i.e. Af, Cf, Gf or Uf) indicates 2′-fluoro modified nucleotide.

(vin) indicates Vinyl-phosphonate 2′-OMe RNA. Therefore, (vinu) indicates Vinyl-phosphonate 2′-OMe uracil

Lower case s represents phosphorothioate internucleotide linkages. The absence of s indicates phosphodiester internucleotide linkages. The chemical modification pattern (also referred to herein as “parent design”) of all compounds in Table 3 is as follows:

Modification pattern of sense strand from 5′ to 3′:
5′-[2′OMe]-PS-[2′OMe]-PS-[2′F]-[2′OMe]-[2′F]-[2′OMe]-[2′F]-[2′OMe]-[2′F]-[2′OMe]-[2′F]-[2′OMe]-[2′F]-[2′OMe]-[2′F]-[2′OMe]-[2′F]-PS-[2′OMe]-PS-[2′F]-3′

Modification pattern of antisense strand from 3′ to 5′:
3′-[2′OMe]-PS-[2′OMe]-PS-[2′OMe]-[2′F]-[2′OMe]-[2′F]-[2′OMe]-[2′F]-[2′OMe]-[2′F]-[2′OMe]-[2′F]-[2′OMe]-[2′F]-[2′OMe]-[2′F]-[2′OMe]-[2′F]-[2′OMe]-PS-[2′F]-PS-[VP-2′-OMe]-5′
with [2′OMe] representing a 2′-OMe RNA,
[2′F] representing a 2′-F RNA,
[VP-2′-OMe] representing a Vinyl-phosphonate 2′-OMe RNA, and
PS representing phosphorothioate internucleotide linkages (in the absence of mention, the internucleotide linkage is a phosphodiester internucleotide linkage).

TABLE 3

| Compound | | | Anti- | |
|---|---|---|---|---|
| # (Start | Sense | | sense | |
| pos. in | strand | | strand | |
| NM_ | SEQ ID | | SEQ ID | |
| 002227.4) | NO | Sense strand (5′-3′) | NO | Antisense strand (5′-3′) |
| 614 | 958 | csgsGfaUfgAfgGfuUfcUfaUfuUfscsAf | 1149 | (vinu)sGfsaAfaUfaGfaAfcCfuCfaUfcCfgsgsu |
| 673 | 959 | gscsGfuCfaUfuCfuCfcAfaAfgAfsasAf | 1150 | (vinu)sUfsuCfuUfuGfgAfgAfaUfgAfcGfcscsa |
| 724 | 960 | asgsAfuGfcAfaCfcCfcUfcUfcCfsusAf | 1151 | (vinu)sAfsgGfaGfaGfgGfgUfuGfcAfuCfusgsg |
| 728 | 961 | gscsAfaCfcCfcUfcUfcCfuUfgAfsusAf | 1152 | (vinu)sAfsuCfaAfgGfaGfaGfgGfgUfuGfcsasu |
| 753 | 962 | csasCfuGfgAfgUfaUfcUfgUfuUfsgsAf | 1153 | (vinu)sCfsaAfaCfaGfaUfaCfuCfcAfgUfgsasg |
| 756 | 963 | usgsGfaGfuAfuCfuGfuUfuGfcUfscsAf | 1154 | (vinu)sGfsaGfcAfaAfcAfgAfuAfcUfcCfasgsu |
| 818 | 964 | cscsCfaAfgAfcCfgAfgCfaGfgAfsusAf | 1155 | (vinu)sAfsuCfcUfgCfuCfgGfuCfuUfgGfgsgsu |
| 874 | 965 | cscsUfgGfcCfaUfcUfcAfcAfcUfsasAf | 1156 | (vinu)sUfsaGfuGfuGfaGfaUfgGfcCfaGfgsasc |
| 875 | 966 | csusGfgCfcAfuCfuCfaCfaCfuAfsusAf | 1157 | (vinu)sAfsuAfgUfgUfgAfgAfuGfgCfcAfgsgsa |
| 876 | 967 | usgsGfcCfaUfcUfcAfcAfcUfaUfsgsAf | 1158 | (vinu)sCfsaUfaGfuGfuGfaGfaUfgGfcCfasgsg |
| 877 | 968 | gsgsCfcAfuCfuCfaCfaCfuAfuGfscsAf | 1159 | (vinu)sGfscAfuAfgUfgUfgAfgAfuGfgCfcsasg |
| 878 | 969 | gscsCfaUfcUfcAfcAfcUfaUfgCfscsAf | 1160 | (vinu)sGfsgCfaUfaGfuGfuGfaGfaUfgGfcscsa |
| 883 | 670 | csusCfaCfaCfuAfuGfcCfaUfgAfsusAf | 1161 | (vinu)sAfsuCfaUfgGfcAfuAfgUfgUfgAfgsasu |
| 884 | 971 | uscsAfcAfcUfaUfgCfcAfuGfaUfsgsAf | 1162 | (vinu)sCfsaUfcAfuGfgCfaUfaGfuGfuGfasgsa |
| 1069 | 972 | csasGfcGfuGfuCfcAfcGfcAfuGfsasAf | 1163 | (vinu)sUfscAfuGfcGfuGfgAfcAfcGfcUfgscsu |
| 1075 | 973 | gsusCfcAfcGfcAfuGfaCfcUfgAfsasAf | 1164 | (vinu)sUfsuCfaGfgUfcAfuGfcGfuGfgAfcsasc |
| 1085 | 974 | gsasCfcUfgAfaGfgUfgAfaAfuAfscsAf | 1165 | (vinu)sGfsuAfuUfuCfaCfcUfuCfaGfgUfcsasu |
| 1107 | 975 | csusAfcCfuUfgGfaAfaCfuUfuGfsasAf | 1166 | (vinu)sUfscAfaAfgUfuUfcCfaAfgGfuAfgscsc |
| 1108 | 976 | usasCfcUfuGfgAfaAfcUfuUfgAfscsAf | 1167 | (vinu)sGfsuCfaAfaGfuUfuCfcAfaGfgUfasgsc |
| 1138 | 977 | usgsCfuGfaAfaUfaUfuUfgAfgAfscsAf | 1168 | (vinu)sGfsuCfuCfaAfaUfaUfuUfcAfgCfascsc |
| 1182 | 978 | asusGfaGfaUfgAfaUfuGfgUfuUfscsAf | 1169 | (vinu)sGfsaAfaCfcAfaUfuCfaUfcUfcAfususu |
| 1189 | 979 | gsasAfuUfgGfuUfuCfaUfuCfgAfsasAf | 1170 | (vinu)sUfsuCfgAfaUfgAfaAfcCfaAfuUfcsasu |
| 1190 | 980 | asasUfuGfgUfuUfcAfuUfcGfaAfsusAf | 1171 | (vinu)sAfsuUfcGfaAfuGfaAfaCfcAfaUfuscsa |
| 1304 | 981 | asasAfaAfuAfaAfcUfgAfaGfcGfsgsAf | 1172 | (vinu)sCfscGfcUfuCfaGfuUfuAfuUfuUfususu |
| 1306 | 982 | asasAfuAfaAfcUfgAfaGfcGfgAfsasAf | 1173 | (vinu)sUfsuCfcGfcUfuCfaGfuUfuAfuUfususu |
| 1311 | 983 | asasCfuGfaAfgCfgGfaAfaAfaAfscsAf | 1174 | (vinu)sGfsuUfuUfuUfcCfgCfuUfcAfgUfususa |
| 1367 | 984 | asusCfcGfgGfaAfgAfgUfgGfaAfscsAf | 1175 | (vinu)sGfsuUfcCfaCfuCfuUfcCfcGfgAfuscsu |
| 1368 | 985 | uscsCfgGfgAfaGfaGfuGfgAfaCfsasAf | 1176 | (vinu)sUfsgUfuCfcAfcUfcUfuCfcCfgGfasusc |
| 1372 | 986 | gsgsAfaGfaGfuGfgAfaCfaAfuUfsusAf | 1177 | (vinu)sAfsaAfuUfgUfuCfcAfcUfcUfuCfcscsg |
| 1412 | 987 | csasCfaUfuGfuAfaUfaAfaGfgAfsgsAf | 1178 | (vinu)sCfsuCfcUfuUfaUfuAfcAfaUfgUfgsasg |
| 1413 | 988 | ascsAfuUfgUfaAfuAfaAfgGfaGfsusAf | 1179 | (vinu)sAfscUfcCfuUfuAfuUfaCfaAfuGfusgsa |
| 1432 | 989 | usgsUfgGfuCfaGfcAfuUfaAfcAfsasAf | 1180 | (vinu)sUfsuGfuUfaAfuGfcUfgAfcCfaCfasgsa |
| 1579 | 990 | cscsCfgUfuGfaUfcGfuCfcAfcAfsasAf | 1181 | (vinu)sUfsuGfuGfgAfcGfaUfcAfaCfgGfgsgsg |
| 1580 | 991 | cscsGfuUfgAfuCfgUfcCfaCfaAfscsAf | 1182 | (vinu)sGfsuUfgUfgGfaCfgAfuCfaAfcGfgsgsg |
| 1581 | 992 | csgsUfuGfaUfcGfuCfcAfcAfaCfsasAf | 1183 | (vinu)sUfsgUfuGfuGfgAfcGfaUfcAfaCfgsgsg |
| 1583 | 993 | ususGfaUfcGfuCfcAfcAfaCfaUfsasAf | 1184 | (vinu)sUfsaUfgUfuGfuGfgAfcGfaUfcAfascsg |
| 1584 | 994 | usgsAfuCfgUfcCfaCfaAfcAfuAfscsAf | 1185 | (vinu)sGfsuAfuGfuUfgUfgGfaCfgAfuCfasasc |
| 1586 | 995 | asusCfgUfcCfaCfaAfcAfuAfcAfsgsAf | 1186 | (vinu)sCfsuGfuAfuGfuUfgUfgGfaCfgAfuscsa |
| 1587 | 996 | uscsGfuCfcAfcAfaCfaUfaCfaGfsasAf | 1187 | (vinu)sUfscUfgUfaUfgUfuGfuGfgAfcGfasusc |
| 1588 | 997 | csgsUfcCfaCfaAfcAfuAfcAfgAfsasAf | 1188 | (vinu)sUfsuCfuGfuAfuGfuUfgUfgGfaCfgsasu |
| 1595 | 998 | asasCfaUfaCfaGfaAfuGfgCfuGfsusAf | 1189 | (vinu)sAfscAfgCfcAfuUfcUfgUfaUfgUfusgsu |
| 1596 | 999 | ascsAfuAfcAfgAfaUfgGfcUfgUfscsAf | 1190 | (vinu)sGfsaCfaGfcCfaUfuCfuGfuAfuGfususg |
| 1601 | 1000 | csasGfaAfuGfgCfuGfuCfaUfgGfsusAf | 1191 | (vinu)sAfscCfaUfgAfcAfgCfcAfuUfcUfgsusa |
| 1602 | 1001 | asgsAfaUfgGfcUfgUfcAfuGfgUfscsAf | 1192 | (vinu)sGfsaCfcAfuGfaCfaGfcCfaUfuCfusgsu |
| 1603 | 1002 | gsasAfuGfgCfuGfuCfaUfgGfuCfscsAf | 1193 | (vinu)sGfsgAfcCfaUfgAfcAfgCfcAfuUfcsusg |
| 1608 | 1003 | gscsUfgUfcAfuGfgUfcCfaAfuCfsusAf | 1194 | (vinu)sAfsgAfuUfgGfaCfcAfuGfaCfaGfcscsa |
| 1609 | 1004 | csusGfuCfaUfgGfuCfcAfaUfcUfsgsAf | 1195 | (vinu)sCfsaGfaUfuGfgAfcCfaUfgAfcAfgscsc |
| 1611 | 1005 | gsusCfaUfgGfuCfcAfaUfcUfgUfsasAf | 1196 | (vinu)sUfsaCfaGfaUfuGfgAfcCfaUfgAfcsasg |
| 1640 | 1006 | asusCfaAfuAfaAfuUfgCfgGfcAfsasAf | 1197 | (vinu)sUfsuGfcCfgCfaAfuUfuAfuUfgAfusgsg |
| 1642 | 1007 | csasAfuAfaAfuUfgCfgGfcAfaGfsasAf | 1198 | (vinu)sUfscUfuGfcCfgCfaAfuUfuAfuUfgsasu |
| 1671 | 1008 | asgsGfgGfaUfgUfaCfgUfgCfuGfsasAf | 1199 | (vinu)sUfscAfgCfaCfgUfaCfaUfcCfcCfuscsc |
| 1672 | 1009 | gsgsGfgAfuGfuAfcGfuGfcUfgAfsgsAf | 1200 | (vinu)sCfsuCfaGfcAfcGfuAfcAfuCfcCfcsusc |
| 1673 | 1010 | gsgsGfaUfgUfaCfgUfgCfuGfaGfsgsAf | 1201 | (vinu)sCfscUfcAfgCfaCfgUfaCfaUfcCfcscsu |
| 1674 | 1011 | gsgsAfuGfuAfcGfuGfcUfgAfgGfsusAf | 1202 | (vinu)sAfscCfuCfaGfcAfcGfuAfcAfuCfcscsc |
| 1677 | 1012 | usgsUfaCfgUfgCfuGfaGfgUfgGfsasAf | 1203 | (vinu)sUfscCfaCfcUfcAfgCfaCfgUfaCfasusc |
| 1678 | 1013 | gsusAfcGfuGfcUfgAfgGfuGfgAfsgsAf | 1204 | (vinu)sCfsuCfcAfcCfuCfaGfcAfcGfuAfcsasu |
| 1690 | 1014 | gsusGfgAfgCfuGfcAfcCfgAfcUfsusAf | 1205 | (vinu)sAfsaGfuCfgGfuGfcAfgCfuCfcAfcscsu |
| 1692 | 1015 | gsgsAfgCfuGfcAfcCfgAfcUfuUfsgsAf | 1206 | (vinu)sCfsaAfaGfuCfgGfuGfcAfgCfuCfcsasc |
| 1698 | 1016 | gscsAfcCfgAfcUfuUfgAfcAfaCfsasAf | 1207 | (vinu)sUfsgUfuGfuCfaAfaGfuCfgGfuGfcsasg |
| 1699 | 1017 | csasCfcGfaCfuUfuGfaCfaAfcAfsusAf | 1208 | (vinu)sAfsuGfuUfgUfcAfaAfgUfcGfgUfgscsa |
| 1723 | 1018 | gsasCfcGfuCfaCfcUfgCfuUfuGfsasAf | 1209 | (vinu)sUfscAfaAfgCfaGfgUfgAfcGfgUfcsasu |
| 1769 | 1019 | asasGfcAfgUfuCfaAfgAfaCfuUfsusAf | 1210 | (vinu)sAfsaAfgUfuCfuUfgAfaCfuGfcUfuscsu |
| 1770 | 1020 | asgsCfaGfuUfcAfaGfaAfcUfuUfscsAf | 1211 | (vinu)sGfsaAfaGfuUfcUfuGfaAfcUfgCfususc |
| 1780 | 1021 | gsasAfcUfuUfcAfgAfuCfgAfgGfsusAf | 1212 | (vinu)sAfscCfuCfgAfuCfuGfaAfaGfuUfcsusu |
| 1798 | 1022 | gscsAfgAfaGfgGfcCfgCfuAfcAfsgsAf | 1213 | (vinu)sCfsuGfuAfgCfgGfcCfcUfuCfuGfcsasc |
| 1876 | 1023 | gscsAfgAfuCfcUfgCfgCfaCfgGfsasAf | 1214 | (vinu)sUfscCfgUfgCfgCfaGfgAfuCfuGfcsusu |
| 1927 | 1024 | gscsCfcAfaGfcCfcCfgAfgAfaAfsusAf | 1215 | (vinu)sAfsuUfuCfuCfgGfgGfcUfuGfgGfcsusg |
| 1928 | 1025 | cscsCfaAfgCfcCfcGfaGfaAfaUfscsAf | 1216 | (vinu)sGfsaUfuUfcUfcGfgGfgCfuUfgGfgscsu |
| 1929 | 1026 | cscsAfaGfcCfcCfgAfgAfaAfuCfsusAf | 1217 | (vinu)sAfsgAfuUfuCfuCfgGfgGfcUfuGfgsgsc |
| 1936 | 1027 | cscsGfaGfaAfaUfcUfcCfaAfcCfsusAf | 1218 | (vinu)sAfsgGfuUfgGfaGfaUfuUfcUfcGfgsgsg |
| 1952 | 1028 | csusGfcUfgGfuGfgCfuAfcUfaAfsgsAf | 1219 | (vinu)sCfsuUfaGfuAfgCfcAfcCfaGfcAfgsgsu |
| 1954 | 1029 | gscsUfgGfuGfgCfuAfcUfaAfgAfsasAf | 1220 | (vinu)sUfsuCfuUfaGfuAfgCfcAfcCfaGfcsasg |
| 1956 | 1030 | usgsGfuGfgCfuAfcUfaAfgAfaAfsgsAf | 1221 | (vinu)sCfsuUfuCfuUfaGfuAfgCfcAfcCfasgsc |
| 1958 | 1031 | gsusGfgCfuAfcUfaAfgAfaAfgCfscsAf | 1222 | (vinu)sGfsgCfuUfuCfuUfaGfuAfgCfcAfcscsa |
| 1978 | 1032 | gsgsAfgUfgGfcAfgCfcCfgUfcUfsasAf | 1223 | (vinu)sUfsaGfaCfgGfgCfuGfcCfaCfuCfcsusg |
| 2066 | 1033 | gsgsCfaCfgAfgAfaCfaCfaCfaUfscsAf | 1224 | (vinu)sGfsaUfgUfgUfgUfuCfuCfgUfgCfcsusc |
| 2068 | 1034 | csasCfgAfgAfaCfaCfaCfaUfcUfsasAf | 1225 | (vinu)sUfsaGfaUfgUfgUfgUfuCfuCfgUfgscsc |
| 2102 | 1035 | gsasUfuAfcAfaGfgAfuGfaCfgAfsasAf | 1226 | (vinu)sUfsuCfgUfcAfuCfcUfuGfuAfaUfcscsa |
| 2111 | 1036 | gsasUfgAfcGfaAfgGfaAfcUfuCfsusAf | 1227 | (vinu)sAfsgAfaGfuUfcCfuUfcGfuCfaUfcscsu |
| 2138 | 1037 | asasGfaUfaAfaAfgUfgAfuCfcUfscsAf | 1228 | (vinu)sGfsaGfgAfuCfaCfuUfuUfaUfcUfuscsu |
| 2146 | 1038 | asgsUfgAfuCfcUfcAfaAfgUfcUfsusAf | 1229 | (vinu)sAfsaGfaCfuUfuGfaGfgAfuCfaCfususu |
| 2148 | 1039 | usgsAfuCfcUfcAfaAfgUfcUfuAfsgsAf | 1230 | (vinu)sCfsuAfaGfaCfuUfuGfaGfgAfuCfascsu |
| 2205 | 1040 | csasGfcCfaGfcAfuGfaUfgAfgAfscsAf | 1231 | (vinu)sGfsuCfuCfaUfcAfuGfcUfgGfcUfgscsc |
| 2206 | 1041 | asgsCfcAfgCfaUfgAfuGfaGfaCfsasAf | 1232 | (vinu)sUfsgUfcUfcAfuCfaUfgCfuGfgCfusgsc |
| 2218 | 1042 | gsasGfaCfaGfgUfcUfcCfcAfcAfsasAf | 1233 | (vinu)sUfsuGfuGfgGfaGfaCfcUfgUfcUfcsasu |
| 2229 | 1043 | cscsCfaCfaAfaCfaCfaUfcGfuGfsusAf | 1234 | (vinu)sAfscAfcGfaUfgUfgUfuUfgUfgGfgsasg |
| 2230 | 1044 | cscsAfcAfaAfcAfcAfuCfgUfgUfsasAf | 1235 | (vinu)sUfsaCfaCfgAfuGfuGfuUfuGfuGfgsgsa |
| 2237 | 1045 | csasCfaUfcGfuGfuAfcCfuCfuAfsusAf | 1236 | (vinu)sAfsuAfgAfgGfuAfcAfcGfaUfgUfgsusu |
| 2238 | 1046 | ascsAfuCfgUfgUfaCfcUfcUfaUfsgsAf | 1237 | (vinu)sCfsaUfaGfaGfgUfaCfaCfgAfuGfusgsu |
| 2239 | 1047 | csasUfcGfuGfuAfcCfuCfuAfuGfsgsAf | 1238 | (vinu)sCfscAfuAfgAfgGfuAfcAfcGfaUfgsusg |
| 2269 | 1048 | csgsAfcGfuGfgAfgAfaUfaUfcAfsusAf | 1239 | (vinu)sAfsuGfaUfaUfuCfuCfcAfcGfuCfgscsg |
| 2308 | 1049 | gsgsGfuCfcUfcUfgGfaUfcUfcUfsusAf | 1240 | (vinu)sAfsaGfaGfaUfcCfaGfaGfgAfcCfcscsc |
| 2317 | 1050 | gsgsAfuCfuCfuUfcAfuGfcAfcCfsgsAf | 1241 | (vinu)sCfsgGfuGfcAfuGfaAfgAfgAfuCfcsasg |
| 2318 | 1051 | gsasUfcUfcUfuCfaUfgCfaCfcGfsgsAf | 1242 | (vinu)sCfscGfgUfgCfaUfgAfaGfaGfaUfcscsa |
| 2319 | 1052 | asusCfuCfuUfcAfuGfcAfcCfgGfsasAf | 1243 | (vinu)sUfscCfgGfuGfcAfuGfaAfgAfgAfuscsc |
| 2320 | 1053 | uscsUfcUfuCfaUfgCfaCfcGfgAfsasAf | 1244 | (vinu)sUfsuCfcGfgUfgCfaUfgAfaGfaGfasusc |
| 2321 | 1054 | csusCfuUfcAfuGfcAfcCfgGfaAfsasAf | 1245 | (vinu)sUfsuUfcCfgGfuGfcAfuGfaAfgAfgsasu |
| 2322 | 1055 | uscsUfuCfaUfgCfaCfcGfgAfaAfsasAf | 1246 | (vinu)sUfsuUfuCfcGfgUfgCfaUfgAfaGfasgsa |
| 2323 | 1056 | csusUfcAfuGfcAfcCfgGfaAfaAfsgsAf | 1247 | (vinu)sCfsuUfuUfcCfgGfuGfcAfuGfaAfgsasg |
| 2520 | 1057 | cscsAfuUfaCfgGfuGfcUfgUfcUfsasAf | 1248 | (vinu)sUfsaGfaCfaGfcAfcCfgUfaAfuGfgsgsg |
| 2527 | 1058 | gsgsUfgCfuGfuCfuAfgGfcAfaGfsasAf | 1249 | (vinu)sUfscUfuGfcCfuAfgAfcAfgCfaCfcsgsu |
| 2647 | 1059 | asasUfcUfgCfuAfcAfaUfgGfcGfsasAf | 1250 | (vinu)sUfscGfcCfaUfuGfuAfgCfaGfaUfususc |
| 2761 | 1060 | csasUfgAfcCfcGfcUfgCfaUfgAfsasAf | 1251 | (vinu)sUfsuCfaUfgCfaGfcGfgGfuCfaUfgsasg |
| 2762 | 1061 | asusGfaCfcCfgCfuGfcAfuGfaAfscsAf | 1252 | (vinu)sGfsuUfcAfuGfcAfgCfgGfgUfcAfusgsa |
| 2763 | 1062 | usgsAfcCfcGfcUfgCfaUfgAfaCfsusAf | 1253 | (vinu)sAfsgUfuCfaUfgCfaGfcGfgGfuCfasusg |
| 2764 | 1063 | gsasCfcCfgCfuGfcAfuGfaAfcUfsasAf | 1254 | (vinu)sUfsaGfuUfcAfuGfcAfgCfgGfgUfcsasu |
| 2811 | 1064 | cscsAfuCfaUfgAfgAfgAfcAfuUfsasAf | 1255 | (vinu)sUfsaAfuGfuCfuCfuCfaUfgAfuGfgscsu |
| 2962 | 1065 | gscsUfcUfgCfaGfgUfaUfgAfcCfscsAf | 1256 | (vinu)sGfsgGfuCfaUfaCfcUfgCfaGfaGfcsusc |
| 2975 | 1066 | gsasCfcCfcGfaAfgGfgGfaCfaAfsusAf | 1257 | (vinu)sAfsuUfgUfcCfcCfuUfcGfgGfgUfcsasu |
| 2977 | 1067 | cscsCfcGfaAfgGfgGfaCfaAfuAfscsAf | 1258 | (vinu)sGfsuAfuUfgUfcCfcCfuUfcGfgGfgsusc |
| 3028 | 1068 | usgsAfgAfgUfgGfaGfgUfaAfcCfsasAf | 1259 | (vinu)sUfsgGfuUfaCfcUfcCfaCfuCfuCfasgsg |
| 3032 | 1069 | asgsUfgGfaGfgUfaAfcCfaCfaUfsasAf | 1260 | (vinu)sUfsaUfgUfgGfuUfaCfcUfcCfaCfuscsu |
| 3081 | 1070 | gsgsAfaCfcUfcUfaUfcAfuGfaGfsasAf | 1261 | (vinu)sUfscUfcAfuGfaUfaGfaGfgUfuCfcsusu |
| 3131 | 1071 | gsasCfgGfaGfgAfaAfuGfgUfaUfsusAf | 1262 | (vinu)sAfsaUfaCfcAfuUfuCfcUfcCfgUfcsusu |
| 3134 | 1072 | gsgsAfgGfaAfaUfgGfuAfuUfaAfsgsAf | 1263 | (vinu)sCfsuUfaAfuAfcCfaUfuUfcCfuCfcsgsu |
| 3141 | 1073 | asusGfgUfaUfuAfaGfcUfcAfuCfsasAf | 1264 | (vinu)sUfsgAfuGfaGfcUfuAfaUfaCfcAfususu |
| 3144 | 1074 | gsusAfuUfaAfgCfuCfaUfcAfuGfsgsAf | 1265 | (vinu)sCfscAfuGfaUfgAfgCfuUfaAfuAfcscsa |
| 3146 | 1075 | asusUfaAfgCfuCfaUfcAfuGfgAfsasAf | 1266 | (vinu)sUfsuCfcAfuGfaUfgAfgCfuUfaAfusasc |
| 3147 | 1076 | ususAfaGfcUfcAfuCfaUfgGfaAfsusAf | 1267 | (vinu)sAfsuUfcCfaUfgAfuGfaGfcUfuAfasusa |
| 3159 | 1077 | usgsGfaAfuUfuCfuGfcCfuUfcGfsgsAf | 1268 | (vinu)sCfscGfaAfgGfcAfgAfaAfuUfcCfasusg |
| 3160 | 1078 | gsgsAfaUfuUfcUfgCfcUfuCfgGfsgsAf | 1269 | (vinu)sCfscCfgAfaGfgCfaGfaAfaUfuCfcsasu |
| 3229 | 1079 | gscsAfgCfuAfaAfaUfaUfgCfcGfsusAf | 1270 | (vinu)sAfscGfgCfaUfaUfuUfuAfgCfuGfcsusg |
| 3247 | 1080 | uscsAfgAfuUfuGfuAfaGfgGfgAfsusAf | 1271 | (vinu)sAfsuCfcCfcUfuAfcAfaAfuCfuGfasasc |
| 3250 | 1081 | gsasUfuUfgUfaAfgGfgGfaUfgGfsasAf | 1272 | (vinu)sUfscCfaUfcCfcCfuUfaCfaAfaUfcsusg |
| 3251 | 1082 | asusUfuGfuAfaGfgGfgAfuGfgAfscsAf | 1273 | (vinu)sGfsuCfcAfuCfcCfcUfuAfcAfaAfuscsu |
| 3252 | 1083 | ususUfgUfaAfgGfgGfaUfgGfaCfsusAf | 1274 | (vinu)sAfsgUfcCfaUfcCfcCfuUfaCfaAfasusc |
| 3254 | 1084 | usgsUfaAfgGfgGfaUfgGfaCfuAfsusAf | 1275 | (vinu)sAfsuAfgUfcCfaUfcCfcCfuUfaCfasasa |
| 3255 | 1085 | gsusAfaGfgGfgAfuGfgAfcUfaUfsusAf | 1276 | (vinu)sAfsaUfaGfuCfcAfuCfcCfcUfuAfcsasa |
| 3258 | 1086 | asgsGfgGfaUfgGfaCfuAfuUfuGfsgsAf | 1277 | (vinu)sCfscAfaAfuAfgUfcCfaUfcCfcCfususa |
| 3259 | 1087 | gsgsGfgAfuGfgAfcUfaUfuUfgGfsgsAf | 1278 | (vinu)sCfscCfaAfaUfaGfuCfcAfuCfcCfcsusu |
| 3260 | 1088 | gsgsGfaUfgGfaCfuAfuUfuGfgGfsusAf | 1279 | (vinu)sAfscCfcAfaAfuAfgUfcCfaUfcCfcscsu |
| 3261 | 1089 | gsgsAfuGfgAfcUfaUfuUfgGfgUfsusAf | 1280 | (vinu)sAfsaCfcCfaAfaUfaGfuCfcAfuCfcscsc |
| 3265 | 1090 | gsgsAfcUfaUfuUfgGfgUfuCfuCfsgsAf | 1281 | (vinu)sCfsgAfgAfaCfcCfaAfaUfaGfuCfcsasu |
| 3268 | 1091 | csusAfuUfuGfgGfuUfcUfcGfgCfsasAf | 1282 | (vinu)sUfsgCfcGfaGfaAfcCfcAfaAfuAfgsusc |
| 3272 | 1092 | ususGfgGfuUfcUfcGfgCfaAfuAfscsAf | 1283 | (vinu)sGfsuAfuUfgCfcGfaGfaAfcCfcAfasasu |
| 3275 | 1093 | gsgsUfuCfuCfgGfcAfaUfaCfgUfsusAf | 1284 | (vinu)sAfsaCfgUfaUfuGfcCfgAfgAfaCfcscsa |
| 3276 | 1094 | gsusUfcUfcGfgCfaAfuAfcGfuUfscsAf | 1285 | (vinu)sGfsaAfcGfuAfuUfgCfcGfaGfaAfcscsc |
| 3278 | 1095 | uscsUfcGfgCfaAfuAfcGfuUfcAfscsAf | 1286 | (vinu)sGfsuGfaAfcGfuAfuUfgCfcGfaGfasasc |
| 3279 | 1096 | csusCfgGfcAfaUfaCfgUfuCfaCfscsAf | 1287 | (vinu)sGfsgUfgAfaCfgUfaUfuGfcCfgAfgsasa |
| 3281 | 1097 | csgsGfcAfaUfaCfgUfuCfaCfcGfsgsAf | 1288 | (vinu)sCfscGfgUfgAfaCfgUfaUfuGfcCfgsasg |
| 3282 | 1098 | gsgsCfaAfuAfcGfuUfcAfcCfgGfsgsAf | 1289 | (vinu)sCfscCfgGfuGfaAfcGfuAfuUfgCfcsgsa |
| 3283 | 1099 | gscsAfaUfaCfgUfuCfaCfcGfgGfsasAf | 1290 | (vinu)sUfscCfcGfgUfgAfaCfgUfaUfuGfcscsg |
| 3284 | 1100 | csasAfuAfcGfuUfcAfcCfgGfgAfscsAf | 1291 | (vinu)sGfsuCfcCfgGfuGfaAfcGfuAfuUfgscsc |
| 3285 | 1101 | asasUfaCfgUfuCfaCfcGfgGfaCfsusAf | 1292 | (vinu)sAfsgUfcCfcGfgUfgAfaCfgUfaUfusgsc |
| 3286 | 1102 | asusAfcGfuUfcAfcCfgGfgAfcUfsusAf | 1293 | (vinu)sAfsaGfuCfcCfgGfuGfaAfcGfuAfususg |
| 3313 | 1103 | asasAfuGfuCfcUfuGfuUfgAfgAfsgsAf | 1294 | (vinu)sCfsuCfuCfaAfcAfaGfgAfcAfuUfuscsu |
| 3314 | 1104 | asasUfgUfcCfuUfgUfuGfaGfaGfsusAf | 1295 | (vinu)sAfscUfcUfcAfaCfaAfgGfaCfaUfususc |
| 3323 | 1105 | gsusUfgAfgAfgUfgAfaCfaCfcAfsasAf | 1296 | (vinu)sUfsuGfgUfgUfuCfaCfuCfuCfaAfcsasa |
| 3353 | 1106 | gsasCfuUfcGfgUfuUfaAfcCfaAfsasAf | 1297 | (vinu)sUfsuUfgGfuUfaAfaCfcGfaAfgUfcsusc |
| 3365 | 1107 | ascsCfaAfaGfcAfaUfuGfaAfaCfscsAf | 1298 | (vinu)sGfsgUfuUfcAfaUfuGfcUfuUfgGfususa |
| 3367 | 1108 | csasAfaGfcAfaUfuGfaAfaCfcGfsasAf | 1299 | (vinu)sUfscGfgUfuUfcAfaUfuGfcUfuUfgsgsu |
| 3368 | 1109 | asasAfgCfaAfuUfgAfaAfcCfgAfsusAf | 1300 | (vinu)sAfsuCfgGfuUfuCfaAfuUfgCfuUfusgsg |
| 3371 | 1110 | gscsAfaUfuGfaAfaCfcGfaUfaAfsgsAf | 1301 | (vinu)sCfsuUfaUfcGfgUfuUfcAfaUfuGfcsusu |
| 3372 | 1111 | csasAfuUfgAfaAfcCfgAfuAfaGfsgsAf | 1302 | (vinu)sCfscUfuAfuCfgGfuUfuCfaAfuUfgscsu |
| 3376 | 1112 | usgsAfaAfcCfgAfuAfaGfgAfgUfsasAf | 1303 | (vinu)sUfsaCfuCfcUfuAfuCfgGfuUfuCfasasu |
| 3409 | 1113 | usgsAfcCfgGfgAfcAfgCfcCfuGfsusAf | 1304 | (vinu)sAfscAfgGfgCfuGfuCfcCfgGfuCfasusc |
| 3505 | 1114 | gscsAfuGfaGfcUfgCfuGfaCfuUfsasAf | 1305 | (vinu)sUfsaAfgUfcAfgCfaGfcUfcAfuGfcsasg |
| 3556 | 1115 | cscsUfgAfaAfaUfgAfuAfgGfcCfscsAf | 1306 | (vinu)sGfsgGfcCfuAfuCfaUfuUfuCfaGfgsasa |
| 3557 | 1116 | csusGfaAfaAfuGfaUfaGfgCfcCfsasAf | 1307 | (vinu)sUfsgGfgCfcUfaUfcAfuUfuUfcAfgsgsa |
| 3558 | 1117 | usgsAfaAfaUfgAfuAfgGfcCfcAfsasAf | 1308 | (vinu)sUfsuGfgGfcCfuAfuCfaUfuUfuCfasgsg |
| 3559 | 1118 | gsasAfaAfuGfaUfaGfgCfcCfaAfscsAf | 1309 | (vinu)sGfsuUfgGfgCfcUfaUfcAfuUfuUfcsasg |
| 3654 | 1119 | csasGfaUfgAfgGfuUfuAfuCfaAfscsAf | 1310 | (vinu)sGfsuUfgAfuAfaAfcCfuCfaUfcUfgsgsa |
| 3662 | 1120 | gsusUfuAfuCfaAfcUfuAfuGfaGfsgsAf | 1311 | (vinu)sCfscUfcAfuAfaGfuUfgAfuAfaAfcscsu |
| 3663 | 1121 | ususUfaUfcAfaCfuUfaUfgAfgGfsasAf | 1312 | (vinu)sUfscCfuCfaUfaAfgUfuGfaUfaAfascsc |
| 3683 | 1122 | usgsCfuGfgGfaAfuUfcCfaAfcCfsasAf | 1313 | (vinu)sUfsgGfuUfgGfaAfuUfcCfcAfgCfasusu |
| 3689 | 1123 | gsasAfuUfcCfaAfcCfaUfcCfaAfsusAf | 1314 | (vinu)sAfsuUfgGfaUfgGfuUfgGfaAfuUfcscsc |
| 3694 | 1124 | cscsAfaCfcAfuCfcAfaUfcGfgAfscsAf | 1315 | (vinu)sGfsuCfcGfaUfuGfgAfuGfgUfuGfgsasa |
| 3695 | 1125 | csasAfcCfaUfcCfaAfuCfgGfaCfsasAf | 1316 | (vinu)sUfsgUfcCfgAfuUfgGfaUfgGfuUfgsgsa |
| 3698 | 1126 | cscsAfuCfcAfaUfcGfgAfcAfaGfscsAf | 1317 | (vinu)sGfscUfuGfuCfcGfaUfuGfgAfuGfgsusu |
| 3702 | 1127 | cscsAfaUfcGfgAfcAfaGfcUfuUfscsAf | 1318 | (vinu)sGfsaAfaGfcUfuGfuCfcGfaUfuGfgsasu |
| 3719 | 1128 | csasGfaAfcCfuUfaUfuGfaAfgGfsasAf | 1319 | (vinu)sUfscCfuUfcAfaUfaAfgGfuUfcUfgsasa |
| 3781 | 1129 | cscsAfcAfgAfuUfaUfcAfaGfuCfscsAf | 1320 | (vinu)sGfsgAfcUfuGfaUfaAfuCfuGfuGfgsasa |
| 3894 | 1130 | csasUfaUfaCfaUfgUfaUfaAfgGfscsAf | 1321 | (vinu)sGfscCfuUfaUfaCfaUfgUfaUfaUfgsusa |
| 4099 | 1131 | csgsAfgGfaGfuUfgAfcCfaAfaAfsusAf | 1322 | (vinu)sAfsuUfuUfgGfuCfaAfcUfcCfuCfgsusu |
| 4169 | 1132 | gscsUfgGfcAfcAfuUfaAfuCfaUfsasAf | 1323 | (vinu)sUfsaUfgAfuUfaAfuGfuGfcCfaGfcsasa |
| 4239 | 1133 | csusAfgAfcCfaUfgCfaUfuCfuUfsasAf | 1324 | (vinu)sUfsaAfgAfaUfgCfaUfgGfuCfuAfgsusa |
| 4305 | 1134 | gsusAfuUfcUfcUfcAfcCfaGfuAfsgsAf | 1325 | (vinu)sCfsuAfcUfgGfuGfaGfaGfaAfuAfcsasg |
| 4374 | 1135 | ascsUfaGfcAfcCfcAfuUfuUfuGfsasAf | 1326 | (vinu)sUfscAfaAfaAfuGfgGfuGfcUfaGfusgsa |
| 4411 | 1136 | gsgsGfgAfuAfgCfuGfuGfgAfaUfsasAf | 1327 | (vinu)sUfsaUfuCfcAfcAfgCfuAfuCfcCfcscsc |
| 4475 | 1137 | gscsUfuUfcCfuAfaGfcAfgUfaUfsasAf | 1328 | (vinu)sUfsaUfaCfuGfcUfuAfgGfaAfaGfcsasc |
| 4612 | 1138 | gsasAfuGfcUfgUfaUfgCfaAfcCfsasAf | 1329 | (vinu)sUfsgGfuUfgCfaUfaCfaGfcAfuUfcsasa |
| 4671 | 1139 | csasUfaCfuUfaCfcAfcCfgAfuCfsusAf | 1330 | (vinu)sAfsgAfuCfgGfuGfgUfaAfgUfaUfgsgsa |
| 4672 | 1140 | asusAfcUfuAfcCfaCfcGfaUfcUfsasAf | 1331 | (vinu)sUfsaGfaUfcGfgUfgGfuAfaGfuAfusgsg |
| 4679 | 1141 | cscsAfcCfgAfuCfuAfcAfaGfgGfsusAf | 1332 | (vinu)sAfscCfcUfuGfuAfgAfuCfgGfuGfgsusa |
| 4682 | 1142 | cscsGfaUfcUfaCfaAfgGfgUfuGfsasAf | 1333 | (vinu)sUfscAfaCfcCfuUfgUfaGfaUfcGfgsusg |
| 4683 | 1143 | csgsAfuCfuAfcAfaGfgGfuUfgAfsusAf | 1334 | (vinu)sAfsuCfaAfcCfcUfuGfuAfgAfuCfgsgsu |
| 4684 | 1144 | gsasUfcUfaCfaAfgGfgUfuGfaUfscsAf | 1335 | (vinu)sGfsaUfcAfaCfcCfuUfgUfaGfaUfcsgsg |
| 4690 | 1145 | csasAfgGfgUfuGfaUfcCfcUfgUfsusAf | 1336 | (vinu)sAfsaCfaGfgGfaUfcAfaCfcCfuUfgsusa |
| 4794 | 1146 | csasGfuUfuGfcUfuGfgAfgGfuAfsgsAf | 1337 | (vinu)sCfsuAfcCfuCfcAfaGfcAfaAfcUfgsasa |
| 4803 | 1147 | usgsGfaGfgUfaGfcUfgGfgUfaAfsusAf | 1338 | (vinu)sAfsuUfaCfcCfaGfcUfaCfcUfcCfasasg |
| 4807 | 1148 | gsgsUfaGfcUfgGfgUfaAfuCfaAfsasAf | 1339 | (vinu)sUfsuUfgAfuUfaCfcCfaGfcUfaCfcsusc |

### Table 4: Preferred Compounds (Including Conjugated Compounds)-HELM Strings

Table 4 below presents twenty compounds of the invention tested in Examples 4 to 8 herein, as well as their individual sense and antisense strands, using Hierarchical Editing Language for Macromolecules (HELM) notation.

For each compound in Table 4 that consists of an siRNA duplex (i.e. with no additional moiety) there are corresponding compounds wherein the siRNA has the same sequence but wherein palimitic acid (a C16 conjugate moiety) or behenic acid (a C22 conjugate moiety) is attached to the 3′ end of the sense strand via a C6 amino alkyl linker as described herein.

For instance, compound 614 is an siRNA duplex; compound 614_C16 is an siRNA duplex with the same sequences for the sense strand and antisense strand wherein palmitic acid is conjugated to the 3′ end of the sense strand via a C6 amino linker; and compound 614_C22 is an siRNA duplex with the same sequences for the sense strand and antisense strand wherein behenic acid is conjugated to the 3′ end of the sense strand via a C6 amino linker.

The chemical modification pattern for the 2′ position of the sugar moieties on both the sense strand and antisense strand is the same in conjugated compounds and non-conjugated compounds.

However, the pattern of phosphorothioate internucleotide linkages in the sense strand of conjugated compounds is slightly different to the pattern in the sense strand of non-conjugated compounds. In particular, in conjugated compounds the 3′-most nucleotide of the sense strand is linked to the C6 amino alkyl linker via an additional phosphorothioate group and the internucleotide linkage between the 17th and 18th nucleotide is a phosphodiester (rather than the phosphorothioate found at this position in the sense strand of non-conjugated compounds).

Thus, the modification pattern of the sense strand of conjugated compounds (i.e., C16 siRNAs and C22 siRNAs) from 5′ to 3′ is as follows:
5′-[2′OMe]-PS-[2′OMe]-PS-[2′F]-[2′OMe]-[2′F]-[2′OMe]-[2′F]-[2′OMe]-[2′F]-[2′OMe]-[2′F]-[2′OMe]-[2′F]-[2′OMe]-[2′F]-[2′OMe]-[2′F]-[2′OMe]-PS-[2′F]-PS-[C6-amino-linker]-3′

In contrast, the modification pattern of the sense strand of non-conjugated compounds (i.e. the “parent design” as also described elsewhere herein) from 5′ to 3′ is as follows:
5′-[2′OMe]-PS-[2′OMe]-PS-[2′F]-[2′OMe]-[2′F]-[2′OMe]-[2′F]-[2′OMe]-[2′F]-[2′OMe]-[2′F]-[2′OMe]-[2′F]-[2′OMe]-[2′F]-[2′OMe]-[2′F]-PS-[2′OMe]-PS-[2′F]-3′

(The variant phosphorothioate group in each pattern is shown in bold.)

In the patterns above, [2′OMe] represents a 2′-OMe RNA,

[2′F] represents a 2′-F RNA,

[VP-2′-OMe] represents a Vinyl-phosphonate 2′-OMe RNA, and

PS represents phosphorothioate internucleotide linkages (in the absence of mention, the internucleotide linkages is a phosphodiester internucleotide linkage).

### HELM Notation

HELM is a notation format designed to depict the structure of macromolecules. Full details of HELM notation may be found at www.pistoiaalliance.org/helm-tools/, in Zhang et al. J. Chem. Inf. Model. 2012, 52, 2796-2806 (which initially described HELM notation) and in Milton et al. J. Chem. Inf. Model. 2017, 57, 1233-1239 (which describes HELM version 2.0).

Briefly, a macromolecule is depicted as a “HELM string”, which is divided into sections. The first section of the HELM string lists the molecules comprised in the macromolecule. The second section lists the connections between molecules within the macromolecule. Third, fourth and fifth sections (which may be used in HELM strings for more complex macromolecules) are not used in the HELM strings herein. One or more dollar sign $ marks the end of a section and a vertical line | defines sub-sections (e.g. separating molecules in the first section, and separating connections in the second section).

Accordingly, compounds of the invention are represented by a HELM string consisting of two sections: the first section defines the structures of antisense strand, the sense strand and (if present) the conjugate moiety, and the second section defines the base-pairing between the strands and how the conjugate moiety (if present) is connected to either strand (typically the sense strand).

Each molecule listed in the first section of a HELM string is given an identifier (e.g. “RNA1” for a nucleic acid, “PEPTIDE1” for an amino acid sequence, “CHEM1” for a chemical structure) and the structure of the molecule is defined by notation in braces { } immediately following the identifier. Thus, in HELM strings depicting compounds of the invention, “RNA1” is the identifier of the antisense strand, “RNA2” is the identifier of the sense strand and “CHEM1” is the identifier of the conjugate moiety (if present).

The notation used to define the structure of each molecule in braces { } in the first section of HELM strings for the present invention are as follows:
demarcates nucleosides,
[mR](A) is a 2′-O-methyl RNA adenine nucleoside,
[mR](C) is a 2′-O-methyl RNA cytosine nucleoside,
[mR](G) is a 2′-O-methyl RNA guanine nucleoside,
[mR](U) is a 2′-O-methyl RNA uracil nucleoside,
[fR](A) is a 2′-fluoro RNA adenine nucleoside,
[fR](C) is a 2′-fluoro RNA cytosine nucleoside,
[fR](G) is a 2′-fluoro RNA guanine nucleoside,
[fR](U) is a 2′-fluoro RNA uracil nucleoside,
[mRVP](U) is a 5′-Vinyl phosphonate 2′-O-Methyl uracil nucleoside,
[A6] is a C6 amino linker,
[Palmitate] is Palmitic acid conjugate moiety,
[C22COOH] is Behenic acid (Docosanoic acid) conjugate moiety,
[P] is a phosphodiester internucleotide linkage, and
[sP] is a phosphorothioate internucleotide linkage.

In the case of HELM strings representing conjugates, there is a connection between the conjugate moiety and sense strand. This connection is represented in all HELM strings herein as follows:
RNA2, CHEM1,57: R2-1: R1.

In some cases the conjugates can be described as two chemical moieties connected together. This connection is represented in all HELM strings herein as follows:
RNA2,CHEM1,57: R2-1: R1|CHEM1, CHEM2, 1: R2-1: R1

“V2.0” indicates that HELM version 2.0 is used.

### Example of HELM Notation

For example, Compound 614_C22 is represented by the following HELM string (as shown in Table 4):
CHEM1{[A6]}|CHEM2{[C22COOH]}|RNA1{[mRVP](U)[sP].[fR](G)[sP].[mR](A)P.[fR](A)P.[mR](A)P.[fR](U)P.[mR](A)P.[fR](G)P.[mR](A)P.[fR](A)P.[mR](C)P.[fR](C)P.[mR](U)P.[fR](C)P.[m R](A)P.[fR](U)P.[mR](C)P.[fR](C)P.[mR](G)[sP].[mR](G)[sP].[mR](U)}|RNA2{[mR](C)[sP].[m R](G)[sP].[fR](G)P.[mR](A)P.[fR](U)P.[mR](G)P.[fR](A)P.[mR](G)P.[fR](G)P.[mR](U)P.[fR](U) P.[mR](C)P.[fR](U)P.[mR](A)P.[fR](U)P.[mR](U)P.[fR](U)P.[mR](C)[sP].[fR](A)[sP]}$RNA2,C HEM1,57: R2-1: R1|CHEM1,CHEM2.1: R2-1: R1|RNA2,RNA1,11: pair-47: pair|RNA2,RNA1, 14: pair-44: pair|RNA2,RNA1,17: pair-41: pair|RNA2,RNA1,20: pair-38: pair|RNA2.RNA1,23: pair-35: pair|RNA2.RNA1,26: pair-32: pair|RNA2,RNA1,29: pair-29: pair|RNA2,RNA1,2: pair-56: pair|RNA2,RNA1,32: pair-26: pair|RNA2,RNA1,35: pair-23: pair|RNA2,RNA1,38: pair-20: pair|RNA2,RNA1,41: pair-17: pair|RNA2, RNA1,44: pair-14: pair|RNA2,RNA1,47: pair-11: pair|RNA2,RNA1,50: pair-8: pair|RNA2,RNA1,53: pair-5: pair|RNA2,RNA1,56: pair-2: pair|RNA2,RNA1,5: pair-53: pair|RNA2,RNA1,8: pair-50: pair$$$V2.0

This HELM string consists of two sections; the end of each section is marked by a $ sign. The first section defines the three components of the compound: the C6 amino linker (A6), the C22 fatty acid (CHEM1), the antisense strand (RNA1 or RNA2) and the sense strand (RNA1 or RNA2). The antisense strand or the sense strand may be referred to as either RNA1 or RNA2 (i.e., “RNA1” could be used to refer to either the antisense strand or sense strand, or “RNA2” could be used to refer to either the antisense strand or sense strand herein). However, in HELM strings wherein the antisense strand is RNA1 the sense strand is RNA2, and in HELM strings wherein the antisense strand is RNA1 the sense strand is RNA2. The structure of each component follows the name in braces { }. The second section defines how C22 fatty acid (CHEM1) is conjugated to the sense strand (RNA2), and how the antisense strand (RNA1) forms base pairs with the sense strand (RNA2). Two further $$ signs mark the end of the HELM string as a whole. “V2.0” indicates that HELM version 2.0 is used.

TABLE 4

| | | Sense | Antisense | |
|---|---|---|---|---|
| Common | | strand | strand | |
| compound | | SEQ | SEQ | |
| number | Compound | ID NO | ID NO | HELM string |
| 614 | 614 | 958 | 1149 | RNA1{[mR](C)[sP].[mR](G)[sP].[fR](G)P.[mR](A)P.[fR](U)P.[mR](G)P.[fR](A)P.[mR](G)P.[fR] |
| | siRNA duplex | | | (G)P.[mR](U)P.[fR](U)P.[mR](C)P.[fR](U)P.[mR](A)P.[fR](U)P.[mR](U)P.[fR](U)[sP].[mR](C) |
| | | | | [sP].[fR](A)}\|RNA2{[mRVP](U)[sP].[fR](G)[P].[mR](A)P.[fR](A)P.[mR](A)P.[fR](U)P.[mR](A) |
| | | | | P.[fR](G)P.[mR](A)P.[fR](A)P.[mR](C)P.[fR](C)P.[mR](U)P.[fR](C)P.[mR](A)P.[fR](U)P.[mR](C) |
| | | | | P.[fR](C)P.[mR](G)[sP].[mR](G)[sP].[mR](U)}$RNA1, RNA2, 11: pair-47: pair\|RNA1, RNA2, 14: |
| | | | | pair-44: pair\|RNA1, RNA2, 17: pair-41: pair\|RNA1, RNA2, 20: pair-38: pair\|RNA1, RNA2, 23: pair- |
| | | | | 35: pair\|RNA1, RNA2, 26: pair-32: pair\|RNA1, RNA2, 29: pair-29: pair\|RNA1, RNA2, 2: pair- |
| | | | | 56: pair\|RNA1, RNA2, 32: pair-26: pair\|RNA1, RNA2, 35: pair-23: pair\|RNA1, RNA2, 38: pair- |
| | | | | 20: pair\|RNA1, RNA2, 41: pair-17: pair\|RNA1, RNA2, 44: pair-14: pair\|RNA1, RNA2, 47: pair- |
| | | | | 11: pair\|RNA1, RNA2, 50: pair-8: pair\|RNA1, RNA2, 53: pair-5: pair\|RNA1, RNA2, 56: pair- |
| | | | | 2: pair\|RNA1, RNA2, 5: pair-53: pair\|RNA1, RNA2, 8: pair-50: pair$$$V2.0 |
| | 614_C16 | 958 | 1149 | CHEM1{[Palmitate]}\|RNA1{[mRVP](U)[sP].[fR](G)[sP].[mR](A)P.[fR](A)P.[mR](A)P.[fR](U) |
| | siRNA duplex | | | P.[mR](A)P.[fR](G)P.[mR](A)P.[fR](A)P.[mR](C)P.[fR](C)P.[mR](U)P.[fR](C)P.[mR](A)P.[fR](U) |
| | conjugated to | | | P.[mR](C)P.[fR](C)P.[mR](G)[sP].[mR](G)[P].[mR](U)}\|RNA2{[mR](C)[sP].[mR](G)[sP].[fR](G) |
| | C16 fatty acid | | | P.[mR](A)P.[fR](U)P.[mR](G)P.[fR](A)P.[mR](G)P.[fR](G)P.[mR](U)P.[fR](U)P.[mR](C)P.[fR](U) |
| | | | | P.[mR](A)P.[fR](U)P.[mR](U)P.[fR](U)P.[mR](C)[sP].[fR](A)[sP]}$RNA2, CHEM1, 57: R2- |
| | | | | 1: R1\|RNA2, RNA1, 11: pair-47: pair\|RNA2, RNA1, 14: pair-44: pair\|RNA2, RNA1, 17: pair- |
| | | | | 41: pair\|RNA2, RNA1, 20: pair-38: pair\|RNA2, RNA1, 23: pair-35: pair\|RNA2, RNA1, 26: pair- |
| | | | | 32: pair\|RNA2, RNA1, 29: pair-29: pair\|RNA2, RNA1, 2: pair-56: pair\|RNA2, RNA1, 32: pair- |
| | | | | 26: pair\|RNA2, RNA1, 35: pair-23: pair\|RNA2, RNA1, 38: pair-20: pair\|RNA2, RNA1, 41: pair- |
| | | | | 17: pair\|RNA2, RNA1, 44: pair-14: pair\|RNA2, RNA1, 47: pair-11: pair\|RNA2, RNA1, 50: pair- |
| | | | | 8: pair\|RNA2, RNA1, 53: pair-5: pair\|RNA2, RNA1, 56: pair-2: pair\|RNA2, RNA 1, 5: pair- |
| | | | | 53: pair\|RNA2, RNA1, 8: pair-50: pair$$$V2.0 |
| | 614_C22 | 958 | 1149 | CHEM1{[A6]}\|CHEM2{[C22COOH]}\|RNA1{[mRVP](U)[sP].[fR](G)[sP].[mR](A)P.[fR](A)P.[mR] |
| | siRNA duplex | | | (A)P.[fR](U)P.[mR](A)P.[fR](G)P.[mR](A)P.[fR](A)P.[mR](C)P.[fR](C)P.[mR](U)P.[fR](C)P.[mR] |
| | conjugated to | | | (A)P.[fR](U)P.[mR](C)P.[fR](C)P.[mR](G)[sP].[mR](G)[sP].[mR](U)}\|RNA2{[mR](C)[sP].[mR](G) |
| | C22 fatty acid | | | [sP].[fR](G)P.[mR](A)P.[fR](U)P.[mR](G)P.[fR](A)P.[mR](G)P.[fR](G)P.[mR](U)P.[fR](U)P.[mR] |
| | | | | (C)P.[fR](U)P.[mR](A)P.[fR](U)P.[mR](U)P.[fR](U)P.[mR](C)[sP].[fR](A)[sP]}$RNA2, CHEM1, |
| | | | | 57: R2-1: R1\|CHEM1, CHEM2, 1: R2-1: R1\|RNA2, RNA1, 11: pair-47: pair\|RNA2, RNA1, 14: pair- |
| | | | | 44: pair\|RNA2, RNA1, 17: pair-41: pair\|RNA2, RNA1, 20: pair-38: pair\|RNA2, RNA1, 23: pair- |
| | | | | 35: pair\|RNA2, RNA1, 26: pair-32: pair\|RNA2, RNA1, 29: pair-29: pair\|RNA2, RNA1, 2: pair- |
| | | | | 56: pair\|RNA2, RNA1, 32: pair-26: pair\|RNA2, RNA1, 35: pair-23: pair\|RNA2, RNA1, 38: pair- |
| | | | | 20: pair\|RNA2, RNA1, 41: pair-17: pair\|RNA2, RNA1, 44: pair-14: pair\|RNA2, RNA1, 47: pair- |
| | | | | 11: pair\|RNA2, RNA1, 50: pair-8: pair\|RNA2, RNA1, 53: pair-5: pair\|RNA2, RNA1, 56: pair- |
| | | | | 2: pair\|RNA2, RNA1, 5: pair-53: pair\|RNA2, RNA1, 8: pair-50: pair$$$V2.0 |
| | 614, 614_C16 | / | 1149 | RNA1{[mRVP](U)[sP].[fR](G)[sP].[mR](A)P.[fR](A)P.[mR](A)P.[fR](U)P.[mR](A)P.[fR](G) |
| | and 614_C22 | | | P.[mR](A)P.[fR](A)P.[mR](C)P.[fR](C)P.[mR](U)P.[fR](C)P.[mR](A)P.[fR](U)P.[mR](C)P.[fR](C) |
| | Antisense | | | P.[mR](G)[sP].[mR](G)[P].[mR](U)}$$$$V2.0 |
| | strand | | | |
| | 614 | 958 | / | RNA1{[mR](C)[sP] [mR](G)[sP].[fR](G)P.[mR](A)P [fR](U)P .[mR](G)P.[fR](A)P.[mR](G)P [fR] |
| | Sense strand | | | (G)P.[mR](U)P.[fR](U)P.[mR](C)P.[fR](U)P.[mR](A)P.[fR](U)P.[mR](U)P.[fR](U)[sP] [mR](C)[sP] |
| | 614_C16 | 958 | / | CHEM 1{[Palmitate]}\|RNA1{[mR](C)[sP] [mR](G)[sP].[fR](G)P.[mR](A)P.[fR](U)P.[mR](G)P.[fR] |
| | Sense strand | | | (A)P.[mR](G)P.[fR](G)P.[mR](U)P.[fR](U)P.[mR](C)P.[fR](U)P.[mR](A)P.[fR](U)P.[mR](U)P.[fR] |
| | | | | (U)P.[mR](C)[sP].[fR](A)[sP]SRNA1, CHEM1, 57: R2-1: R1$$$V2.0 |
| | 614_C22 | 958 | / | CHEM1{[A6]\|CHEM2{[C22COOH]}\|RNA1{[mR](C)[sP].[mR](G)[sP].[IR](G)P.[mR](A)P.[IR](U) |
| | Sense strand | | | P.[mR](G)P.[fR](A)P.[mR](G)P.[fR](G)P.[mR](U)P.[fR](U)P.[mR](C)P.[fR](U)P.[mR](A)P. [fR](U) |
| | | | | P.[mR](U)P.[fR](U)P.[mR](C)[sP].[fR](A)[sP]SRNA1, CHEM1, 57: R2- |
| | | | | 1: R1\|CHEM1, CHEM2, 1: R2-1: R1$$$V2.0 |
| 673 | 673 | 959 | 1150 | RNA1{[mR](G)[sP].[mR](C)[sP].[fR](G)P.[mR](U)P.[fR](C)P.[mR](A)P.[fR](U)P.[mR](U)P.[fR] |
| | siRNA duplex | | | (C)P.[mR](U)P.[fR](C)P.[mR](C)P.[fR](A)P.[mR](A)P.[fR](A)P.[mR](G)P.[fR](A)[P].[mR](A) |
| | | | | [sP].[fR](A)}\|RNA2{[mRVP](U)[sP].[fR](U)[P].[mR](U)P.[fR](C)P.[mR](U)P.[fR](U)P.[mR](U) |
| | | | | P.[fR](G)P.[mR](G)P.[fR](A)P.[mR](G)P.[fR](A)P.[mR](A)P.[fR](U)P.[mR](G)P.[fR](A)P.[mR](C) |
| | | | | P.[fR](G)P.[mR](C)[sP].[mR](C)[sP].[mR](A)}$RNA1, RNA2, 11: pair-47: pair\|RNA1, RNA2, 14: |
| | | | | pair-44: pair\|RNA1, RNA2, 17: pair-41: pair\|RNA1, RNA2, 20: pair-38: pair\|RNA1, RNA2, 23: pair- |
| | | | | 35: pair\|RNA1, RNA2, 26: pair-32: pair\|RNA1, RNA2, 29: pair-29: pair\|RNA1, RNA2, 2: pair- |
| | | | | 56: pair\|RNA1, RNA2, 32: pair-26: pair\|RNA1, RNA2, 35: pair-23: pair\|RNA1, RNA2, 38: pair- |
| | | | | 20: pair\|RNA1, RNA2, 41: pair-17: pair\|RNA1, RNA2, 44: pair-14: pair\|RNA1, RNA2, 47: pair- |
| | | | | 11: pair\|RNA1, RNA2, 50: pair-8: pair\|RNA1, RNA2, 53: pair-5: pair\|RNA1, RNA2, 56: pair- |
| | | | | 2: pair\|RNA1, RNA2, 5: pair-53: pair\|RNA1, RNA2, 8: pair-50: pair$$$V2.0 |
| | 673_C16 | 959 | 1150 | CHEM1{[Palmitate]}\|RNA1{[mR](G)[sP].[mR](C)[sP].[fR](G)P.[mR](U)P.[fR](C)P.[mR](A)P.[fR] |
| | siRNA duplex | | | (U)P.[mR](U)P.[fR](C)P.[mR](U)P.[fR](C)P.[mR](C)P.[fR](A)P.[mR](A)P.[fR](A)P.[mR](G)P.[fR] |
| | conjugated to | | | (A)P.[mR](A)[sP].[fR](A)[sP]}\|RNA2{[mRVP](U)[sP].[fR](U)[sP].[mR](U)P.[fR](C)P.[mR](U) |
| | C16 fatty acid | | | P.[fR](U)P.[mR](U)P.[fR](G)P.[mR](G)P.[fR](A)P.[mR](G)P.[fR](A)P.[mR](A)P.[fR](U)P.[mR](G) |
| | | | | P.[fR](A)P.[mR](C)P.[fR](G)P.[mR](C)[sP].[mR](C)[sP].[mR](A)}$CHEM1, RNA1, 1: R1- |
| | | | | 57: R2\|RNA1, RNA2, 11: pair-47: pair\|RNA1, RNA2, 14: pair-44: pair\|RNA1, RNA2, 17: pair- |
| | | | | 41: pair\|RNA1, RNA2, 20: pair-38: pair\|RNA1, RNA2, 23: pair-35: pair\|RNA1, RNA2, 26: pair- |
| | | | | 32: pair\|RNA1, RNA2, 29: pair-29: pair\|RNA1, RNA2, 2: pair-56: pair\|RNA1, RNA2, 32: pair- |
| | | | | 26: pair\|RNA1, RNA2, 35: pair-23: pair\|RNA1, RNA2, 38: pair-20: pair\|RNA1, RNA2, 41: pair- |
| | | | | 17: pair\|RNA1, RNA2, 44: pair-14: pair\|RNA1, RNA2, 47: pair-11: pair\|RNA1, RNA2, 50: pair- |
| | | | | 8: pair\|RNA1, RNA2, 53: pair-5: pair\|RNA1, RNA2, 56: pair-2: pair\|RNA1, RNA2, 5: pair- |
| | | | | 53: pair\|RNA1, RNA2, 8: pair-50: pair$$$V2.0 |
| | 673_C22 | 959 | 1150 | CHEM1{[ A6]}\|CHEM2{[C22COOH]}\|RNA1{[mRVP](U)[sP].[fR](U)[sP].[mR](U)P.[fR](C)P.[mR] |
| | siRNA duplex | | | (U)P.[fR](U)P.[mR](U)P.[fR](G)P.[mR](G)P.[fR](A)P.[mR](G)P.[fR](A)P.[mR](A)P. [fR](U)P.[mR] |
| | conjugated to | | | (G)P.[fR](A)P.[mR](C)P.[fR](G)P.[mR](C)[sP].[mR](C)[sP].[mR](A)}\|RNA2{[mR](G)[sP].[mR](C) |
| | C22 fatty acid | | | [sP].[fR](G)P.[mR](U)P.[fR](C)P.[mR](A)P.[fR](U)P.[mR](U)P.[fR](C)P.[mR](U)P.[fR](C)P.[mR] |
| | | | | (C)P.[fR](A)P.[mR](A)P.[fR](A)P.[mR](G)P.[fR](A)P.[mR](A)[sP].[fR](A)[sP]}$RNA2, CHEM1, 57: |
| | | | | R2-1: R1\|CHEM1, CHEM2, 1: R2-1: R1\|RNA2, RNA1, 11: pair-47: pair\|RNA2, RNA1, 14: pair- |
| | | | | 44: pair\|RNA2, RNA1, 17: pair-41: pair\|RNA2, RNA1, 20: pair-38: pair\|RNA2, RNA1, 23: pair- |
| | | | | 35: pair\|RNA2, RNA1, 26: pair-32: pair\|RNA2, RNA1, 29: pair-29: pair\|RNA2, RNA1, 2: pair- |
| | | | | 56: pair\|RNA2, RNA1, 32: pair-26: pair\|RNA2, RNA1, 35: pair-23: pair\|RNA2, RNA1, 38: pair- |
| | | | | 20: pair\|RNA2, RNA1, 41: pair-17: pair\|RNA2, RNA1, 44: pair-14: pair\|RNA2, RNA1, 47: pair- |
| | | | | 11: pair\|RNA2, RNA1, 50: pair-8: pair\|RNA2, RNA1, 53: pair-5: pair\|RNA2, RNA1, 56: pair- |
| | | | | 2: pair\|RNA2, RNA1, 5: pair-53: pair\|RNA2, RNA1, 8: pair-50: pair$$$V2.0 |
| | 673, 673_C16 | / | 1150 | RNA1{[mRVP](U)[sP].[fR](U)[sP].[mR](U)P.[fR](C)P.[mR](U)P.[fR](U)P.[mR](U)P.[fR](G) |
| | and 673_C22 | | | P.[mR](G)P.[fR](A)P.[mR](G)P.[fR](A)P.[mR](A)P.[fR](U)P.[mR](G)P.[fR](A)P.[mR](C)P.[fR](G) |
| | Antisense | | | P.[mR](C)[sP].[mR](C)[P].[mR](A)}$$$$V2.0 |
| | strand | | | |
| | 673 | 959 | / | RNA1{[mR](G)[sP].[mR](C)[sP][fR](G)P.[mR](U)P.[fR](C)P.[mR](A)P.[fR](U)P.[mR](U)P.[fR] |
| | Sense strand | | | (C)P.[mR](U)P.[fR](C)P.[mR](C)P.[fR](A)P.[mR](A)P.[fR](A)P.[mR](G)P.[fR](A)[sP].[mR](A) |
| | | | | [sP].[fR](A)}$$$$V2.0 |
| | 673_C16 | 959 | / | CHEM1{[Palmitate]}\|RNA1{[mR](G)[sP].[mR](C)[sP].[fR](G)P.[mR](U)P.[fR](C)P.[mR](A)P.[fR] |
| | Sense strand | | | (U)P.[mR](U)P.[fR](C)P.[mR](U)P.[fR](C)P.[mR](C)P.[fR](A)P.[mR](A)P.[fR](A)P.[mR](G)P.[fR] |
| | | | | (A)P.[mR](A)[sP].[fR](A)[sP]}$RNA1, CHEM1, 57: R2-1: R1$$$V2.0 |
| | 673_C22 | 959 | / | CHEM1{[ A6]}\|CHEM2{[C22COOH]}\|RNA1{[mR](G)[sP].[mR](C)[sP].[fR](G)P.[mR](U)P.[fR](C) |
| | Sense strand | | | P.[mR](A)P.[fR](U)P.[mR](U)P.[fR](C)P.[mR](U)P.[fR](C)P.[mR](C)P.[fR](A)P.[mR](A)P.[fR](A) |
| | | | | P.[mR](G)P.[fR](A)P.[mR](A)[sP].[fR](A)[sP]}$RNA1, CHEM1, 57: R2-1: R1\|CHEM1, CHEM2, |
| | | | | 1: R2-1: R1$$$V2.0 |
| 1182 | 1182 | 978 | 1169 | RNA1{[mR](A)[P].[mR](U)[sP].[fR](G)P.[mR](A)P.[fR](G)P.[mR](A)P.[fR](U)P.[mR](G)P.[fR](A) |
| | siRNA duplex | | | P.[mR](A)P.[fR](U)P.[mR](U)P.[fR](G)P.[mR](G)P.[fR](U)P.[mR](U)P.[fR](U)[sP].[mR](C)[sP]. |
| | | | | [fR](A)}\|RNA2{[mRVP](U)[sP].[fR](G)[sP].[mR](A)P.[fR](A)P.[mR](A)P.[fR](C)P.[mR](C)P.[fR] |
| | | | | (A)P.[mR](A)P.[fR](U)P.[mR](U)P.[fR](C)P.[mR](A)P.[fR](U)P.[mR](C)P.[fR](U)P.[mR](C)P.[fR] |
| | | | | (A)P.[mR](U)[sP].[mR](U)[sP].[mR](U)}$RNA1, RNA2, 11: pair-47: pair\|RNA1, RNA2, 14: pair- |
| | | | | 44: pair\|RNA1, RNA2, 17: pair-41: pair\|RNA1, RNA2, 20: pair-38: pair\|RNA1, RNA2, 23: pair- |
| | | | | 35: pair\|RNA1, RNA2, 26: pair-32: pair\|RNA1, RNA2, 29: pair-29: pair\|RNA1, RNA2, 2: pair- |
| | | | | 56: pair\|RNA1, RNA2, 32: pair-26: pair\|RNA1, RNA2, 35: pair-23: pair\|RNA1, RNA2, 38: pair- |
| | | | | 20: pair\|RNA1, RNA2, 41: pair-17: pair\|RNA1, RNA2, 44: pair-14: pair\|RNA1, RNA2, 47: pair- |
| | | | | 11: pair\|RNA1, RNA2, 50: pair-8: pair\|RNA1, RNA2, 53: pair-5: pair\|RNA1, RNA2, 56: pair- |
| | | | | 2: pair\|RNA1, RNA2, 5: pair-53: pair\|RNA1, RNA2, 8: pair-50: pair$$$V2.0 |
| | 1182_C16 | 978 | 1169 | CHEM1{[Palmitate]}\|RNA1{[mR](A)[sP].[mR](U)[sP].[fR](G)P.[mR](A)P.[fR](G)P.[mR](A)P.[fR] |
| | siRNA duplex | | | (U)P.[mR](G)P.[fR](A)P.[mR](A)P.[fR](U)P.[mR](U)P.[fR](G)P.[mR](G)P.[fR](U)P.[mR](U)P.[fR] |
| | conjugated to | | | (U)P.[mR](C)[sP].[fR](A)[sP]}\|RNA2{[mRVP](U)[sP].[fR](G)[sP].[mR](A)P.[fR](A)P.[mR](A)P.[fR] |
| | C16 fatty acid | | | (C)P.[mR](C)P.[fR](A)P.[mR](A)P.[fR](U)P.[mR](U)P.[fR](C)P.[mR](A)P.[fR](U)P.[mR](C)P.[fR] |
| | | | | (U)P.[mR](C)P.[fR](A)P.[mR](U)[sP].[mR](U)[sP].[mR](U)}$CHEM1, RNA1, 1: R1- |
| | | | | 57: R2\|RNA1, RNA2, 11: pair-47: pair\|RNA1, RNA2, 14: pair-44: pair\|RNA1, RNA2, 17: pair- |
| | | | | 41: pair\|RNA1, RNA2, 20: pair-38: pair\|RNA1, RNA2, 23: pair-35: pair\|RNA1, RNA2, 26: pair- |
| | | | | 32: pair\|RNA1, RNA2, 29: pair-29: pair\|RNA1, RNA2, 2: pair-56: pair\|RNA1, RNA2, 32: pair- |
| | | | | 26: pair\|RNA1, RNA2, 35: pair-23: pair\|RNA1, RNA2, 38: pair-20: pair\|RNA1, RNA2, 41: pair- |
| | | | | 17: pair\|RNA1, RNA2, 44: pair-14: pair\|RNA1, RNA2, 47: pair-11: pair\|RNA1, RNA2, 50: pair- |
| | | | | 8: pair\|RNA1, RNA2, 53: pair-5: pair\|RNA1, RNA2, 56: pair-2: pair\|RNA1, RNA2, 5: pair- |
| | | | | 53: pair\|RNA1, RNA2, 8: pair-50: pair$$$V2.0 |
| | 1182_C22 | 978 | 1169 | CHEM1{[A6]}\|CHEM2{[C22COOH]}\|RNA1{[mRVP](U)[P].[fR](G)[sP].[mR](A)P.[fR](A)P.[mR] |
| | siRNA duplex | | | (A)P.[fR](C)P.[mR](C)P.[fR](A)P.[mR](A)P.[fR](U)P.[mR](U)P.[fR](C)P.[mR](A)P.[fR](U)P.[mR] |
| | conjugated to | | | (C)P.[fR](U)P.[mR](C)P.[fR](A)P.[mR](U)[sP].[mR](U)[sP].[mR](U)}\|RNA2{[mR](A)[sP].[mR](U) |
| | C22 fatty acid | | | [sP].[fR](G)P.[mR](A)P.[fR](G)P.[mR](A)P.[fR](U)P.[mR](G)P.[fR](A)P.[mR](A)P.[fR](U)P.[mR] |
| | | | | (U)P.[fR](G)P.[mR](G)P.[fR](U)P.[mR](U)P.[fR](U)P.[mR](C)[sP].[fR](A)[sP]}$RNA2, CHEM1, |
| | | | | 57: R2-1: R1\|CHEM1, CHEM2, 1: R2-1: R1\|RNA2, RNA1, 11: pair-47: pair\|RNA2, RNA1, 14: pair- |
| | | | | 44: pair\|RNA2, RNA1, 17: pair-41: pair\|RNA2, RNA1, 20: pair-38: pair\|RNA2, RNA1, 23: pair- |
| | | | | 35: pair\|RNA2, RNA1, 26: pair-32: pair\|RNA2, RNA1, 29: pair-29: pair\|RNA2, RNA1, 2: pair- |
| | | | | 56: pair\|RNA2, RNA1, 32: pair-26: pair\|RNA2, RNA1, 35: pair-23: pair\|RNA2, RNA1, 38: pair- |
| | | | | 20: pair\|RNA2, RNA1, 41: pair-17: pair\|RNA2, RNA1, 44: pair-14: pair\|RNA2, RNA1, 47: pair- |
| | | | | 11: pair\|RNA2, RNA1, 50: pair-8: pair\|RNA2, RNA1, 53: pair-5: pair\|RNA2, RNA1, 56: pair- |
| | | | | 2: pair\|RNA2, RNA1, 5: pair-53: pair\|RNA2, RNA1, 8: pair-50: pair$$$V2.0 |
| | 1182, 1182_C16 | / | 1169 | RNA1{[mRVP](U)[sP].[fR](G)[sP].[mR](A)P.[fR](A)P.[mR](A)P.[fR](C)P.[mR](C)P.[fR](A)P.[mR] |
| | and 1182_C22 | | | (A)P.[fR](U)P.[mR](U)P.[fR](C)P.[mR](A)P.[fR](U)P.[mR](C)P.[fR](U)P.[mR](C)P.[fR](A)P.[mR] |
| | Antisense | | | (U)[sP].[mR](U)[sP].[mR](U)}$$$$V2.0 |
| | strand | | | |
| | 1182 | 978 | / | RNA1{[mR](A)[sP].[mR](U)[P].[fR](G)P.[mR](A)P.[fR](G)P.[mR](A)P.[fR](U)P.[mR](G)P. [fR](A) |
| | Sense strand | | | P.[mR](A)P.[fR](U)P.[mR](U)P.[fR](G)P.[mR](G)P.[fR](U)P.[mR](U)P.[fR](U)[sP].[mR](C)[sP].[fR] |
| | | | | (A)}$$$$V2.0 |
| | 1182_C16 | 978 | / | CHEM1{[Palmitate]}\|RNA1{[mR](A)[sP].[mR](U)[sP].[fR](G)P.[mR](A)P.[fR](G)P.[mR](A)P.[fR] |
| | Sense strand | | | (U)P.[mR](G)P.[fR](A)P.[mR](A)P.[fR](U)P.[mR](U)P.[fR](G)P.[mR](G)P.[fR](U)P.[mR](U)P.[fR] |
| | | | | (U)P.[mR](C)[sP].[fR](A)[sP]}$RNA1, CHEM1, 57: R2-1: R1$$$V2.0 |
| | 1182_C22 | 978 | / | CHEM1{[A6]}\|CHEM2{[C22COOH]}\|RNA1{[mR](A)[sP].[mR](U)[sP].[fR](G)P.[mR](A)P.[fR](G) |
| | Sense strand | | | P.[mR](A)P.[fR](U)P.[mR](G)P.[fR](A)P.[mR](A)P.[fR](U)P.[mR](U)P.[fR](G)P.[mR](G)P.[fR](U) |
| | | | | P.[mR](U)P.[fR](U)P.[mR](C)[sP].[fR](A)[sP]}$RNA1, CHEM1, 57: R2-1: R1\|CHEM1, CHEM2, |
| | | | | 1: R2-1: R1$$$V2.0 |
| 1770 | 1770 | 1020 | 1211 | RNA1{[mR](A)[sP].[mR](G)[sP].[fR](C)P.[mR](A)P.[fR](G)P.[mR](U)P.[fR](U)P.[mR](C)P.[fR](A) |
| | siRNA duplex | | | P.[mR](A)P.[fR](G)P.[mR](A)P.[fR](A)P.[mR](C)P.[fR](U)P.[mR](U)P.[fR](U)[sP].[mR](C)[sP].[fR] |
| | | | | (A)}\|RNA2{[mRVP](U)[sP].[fR](G)[sP].[mR](A)P.[fR](A)P.[mR](A)P.[fR](G)P.[mR](U)P.[fR](U) |
| | | | | P.[mR](C)P.[fR](U)P.[mR](U)P.[fR](G)P.[mR](A)P.[fR](A)P.[mR](C)P.[fR](U)P.[mR](G)P.[fR](C) |
| | | | | P.[mR](U)[sP].[mR](U)[sP].[mR](C)}$RNA1, RNA2, 11: pair-47: pair\|RNA1, RNA2, 14: pair- |
| | | | | 44: pair\|RNA1, RNA2, 17: pair-41: pair\|RNA1, RNA2, 20: pair-38: pair\|RNA1, RNA2, 23: pair- |
| | | | | 35: pair\|RNA1, RNA2, 26: pair-32: pair\|RNA1, RNA2, 29: pair-29: pair\|RNA1, RNA2, 2: pair- |
| | | | | 56: pair\|RNA1, RNA2, 32: pair-26: pair\|RNA1, RNA2, 35: pair-23: pair\|RNA1, RNA2, 38: pair- |
| | | | | 20: pair\|RNA1, RNA2, 41: pair-17: pair\|RNA1, RNA2, 44: pair-14: pair\|RNA1, RNA2, 47: pair- |
| | | | | 11: pair\|RNA1, RNA2, 50: pair-8: pair\|RNA1, RNA2, 53: pair-5: pair\|RNA1, RNA2, 56: pair- |
| | | | | 2: pair\|RNA1, RNA2, 5: pair-53: pair\|RNA1, RNA2, 8: pair-50: pair$$$V2.0 |
| | 1770_C16 | 1020 | 1211 | CHEM1{[Palmitate]}\|RNA1{[mR](A)[sP].[fR](G)[sP].[fR](C)P.[mR](A)P.[fR](G)P.[mR](U)P.[fR](U) |
| | siRNA duplex | | | P.[mR](C)P.[fR](A)P.[mR](A)P.[fR](G)P.[mR](A)P.[fR](A)P.[mR](C)P.[fR](U)P.[mR](U)P.[fR](U) |
| | conjugated to | | | P.[mR](C)[sP].[fR](A)[sP]}\|RNA2{[mRVP](U)[sP].[fR](G)[sP].[mR](A)P.[fR](A)P.[mR](A)P.[fR] |
| | C16 fatty acid | | | (G)P.[mR](U)P.[fR](U)P.[mR](C)P.[fR](U)P.[mR](U)P.[fR](G)P.[mR](A)P.[fR](A)P.[mR](C)P.[fR] |
| | | | | (U)P.[mR](G)P.[fR](C)P.[mR](U)[sP].[mR](U)[sP].[mR](C)}$CHEM1, RNA1, 1: R1-57: R2\|RNA1, |
| | | | | RNA2, 11: pair-47: pair\|RNA1, RNA2, 14: pair-44: pair\|RNA1, RNA2, 17: pair-41: pair\|RNA1, |
| | | | | RNA2, 20: pair-38: pair\|RNA1, RNA2, 23: pair-35: pair\|RNA1, RNA2, 26: pair-32: pair\|RNA1, |
| | | | | RNA2, 29: pair-29: pair\|RNA1, RNA2, 2: pair-56: pair\|RNA1, RNA2, 32: pair-26: pair\|RNA1, |
| | | | | RNA2, 35: pair-23: pair\|RNA1, RNA2, 38: pair-20: pair\|RNA1, RNA2, 41: pair-17: pair\|RNA1, |
| | | | | RNA2, 44: pair-14: pair\|RNA1, RNA2, 47: pair-11: pair\|RNA1, RNA2, 50: pair-8: pair\|RNA1, |
| | | | | RNA2, 53: pair-5: pair\|RNA1, RNA2, 56: pair-2: pair\|RNA1, RNA2, 5: pair-53: pair\|RNA1, |
| | | | | RNA2, 8: pair-50: pair$$$V2.0 |
| | 1770_C22 | 1020 | 1211 | CHEM1{[A6]}\|CHEM2{[C22COOH]}\|RNA1{[mRVP](U)[sP].[fR](G)[sP].[mR](A)P.[fR](A)P.[mR] |
| | siRNA duplex | | | (A)P.[fR](G)P.[mR](U)P.[fR](U)P.[mR](C)P.[fR](U)P.[mR](U)P.[fR](G)P.[mR](A)P.[fR](A)P.[mR] |
| | conjugated to | | | (C)P.[fR](U)P.[mR](G)P.[fR](C)P.[mR](U)[P].[mR](U)[sP].[mR](C)}\|RNA2{[mR](A)[sP].[mR](G) |
| | C22 fatty acid | | | [sP].[fR](C)P.[mR](A)P.[fR](G)P.[mR](U)P.[fR](U)P.[mR](C)P.[fR](A)P.[mR](A)P.[fR](G)P.[mR] |
| | | | | (A)P.[fR](A)P.[mR](C)P.[fR](U)P.[mR](U)P.[fR](U)P.[mR](C)[sP].[fR](A)[sP]}$RNA2, CHEM1, |
| | | | | 57: R2-1: R1\|CHEM1, CHEM2, 1: R2-1: R1\|RNA2, RNA1, 11: pair-47: pair\|RNA2, RNA1, 14: pair- |
| | | | | 44: pair\|RNA2, RNA1, 17: pair-41: pair\|RNA2, RNA1, 20: pair-38: pair\|RNA2, RNA1, 23: pair- |
| | | | | 35: pair\|RNA2, RNA1, 26: pair-32: pair\|RNA2, RNA1, 29: pair-29: pair\|RNA2, RNA1, 2: pair- |
| | | | | 56: pair\|RNA2, RNA1, 32: pair-26: pair\|RNA2, RNA1, 35: pair-23: pair\|RNA2, RNA1, 38: pair- |
| | | | | 20: pair\|RNA2, RNA1, 41: pair-17: pair\|RNA2, RNA1, 44: pair-14: pair\|RNA2, RNA1, 47: pair- |
| | | | | 11: pair\|RNA2, RNA1, 50: pair-8: pair\|RNA2, RNA1, 53: pair-5: pair\|RNA2, RNA1, 56: pair- |
| | | | | 2: pair\|RNA2, RNA1, 5: pair-53: pair\|RNA2, RNA1, 8: pair-50: pair$$$V2.0 |
| | 1770, 1770_C16 | / | 1211 | RNA1{[mRVP](U)[sP].[fR](G)[sP].[mR](A)P.[fR](A)P.[mR](A)P.[fR](G)P.[mR](U)P.[fR](U)P. |
| | and 1770_C22 | | | [mR](C)P.[fR](U)P.[mR](U)P.[fR](G)P.[mR](A)P.[fR](A)P.[mR](C)P.[fR](U)P.[mR](G)P.[fR](C) |
| | Antisense | | | P.[mR](U)[sP].[mR](U)[sP].[mR](C)}$$$$V2.0 |
| | strand | | | |
| | 1770 | 1020 | / | RNA1{[mR](A)[sP].[mR](G)[sP].[fR](C)P.[mR](A)P.[fR](G)P.[mR](U)P.[fR](U)P.[mR](C)P.[fR](A) |
| | Sense strand | | | P.[mR](A)P.[fR](G)P.[mR](A)P.[fR](A)P.[mR](C)P.[fR](U)P.[mR](U)P.[fR](U)[sP].[mR](C)[sP].[fR] |
| | | | | (A)}$$$$V2.0 |
| | 1770_C16 | 1020 | / | CHEM1{[Palmitate]}\|RNA1{[mR](A)[P].[mR](G)[sP].[fR](C)P.[mR](A)P.[fR](G)P.[mR](U)P.[fR] |
| | Sense strand | | | (U)P.[mR](C)P.[fR](A)P.[mR](A)P.[fR](G)P.[mR](A)P.[fR](A)P.[mR](C)P.[fR](U)P.[mR](U)P.[fR] |
| | | | | (U)P.[mR](C)[sP].[fR](A)[sP]}$RNA1, CHEM1, 57: R2-1: R1$$$V2.0 |
| | 1770_C22 | 1020 | / | CHEM1{[ A6]}\|CHEM2{[C22COOH]}\|RNA1{[mR](A)[sP].[mR](G)[sP].[fR](C)P.[mR](A)P.[fR](G) |
| | Sense strand | | | P.[mR](U)P.[fR](U)P.[mR](C)P.[fR](A)P.[mR](A)P.[fR](G)P.[mR](A)P.[fR](A)P.[mR](C)P.[fR](U) |
| | | | | P.[mR](U)P.[fR](U)P.[mR](C)[sP].[fR](A)[sP]}$RNA1, CHEM1, 57: R2-1: R1\|CHEM1, CHEM2, |
| | | | | 1: R2-1: R1$$$V2.0 |
| 1954 | 1954 | 1029 | 1220 | RNA1{[mR](G)[sP].[mR](C)[sP].[fR](U)P.[mR](G)P.[fR](G)P.[mR](U)P.[fR](G)P.[mR](G)P.[fR](C) |
| | siRNA duplex | | | P.[mR](U)P.[fR](A)P.[mR](C)P.[fR](U)P.[mR](A)P.[fR](A)P.[mR](G)P. [fR](A)[sP].[mR](A)[sP].[fR] |
| | | | | (A)}\|RNA2{[mRVP](U)[sP].[fR](U)[sP].[mR](U)P.[fR](C)P.[mR](U)P.[fR](U)P.[mR](A)P.[fR](G) |
| | | | | P.[mR](U)P.[fR](A)P.[mR](G)P.[fR](C)P.[mR](C)P.[fR](A)P.[mR](C)P.[fR](C)P.[mR](A)P.[fR](G) |
| | | | | P.[mR](C)[sP].[mR](A)[sP].[mR](G)}$RNA1, RNA2, 11: pair-47: pair\|RNA1, RNA2, 14: pair- |
| | | | | 44: pair\|RNA1, RNA2, 17: pair-41: pair\|RNA1, RNA2, 20: pair-38: pair\|RNA1, RNA2, 23: pair- |
| | | | | 35: pair\|RNA1, RNA2, 26: pair-32: pair\|RNA1, RNA2, 29: pair-29: pair\|RNA1, RNA2, 2: pair- |
| | | | | 56: pair\|RNA1, RNA2, 32: pair-26: pair\|RNA1, RNA2, 35: pair-23: pair\|RNA1, RNA2, 38: pair- |
| | | | | 20: pair\|RNA1, RNA2, 41: pair-17: pair\|RNA1, RNA2, 44: pair-14: pair\|RNA1, RNA2, 47: pair- |
| | | | | 11: pair\|RNA1, RNA2, 50: pair-8: pair\|RNA1, RNA2, 53: pair-5: pair\|RNA1, RNA2, 56: pair- |
| | | | | 2: pair\|RNA1, RNA2, 5: pair-53: pair\|RNA1, RNA2, 8: pair-50: pair$$$V2.0 |
| | 1954_C16 | 1029 | 1220 | CHEM1{[Palmitate]}\|RNA1{[mR](G)[sP].[mR](C)[sP].[fR](U)P.[mR](G)P.[fR](G)P.[mR](U)P.[fR] |
| | siRNA duplex | | | (G)P.[mR](G)P.[fR](C)P.[mR](U)P.[fR](A)P.[mR](C)P.[fR](U)P.[mR](A)P.[fR](A)P.[mR](G)P.[fR] |
| | conjugated to | | | (A)P.[mR](A)[sP].[fR](A)[sP]}\|RNA2{[mRVP](U)[sP].[fR](U)[sP].[mR](U)P.[fR](C)P.[mR](U)P.[fR] |
| | C16 fatty acid | | | (U)P.[mR](A)P.[fR](G)P.[mR](U)P.[fR](A)P.[mR](G)P.[fR](C)P.[mR](C)P.[fR](A)P.[mR](C)P.[fR] |
| | | | | (C)P.[mR](A)P.[fR](G)P.[mR](C)[sP].[mR](A)[sP].[mR](G)}$CHEM1, RNA1, 1: R1- |
| | | | | 57: R2\|RNA1, RNA2, 11: pair-47: pair\|RNA1, RNA2, 14: pair-44: pair\|RNA1, RNA2, 17: pair- |
| | | | | 41: pair\|RNA1, RNA2, 20: pair-38: pair\|RNA1, RNA2, 23: pair-35: pair\|RNA1, RNA2, 26: pair- |
| | | | | 32: pair\|RNA1, RNA2, 29: pair-29: pair\|RNA1, RNA2, 2: pair-56: pair\|RNA1, RNA2, 32: pair- |
| | | | | 26: pair\|RNA1, RNA2, 35: pair-23: pair\|RNA1, RNA2, 38: pair-20: pair\|RNA1, RNA2, 41: pair- |
| | | | | 17: pair\|RNA1, RNA2, 44: pair-14: pair\|RNA1, RNA2, 47: pair-11: pair\|RNA1, RNA2, 50: pair- |
| | | | | 8: pair\|RNA1, RNA2, 53: pair-5: pair\|RNA1, RNA2, 56: pair-2: pair\|RNA1, RNA2, 5: pair- |
| | | | | 53: pair\|RNA1, RNA2, 8: pair-50: pair$$$V2.0 |
| | 1954_C22 | 1029 | 1220 | CHEM1{[A6]}\|CHEM2{[C22COOH]}\|RNA1{[mRVP](U)[P].[fR](U)[sP].[mR](U)P.[fR](C)P.[mR] |
| | siRNA duplex | | | (U)P.[fR](U)P.[mR](A)P.[fR](G)P.[mR](U)P.[fR](A)P.[mR](G)P.[fR](C)P.[mR](C)P.[fR](A)P.[mR] |
| | conjugated to | | | (C)P.[fR](C)P.[mR](A)P.[fR](G)P.[mR](C)[sP].[mR](A)[sP].[mR](G)}\|RNA2{[mR](G)[sP].[mR](C) |
| | C22 fatty acid | | | [sP].[fR](U)P.[mR](G)P.[fR](G)P.[mR](U)P.[fR](G)P.[mR](G)P.[fR](C)P.[mR](U)P.[fR](A)P.[mR] |
| | | | | (C)P.[fR](U)P.[mR](A)P.[fR](A)P.[mR](G)P.[fR](A)P.[mR](A)[sP].[fR](A)[sP]}$RNA2, CHEM1, |
| | | | | 57: R2-1: R1\|CHEM1, CHEM2, 1: R2-1: R1\|RNA2, RNA1, 11: pair-47: pair\|RNA2, RNA1, 14: pair- |
| | | | | 44: pair\|RNA2, RNA1, 17: pair-41: pair\|RNA2, RNA1, 20: pair-38: pair\|RNA2, RNA1, 23: pair- |
| | | | | 35: pair\|RNA2, RNA1, 26: pair-32: pair\|RNA2, RNA1, 29: pair-29: pair\|RNA2, RNA1, 2: pair- |
| | | | | 56: pair\|RNA2, RNA1, 32: pair-26: pair\|RNA2, RNA1, 35: pair-23: pair\|RNA2, RNA1, 38: pair- |
| | | | | 20: pair\|RNA2, RNA1, 41: pair-17: pair\|RNA2, RNA1, 44: pair-14: pair\|RNA2, RNA1, 47: pair- |
| | | | | 11: pair\|RNA2, RNA1, 50: pair-8: pair\|RNA2, RNA1, 53: pair-5: pair\|RNA2, RNA1, 56: pair- |
| | | | | 2: pair\|RNA2, RNA1, 5: pair-53: pair\|RNA2, RNA1, 8: pair-50: pair$$$V2.0 |
| | 1954, 1954_C16 | / | 1220 | RNA1{[mRVP](U)[sP].[fR](U)[sP].[mR](U)P.[fR](C)P.[mR](U)P.[fR](U)P.[mR](A)P.[fR](G)P.[mR] |
| | and 1954_C22 | | | (U)P.[fR](A)P.[mR](G)P.[fR](C)P.[mR](C)P.[fR](A)P.[mR](C)P.[fR](C)P.[mR](A)P.[fR](G)P.[mR] |
| | Antisense | | | (C)[sP].[mR](A)[sP].[mR](G)}$$$$V2.0 |
| | strand | | | |
| | 1954 | 1029 | / | RNA1{[mR](G)[sP].[mR](C)[sP].[fR](U)P.[mR](G)P.[fR](G)P.[mR](U)P.[fR](G)P.[mR](G)P.[fR] |
| | Sense strand | | | (C)P.[mR](U)P.[fR](A)P.[mR](C)P.[fR](U)P.[mR](A)P.[fR](A)P.[mR](G)P.[fR](A)[sP].[mR](A) |
| | | | | [sP].[fR](A)}$$$$V2.0 |
| | 1954_C16 | 1029 | / | CHEM1{[Palmitate]}\|RNA1{[mR](G)[sP].[mR](C)[sP].[fR](U)P.[mR](G)P.[fR](G)P.[mR](U)P.[fR] |
| | sense strand | | | (G)P.[mR](G)P.[fR](C)P.[mR](U)P.[fR](A)P.[mR](C)P.[fR](U)P.[mR](A)P.[fR](A)P.[mR](G)P.[fR] |
| | | | | (A)P.[mR](A)[sP].[fR](A)[sP]}$RNA1, CHEM1, 57: R2-1: R1$$$V2.0 |
| | 1954_C22 | 1029 | / | CHEM1{[A6]}\|CHEM2{[C22COOH]}\|RNA1{[mR](G)[sP].[mR](C)[sP].[fR](U)P.[mR](G)P.[fR](G) |
| | sense strand | | | P.[mR](U)P.[fR](G)P.[mR](G)P.[fR](C)P.[mR](U)P.[fR](A)P.[mR](C)P.[fR](U)P.[mR](A)P.[fR](A) |
| | | | | P.[mR](G)P.[fR](A)P.[mR](A)[sP].[fR](A)[sP]}$RNA1, CHEM1, 57: R2-1: R1\|CHEM1, CHEM2, |
| | | | | 1: R2-1: R1$$$V2.0 |
| 2319 | 2319 | 1052 | 1243 | RNA1{[mR](A)[sP].[mR](U)[P].[fR](C)P.[mR](U)P.[fR](C)P.[mR](U)P.[fR](U)P.[mR](C)P.[fR](A) |
| | siRNA duplex | | | P.[mR](U)P.[fR](G)P.[mR](C)P.[fR](A)P.[mR](C)P.[fR](C)P.[mR](G)P.[fR](G)[sP].[mR](A)[sP].[fR] |
| | | | | (A)}\|RNA2{[mRVP](U)[sP].[fR](U)[sP].[mR](C)P.[fR](C)P.[mR](G)P.[fR](G)P.[mR](U)P.[fR](G) |
| | | | | P.[mR](C)P.[fR](A)P.[mR](U)P.[fR](G)P.[mR](A)P.[fR](A)P.[mR](G)P.[fR](A)P.[mR](G)P.[fR](A) |
| | | | | P.[mR](U)[sP].[mR](C)[sP].[mR](C)}$RNA1, RNA2, 11: pair-47: pair\|RNA1, RNA2, 14: pair- |
| | | | | 44: pair\|RNA1, RNA2, 17: pair-41: pair\|RNA1, RNA2, 20: pair-38: pair\|RNA1, RNA2, 23: pair- |
| | | | | 35: pair\|RNA1, RNA2, 26: pair-32: pair\|RNA1, RNA2, 29: pair-29: pair\|RNA1, RNA2, 2: pair- |
| | | | | 56: pair\|RNA1, RNA2, 32: pair-26: pair\|RNA1, RNA2, 35: pair-23: pair\|RNA1, RNA2, 38: pair- |
| | | | | 20: pair\|RNA1, RNA2, 41: pair-17: pair\|RNA1, RNA2, 44: pair-14: pair\|RNA1, RNA2, 47: pair- |
| | | | | 11: pair\|RNA1, RNA2, 50: pair-8: pair\|RNA1, RNA2, 53: pair-5: pair\|RNA1, RNA2, 56: pair- |
| | | | | 2: pair\|RNA1, RNA2, 5: pair-53: pair\|RNA1, RNA2, 8: pair-50; pair$$$V2.0 |
| | 2319_C16 | 1052 | 1243 | CHEM1{[Palmitate]}\|RNA1{[mR](A)[sP].[mR](U)[sP].[fR](C)P.[mR](U)P.[fR](C)P.[mR](U)P.[fR] |
| | siRNA duplex | | | (U)P.[mR](C)P.[fR](A)P.[mR](U)P.[fR](G)P.[mR](C)P.[fR](A)P.[mR](C)P.[fR](C)P.[mR](G)P.[fR] |
| | conjugated to | | | (G)P.[mR](A)[sP].[fR](A)[sP]}\|RNA2{[mRVP](U)[sP].[fR](U)[sP].[mR](C)P.[fR](C)P.[mR](G)P.[fR] |
| | C16 fatty acid | | | (G)P.[mR](U)P.[fR](G)P.[mR](C)P.[fR](A)P.[mR](U)P.[fR](G)P.[mR](A)P.[fR](A)P.[mR](G)P.[fR] |
| | | | | (A)P.[mR](G)P.[fR](A)P.[mR](U)[sP].[mR](C)[sP].[mR](C)}$CHEM1, RNA1, 1: R1- |
| | | | | 57: R2\|RNA1, RNA2, 11: pair-47: pair\|RNA1, RNA2, 14: pair-44: pair\|RNA1, RNA2, 17: pair- |
| | | | | 41: pair\|RNA1, RNA2, 20: pair-38: pair\|RNA1, RNA2, 23: pair-35: pair\|RNA1, RNA2, 26: pair- |
| | | | | 32: pair\|RNA1, RNA2, 29: pair-29: pair\|RNA1, RNA2, 2: pair-56: pair\|RNA1, RNA2, 32: pair- |
| | | | | 26: pair\|RNA1, RNA2, 35: pair-23: pair\|RNA1, RNA2, 38: pair-20: pair\|RNA1, RNA2, 41: pair- |
| | | | | 17: pair\|RNA1, RNA2, 44: pair-14: pair\|RNA1, RNA2, 47: pair-11: pair\|RNA1, RNA2, 50: pair- |
| | | | | 8: pair\|RNA1, RNA2, 53: pair-5: pair\|RNA1, RNA2, 56: pair-2: pair\|RNA1, RNA2, 5: pair- |
| | | | | 53: pair\|RNA1, RNA2, 8: pair-50: pair$$$V2.0 |
| | 2319_C22 | 1052 | 1243 | CHEM1{[ A6]}\|CHEM2{[C22COOH]}\|RNA1{[mRVP](U)[sP].[fR](U)[sP].[mR](C)P.[fR](C)P.[mR] |
| | siRNA duplex | | | (G)P.[fR](G)P.[mR](U)P.[fR](G)P.[mR](C)P.[fR](A)P.[mR](U)P.[fR](G)P.[mR](A)P.[fR](A)P.[mR] |
| | conjugated to | | | (G)P.[fR](A)P.[mR](G)P.[fR](A)P.[mR](U)[sP].[mR](C)[sP].[mR](C)}\|RNA2{[mR](A)[sP].[mR](U) |
| | C22 fatty acid | | | [sP].[fR](C)P.[mR](U)P.[fR](C)P.[mR](U)P.[fR](U)P.[mR](C)P.[fR](A)P.[mR](U)P.[fR](G)P.[mR] |
| | | | | (C)P.[fR](A)P.[mR](C)P.[fR](C)P.[mR](G)P.[fR](G)P.[mR](A)[sP].[fR](A)[sP]}$RNA2, CHEM1, |
| | | | | 57: R2-1: R1\|CHEM1, CHEM2, 1: R2-1: R1\|RNA2, RNA1, 11: pair-47: pair\|RNA2, RNA1, 14: pair- |
| | | | | 44: pair\|RNA2, RNA1, 17: pair-41: pair\|RNA2, RNA1, 20: pair-38: pair\|RNA2, RNA1, 23: pair- |
| | | | | 35: pair\|RNA2, RNA1, 26: pair-32: pair\|RNA2, RNA1, 29: pair-29: pair\|RNA2, RNA1, 2: pair- |
| | | | | 56: pair\|RNA2, RNA1, 32: pair-26: pair\|RNA2, RNA1, 35: pair-23: pair\|RNA2, RNA1, 38: pair- |
| | | | | 20: pair\|RNA2, RNA1, 41: pair-17: pair\|RNA2, RNA1, 44: pair-14: pair\|RNA2, RNA1, 47: pair- |
| | | | | 11: pair\|RNA2, RNA1, 50: pair-8: pair\|RNA2, RNA1, 53: pair-5: pair\|RNA2, RNA1, 56: pair- |
| | | | | 2: pair\|RNA2, RNA1, 5: pair-53: pair\|RNA2, RNA1, 8: pair-50: pair$$$V2.0 |
| | 2319, 2319_C16 | / | 1243 | RNA1{[mRVP](U)[sP].[fR](U)[sP].[mR](C)P.[fR](C)P.[mR](G)P.[fR](G)P.[mR](U)P.[fR](G)P.[mR] |
| | and 2319_C22 | | | (C)P.[fR](A)P.[mR](U)P.[fR](G)P.[mR](A)P.[fR](A)P.[mR](G)P.[fR](A)P.[mR](G)P.[fR](A)P.[mR] |
| | Antisense | | | (U)[sP].[mR](C)[sP].[mR](C)}$$$$V2.0 |
| | strand | | | |
| | 2319 | 1052 | / | RNA1{[mR](A)[sP].[mR](U)[sP].[fR](C)P.[mR](U)P.[fR](C)P.[mR](U)P.[fR](U)P.[mR](C)P.[fR](A) |
| | Sense strand | | | P.[mR](U)P.[fR](G)P.[mR](C)P.[fR](A)P.[mR](C)P.[fR](C)P.[mR](G)P.[fR](G)[sP].[mR](A)[sP].[fR] |
| | | | | (A)}$$$$V2.0 |
| | 2319_C16 | 1052 | / | CHEM1{[Palmitate]}\|RNA1{[mR](A)[sP].[mR](U)[sP].[fR](C)P.[mR](U)P.[fR](C)P.[mR](U)P.[fR] |
| | Sense strand | | | (U)P.[mR](C)P.[fR](A)P.[mR](U)P.[fR](G)P.[mR](C)P.[fR](A)P.[mR](C)P.[fR](C)P.[mR](G)P.[fR] |
| | | | | (G)P.[mR](A)[sP].[fR](A)[sP]}$RNA1, CHEM1, 57: R2-1: R1$$$V2.0 |
| | 2319_C22 | 1052 | / | CHEM1{[ A6]}\|CHEM2{[C22COOH]}\|RNA1{[mR](A)[sP].[mR](U)[sP].[fR](C)P.[mR](U)P.[fR](C) |
| | Sense strand | | | P.[mR](U)P.[fR](U)P.[mR](C)P.[fR](A)P.[mR](U)P.[fR](G)P.[mR](C)P.[fR](A)P.[mR](C)P.[fR](C) |
| | | | | P.[mR](G)P.[fR](G)P.[mR](A)[sP].[fR](A)[sP]}$RNA1, CHEM1, 57: R2-1: R1\|CHEM1, CHEM2, |
| | | | | 1: R2-1: R1$$$V2.0 |
| 3131 | 3131 | 1071 | 1262 | RNA1{[mR](G)[sP].[mR](A)[sP].[fR](C)P.[mR](G)P.[fR](G)P.[mR](A)P.[fR](G)P.[mR](G)P.[fR](A) |
| | siRNA duplex | | | P.[mR](A)P.[fR](A)P.[mR](U)P.[fR](G)P.[mR](G)P.[fR](U)P.[mR](A)P.[fR](U)[sP].[mR](U)[sP].[fR] |
| | | | | (A)}\|RNA2{[mRVP](U)[sP].[fR](A)[sP].[mR](A)P.[fR](U)P.[mR](A)P.[fR](C)P.[mR](C)P.[fR](A) |
| | | | | P.[mR](U)P.[fR](U)P.[mR](U)P.[fR](C)P.[mR](C)P.[fR](U)P.[mR](C)P.[fR](C)P.[mR](G)P.[fR](U) |
| | | | | P.[mR](C)[sP].[mR](U)[sP].[mR](U)}$RNA1, RNA2, 11: pair-47: pair\|RNA1, RNA2, 14: pair- |
| | | | | 44: pair\|RNA1, RNA2, 17: pair-41: pair\|RNA1, RNA2, 20: pair-38: pair\|RNA1, RNA2, 23: pair- |
| | | | | 35: pair\|RNA1, RNA2, 26: pair-32: pair\|RNA1, RNA2, 29: pair-29: pair\|RNA1, RNA2, 2: pair- |
| | | | | 56: pair\|RNA1, RNA2, 32: pair-26: pair\|RNA1, RNA2, 35: pair-23: pair\|RNA1, RNA2, 38: pair- |
| | | | | 20: pair\|RNA1, RNA2, 41: pair-17: pair\|RNA1, RNA2, 44: pair-14: pair\|RNA1, RNA2, 47: pair- |
| | | | | 11: pair\|RNA1, RNA2, 50: pair-8: pair\|RNA1, RNA2, 53: pair-5: pair\|RNA1, RNA2, 56: pair- |
| | | | | 2: pair\|RNA1, RNA2, 5: pair-53: pair\|RNA1, RNA2, 8: pair-50: pair$$$V2.0 |
| | 3131_C16 | 1071 | 1262 | CHEM1{[Palmitate]}\|RNA1{[mR](G)[sP].[mR](A)[sP].[fR](C)P.[mR](G)P.[fR](G)P.[mR](A)P.[fR] |
| | siRNA duplex | | | (G)P.[mR](G)P.[fR](A)P.[mR](A)P.[fR](A)P.[mR](U)P.[fR](G)P.[mR](G)P.[fR](U)P.[mR](A)P.[fR] |
| | conjugated to | | | (U)P.[mR](U)[sP].[fR](A)[P]}\|RNA2{[mRVP](U)[sP].[fR](A)[sP].[mR](A)P.[fR](U)P.[mR](A)P.[fR] |
| | C16 fatty acid | | | (C)P.[mR](C)P.[fR](A)P.[mR](U)P.[fR](U)P.[mR](U)P.[fR](C)P.[mR](C)P.[fR](U)P.[mR](C)P.[fR] |
| | | | | (C)P.[mR](G)P.[fR](U)P.[mR](C)[sP].[mR](U)[sP].[mR](U)}$CHEM1, RNA1, 1: R1- |
| | | | | 57: R2\|RNA1, RNA2, 11: pair-47: pair\|RNA1, RNA2, 14: pair-44: pair\|RNA1, RNA2, 17: pair- |
| | | | | 41: pair\|RNA1, RNA2, 20: pair-38: pair\|RNA1, RNA2, 23: pair-35: pair\|RNA1, RNA2, 26: pair- |
| | | | | 32: pair\|RNA1, RNA2, 29: pair-29: pair\|RNA1, RNA2, 2: pair-56: pair\|RNA1, RNA2, 32: pair- |
| | | | | 26: pair\|RNA1, RNA2, 35: pair-23: pair\|RNA1, RNA2, 38: pair-20: pair\|RNA1, RNA2, 41: pair- |
| | | | | 17: pair\|RNA1, RNA2, 44: pair-14: pair\|RNA1, RNA2, 47: pair-11: pair\|RNA1, RNA2, 50: pair- |
| | | | | 8: pair\|RNA1, RNA2, 53: pair-5: pair\|RNA1, RNA2, 56: pair-2: pair\|RNA1, RNA2, 5: pair- |
| | | | | 53: pair\|RNA1, RNA2, 8: pair-50: pair$$$V2.0 |
| | 3131_C22 | 1071 | 1262 | CHEM1{[ A6]}\|CHEM2{[C22COOH]}\|RNA1{[mRVP](U)[sP].[fR](A)[sP].[mR](A)P.[fR](U)P.[mR] |
| | siRNA duplex | | | (A)P.[fR](C)P.[mR](C)P.[fR](A)P.[mR](U)P.[fR](U)P.[mR](U)P.[fR](C)P.[mR](C)P.[fR](U)P.[mR] |
| | conjugated to | | | (C)P.[fR](C)P.[mR](G)P.[fR](U)P.[mR](C)[sP].[mR](U)[sP].[mR](U)}\|RNA2{[mR](G)[sP].[mR](A) |
| | C22 fatty acid | | | [sP].[fR](C)P.[mR](G)P.[fR](G)P.[mR](A)P.[fR](G)P.[mR](G)P.[fR](A)P.[mR](A)P.[fR](A)P.[mR] |
| | | | | (U)P.[fR](G)P.[mR](G)P.[fR](U)P.[mR](A)P.[fR](U)P.[mR](U)[sP].[fR](A)[sP]}$RNA2, CHEM1, |
| | | | | 57: R2-1: R1\|CHEM1, CHEM2, 1: R2-1: R1\|RNA2, RNA1, 11: pair-47: pair\|RNA2, RNA1, 14: pair- |
| | | | | 44: pair\|RNA2, RNA1, 17: pair-41: pair\|RNA2, RNA1, 20: pair-38: pair\|RNA2, RNA1, 23: pair- |
| | | | | 35: pair\|RNA2, RNA1, 26: pair-32: pair\|RNA2, RNA1, 29: pair-29: pair\|RNA2, RNA1, 2: pair- |
| | | | | 56: pair\|RNA2, RNA1, 32: pair-26: pair\|RNA2, RNA1, 35: pair-23: pair\|RNA2, RNA1, 38: pair- |
| | | | | 20: pair\|RNA2, RNA1, 41: pair-17: pair\|RNA2, RNA1, 44: pair-14: pair\|RNA2, RNA1, 47: pair- |
| | | | | 11: pair\|RNA2, RNA1, 50: pair-8: pair\|RNA2, RNA1, 53: pair-5: pair\|RNA2, RNA1, 56: pair- |
| | | | | 2: pair\|RNA2, RNA1, 5: pair-53: pair\|RNA2, RNA1, 8: pair-50: pair$$$V2.0 |
| | 3131, 3131_C16 | / | 1262 | RNA1{[mRVP](U)[sP].[fR](A)[sP].[mR](A)P.[fR](U)P.[mR](A)P.[fR](C)P.[mR](C)P.[fR](A)P.[mR] |
| | and 3131_C22 | | | (U)P.[fR](U)P.[mR](U)P.[fR](C)P.[mR](C)P.[fR](U)P.[mR](C)P.[fR](C)P.[mR](G)P.[fR](U)P.[mR] |
| | Antisense | | | (C)[sP].[mR](U)[sP].[mR](U)}$$$$V2.0 |
| | strand | | | |
| | 3131 | 1071 | / | RNA1{[mR](G)[sP].[mR](A)[sP].[fR](C)P.[mR](G)P.[fR](G)P.[mR](A)P.[fR](G)P.[mR](G)P.[fR](A) |
| | Sense strand | | | P.[mR](A)P.[fR](A)P.[mR](U)P.[fR](G)P.[mR](G)P.[fR](U)P.[mR](A)P.[fR](U)[P].[mR](U)[sP].[fR] |
| | | | | (A)}$$$$V2.0 |
| | 3131_C16 | 1071 | / | CHEM1{[Palmitate]}\|RNA1{[mR](G)[sP].[mR](A)[sP].[fR](C)P.[mR](G)P.[fR](G)P.[mR](A)P.[fR] |
| | Sense strand | | | (G)P.[mR](G)P.[fR](A)P.[mR](A)P.[fR](A)P.[mR](U)P.[fR](G)P.[mR](G)P.[fR](U)P.[mR](A)P.[fR] |
| | | | | (U)P.[mR](U)[sP].[fR](A)[sP]}$RNA1, CHEM1, 57: R2-1: R1$$$V2.0 |
| | 3131_C22 | 1071 | / | CHEM1{[A6]}\|CHEM2{[C22COOH]}\|RNA1{[mR](G)[sP].[mR](A)[sP].[fR](C)P.[mR](G)P.[fR](G) |
| | Sense strand | | | P.[mR](A)P.[fR](G)P.[mR](G)P.[fR](A)P.[mR](A)P.[fR](A)P.[mR](U)P.[fR](G)P.[mR](G)P.[fR](U) |
| | | | | P.[mR](A)P.[fR](U)P.[mR](U)[sP].[fR](A)[sP]}$RNA1, CHEM1, 57: R2-1: R1\|CHEM1, CHEM2, |
| | | | | 1: R2-1: R1$$$V2.0 |
| 3255 | 3255 | 1085 | 1276 | RNA1{[mR](G)[sP].[mR](U)[sP].[fR](A)P.[mR](A)P.[fR](G)P.[mR](G)P.[fR](G)P.[mR](G)P.[fR](A) |
| | siRNA duplex | | | P.[mR](U)P.[fR](G)P.[mR](G)P.[fR](A)P.[mR](C)P.[fR](U)P.[mR](A)P.[fR](U)[sP].[mR](U)[sP].[fR] |
| | | | | (A)}\|RNA2{[mRVP](U)[sP].[fR](A)[sP].[mR](A)P.[fR](U)P.[mR](A)P.[fR](G)P.[mR](U)P.[fR](C) |
| | | | | P.[mR](C)P.[fR](A)P.[mR](U)P.[fR](C)P.[mR](C)P.[fR](C)P.[mR](C)P.[fR](U)P.[mR](U)P.[fR](A) |
| | | | | P.[mR](C)[sP].[mR](A)[sP].[mR](A)}$RNA1, RNA2, 11: pair-47: pair\|RNA1, RNA2, 14: pair- |
| | | | | 44: pair\|RNA1, RNA2, 17: pair-41: pair\|RNA1, RNA2, 20: pair-38: pair\|RNA1, RNA2, 23: pair- |
| | | | | 35: pair\|RNA1, RNA2, 26: pair-32: pair\|RNA1, RNA2, 29: pair-29: pair\|RNA1, RNA2, 2: pair- |
| | | | | 56: pair\|RNA1, RNA2, 32: pair-26: pair\|RNA1, RNA2, 35: pair-23: pair\|RNA1, RNA2, 38: pair- |
| | | | | 20: pair\|RNA1, RNA2, 41: pair-17: pair\|RNA1, RNA2, 44: pair-14: pair\|RNA1, RNA2, 47: pair- |
| | | | | 11: pair\|RNA1, RNA2, 50: pair-8: pair\|RNA1, RNA2, 53: pair-5: pair\|RNA1, RNA2, 56: pair- |
| | | | | 2: pair\|RNA1, RNA2, 5: pair-53: pair\|RNA1, RNA2, 8: pair-50: pair$$$V2.0 |
| | 3255_C16 | 1085 | 1276 | CHEM1{[Palmitate]}\|RNA1{[mR](G)[sP].[mR](U)[sP].[fR](A)P.[mR](A)P.[fR](G)P.[mR](G)P.[fR] |
| | siRNA duplex | | | (G)P.[mR](G)P.[fR](A)P.[mR](U)P.[fR](G)P.[mR](G)P.[fR](A)P.[mR](C)P.[fR](U)P.[mR](A)P.[fR] |
| | conjugated to | | | (U)P.[mR](U)[sP].[fR](A)[sP]}\|RNA2{[mRVP](U)[sP].[fR](A)[P].[mR](A)P.[fR](U)P.[mR](A)P.[fR] |
| | C16 fatty acid | | | (G)P.[mR](U)P.[fR](C)P.[mR](C)P.[fR](A)P.[mR](U)P.[fR](C)P.[mR](C)P.[fR](C)P.[mR](C)P.[fR] |
| | | | | (U)P.[mR](U)P.[fR](A)P.[mR](C)[sP].[mR](A)[sP].[mR](A)}$CHEM1, RNA1, 1: R1- |
| | | | | 57: R2\|RNA1, RNA2, 11: pair-47: pair\|RNA1, RNA2, 14: pair-44: pair\|RNA1, RNA2, 17: pair- |
| | | | | 41: pair\|RNA1, RNA2, 20: pair-38: pair\|RNA1, RNA2, 23: pair-35: pair\|RNA1, RNA2, 26: pair- |
| | | | | 32: pair\|RNA1, RNA2, 29: pair-29: pair\|RNA1, RNA2, 2: pair-56: pair\|RNA1, RNA2, 32: pair- |
| | | | | 26: pair\|RNA1, RNA2, 35: pair-23: pair\|RNA1, RNA2, 38: pair-20: pair\|RNA1, RNA2, 41: pair- |
| | | | | 17: pair\|RNA1, RNA2, 44: pair-14: pair\|RNA1, RNA2, 47: pair-11: pair\|RNA1, RNA2, 50: pair- |
| | | | | 8: pair\|RNA1, RNA2, 53: pair-5: pair\|RNA1, RNA2, 56: pair-2: pair\|RNA1, RNA2, 5: pair- |
| | | | | 53: pair\|RNA1, RNA2, 8: pair-50: pair$$$V2.0 |
| | 3255_C22 | 1085 | 1276 | CHEM1{[A6]}\|CHEM2{[C22COOH]}\|RNA1{[mRVP](U)[sP].[fR](A)[sP].[mR](A)P.[fR](U)P.[mR] |
| | siRNA duplex | | | (A)P.[fR](G)P.[mR](U)P.[fR](C)P.[mR](C)P.[fR](A)P.[mR](U)P.[fR](C)P.[mR](C)P.[fR](C)P.[mR] |
| | conjugated to | | | (C)P.[fR](U)P.[mR](U)P.[fR](A)P.[mR](C)[sP].[mR](A)[sP].[mR](A)}\|RNA2{[mR](G)[sP].[mR](U) |
| | C22 fatty acid | | | [sP].[fR](A)P.[mR](A)P.[fR](G)P.[mR](G)P.[fR](G)P.[mR](G)P.[fR](A)P.[mR](U)P.[fR](G)P.[mR] |
| | | | | (G)P.[fR](A)P.[mR](C)P.[fR](U)P.[mR](A)P.[fR](U)P.[mR](U)[sP].[fR](A)[sP]}$RNA2, CHEM1, |
| | | | | 57: R2-1: R1\|CHEM1, CHEM2, 1: R2-1: R1\|RNA2, RNA1, 11: pair-47: pair\|RNA2, RNA1, 14: pair- |
| | | | | 44: pair\|RNA2, RNA1, 17: pair-41: pair\|RNA2, RNA1, 20: pair-38: pair\|RNA2, RNA1, 23: pair- |
| | | | | 35: pair\|RNA2, RNA1, 26: pair-32: pair\|RNA2, RNA1, 29: pair-29: pair\|RNA2, RNA1, 2: pair- |
| | | | | 56: pair\|RNA2, RNA1, 32: pair-26: pair\|RNA2, RNA1, 35: pair-23: pair\|RNA2, RNA1, 38: pair- |
| | | | | 20: pair\|RNA2, RNA1, 41: pair-17: pair\|RNA2, RNA1, 44: pair-14: pair\|RNA2, RNA1, 47: pair- |
| | | | | 11: pair\|RNA2, RNA1, 50: pair-8: pair\|RNA2, RNA1, 53: pair-5: pair\|RNA2, RNA1, 56: pair- |
| | | | | 2: pair\|RNA2, RNA1, 5: pair-53: pair\|RNA2, RNA1, 8: pair-50: pair$$$V2.0 |
| | 3255, 3255_C16 | / | 1276 | RNA1{[mRVP](U)[sP].[fR](A)[sP].[mR](A)P.[fR](U)P.[mR](A)P.[fR](G)P.[mR](U)P.[fR](C)P.[mR] |
| | and 3255_C22 | | | (C)P.[fR](A)P.[mR](U)P.[fR](C)P.[mR](C)P.[fR](C)P.[mR](C)P.[fR](U)P.[mR](U)P.[fR](A)P.[mR] |
| | Antisense | | | (C)[sP].[mR](A)[sP].[mR](A)}$$$$V2.0 |
| | strand | | | |
| | 3255 | 1085 | / | RNA1{[mR](G)[sP].[mR](U)[sP].[fR](A)P.[mR](A)P.[fR](G)P.[mR](G)P.[fR](G)P.[mR](G)P.[fR](A) |
| | Sense strand | | | P.[mR](U)P.[fR](G)P.[mR](G)P.[fR](A)P.[mR](C)P.[fR](U)P.[mR](A)P.[fR](U)[sP].[mR](U)[sP].[fR] |
| | | | | (A)}$$$$V2.0 |
| | 3255_C16 | 1085 | / | CHEM1{[Palmitate]}\|RNA1{[mR](G)[sP].[mR](U)[sP].[fR](A)P.[mR](A)P.[fR](G)P.[mR](G)P.[fR] |
| | Sense strand | | | (G)P.[mR](G)P.[fR](A)P.[mR](U)P.[fR](G)P.[mR](G)P.[fR](A)P.[mR](C)P.[fR](U)P.[mR](A)P.[fR] |
| | | | | (U)P.[mR](U)[sP].[fR](A)[sP]}$RNA1, CHEM1, 57: R2-1: R1$$$V2.0 |
| | 3255_C22 | 1085 | / | CHEM1{[ A6]}\|CHEM2{[C22COOH]}\|RNA1{[mR](G)[sP].[mR](U)[P].[fR](A)P.[mR](A)P.[fR](G) |
| | Sense strand | | | P.[mR](G)P.[fR](G)P.[mR](G)P.[fR](A)P.[mR](U)P.[fR](G)P.[mR](G)P.[fR](A)P.[mR](C)P.[fR](U) |
| | | | | P.[mR](A)P.[fR](U)P.[mR](U)[sP].[fR](A)[sP]}$RNA1, CHEM1, 57: R2-1: R1\|CHEM1, CHEM2, |
| | | | | 1: R2-1: R1$$$V2.0 |
| 3265 | 3265 | 1090 | 1281 | RNA1{[mR](G)[sP].[mR](G)[sP].[fR](A)P.[mR](C)P.[fR](U)P.[mR](A)P.[fR](U)P.[mR](U)P. [fR](U) |
| | siRNA duplex | | | P.[mR](G)P.[fR](G)P.[mR](G)P.[fR](U)P.[mR](U)P.[fR](C)P.[mR](U)P.[fR](C)[sP].[mR](G)[sP].[fR] |
| | | | | (A)}\|RNA2{[mRVP](U)[sP].[fR](C)[sP].[mR](G)P.[fR](A)P.[mR](G)P.[fR](A)P.[mR](A)P.[fR](C) |
| | | | | P.[mR](C)P.[fR](C)P.[mR](A)P.[fR](A)P.[mR](A)P.[fR](U)P.[mR](A)P.[fR](G)P.[mR](U)P. [fR](C) |
| | | | | P.[mR](C)[sP].[mR](A)[sP].[mR](U)}$RNA1, RNA2, 11: pair-47: pair\|RNA1, RNA2, 14: pair- |
| | | | | 44: pair\|RNA1, RNA2, 17: pair-41: pair\|RNA1, RNA2, 20: pair-38: pair\|RNA1, RNA2, 23: pair- |
| | | | | 35: pair\|RNA1, RNA2, 26: pair-32: pair\|RNA1, RNA2, 29: pair-29: pair\|RNA1, RNA2, 2: pair- |
| | | | | 56: pair\|RNA1, RNA2, 32: pair-26: pair\|RNA1, RNA2, 35: pair-23: pair\|RNA1, RNA2, 38: pair- |
| | | | | 20: pair\|RNA1, RNA2, 41: pair-17: pair\|RNA1, RNA2, 44: pair-14: pair\|RNA1, RNA2, 47: pair- |
| | | | | 11: pair\|RNA1, RNA2, 50: pair-8: pair\|RNA1, RNA2, 53: pair-5: pair\|RNA1, RNA2, 56: pair- |
| | | | | 2: pair\|RNA1, RNA2, 5: pair-53: pair\|RNA1, RNA2, 8: pair-50: pair$$$V2.0 |
| | 3265_C16 | 1090 | 1281 | CHEM1{[Palmitate]}\|RNA1{[mR](G)[sP].[mR](G)[sP].[fR](A)P.[mR](C)P.[fR](U)P.[mR](A)P.[fR] |
| | siRNA duplex | | | (U)P.[mR](U)P.[fR](U)P.[mR](G)P.[fR](G)P.[mR](G)P.[fR](U)P.[mR](U)P.[fR](C)P.[mR](U)P.[fR] |
| | conjugated to | | | (C)P.[mR](G)[sP].[fR](A)[sP]}\|RNA2{[mRVP](U)[sP].[fR](C)[sP].[mR](G)P.[fR](A)P.[mR](G)P.[fR] |
| | C16 fatty acid | | | (A)P.[mR](A)P.[fR](C)P.[mR](C)P.[fR](C)P.[mR](A)P.[fR](A)P.[mR](A)P.[fR](U)P.[mR](A)P.[fR] |
| | | | | (G)P.[mR](U)P.[fR](C)P.[mR](C)[sP].[mR](A)[sP].[mR](U)}$CHEM1, RNA1, 1: R1- |
| | | | | 57: R2\|RNA1, RNA2, 11: pair-47: pair\|RNA1, RNA2, 14: pair-44: pair\|RNA1, RNA2, 17: pair- |
| | | | | 41: pair\|RNA1, RNA2, 20: pair-38: pair\|RNA1, RNA2, 23: pair-35: pair\|RNA1, RNA2, 26: pair- |
| | | | | 32: pair\|RNA1, RNA2, 29: pair-29: pair\|RNA1, RNA2, 2: pair-56: pair\|RNA1, RNA2, 32: pair- |
| | | | | 26: pair\|RNA1, RNA2, 35: pair-23: pair\|RNA1, RNA2, 38: pair-20: pair\|RNA1, RNA2, 41: pair- |
| | | | | 17: pair\|RNA1, RNA2, 44: pair-14: pair\|RNA1, RNA2, 47: pair-11: pair\|RNA1, RNA2, 50: pair- |
| | | | | 8: pair\|RNA1, RNA2, 53: pair-5: pair\|RNA1, RNA2, 56: pair-2: pair\|RNA1, RNA2, 5: pair- |
| | | | | 53: pair\|RNA1, RNA2, 8: pair-50: pair$$$V2.0 |
| | 3265_C22 | 1090 | 1281 | CHEM1{[A6]}\|CHEM2{[C22COOH]}\|RNA1{[mRVP](U)[sP].[fR](C)[sP].[mR](G)P.[fR](A)P.[mR] |
| | siRNA duplex | | | (G)P.[fR](A)P.[mR](A)P.[fR](C)P.[mR](C)P.[fR](C)P.[mR](A)P.[fR](A)P.[mR](A)P.[fR](U)P.[mR] |
| | conjugated to | | | (A)P.[fR](G)P.[mR](U)P.[fR](C)P.[mR](C)[sP].[mR](A)[P].[mR](U)}\|RNA2{[mR](G)[sP].[mR](G) |
| | C22 fatty acid | | | [sP].[fR](A)P.[mR](C)P.[fR](U)P.[mR](A)P.[fR](U)P.[mR](U)P.[fR](U)P.[mR](G)P.[fR](G)P.[mR] |
| | | | | (G)P.[fR](U)P.[mR](U)P.[fR](C)P.[mR](U)P.[fR](C)P.[mR](G)[sP].[fR](A)[sP]}$RNA2, CHEM1, |
| | | | | 57: R2-1: R1\|CHEM1, CHEM2, 1: R2-1: R1\|RNA2, RNA1, 11: pair-47: pair\|RNA2, RNA1, 14: pair- |
| | | | | 44: pair\|RNA2, RNA1, 17: pair-41: pair\|RNA2, RNA1, 20: pair-38: pair\|RNA2, RNA1, 23: pair- |
| | | | | 35: pair\|RNA2, RNA1, 26: pair-32: pair\|RNA2, RNA1, 29: pair-29: pair\|RNA2, RNA1, 2: pair- |
| | | | | 56: pair\|RNA2, RNA1, 32: pair-26: pair\|RNA2, RNA1, 35: pair-23: pair\|RNA2, RNA1, 38: pair- |
| | | | | 20: pair\|RNA2, RNA1, 41: pair-17: pair\|RNA2, RNA1, 44: pair-14: pair\|RNA2, RNA1, 47: pair- |
| | | | | 11: pair\|RNA2, RNA1, 50: pair-8: pair\|RNA2, RNA1, 53: pair-5: pair\|RNA2, RNA1, 56: pair- |
| | | | | 2: pair\|RNA2, RNA1, 5: pair-53: pair\|RNA2, RNA1, 8: pair-50: pair$$$V2.0 |
| | 3265, 3265_C16 | / | 1281 | RNA1{[mRVP](U)[sP].[fR](C)[sP].[mR](G)P.[fR](A)P.[mR](G)P.[fR](A)P.[mR](A)P.[fR](C)P.[mR] |
| | and 3265_C22 | | | (C)P.[fR](C)P.[mR](A)P.[fR](A)P.[mR](A)P.[fR](U)P.[mR](A)P.[fR](G)P.[mR](U)P.[fR](C)P.[mR] |
| | Antisense | | | (C)[sP].[mR](A)[sP].[mR](U)}$$$$V2.0 |
| | strand | | | |
| | 3265 | 1090 | / | RNA1{[mR](G)[sP].[mR](G)[sP].[fR](A)P.[mR](C)P.[fR](U)P.[mR](A)P.[fR](U)P.[mR](U)P.[fR](U) |
| | Sense strand | | | P.[mR](G)P.[fR](G)P.[mR](G)P.[fR](U)P.[mR](U)P.[fR](C)P.[mR](U)P.[fR](C)[sP].[mR](G)[sP].[fR] |
| | | | | (A)}$$$$V2.0 |
| | 3265_C16 | 1090 | / | CHEM1{[Palmitate]}\|RNA1{[mR](G)[sP].[mR](G)[sP].[fR](A)P.[mR](C)P.[fR](U)P.[mR](A)P.[fR] |
| | sense strand | | | (U)P.[mR](U)P.[fR](U)P.[mR](G)P.[fR](G)P.[mR](G)P.[fR](U)P.[mR](U)P.[fR](C)P.[mR](U)P.[fR] |
| | | | | (C)P.[mR](G)[sP].[fR](A)[sP]}$RNA1, CHEM1, 57: R2-1: R1$$$V2.0 |
| | 3265_C22 | 1090 | / | CHEM1{[A6]}\|CHEM2{[C22COOH]}\|RNA1{[mR](G)[sP].[mR](G)[sP].[fR](A)P.[mR](C)P.[fR](U) |
| | sense strand | | | P.[mR](A)P.[fR](U)P.[mR](U)P.[fR](U)P.[mR](G)P.[fR](G)P.[mR](G)P.[fR](U)P.[mR](U)P.[fR](C) |
| | | | | P.[mR](U)P.[fR](C)P.[mR](G)[sP].[fR](A)[P]}$RNA1, CHEM1, 57: R2-1: R1\|CHEM1, CHEM2, |
| | | | | 1: R2-1: R1$$$V2.0 |
| 3313 | 3313 | 1105 | 1296 | RNA1{[mR](A)[sP].[mR](A)[sP].[fR](A)P.[mR](U)P.[fR](G)P.[mR](U)P.[fR](C)P.[mR](C)P.[fR](U) |
| | siRNA duplex | | | P.[mR](U)P.[fR](G)P.[mR](U)P.[fR](U)P.[mR](G)P.[fR](A)P.[mR](G)P.[fR](A)[sP].[mR](G)[sP].[fR] |
| | | | | (A)}\|RNA2{[mRVP](U)[sP].[fR](C)[sP].[mR](U)P.[fR](C)P.[mR](U)P.[fR](C)P.[mR](A)P.[fR](A) |
| | | | | P.[mR](C)P.[fR](A)P.[mR](A)P.[fR](G)P.[mR](G)P.[fR](A)P.[mR](C)P.[fR](A)P.[mR](U)P.[fR](U) |
| | | | | P.[mR](U)[sP].[mR](C)[sP].[mR](U)}$RNA1, RNA2, 11: pair-47: pair\|RNA1, RNA2, 14: pair- |
| | | | | 44: pair\|RNA1, RNA2, 17: pair-41: pair\|RNA1, RNA2, 20: pair-38: pair\|RNA1, RNA2, 23: pair- |
| | | | | 35: pair\|RNA1, RNA2, 26: pair-32: pair\|RNA1, RNA2, 29: pair-29: pair\|RNA1, RNA2, 2: pair- |
| | | | | 56: pair\|RNA1, RNA2, 32: pair-26: pair\|RNA1, RNA2, 35: pair-23: pair\|RNA1, RNA2, 38: pair- |
| | | | | 20: pair\|RNA1, RNA2, 41: pair-17: pair\|RNA1, RNA2, 44: pair-14: pair\|RNA1, RNA2, 47: pair- |
| | | | | 11: pair\|RNA1, RNA2, 50: pair-8: pair\|RNA1, RNA2, 53: pair-5: pair\|RNA1, RNA2, 56: pair- |
| | | | | 2: pair\|RNA1, RNA2, 5: pair-53: pair\|RNA1, RNA2, 8: pair-50: pair$$$V2.0 |
| | 3313_C16 | 1105 | 1296 | CHEM1{[Palmitate]}\|RNA1{[mR](A)[sP].[mR](A)[P].[fR](A)P.[mR](U)P.[fR](G)P.[mR](U)P.[fR] |
| | siRNA duplex | | | (C)P.[mR](C)P.[fR](U)P.[mR](U)P.[fR](G)P.[mR](U)P.[fR](U)P.[mR](G)P.[fR](A)P.[mR](G)P.[fR] |
| | conjugated to | | | (A)P.[mR](G)[sP].[fR](A)[sP]}\|RNA2{[mRVP](U)[sP].[fR](C)[sP].[mR](U)P.[fR](C)P.[mR](U)P.[fR] |
| | C16 fatty acid | | | (C)P.[mR](A)P.[fR](A)P.[mR](C)P.[fR](A)P.[mR](A)P.[fR](G)P.[mR](G)P.[fR](A)P.[mR](C)P.[fR] |
| | | | | (A)P.[mR](U)P.[fR](U)P.[mR](U)[sP].[mR](C)[sP].[mR](U)}$CHEM1, RNA1, 1: R1- |
| | | | | 57: R2\|RNA1, RNA2, 11: pair-47: pair\|RNA1, RNA2, 14: pair-44: pair\|RNA1, RNA2, 17: pair- |
| | | | | 41: pair\|RNA1, RNA2, 20: pair-38: pair\|RNA1, RNA2, 23: pair-35: pair\|RNA1, RNA2, 26: pair- |
| | | | | 32: pair\|RNA1, RNA2, 29: pair-29: pair\|RNA1, RNA2, 2: pair-56: pair\|RNA1, RNA2, 32: pair- |
| | | | | 26: pair\|RNA1, RNA2, 35: pair-23: pair\|RNA1, RNA2, 38: pair-20: pair\|RNA1, RNA2, 41: pair- |
| | | | | 17: pair\|RNA1, RNA2, 44: pair-14: pair\|RNA1, RNA2, 47: pair-11: pair\|RNA1, RNA2, 50: pair- |
| | | | | 8: pair\|RNA1, RNA2, 53: pair-5: pair\|RNA1, RNA2, 56: pair-2: pair\|RNA1, RNA2, 5: pair- |
| | | | | 53: pair\|RNA1, RNA2, 8: pair-50: pair$$$V2.0 |
| | 3313_C22 | 1105 | 1296 | CHEM1{[ A6]}\|CHEM2{[C22COOH]}\|RNA1{[mRVP](U)[sP].[fR](C)[sP].[mR](U)P.[fR](C)P.[mR] |
| | siRNA duplex | | | (U)P.[fR](C)P.[mR](A)P.[fR](A)P.[mR](C)P.[fR](A)P.[mR](A)P.[fR](G)P.[mR](G)P.[fR](A)P.[mR] |
| | conjugated to | | | (C)P.[fR](A)P.[mR](U)P.[fR](U)P.[mR](U)[sP].[mR](C)[sP].[mR](U)}\|RNA2{[mR](A)[sP].[mR](A) |
| | C22 fatty acid | | | [sP].[fR](A)P.[mR](U)P.[fR](G)P.[mR](U)P.[fR](C)P.[mR](C)P.[fR](U)P.[mR](U)P.[fR](G)P.[mR] |
| | | | | (U)P.[fR](U)P.[mR](G)P.[fR](A)P.[mR](G)P.[fR](A)P.[mR](G)[P].[fR](A)[sP]}$RNA2, CHEM1, |
| | | | | 57: R2-1: R1\|CHEM1, CHEM2, 1: R2-1: R1\|RNA2, RNA1, 11: pair-47: pair\|RNA2, RNA1, 14: pair- |
| | | | | 44: pair\|RNA2, RNA1, 17: pair-41: pair\|RNA2, RNA1, 20: pair-38: pair\|RNA2, RNA1, 23: pair- |
| | | | | 35: pair\|RNA2, RNA1, 26: pair-32: pair\|RNA2, RNA1, 29: pair-29: pair\|RNA2, RNA1, 2: pair- |
| | | | | 56: pair\|RNA2, RNA1, 32: pair-26: pair\|RNA2, RNA1, 35: pair-23: pair\|RNA2, RNA1, 38: pair- |
| | | | | 20: pair\|RNA2, RNA1, 41: pair-17: pair\|RNA2, RNA1, 44: pair-14: pair\|RNA2, RNA1, 47: pair- |
| | | | | 11: pair\|RNA2, RNA1, 50: pair-8: pair\|RNA2, RNA1, 53: pair-5: pair\|RNA2, RNA1, 56: pair- |
| | | | | 2: pair\|RNA2, RNA1, 5: pair-53: pair\|RNA2, RNA1, 8: pair-50: pair$$$V2.0 |
| | 3313, 3313_C16 | / | 1296 | RNA1{[mRVP](U)[sP].[fR](C)[sP].[mR](U)P.[fR](C)P.[mR](U)P.[fR](C)P.[mR](A)P.[fR](A)P.[mR] |
| | and 3313_C22 | | | (C)P.[fR](A)P.[mR](A)P.[fR](G)P.[mR](G)P.[fR](A)P.[mR](C)P.[fR](A)P.[mR](U)P.[fR](U)P.[mR] |
| | Antisense | | | (U)[sP].[mR](C)[sP].[mR](U)}$$$$V2.0 |
| | strand | | | |
| | 3313 | 1105 | / | RNA1{[mR](A)[sP].[mR](A)[sP].[fR](A)P.[mR](U)P.[fR](G)P.[mR](U)P.[fR](C)P.[mR](C)P. [fR](U) |
| | Sense strand | | | P.[mR](U)P.[fR](G)P.[mR](U)P.[fR](U)P.[mR](G)P.[fR](A)P.[mR](G)P.[fR](A)[sP].[mR](G)[sP].[fR] |
| | | | | (A)}$$$$V2.0 |
| | 3313_C16 | 1105 | / | CHEM1{[Palmitate]}\|RNA1{[mR](A)[sP].[mR](A)[sP].[fR](A)P.[mR](U)P.[fR](G)P.[mR](U)P.[fR] |
| | Sense strand | | | (C)P.[mR](C)P.[fR](U)P.[mR](U)P.[fR](G)P.[mR](U)P.[fR](U)P.[mR](G)P.[fR](A)P.[mR](G)P.[fR] |
| | | | | (A)P.[mR](G)[sP].[fR](A)[sP]}$RNA1, CHEM1, 57: R2-1: R1$$$V2.0 |
| | 3313_C22 | 1105 | / | CHEM1{[ A6]}\|CHEM2{[C22COOH]}\|RNA1{[mR](A)[sP].[mR](A)[sP].[fR](A)P.[mR](U)P.[fR](G) |
| | Sense strand | | | P.[mR](U)P.[fR](C)P.[mR](C)P.[fR](U)P.[mR](U)P.[fR](G)P.[mR](U)P.[fR](U)P.[mR](G)P.[fR](A) |
| | | | | P.[mR](G)P.[fR](A)P.[mR](G)[sP].[fR](A)[sP]}$RNA1, CHEM1, 57: R2-1: R1\|CHEM1, CHEM2, |
| | | | | 1: R2-1: R1$$$V2.0 |

In some embodiments, the compound of the invention is selected from compounds 614, 673, 724, 728, 753, 756, 818, 874, 875, 876, 877, 878, 883, 884, 1069, 1075, 1085, 1107, 1108, 1138, 1182, 1189, 1190, 1304, 1306, 1311, 1367, 1368, 1372, 1412, 1413, 1432, 1579, 1580, 1581, 1583, 1584, 1586, 1587, 1588, 1595, 1596, 1601, 1602, 1603, 1608, 1609, 1611, 1640, 1642, 1671, 1672, 1673, 1674, 1677, 1678, 1690, 1692, 1698, 1699, 1723, 1769, 1770, 1780, 1798, 1876, 1927, 1928, 1929, 1936, 1952, 1954, 1956, 1958, 1978, 2066, 2068, 2102, 2111, 2138, 2146, 2148, 2205, 2206, 2218, 2229, 2230, 2237, 2238, 2239, 2269, 2308, 2317, 2318, 2319, 2320, 2321, 2322, 2323, 2520, 2527, 2647, 2761, 2762, 2763, 2764, 2811, 2962, 2975, 2977, 3028, 3032, 3081, 3131, 3134, 3141, 3144, 3146, 3147, 3159, 3160, 3229, 3247, 3250, 3251, 3252, 3254, 3255, 3258, 3259, 3260, 3261, 3265, 3268, 3272, 3275, 3276, 3278, 3279, 3281, 3282, 3283, 3284, 3285, 3286, 3313, 3314, 3323, 3353, 3365, 3367, 3368, 3371, 3372, 3376, 3409, 3505, 3556, 3557, 3558, 3559, 3654, 3662, 3663, 3683, 3689, 3694, 3695, 3698, 3702, 3719, 3781, 3894, 4099, 4169, 4239, 4305, 4374, 4411, 4475, 4612, 4671, 4672, 4679, 4682, 4683, 4684, 4690, 4794, 4803, and 4807, as shown in Table 3.

In some embodiments the compound of the invention is selected from compounds 614, 673, 1182, 1770, 1954, 2319, 3131, 3255, 3265, and 3313 as shown in Table 3 herein.

In some embodiments the compound of the invention is compound 614 as shown in Table 3 herein. In some embodiments the compound of the invention is compound 614 as shown in Table 3 herein, conjugated to a C16 fatty acid, preferably palmitic acid. In some embodiments the compound of the invention is compound 614 as shown in Table 3 herein, conjugated to a C22 fatty acid, preferably behenic acid.

In some embodiments the compound of the invention is compound 673 as shown in Table 3 herein. In some embodiments the compound of the invention is compound 673 as shown in Table 3 herein, conjugated to a C16 fatty acid, preferably palmitic acid. In some embodiments the compound of the invention is compound 673 as shown in Table 3 herein, conjugated to a C22 fatty acid, preferably behenic acid.

In some embodiments the compound of the invention is compound 1182 as shown in Table 3 herein. In some embodiments the compound of the invention is compound 1182 as shown in Table 3 herein, conjugated to a C16 fatty acid, preferably palmitic acid. In some embodiments the compound of the invention is compound 1182 as shown in Table 3 herein, conjugated to a C22 fatty acid, preferably behenic acid.

In some embodiments the compound of the invention is compound 1770 as shown in Table 3 herein. In some embodiments the compound of the invention is compound 1770 as shown in Table 3 herein, conjugated to a C16 fatty acid, preferably palmitic acid. In some embodiments the compound of the invention is compound 1770 as shown in Table 3 herein, conjugated to a C22 fatty acid, preferably behenic acid.

In some embodiments the compound of the invention is compound 1954 as shown in Table 3 herein. In some embodiments the compound of the invention is compound 1954 as shown in Table 3 herein, conjugated to a C16 fatty acid, preferably palmitic acid. In some embodiments the compound of the invention is compound 1954 as shown in Table 3 herein, conjugated to a C22 fatty acid, preferably behenic acid.

In some embodiments the compound of the invention is compound 2319 as shown in Table 3 herein. In some embodiments the compound of the invention is compound 2319 as shown in Table 3 herein, conjugated to a C16 fatty acid, preferably palmitic acid. In some embodiments the compound of the invention is compound 2319 as shown in Table 3 herein, conjugated to a C22 fatty acid, preferably behenic acid.

In some embodiments the compound of the invention is compound 3131 as shown in Table 3 herein. In some embodiments the compound of the invention is compound 3131 as shown in Table 3 herein, conjugated to a C16 fatty acid, preferably palmitic acid. In some embodiments the compound of the invention is compound 3131 as shown in Table 3 herein, conjugated to a C22 fatty acid, preferably behenic acid.

In some embodiments the compound of the invention is compound 3255 as shown in Table 3 herein. In some embodiments the compound of the invention is compound 3255 as shown in Table 3 herein, conjugated to a C16 fatty acid, preferably palmitic acid. In some embodiments the compound of the invention is compound 3255 as shown in Table 3 herein, conjugated to a C22 fatty acid, preferably behenic acid.

In some embodiments the compound of the invention is compound 3265 as shown in Table 3 herein. In some embodiments the compound of the invention is compound 3265 as shown in Table 3 herein, conjugated to a C16 fatty acid, preferably palmitic acid. In some embodiments the compound of the invention is compound 3265 as shown in Table 3 herein, conjugated to a C22 fatty acid, preferably behenic acid.

In some embodiments the compound of the invention is compound 3313 as shown in Table 3 herein. In some embodiments the compound of the invention is compound 3313 as shown in Table 3 herein, conjugated to a C16 fatty acid, preferably palmitic acid. In some embodiments the compound of the invention is compound 3313 as shown in Table 3 herein, conjugated to a C22 fatty acid, preferably behenic acid.

In some embodiments the compound of the invention is selected from compounds 614, 673, 1182, 1770, 1954, 2319, 3131, 3255, 3265, and 3313 as shown in Table 4 herein (using HELM strings) or as depicted using chemical structures herein.

In some embodiments the compound of the invention is compound 614 as shown in Table 4 herein (using HELM strings) or as depicted using chemical structures herein. In some embodiments the compound of the invention is compound 614 as shown in Table 4 herein (using HELM strings) or as depicted using chemical structures herein, conjugated to a C16 fatty acid, preferably palmitic acid. In some embodiments the compound of the invention is compound 614 as shown in Table 4 herein (using HELM strings) or as depicted using chemical structures herein, conjugated to a C22 fatty acid, preferably behenic acid.

In some embodiments the compound of the invention is compound 673 as shown in Table 4 herein (using HELM strings) or as depicted using chemical structures herein. In some embodiments the compound of the invention is compound 673 as shown in Table 4 herein (using HELM strings) or as depicted using chemical structures herein, conjugated to a C16 fatty acid, preferably palmitic acid. In some embodiments the compound of the invention is compound 673 as shown in Table 4 herein (using HELM strings) or as depicted using chemical structures herein, conjugated to a C22 fatty acid, preferably behenic acid.

In some embodiments the compound of the invention is compound 1182 as shown in Table 4 herein (using HELM strings) or as depicted using chemical structures herein. In some embodiments the compound of the invention is compound 1182 as shown in Table 4 herein (using HELM strings) or as depicted using chemical structures herein, conjugated to a C16 fatty acid, preferably palmitic acid. In some embodiments the compound of the invention is compound 1182 as shown in Table 3 herein (using HELM strings) or as depicted using chemical structures herein, conjugated to a C22 fatty acid, preferably behenic acid.

In some embodiments the compound of the invention is compound 1770 as shown in Table 4 herein (using HELM strings) or as depicted using chemical structures herein. In some embodiments the compound of the invention is compound 1770 as shown in Table 4 herein (using HELM strings) or as depicted using chemical structures herein, conjugated to a C16 fatty acid, preferably palmitic acid. In some embodiments the compound of the invention is compound 1770 as shown in Table 3 herein (using HELM strings) or as depicted using chemical structures herein, conjugated to a C22 fatty acid, preferably behenic acid.

In some embodiments the compound of the invention is compound 1954 as shown in Table 4 herein (using HELM strings) or as depicted using chemical structures herein. In some embodiments the compound of the invention is compound 1954 as shown in Table 4 herein (using HELM strings) or as depicted using chemical structures herein, conjugated to a C16 fatty acid, preferably palmitic acid. In some embodiments the compound of the invention is compound 1954 as shown in Table 3 herein (using HELM strings) or as depicted using chemical structures herein, conjugated to a C22 fatty acid, preferably behenic acid.

In some embodiments the compound of the invention is compound 2319 as shown in Table 4 herein (using HELM strings) or as depicted using chemical structures herein. In some embodiments the compound of the invention is compound 2319 as shown in Table 4 herein (using HELM strings) or as depicted using chemical structures herein, conjugated to a C16 fatty acid, preferably palmitic acid. In some embodiments the compound of the invention is compound 2319 as shown in Table 3 herein (using HELM strings) or as depicted using chemical structures herein, conjugated to a C22 fatty acid, preferably behenic acid.

In some embodiments the compound of the invention is compound 3131 as shown in Table 4 herein (using HELM strings) or as depicted using chemical structures herein. In some embodiments the compound of the invention is compound 3131 as shown in Table 4 herein (using HELM strings) or as depicted using chemical structures herein, conjugated to a C16 fatty acid, preferably palmitic acid. In some embodiments the compound of the invention is compound 3131 as shown in Table 3 herein (using HELM strings) or as depicted using chemical structures herein, conjugated to a C22 fatty acid, preferably behenic acid.

In some embodiments the compound of the invention is compound 3255 as shown in Table 4 herein (using HELM strings) or as depicted using chemical structures herein. In some embodiments the compound of the invention is compound 3255 as shown in Table 4 herein (using HELM strings) or as depicted using chemical structures herein, conjugated to a C16 fatty acid, preferably palmitic acid. In some embodiments the compound of the invention is compound 3255 as shown in Table 3 herein (using HELM strings) or as depicted using chemical structures herein, conjugated to a C22 fatty acid, preferably behenic acid.

In some embodiments the compound of the invention is compound 3265 as shown in Table 4 herein (using HELM strings) or as depicted using chemical structures herein. In some embodiments the compound of the invention is compound 3265 as shown in Table 4 herein (using HELM strings) or as depicted using chemical structures herein, conjugated to a C16 fatty acid, preferably palmitic acid. In some embodiments the compound of the invention is compound 3265 as shown in Table 3 herein (using HELM strings) or as depicted using chemical structures herein, conjugated to a C22 fatty acid, preferably behenic acid.

In some embodiments the compound of the invention is compound 3313 as shown in Table 4 herein (using HELM strings) or as depicted using chemical structures herein. In some embodiments the compound of the invention is compound 3313 as shown in Table 4 herein (using HELM strings) or as depicted using chemical structures herein, conjugated to a C16 fatty acid, preferably palmitic acid. In some embodiments the compound of the invention is compound 3313 as shown in Table 3 herein (using HELM strings) or as depicted using chemical structures herein, conjugated to a C22 fatty acid, preferably behenic acid.

In some embodiments the compound of the invention is selected from compound 614_C16, 673_C16, 1182_C16, 1770_C16, 1954_C16, 2319_C16, 3131_C16, 3255_C16, 3265_C16, and 3313_C16 as shown in Table 4 herein (using HELM strings) or as depicted using chemical structures herein.

In some embodiments the compound of the invention is selected from compound 614_C22, 673_C22, 1182_C22, 1770_C22, 1954_C22, 2319_C22, 3131_C22, 3255_C22, 3265_C22, and 3313_C22 as shown in Table 4 herein (using HELM strings) or as depicted using chemical structures herein, preferably from compounds 614_C22 and 1182_C22.

In some embodiments the compound of the invention is compound 614_C16 as shown in Table 4 herein (using HELM strings).

In some embodiments the compound of the invention is compound 673_C16 as shown in Table 4 herein (using HELM strings).

In some embodiments the compound of the invention is compound 1182_C16 as shown in Table 4 herein (using HELM strings).

In some embodiments the compound of the invention is compound 1770_C16 as shown in Table 4 herein (using HELM strings).

In some embodiments the compound of the invention is compound 1954_C16 as shown in Table 4 herein (using HELM strings).

In some embodiments the compound of the invention is compound 2319_C16 as shown in Table 4 herein (using HELM strings).

In some embodiments the compound of the invention is compound 3131_C16 as shown in Table 4 herein (using HELM strings).

In some embodiments the compound of the invention is compound 3255_C16 as shown in Table 4 herein (using HELM strings).

In some embodiments the compound of the invention is compound 3265_C16 as shown in Table 4 herein (using HELM strings).

In some embodiments the compound of the invention is compound 3313_C16 as shown in Table 4 herein (using HELM strings).

In some embodiments the compound of the invention is compound 614_C22 as shown in Table 4 herein (using HELM strings).

In some embodiments the compound of the invention is compound 673_C22 as shown in Table 4 herein (using HELM strings).

In some embodiments the compound of the invention is compound 1182_C22 as shown in Table 4 herein (using HELM strings).

In some embodiments the compound of the invention is compound 1770_C22 as shown in Table 4 herein (using HELM strings).

In some embodiments the compound of the invention is compound 1954_C22 as shown in Table 4 herein (using HELM strings).

In some embodiments the compound of the invention is compound 2319_C22 as shown in Table 4 herein (using HELM strings).

In some embodiments the compound of the invention is compound 3131_C22 as shown in Table 4 herein (using HELM strings).

In some embodiments the compound of the invention is compound 3255_C22 as shown in Table 4 herein (using HELM strings).

In some embodiments the compound of the invention is compound 3265_C22 as shown in Table 4 herein (using HELM strings).

In some embodiments the compound of the invention is compound 3313_C22 as shown in Table 4 herein (using HELM strings).

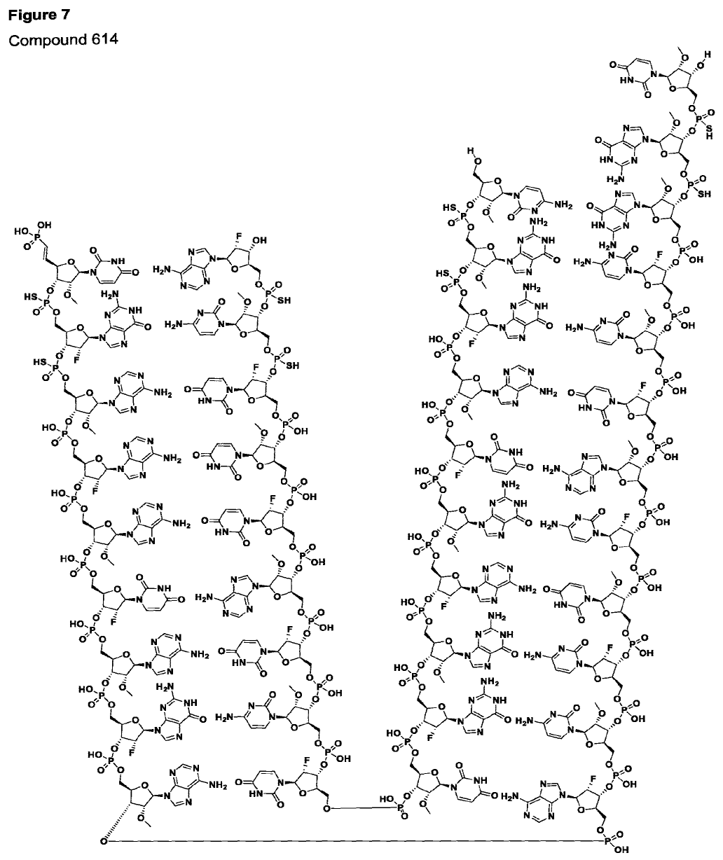

The invention provides a compound comprising the structure as shown in FIG. 7 (compound 614). The invention provides a compound consisting of the structure as shown in FIG. 7 (compound 614).

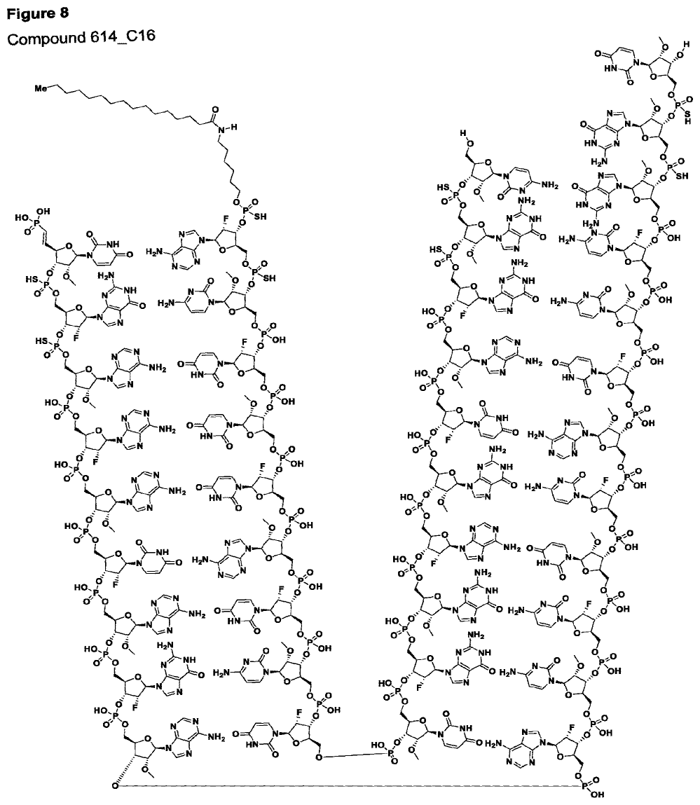

The invention provides a compound comprising the structure as shown in FIG. 8 (compound 614_C16). The invention provides a compound consisting of the structure as shown in FIG. 8 (compound 614_C16).

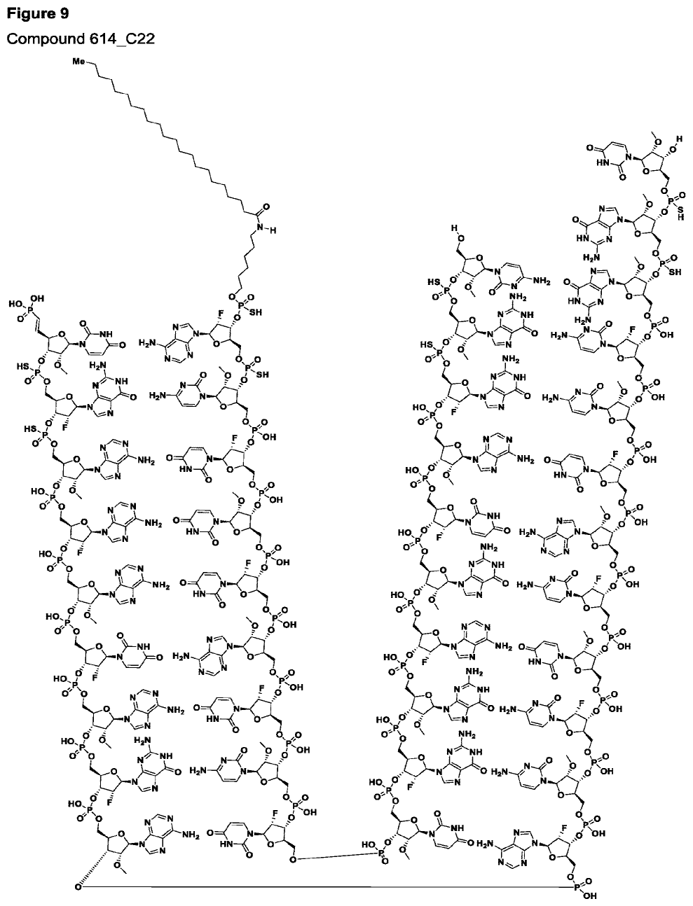

The invention provides a compound comprising the structure as shown in FIG. 9 (compound 614_C22). The invention provides a compound consisting of the structure as shown in FIG. 9 (compound 614_C22).

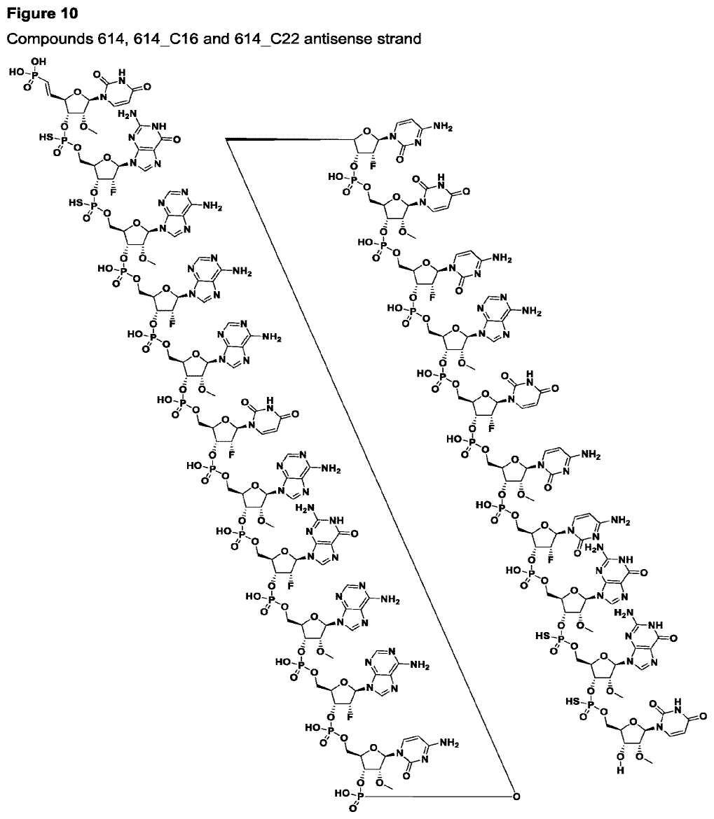

The invention provides a compound comprising the structure as shown in FIG. 10 (antisense strand of compounds 614, 614_C16 and 614_C22).

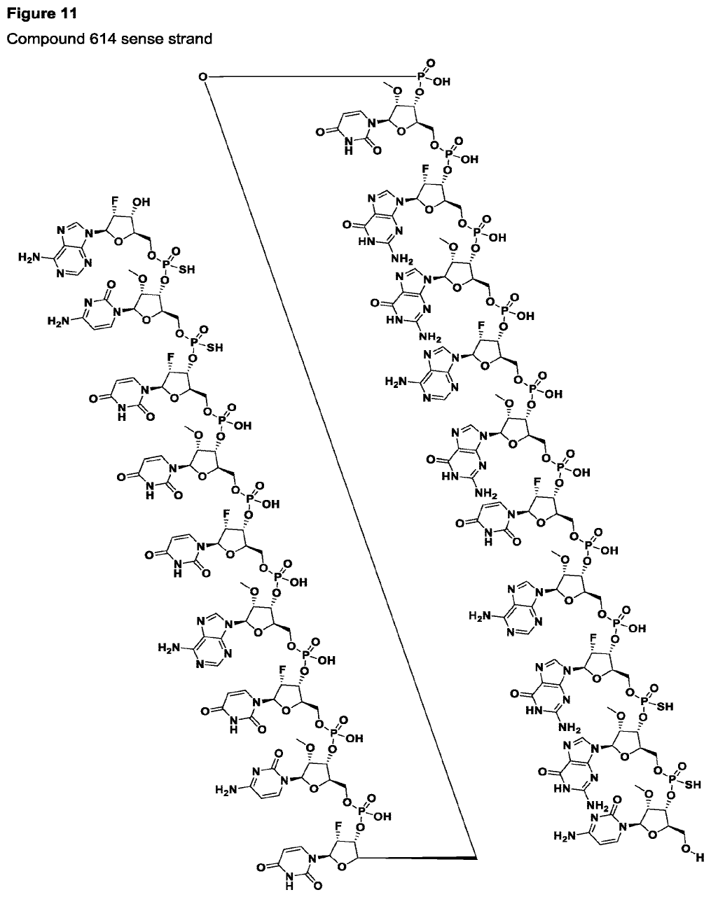

The invention provides a compound comprising the structure as shown in FIG. 11 (sense strand of compound 614).

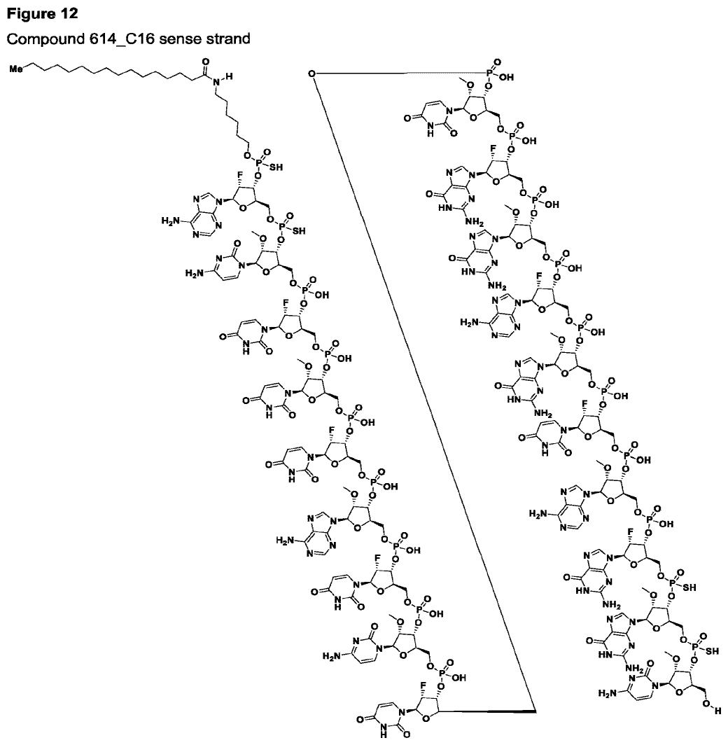

The invention provides a compound comprising the structure as shown in FIG. 12 (the sense strand of compound 614_C16).

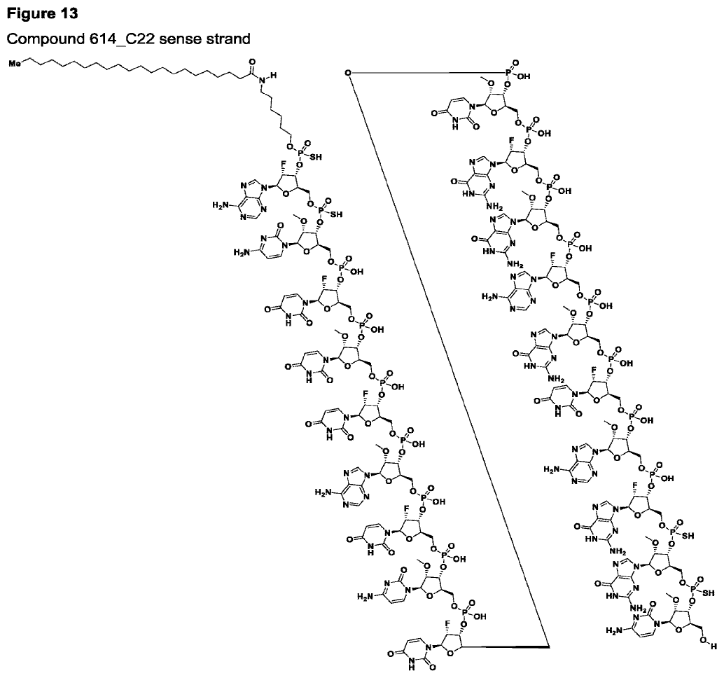

The invention provides a compound comprising the structure as shown in FIG. 13 (the sense strand of compound 614_C22).

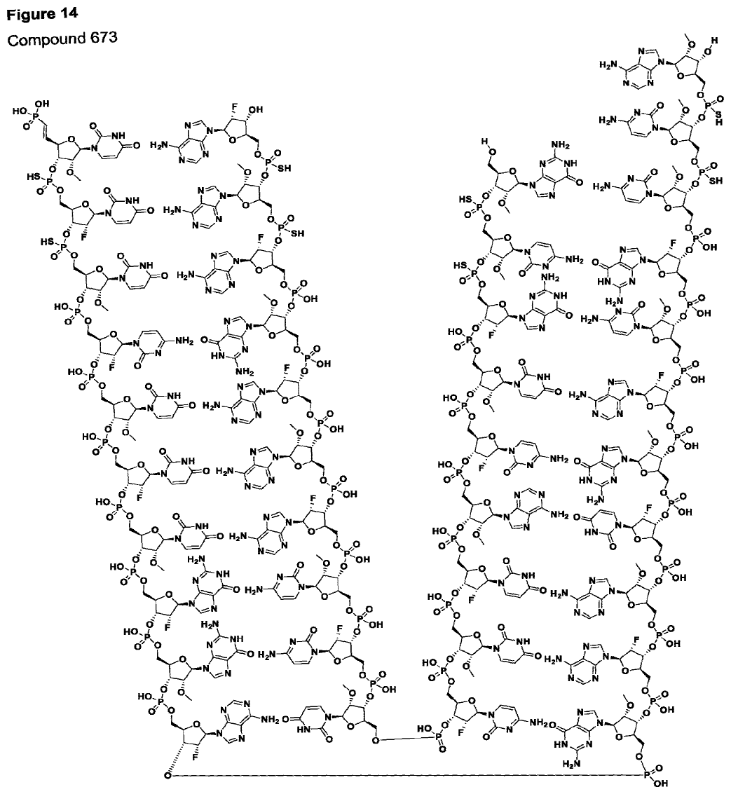

The invention provides a compound comprising the structure as shown in FIG. 14 (compound 673). The invention provides a compound consisting of the structure as shown in FIG. 14 (compound 673).

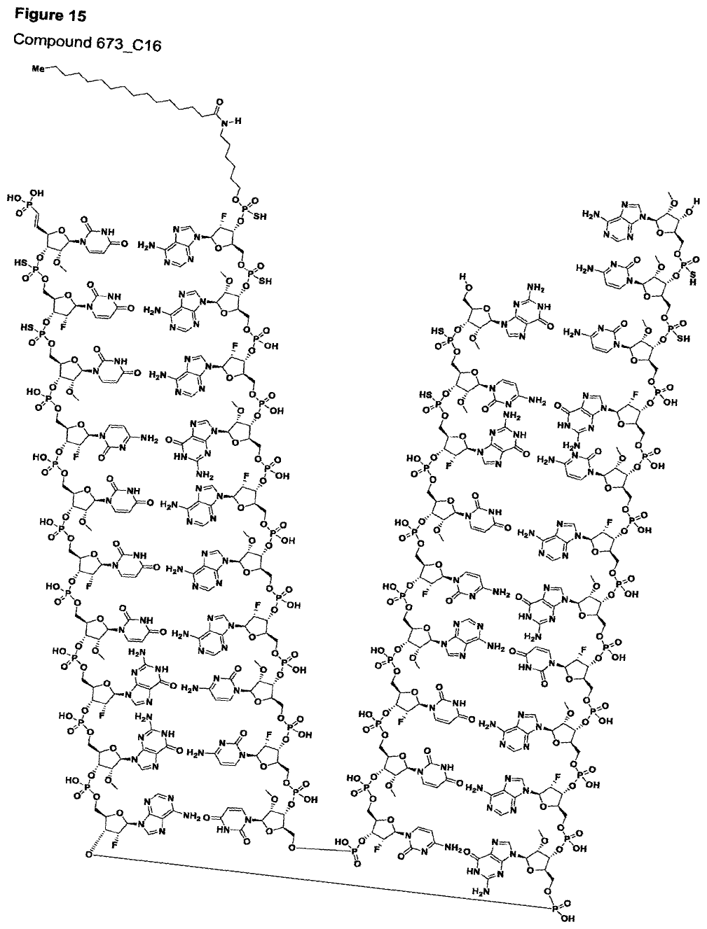

The invention provides a compound comprising the structure as shown in FIG. 15 (compound 673_C16). The invention provides a compound consisting of the structure as shown in FIG. 15 (compound 673_C16).

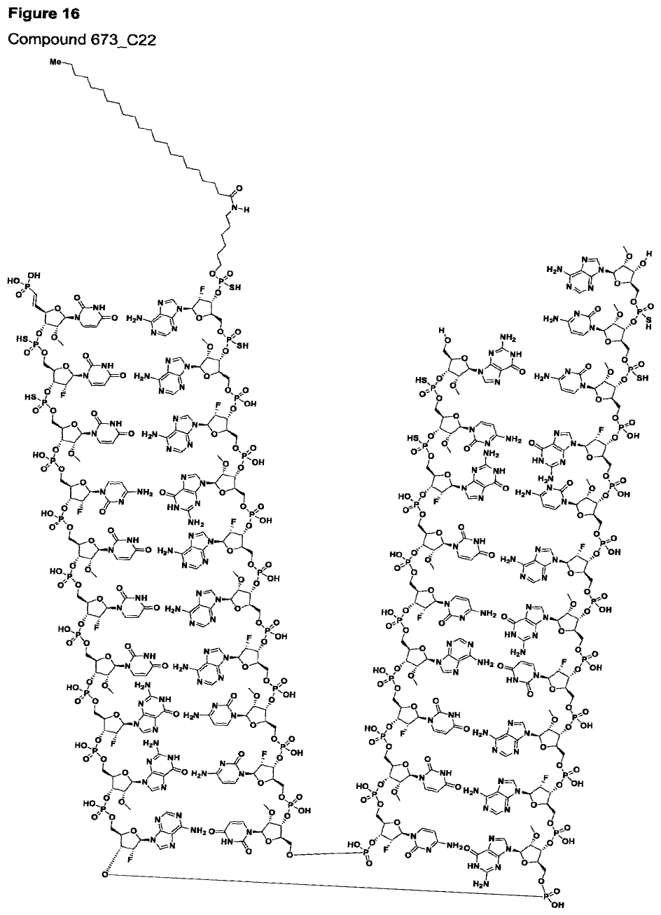

The invention provides a compound comprising the structure as shown in FIG. 16 (compound 673_C22). The invention provides a compound consisting of the structure as shown in FIG. 16 (compound 673_C22).

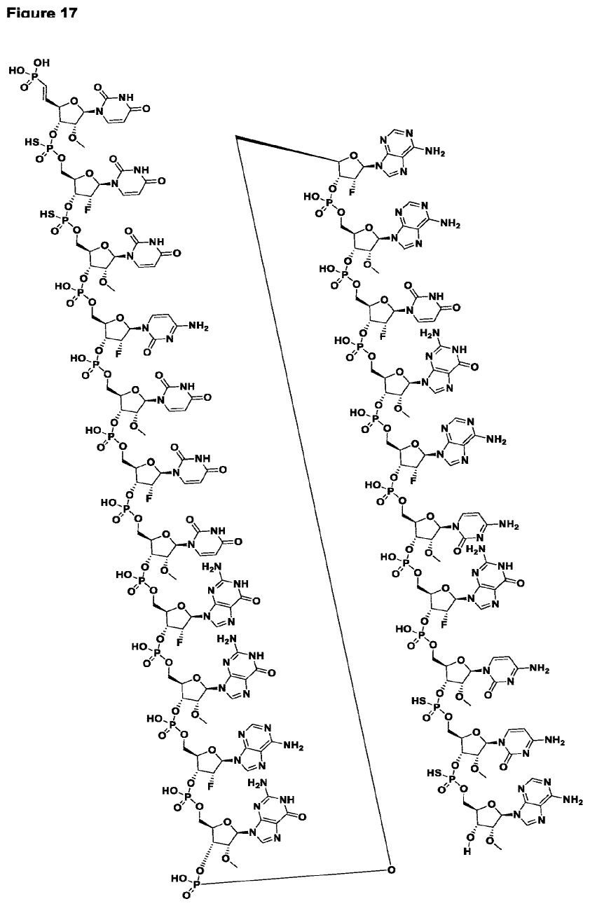

The invention provides a compound comprising the structure as shown in FIG. 17 (antisense strand of compounds 673, 673_C16 and 673_C22).

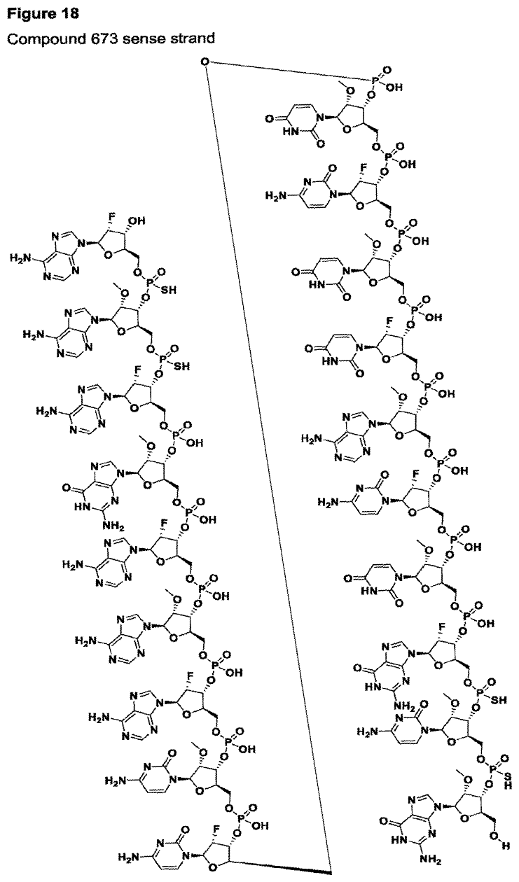

The invention provides a compound comprising the structure as shown in FIG. 18 (sense strand of compound 673).

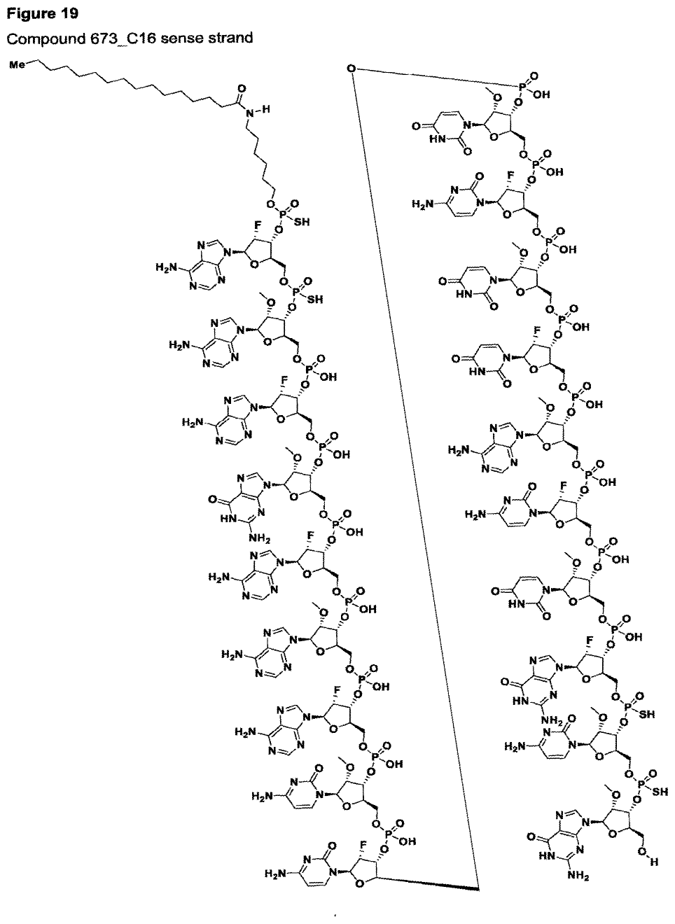

The invention provides a compound comprising the structure as shown in FIG. 19 (sense strand of compound 673_C16).

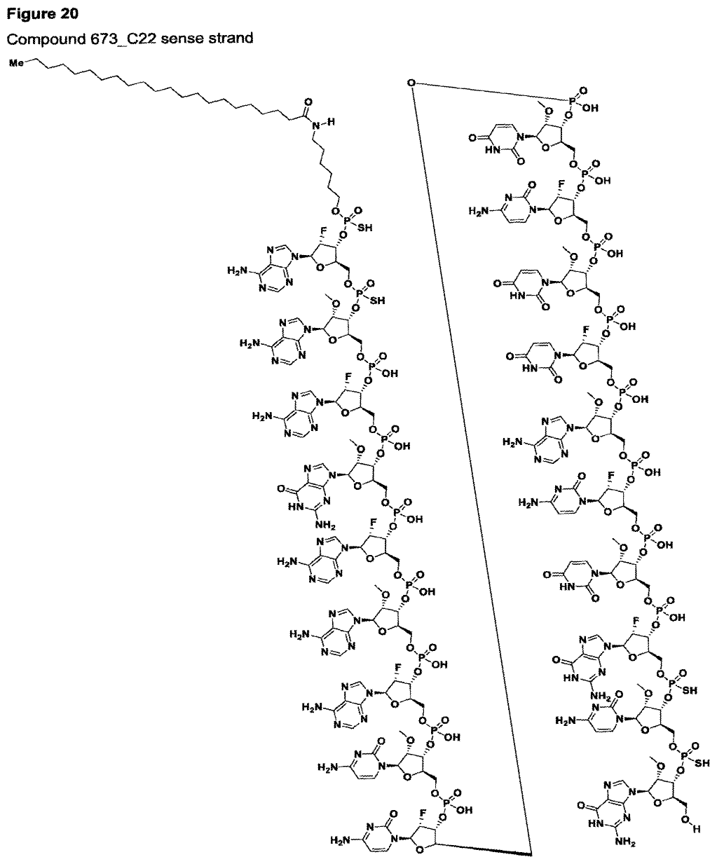

The invention provides a compound comprising the structure as shown in FIG. 20 (the sense strand of compound 673_C22).

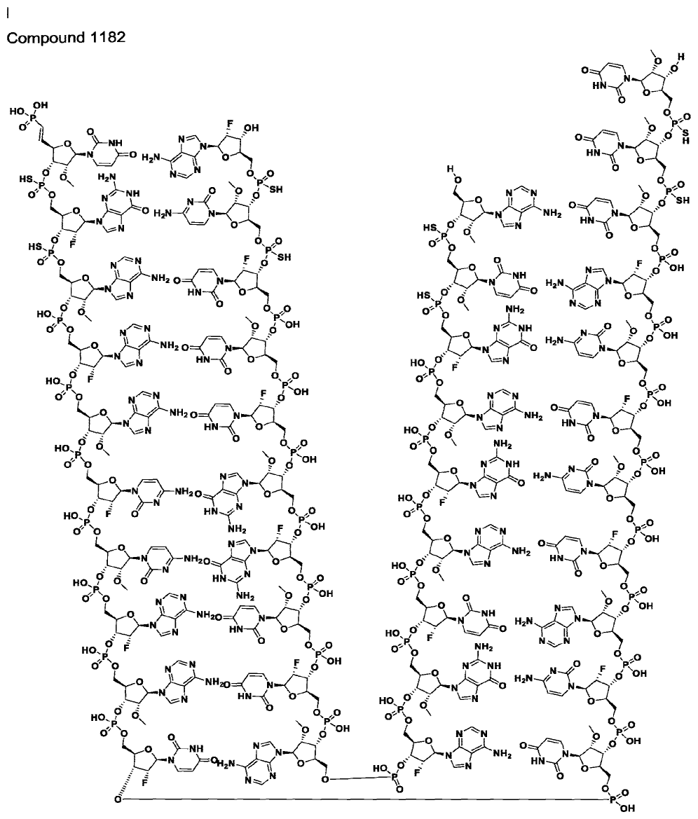

The invention provides a compound comprising the structure as shown in FIG. 21 (compound 1182). The invention provides a compound consisting of the structure as shown in FIG. 21 (compound 1182).

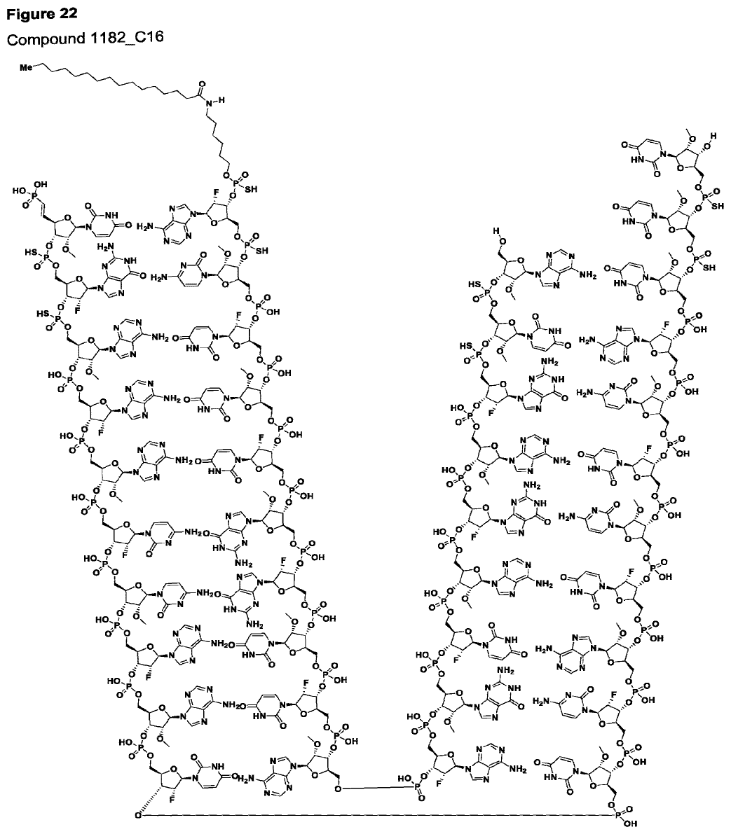

The invention provides a compound comprising the structure as shown in FIG. 22 (compound 1182_C16). The invention provides a compound consisting of the structure as shown in FIG. 22 (compound 1182_C16).

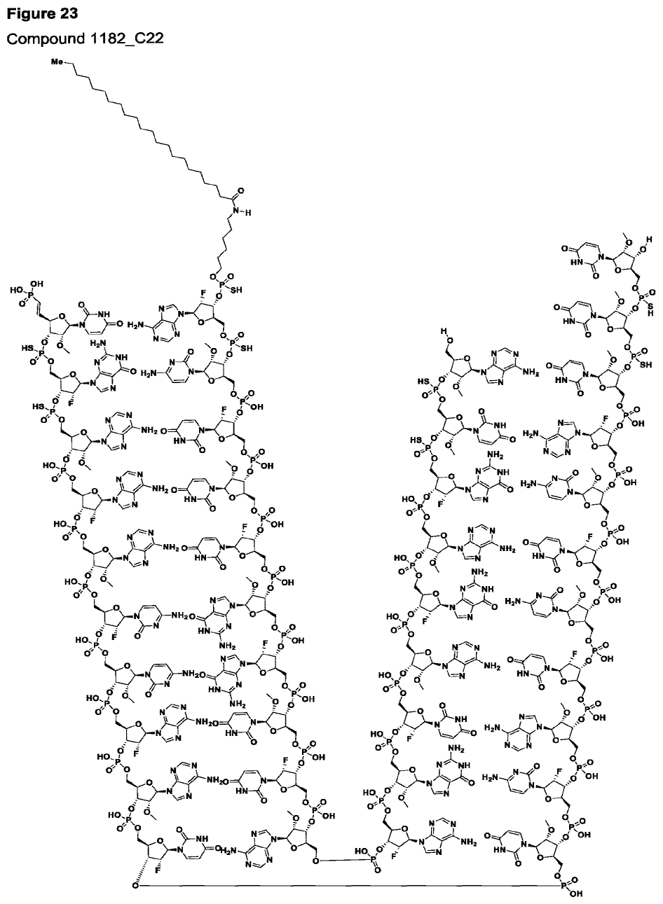

The invention provides a compound comprising the structure as shown in FIG. 23 (compound 1182_C22). The invention provides a compound consisting of the structure as shown in FIG. 23 (compound 1182_C22).

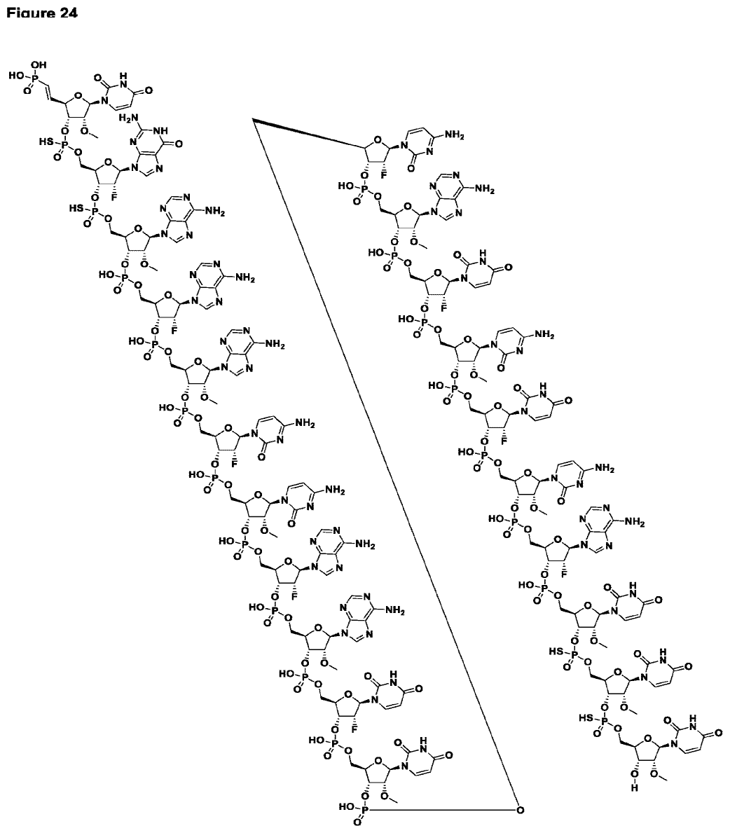

The invention provides a compound comprising the structure as shown in FIG. 24 (antisense strand of compounds 1182, 1182_C16 and 1182_C22).

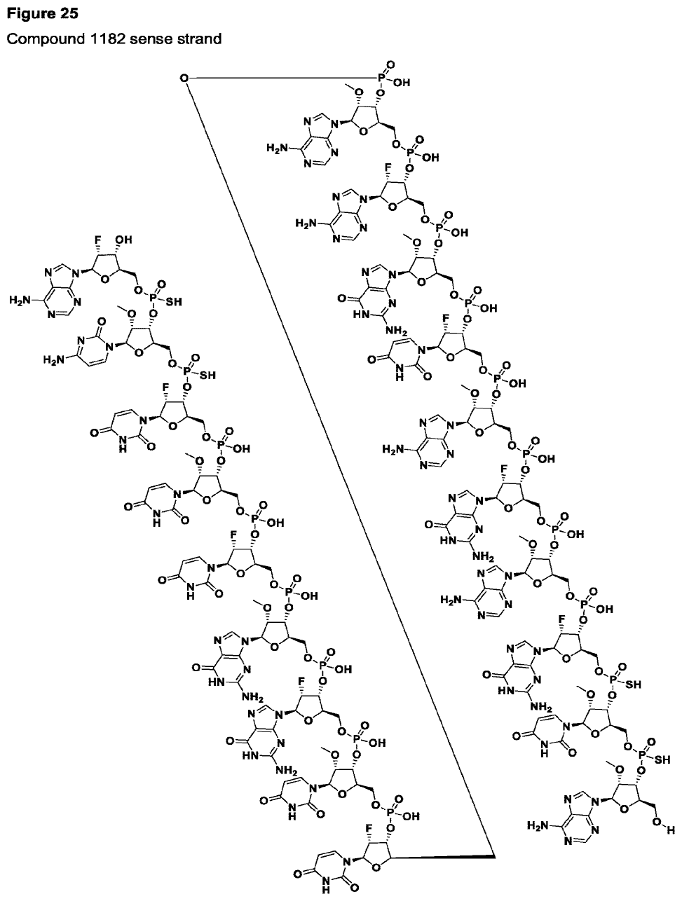

The invention provides a compound comprising the structure as shown in FIG. 25 (sense strand of compound 1182).

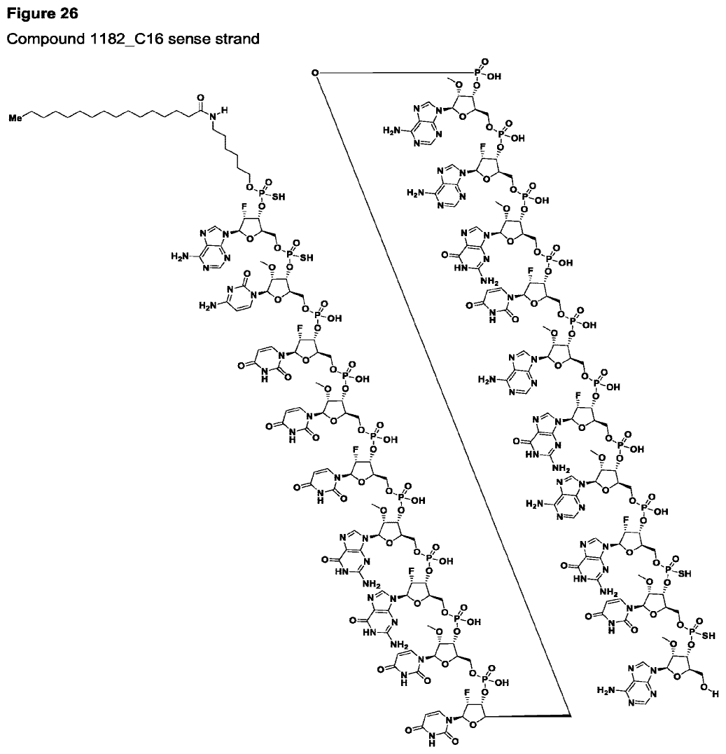

The invention provides a compound comprising the structure as shown in FIG. 26 (the sense strand of compound 1182_C16).

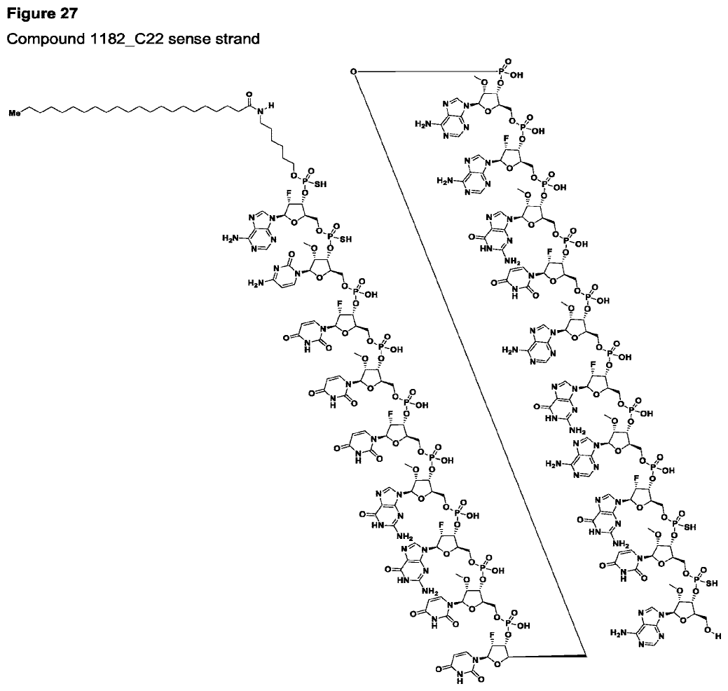

The invention provides a compound comprising the structure as shown in FIG. 27 (the sense strand of compound 1182_C22).

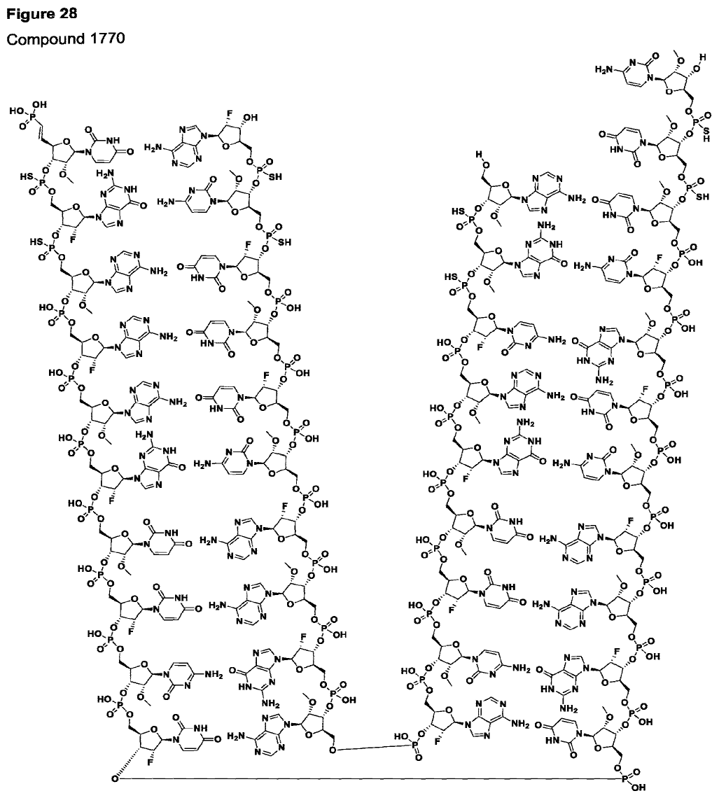

The invention provides a compound comprising the structure as shown in FIG. 28 (compound 1770). The invention provides a compound consisting of the structure as shown in FIG. 28 (compound 1770).

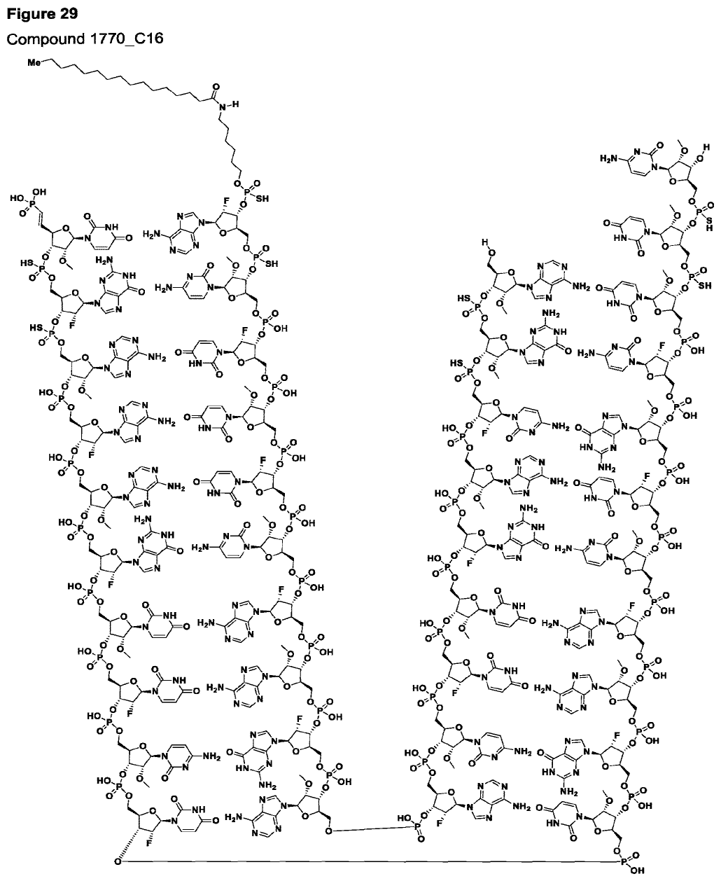

The invention provides a compound comprising the structure as shown in FIG. 29 (compound 1770_C16). The invention provides a compound consisting of the structure as shown in FIG. 29 (compound 1770_C16).

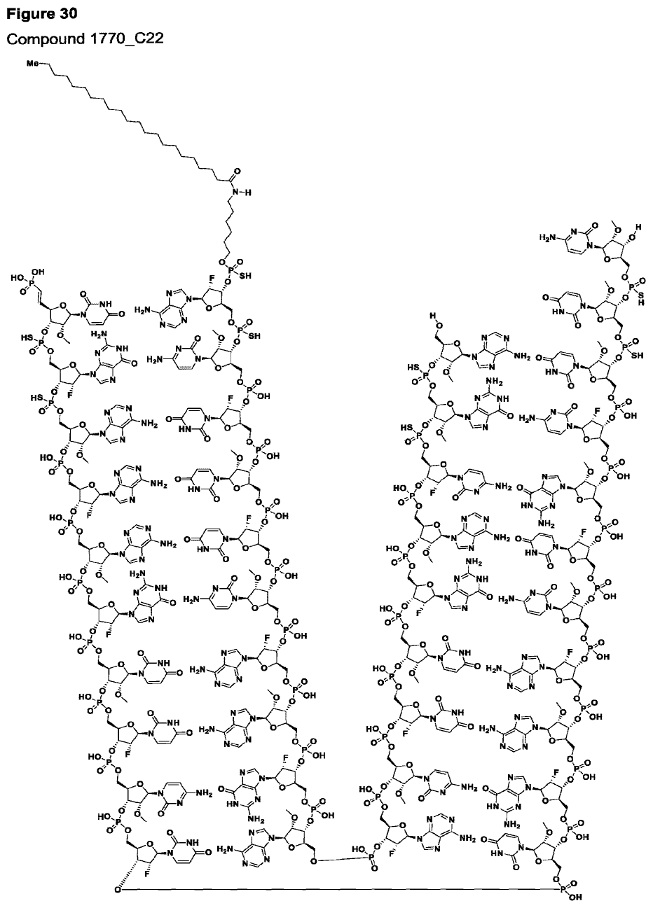

The invention provides a compound comprising the structure as shown in FIG. 30 (compound 1770_C22). The invention provides a compound consisting of the structure as shown in FIG. 30 (compound 1770_C22).

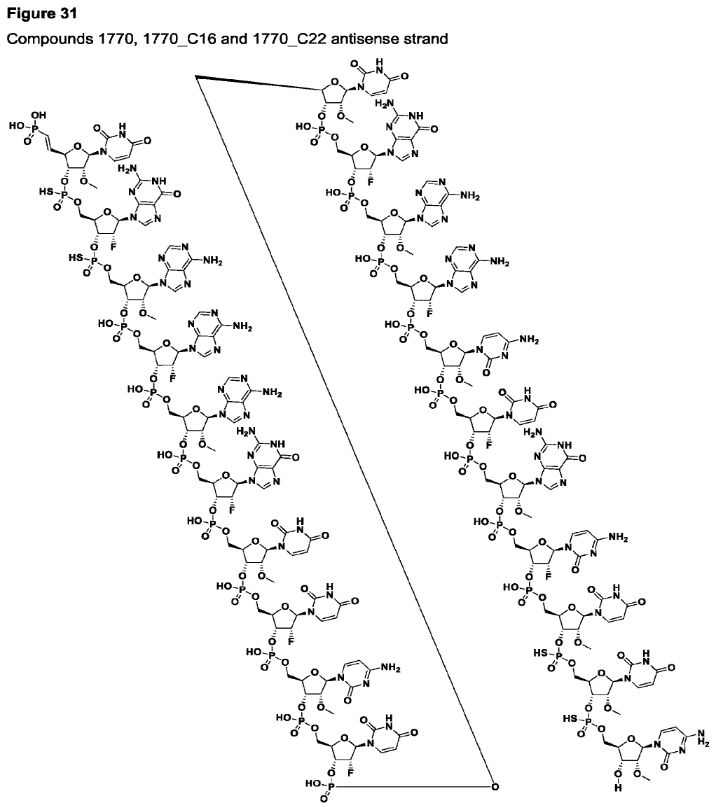

The invention provides a compound comprising the structure as shown in FIG. 31 (antisense strand of compounds 1770, 1770_C16 and 1770_C22).

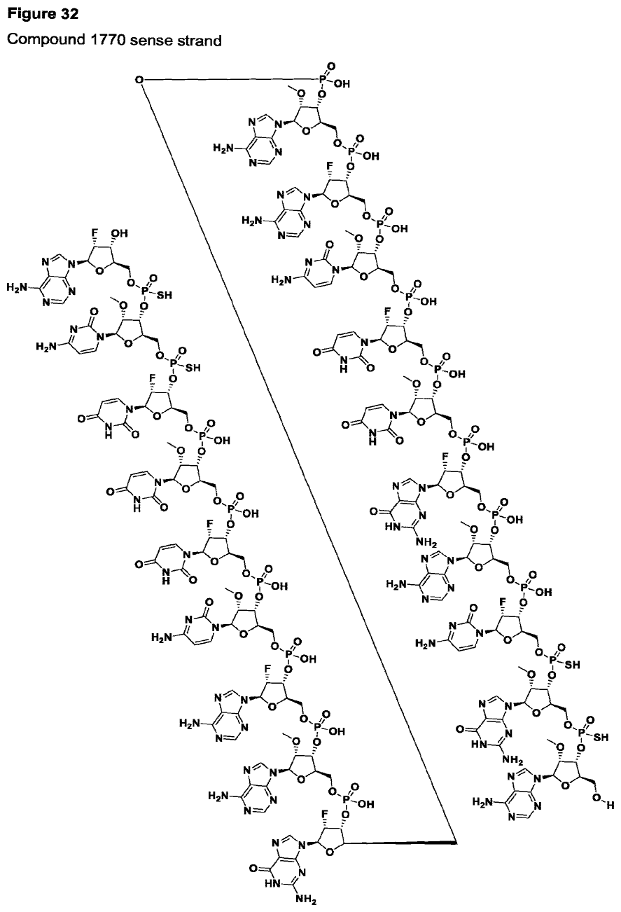

The invention provides a compound comprising the structure as shown in FIG. 32 (sense strand of compound 1770).

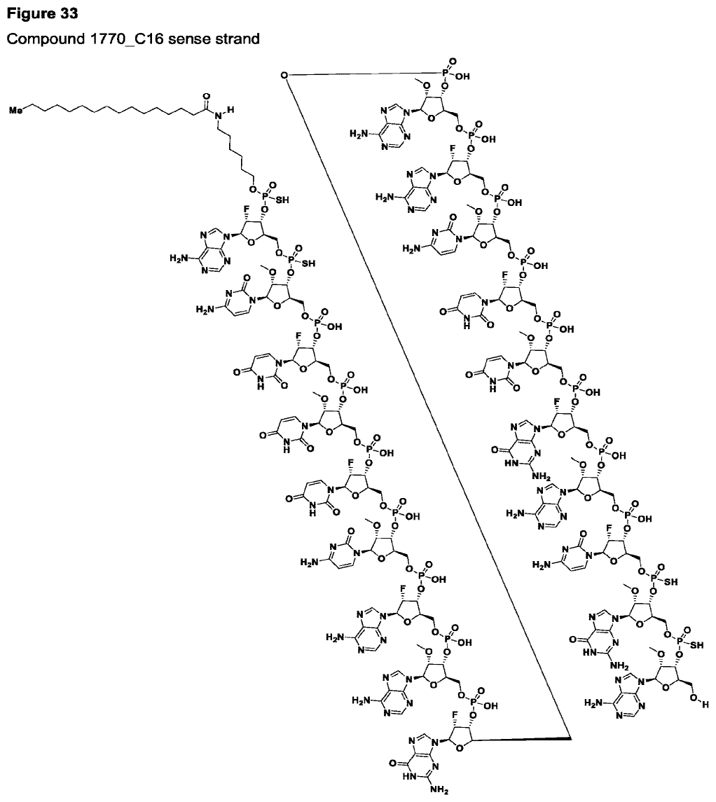

The invention provides a compound comprising the structure as shown in FIG. 33 (the sense strand of compound 1770_C16).

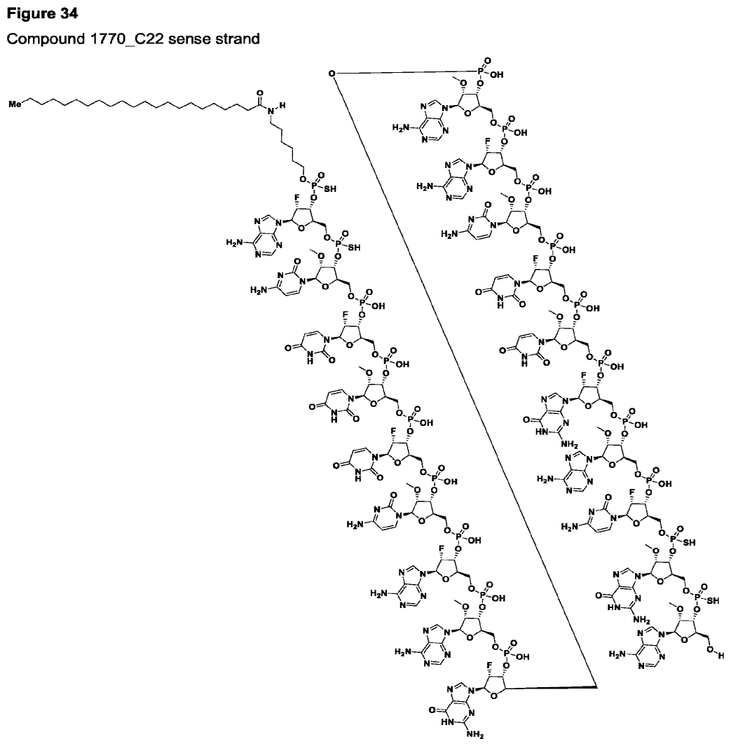

The invention provides a compound comprising the structure as shown in FIG. 34 (the sense strand of compound 1770_C22).

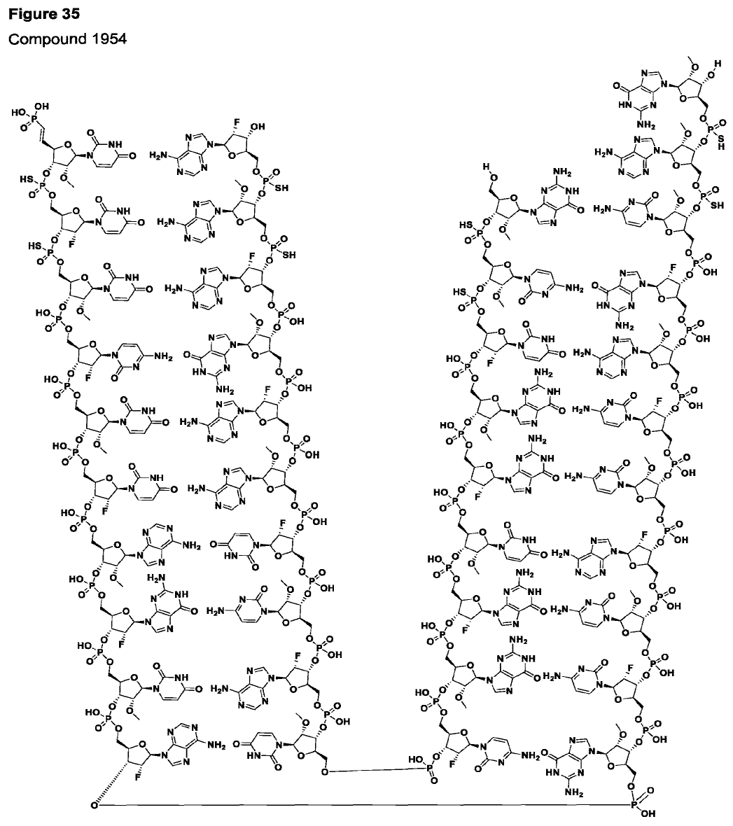

The invention provides a compound comprising the structure as shown in FIG. 35 (compound 1954). The invention provides a compound consisting of the structure as shown in FIG. 35 (compound 1954).

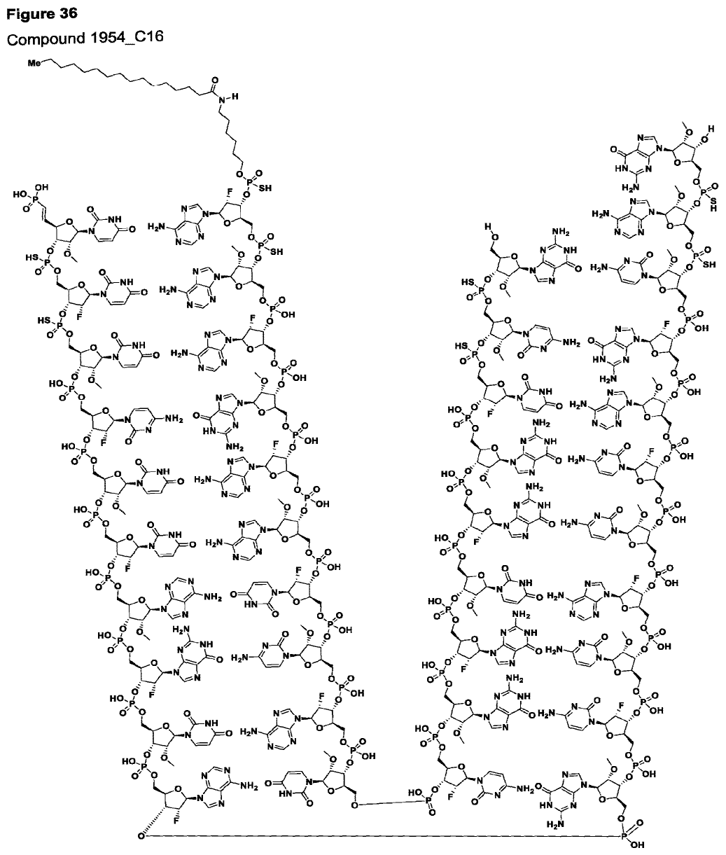

The invention provides a compound comprising the structure as shown in FIG. 36 (compound 1954_C16). The invention provides a compound consisting of the structure as shown in FIG. 36 (compound 1954_C16).

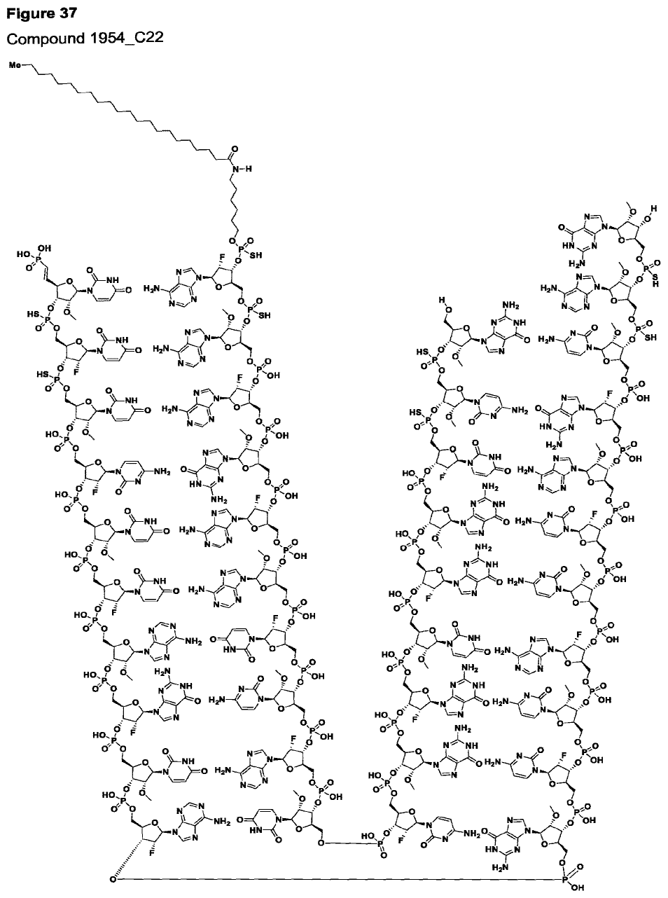

The invention provides a compound comprising the structure as shown in FIG. 37 (compound 1954_C22). The invention provides a compound consisting of the structure as shown in FIG. 37 (compound 1954_C22).

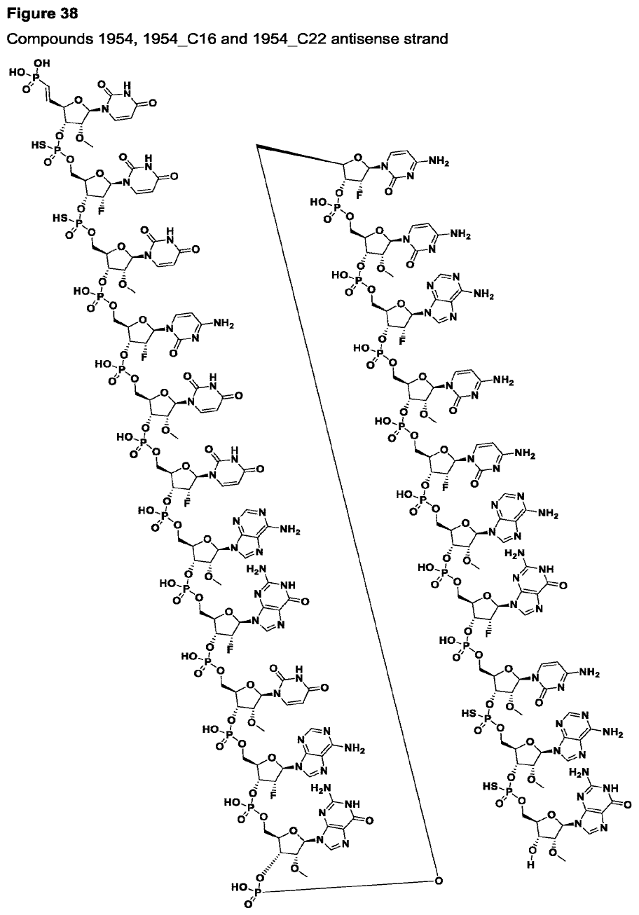

The invention provides a compound comprising the structure as shown in FIG. 38 (antisense strand of compounds 1954, 1954_C16 and 1954_C22).

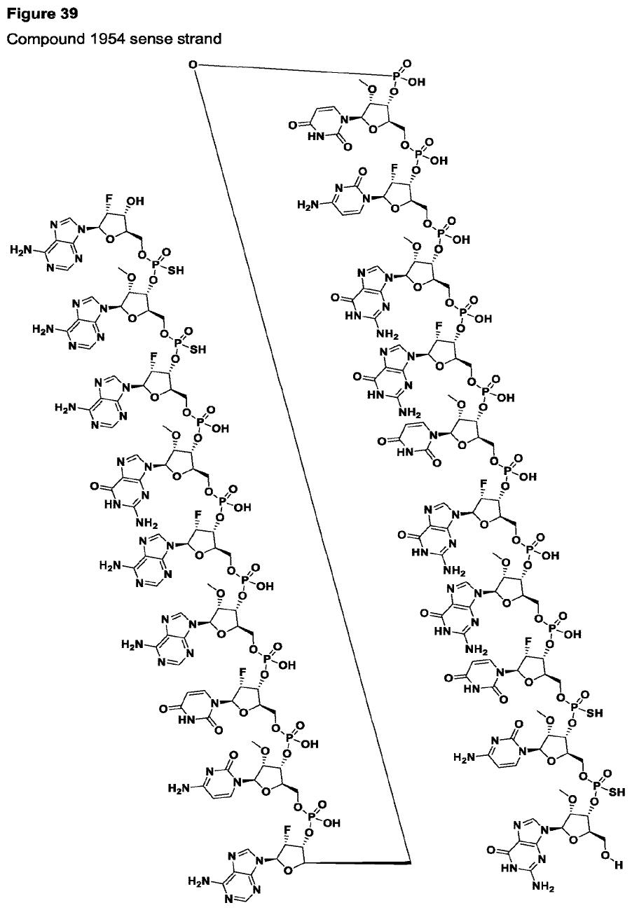

The invention provides a compound comprising the structure as shown in FIG. 39 (sense strand of compound 1954).

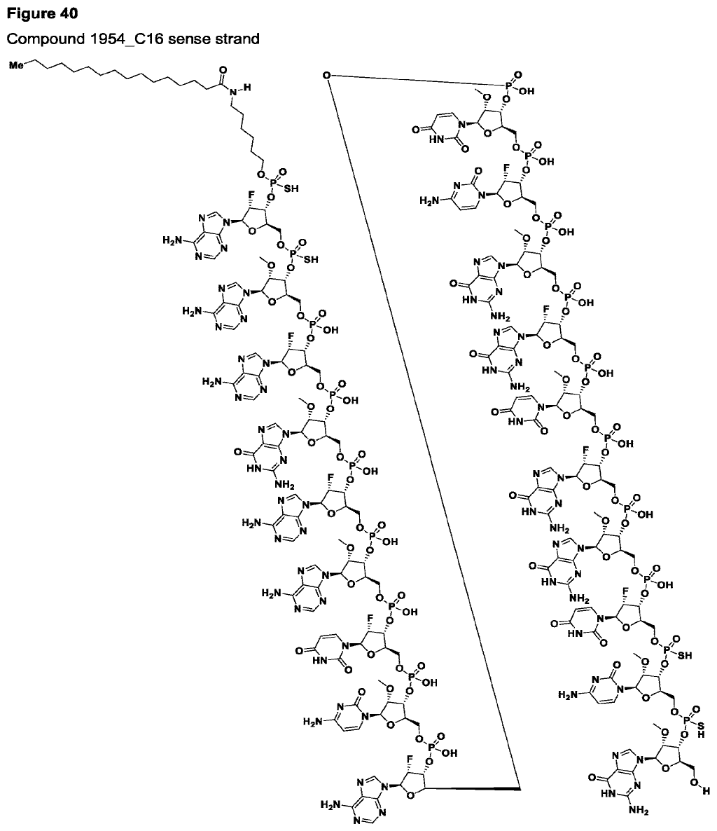

The invention provides a compound comprising the structure as shown in FIG. 40 (the sense strand of compound 1954_C16).

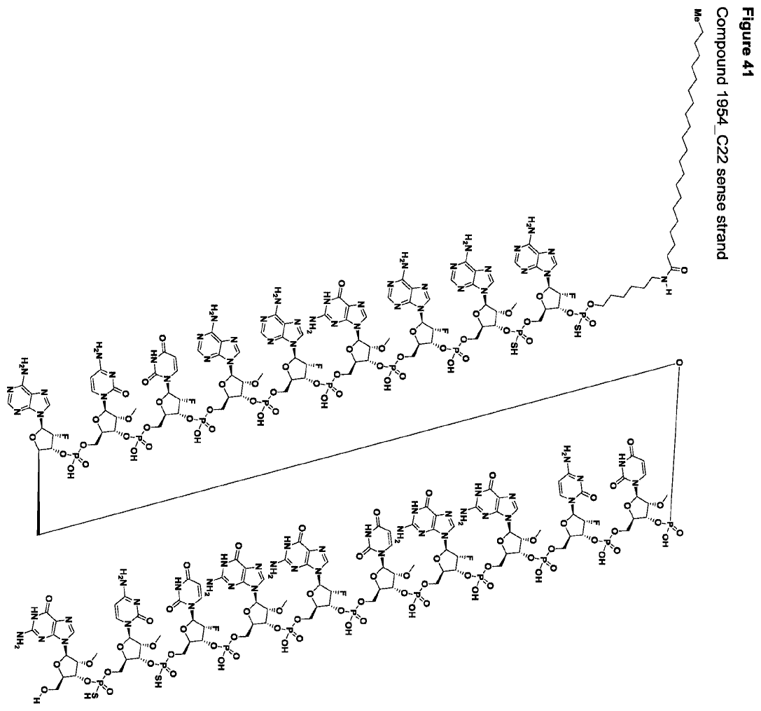

The invention provides a compound comprising the structure as shown in FIG. 41 (the sense strand of compound 1954_C22).

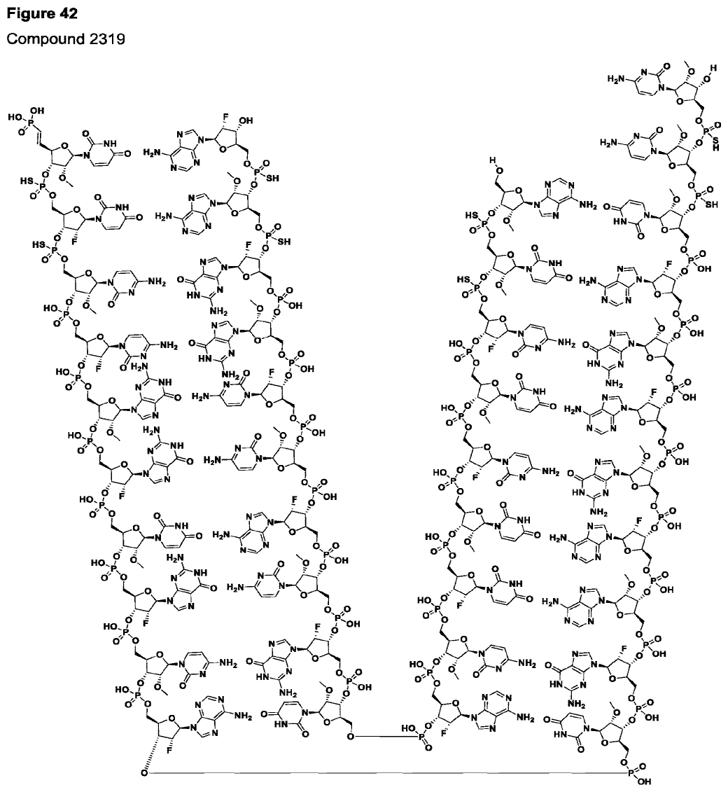

The invention provides a compound comprising the structure as shown in FIG. 42 (compound 2319). The invention provides a compound consisting of the structure as shown in FIG. 42 (compound 2319).

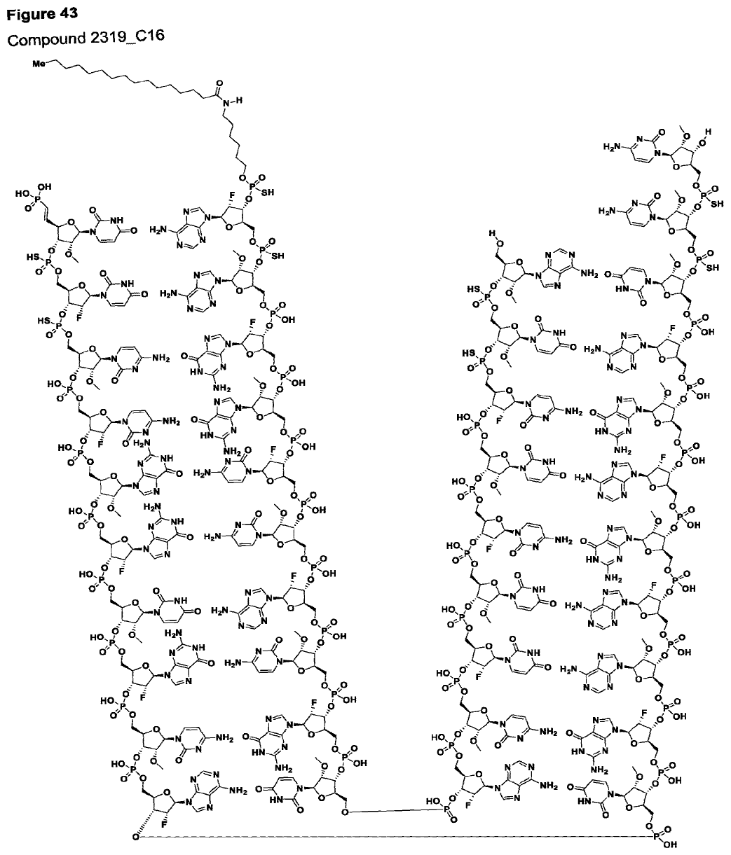

The invention provides a compound comprising the structure as shown in FIG. 43 (compound 2319_C16). The invention provides a compound consisting of the structure as shown in FIG. 43 (compound 2319_C16).

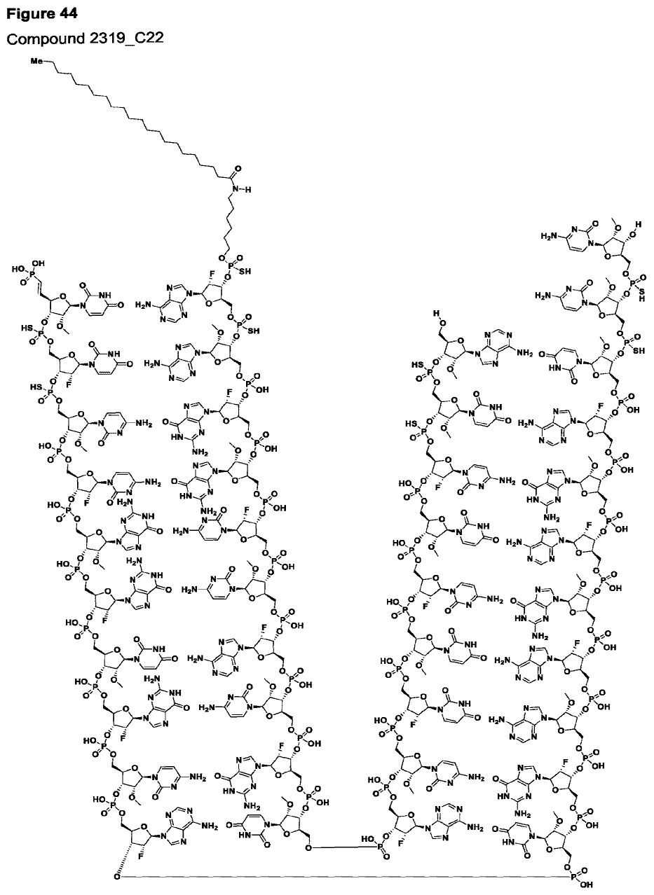

The invention provides a compound comprising the structure as shown in FIG. 44 (compound 2319_C22). The invention provides a compound consisting of the structure as shown in FIG. 44 (compound 2319_C22).

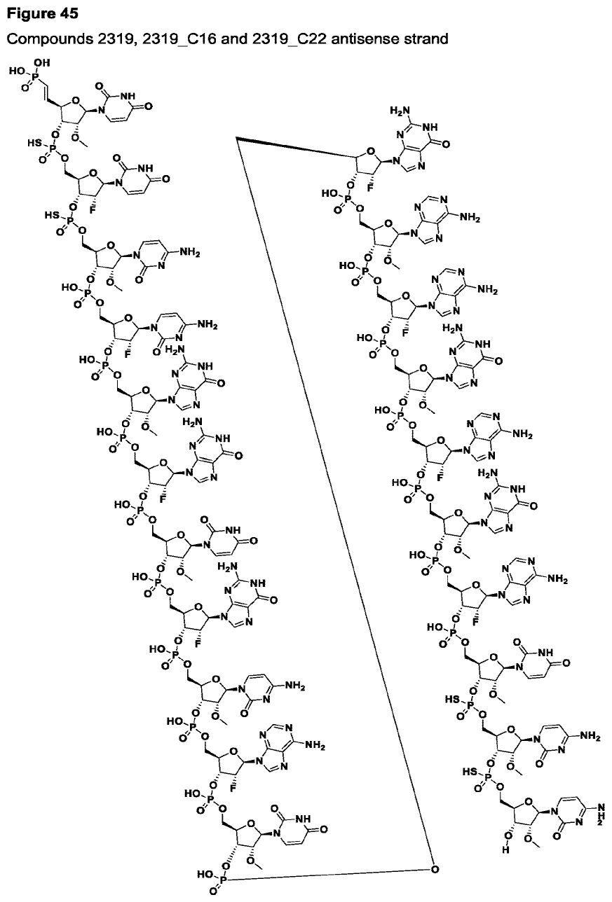

The invention provides a compound comprising the structure as shown in FIG. 45 (antisense strand of compounds 2319, 2319_C16 and 2319_C22).

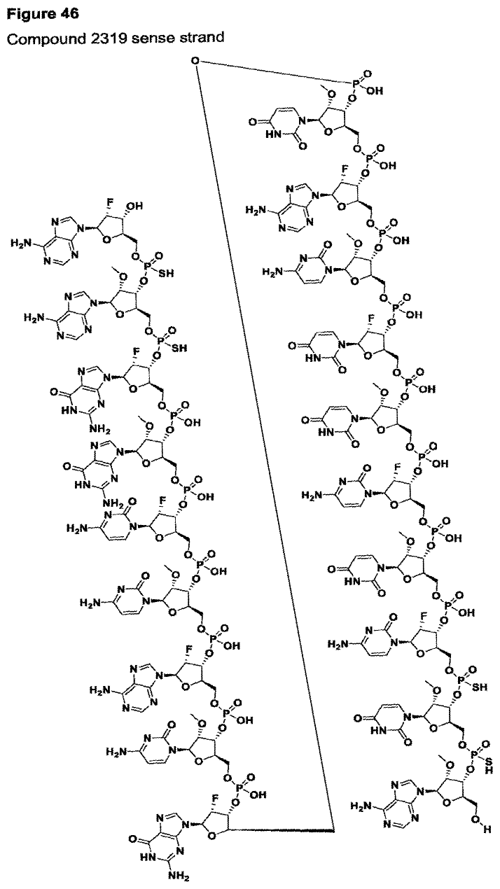

The invention provides a compound comprising the structure as shown in FIG. 46 (sense strand of compound 2319).

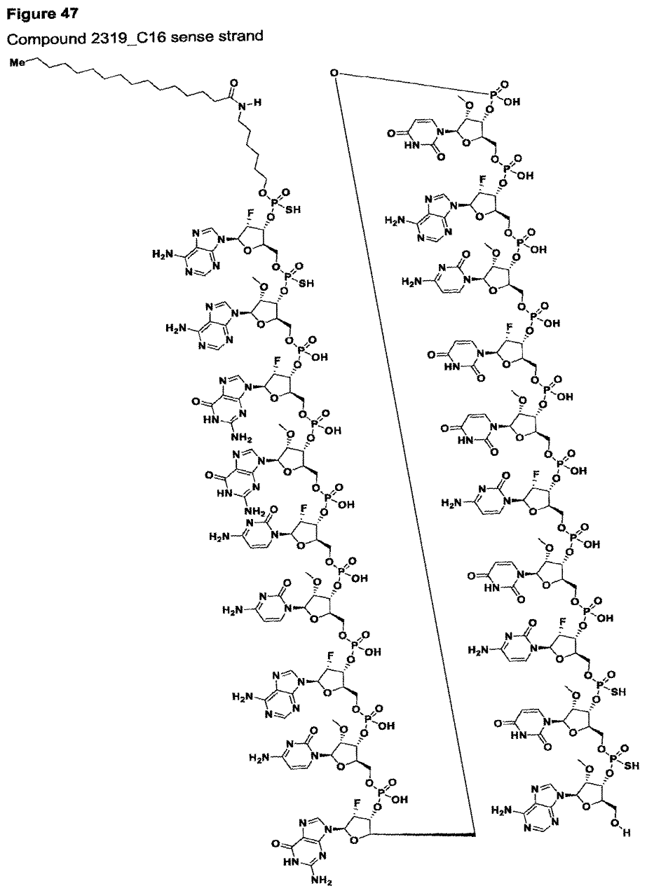

The invention provides a compound comprising the structure as shown in FIG. 47 (the sense strand of compound 2319_C16).

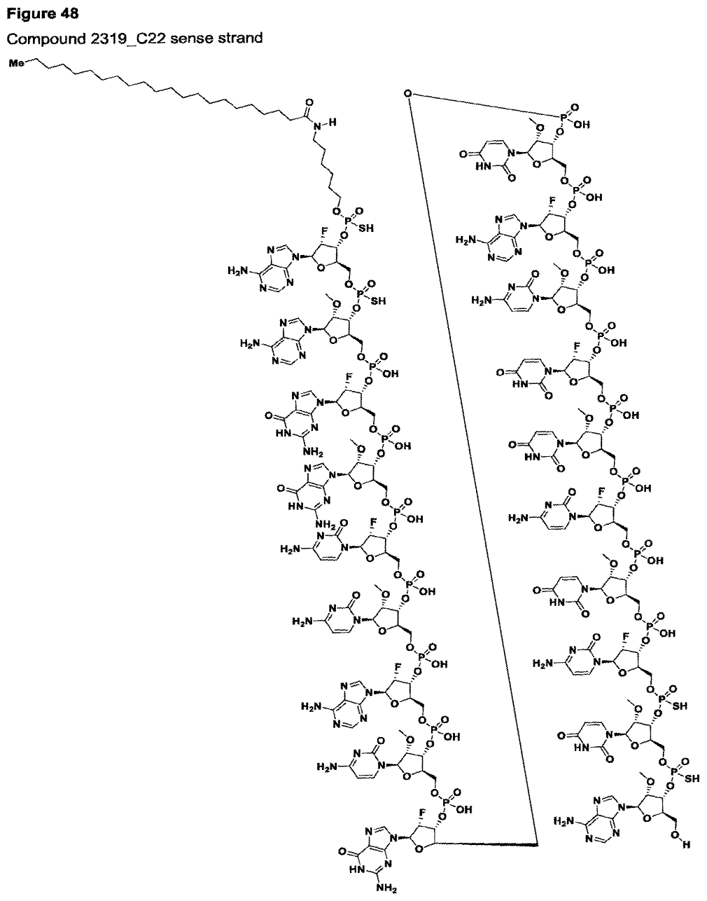

The invention provides a compound comprising the structure as shown in FIG. 48 (the sense strand of compound 2319_C22).

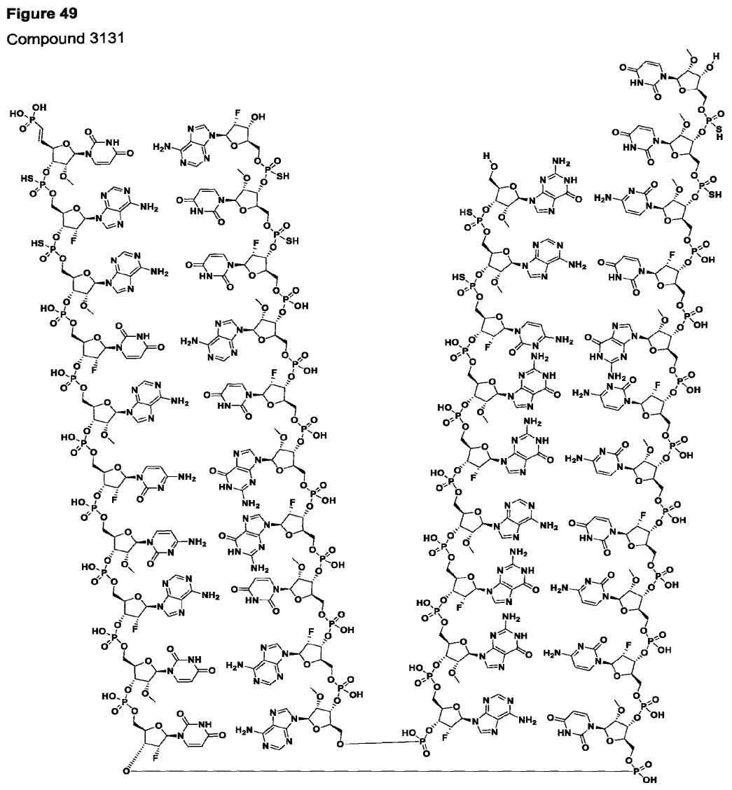

The invention provides a compound comprising the structure as shown in FIG. 49 (compound 3131). The invention provides a compound consisting of the structure as shown in FIG. 49 (compound 3131).

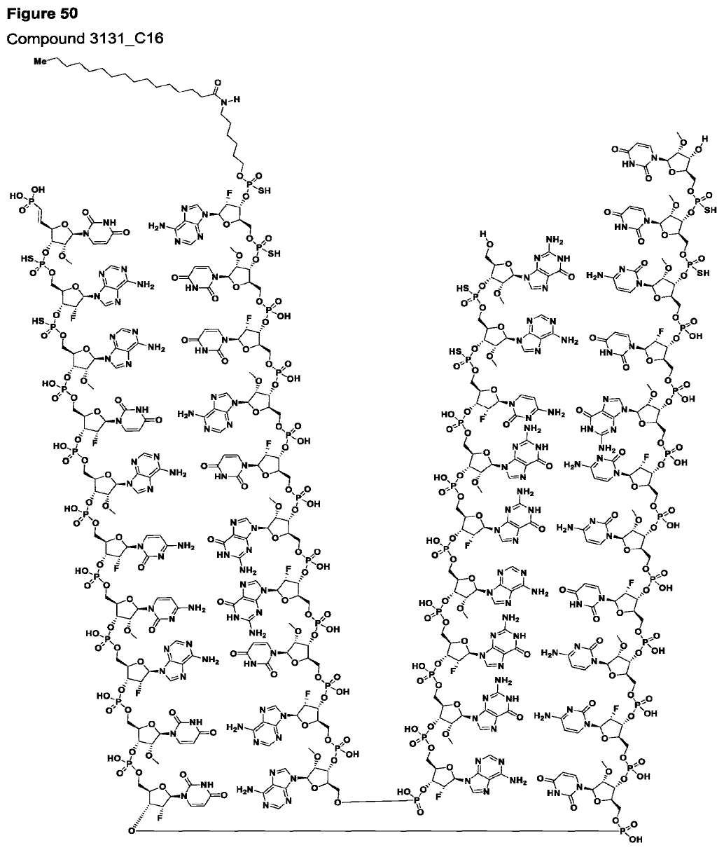

The invention provides a compound comprising the structure as shown in FIG. 50 (compound 3131_C16). The invention provides a compound consisting of the structure as shown in FIG. 50 (compound 3131_C16).

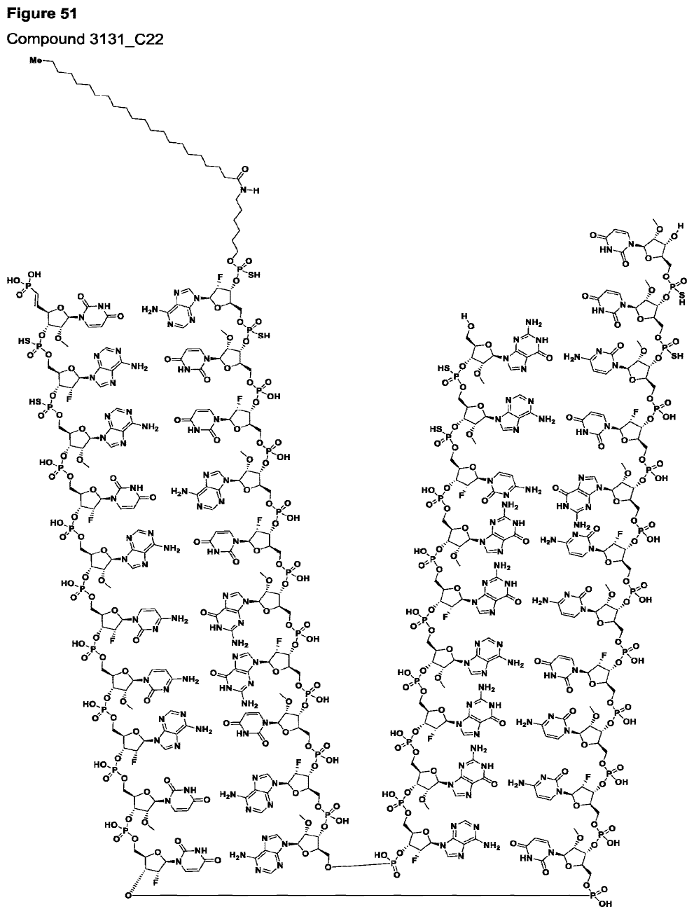

The invention provides a compound comprising the structure as shown in FIG. 51 (compound 3131_C22). The invention provides a compound consisting of the structure as shown in FIG. 51 (compound 3131_C22).

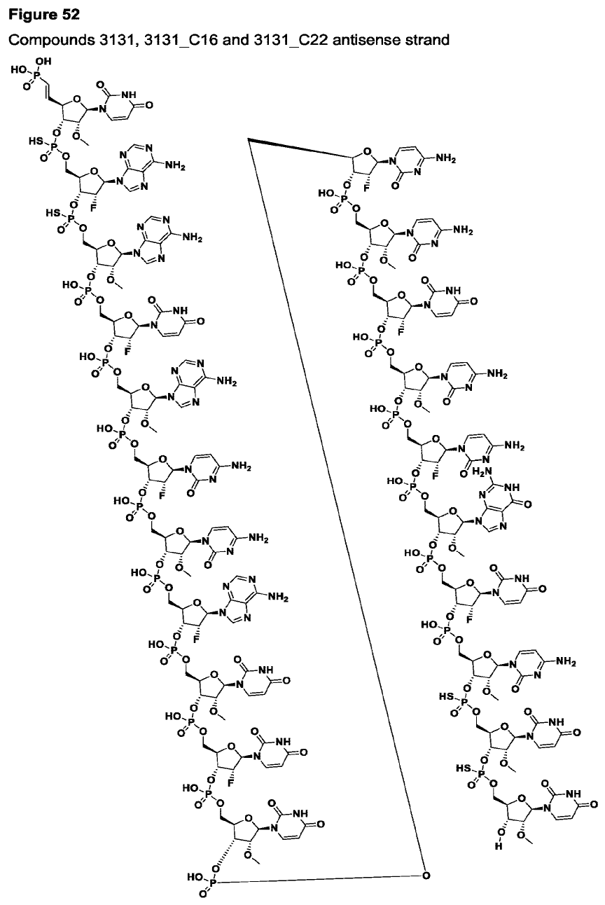

The invention provides a compound comprising the structure as shown in FIG. 52 (antisense strand of compounds 3131, 3131_C16 and 3131_C22).

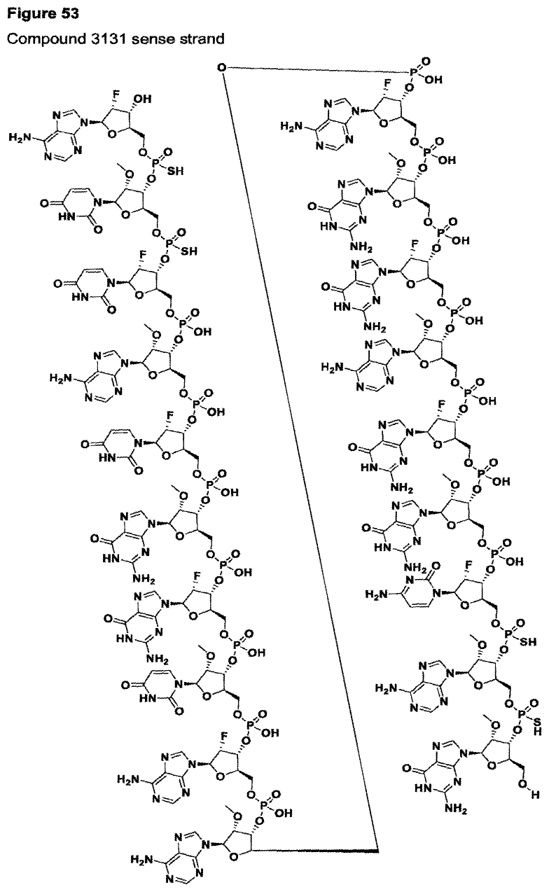

The invention provides a compound comprising the structure as shown in FIG. 53 (sense strand of compound 3131).

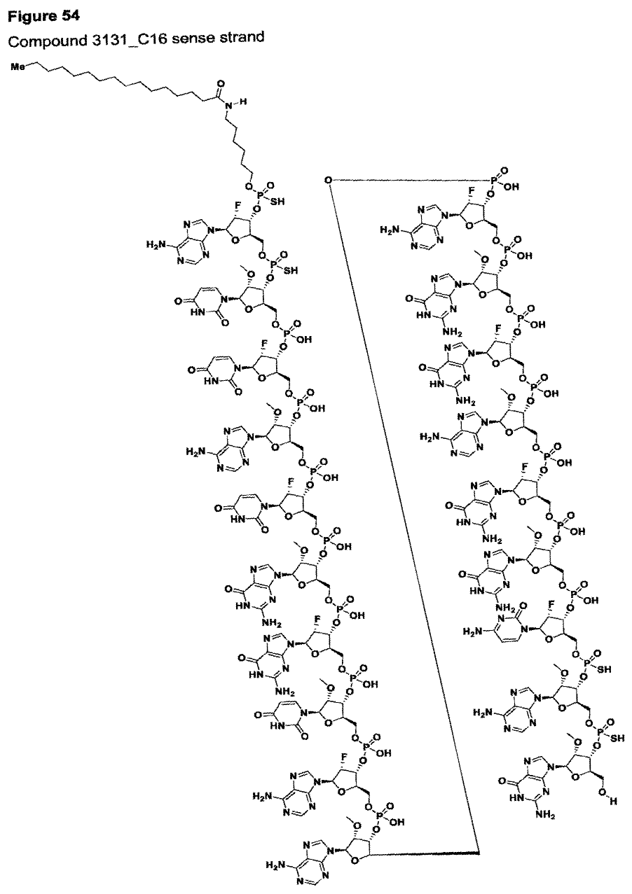

The invention provides a compound comprising the structure as shown in FIG. 54 (the sense strand of compound 3131_C16).

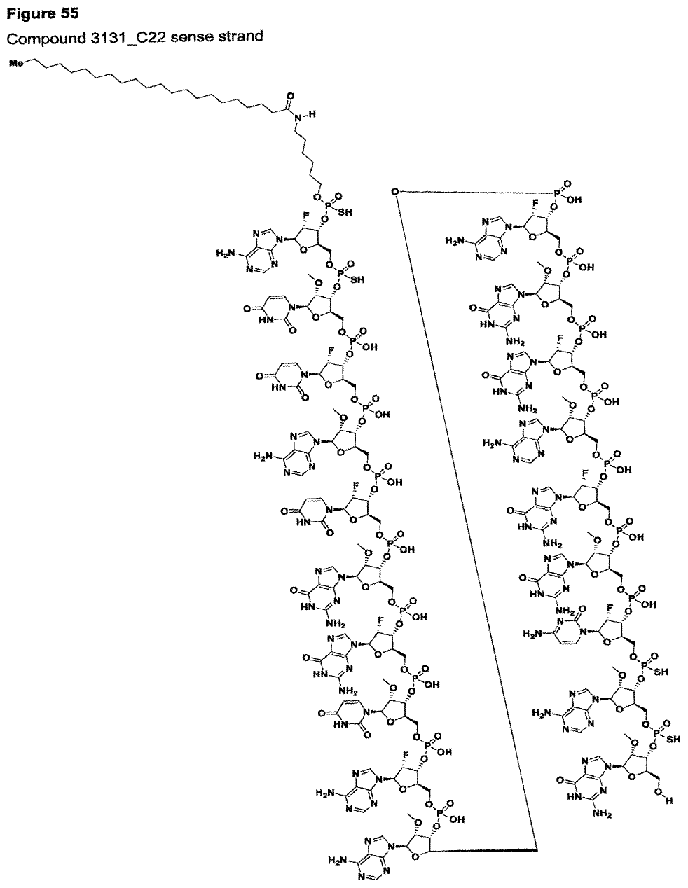

The invention provides a compound comprising the structure as shown in FIG. 55 (the sense strand of compound 3131_C22).

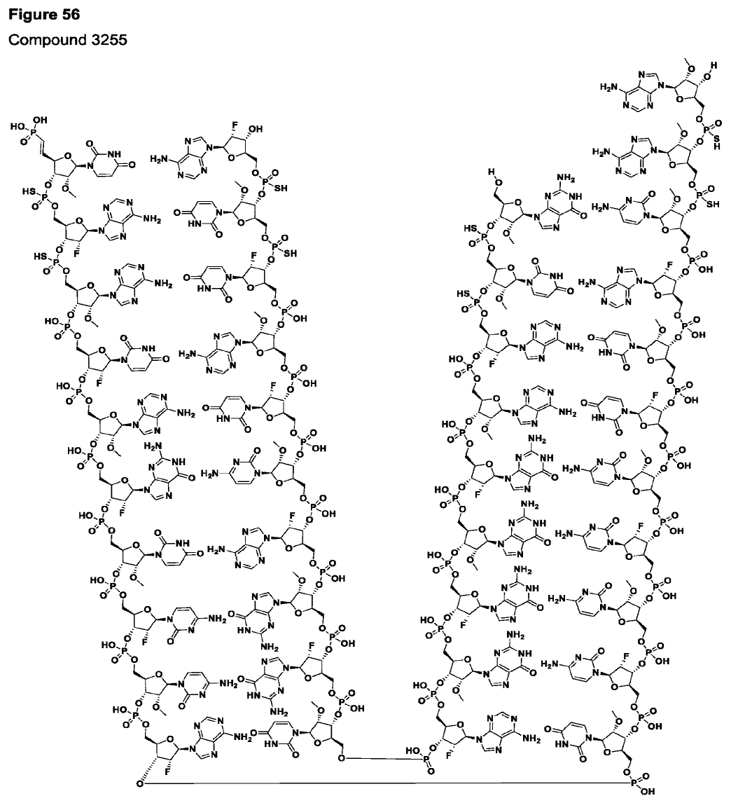

The invention provides a compound comprising the structure as shown in FIG. 56 (compound 3255). The invention provides a compound consisting of the structure as shown in FIG. 56 (compound 3255).

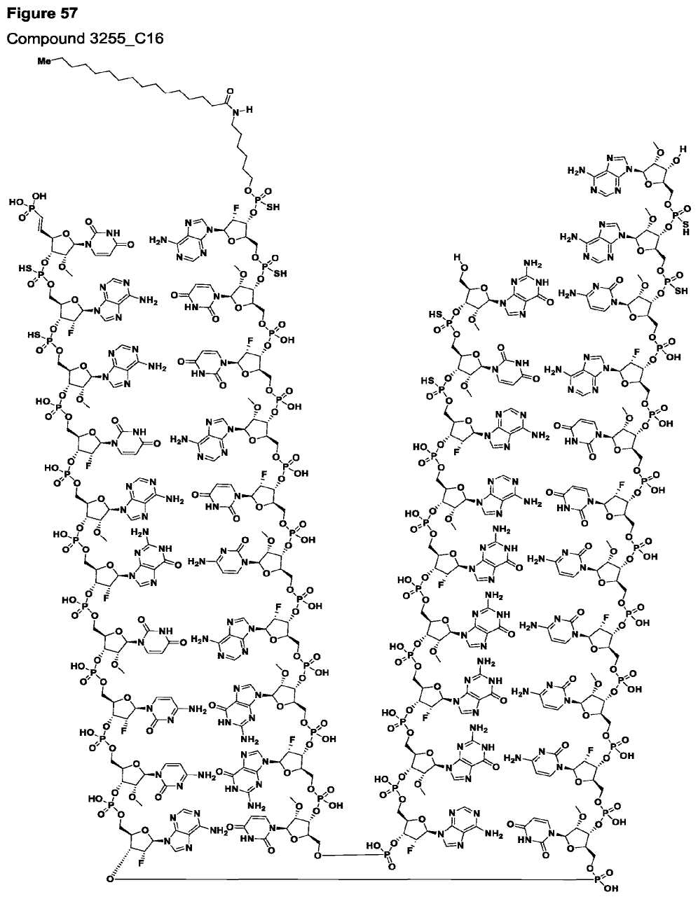

The invention provides a compound comprising the structure as shown in FIG. 57 (compound 3255_C16). The invention provides a compound consisting of the structure as shown in FIG. 57 (compound 3255_C16).

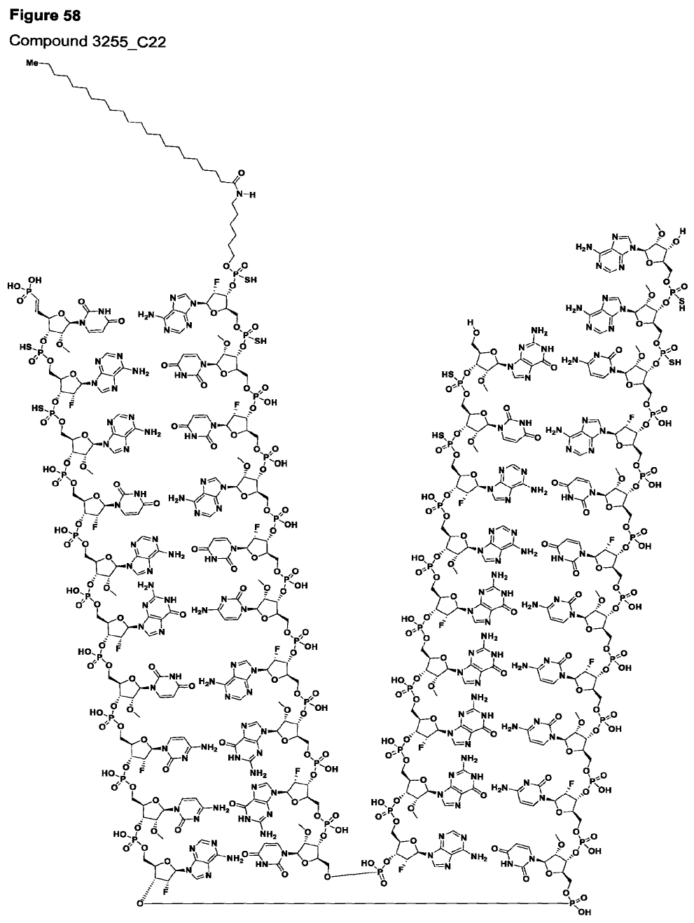

The invention provides a compound comprising the structure as shown in FIG. 58 (compound 3255_C22). The invention provides a compound consisting of the structure as shown in FIG. 58 (compound 3255_C22).

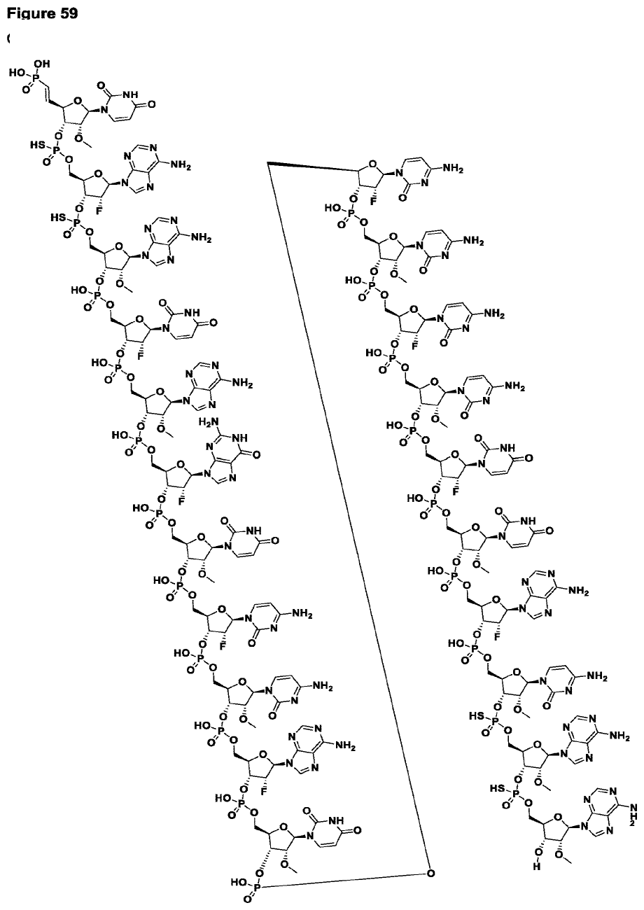

The invention provides a compound comprising the structure as shown in FIG. 59 (antisense strand of compounds 3255, 3255_C16 and 3255_C22).

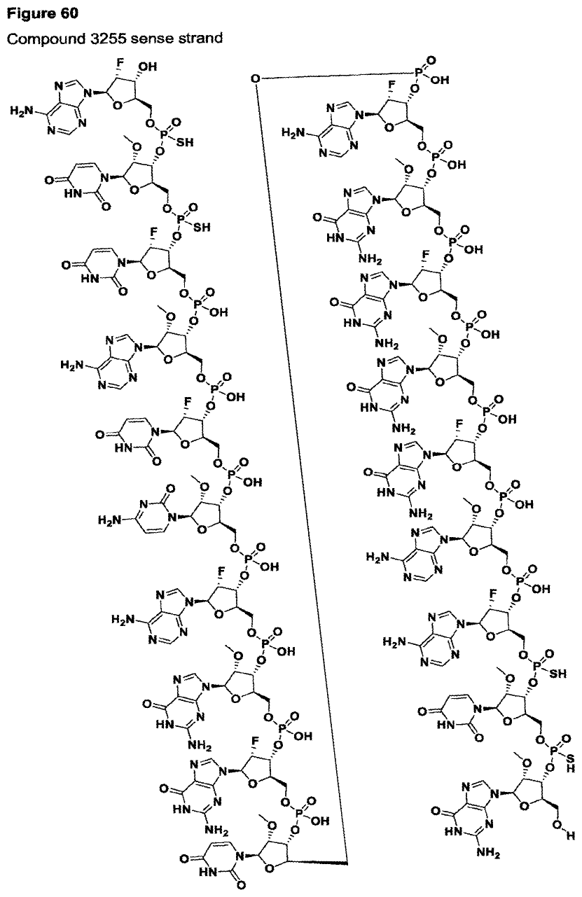

The invention provides a compound comprising the structure as shown in FIG. 60 (sense strand of compound 3255).

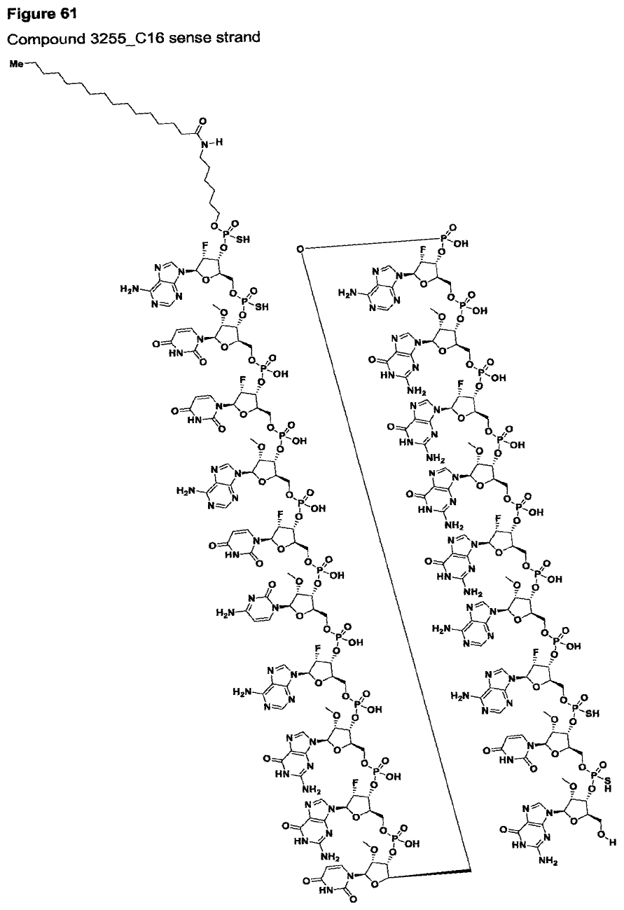

The invention provides a compound comprising the structure as shown in FIG. 61 (the sense strand of compound 3255_C16).

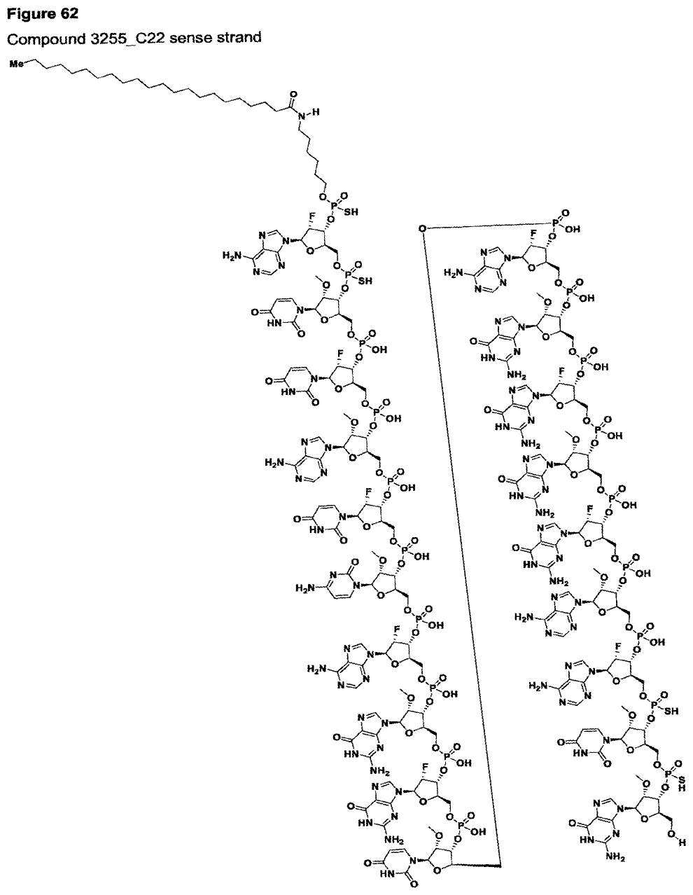

The invention provides a compound comprising the structure as shown in FIG. 62 (the sense strand of compound 3255_C22).

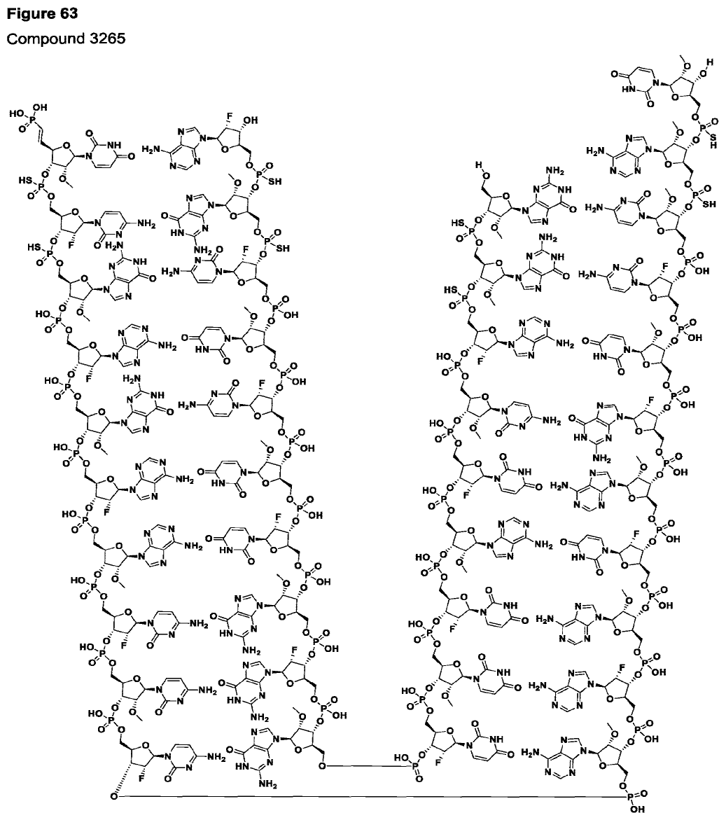

The invention provides a compound comprising the structure as shown in FIG. 63 (compound 3265). The invention provides a compound consisting of the structure as shown in FIG. 63 (compound 3265).

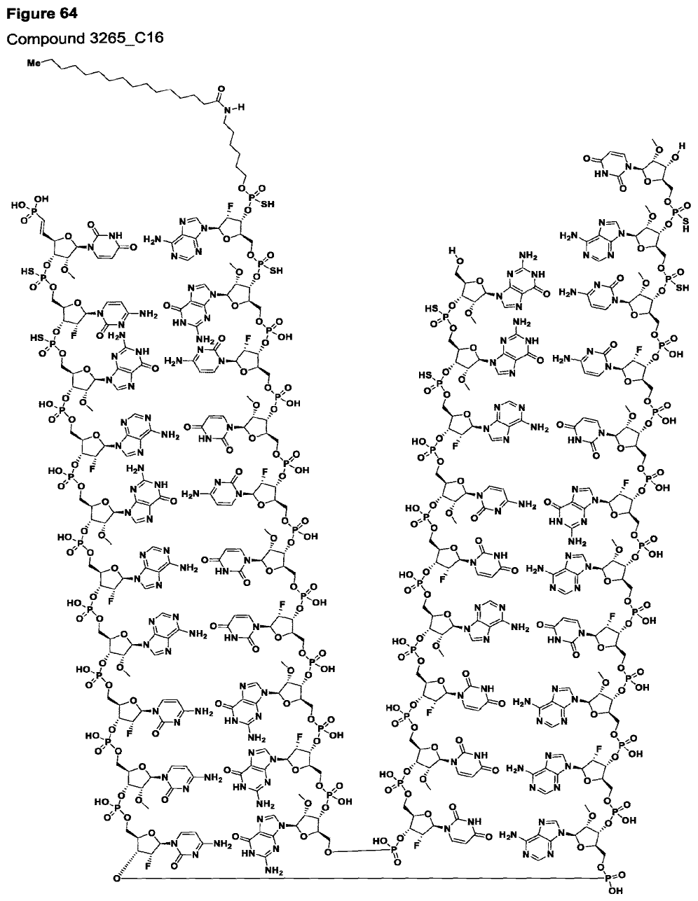

The invention provides a compound comprising the structure as shown in FIG. 64 (compound 3265_C16). The invention provides a compound consisting of the structure as shown in FIG. 64 (compound 3265_C16).

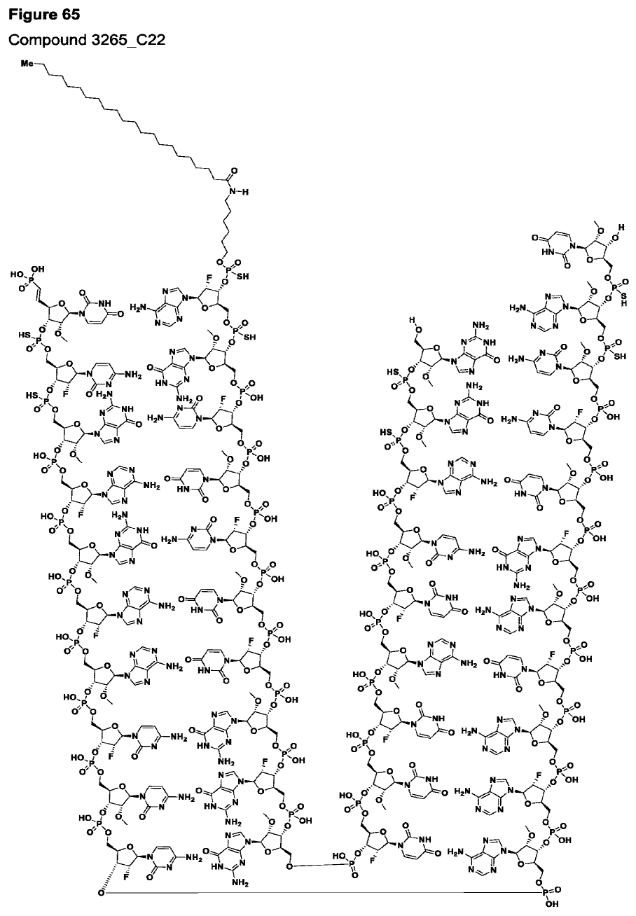

The invention provides a compound comprising the structure as shown in FIG. 65 (compound 3265_C22). The invention provides a compound consisting of the structure as shown in FIG. 65 (compound 3265_C22).

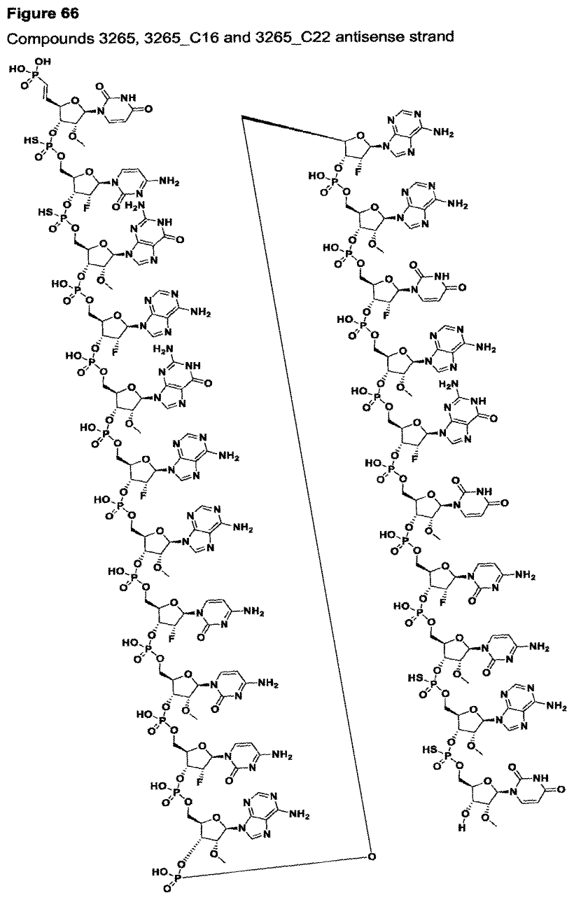

The invention provides a compound comprising the structure as shown in FIG. 66 (antisense strand of compounds 3265, 3265_C16 and 3265_C22).

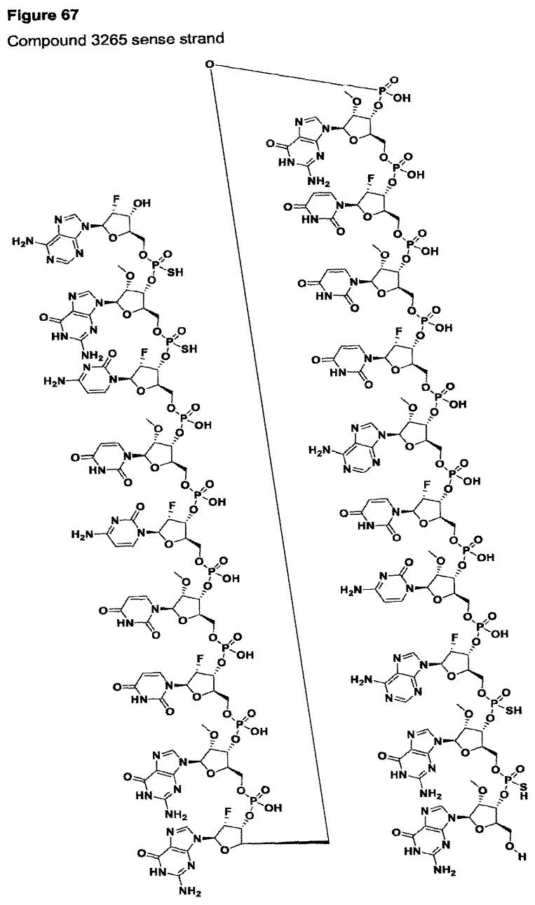

The invention provides a compound comprising the structure as shown in FIG. 67 (sense strand of compound 3265).

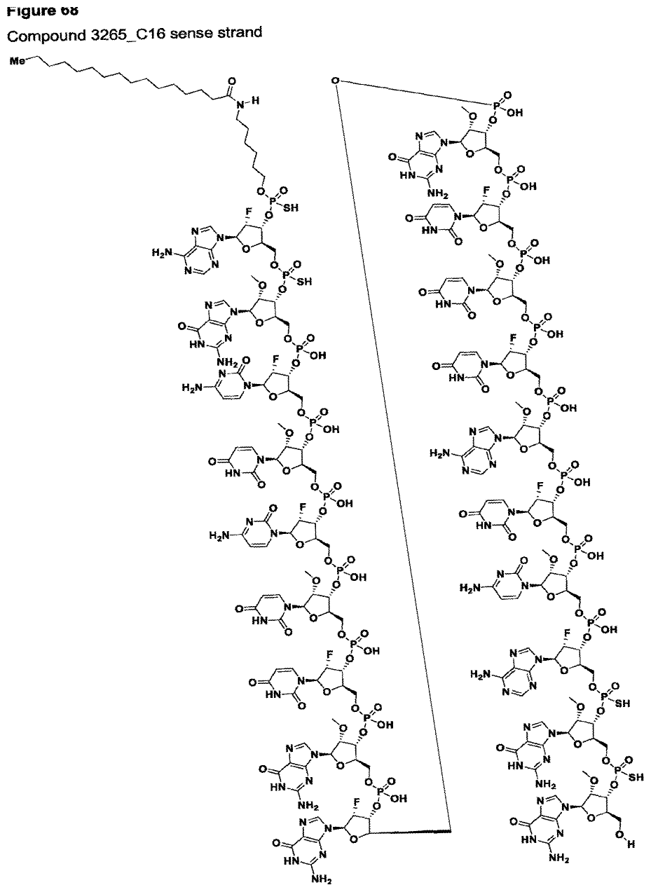

The invention provides a compound comprising the structure as shown in FIG. 68 (the sense strand of compound 3265_C16).

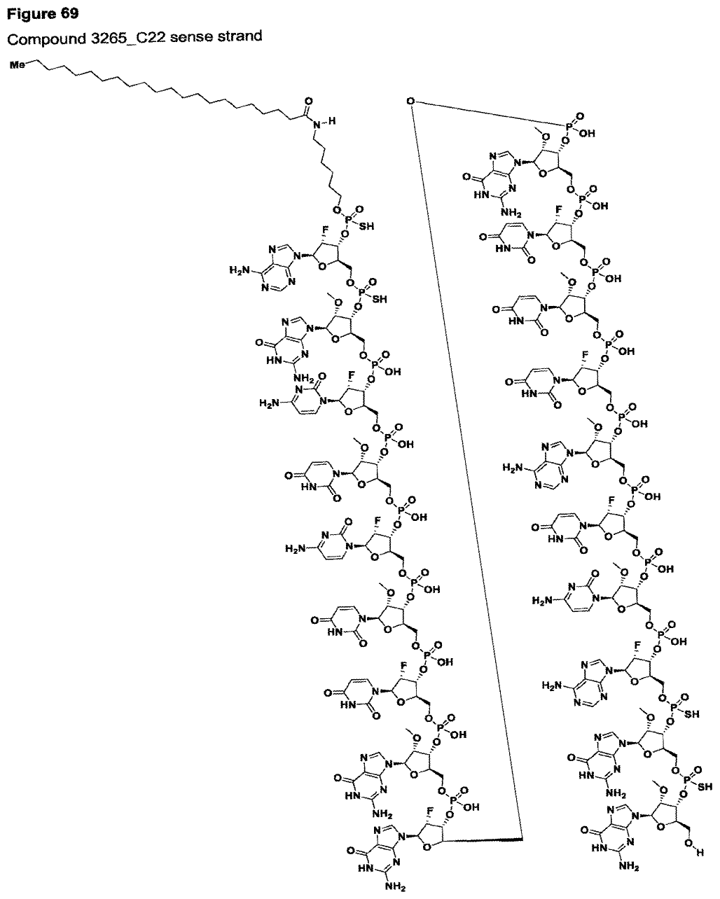

The invention provides a compound comprising the structure as shown in FIG. 69 (the sense strand of compound 3265_C22).

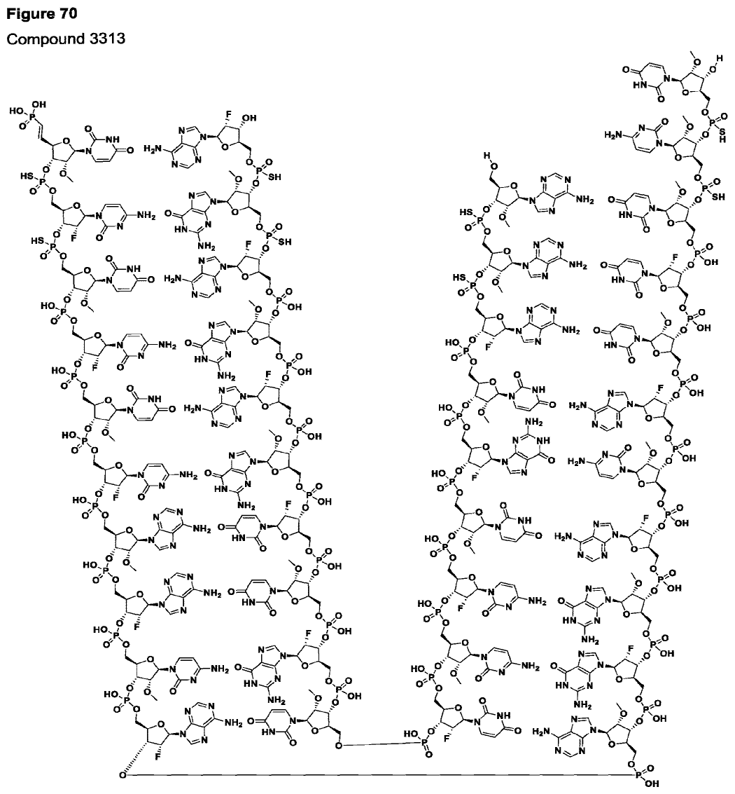

The invention provides a compound comprising the structure as shown in FIG. 70 (compound 3313). The invention provides a compound consisting of the structure as shown in FIG. 70 (compound 3313).

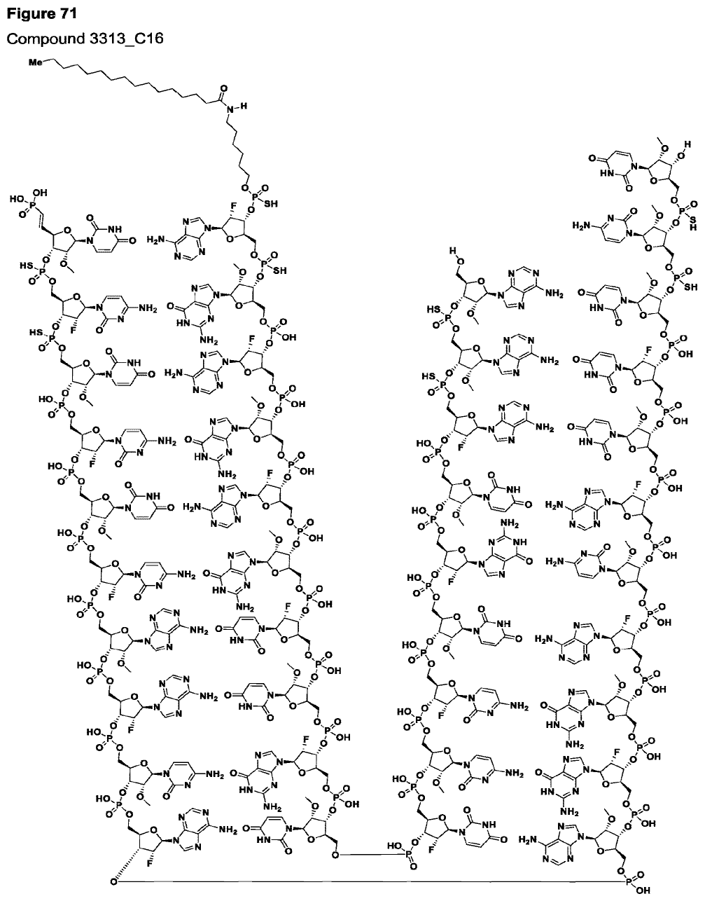

The invention provides a compound comprising the structure as shown in FIG. 71 (compound 3313_C16). The invention provides a compound consisting of the structure as shown in FIG. 71 (compound 3313_C16).

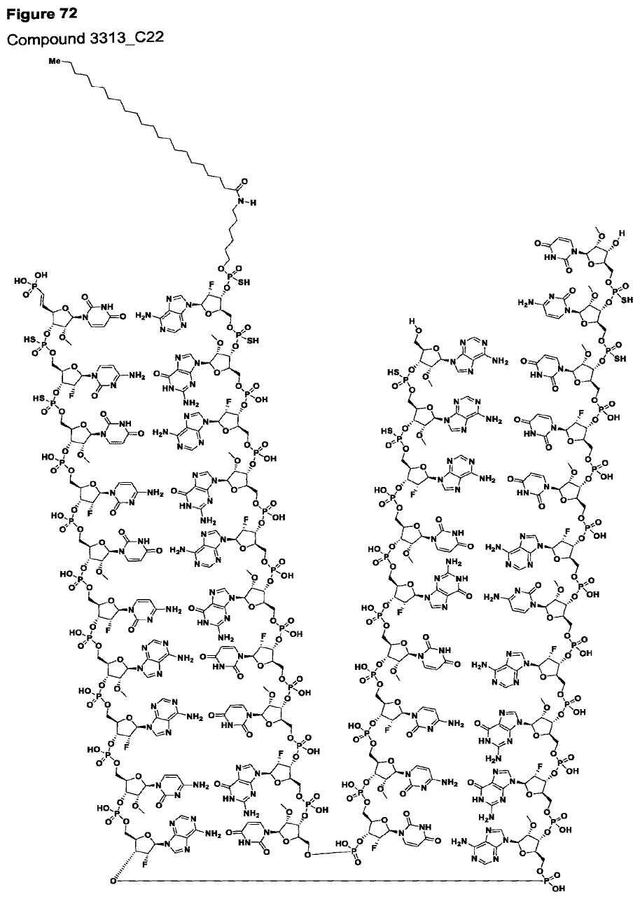

The invention provides a compound comprising the structure as shown in FIG. 72 (compound 3313_C22). The invention provides a compound consisting of the structure as shown in FIG. 72 (compound 3313_C22).

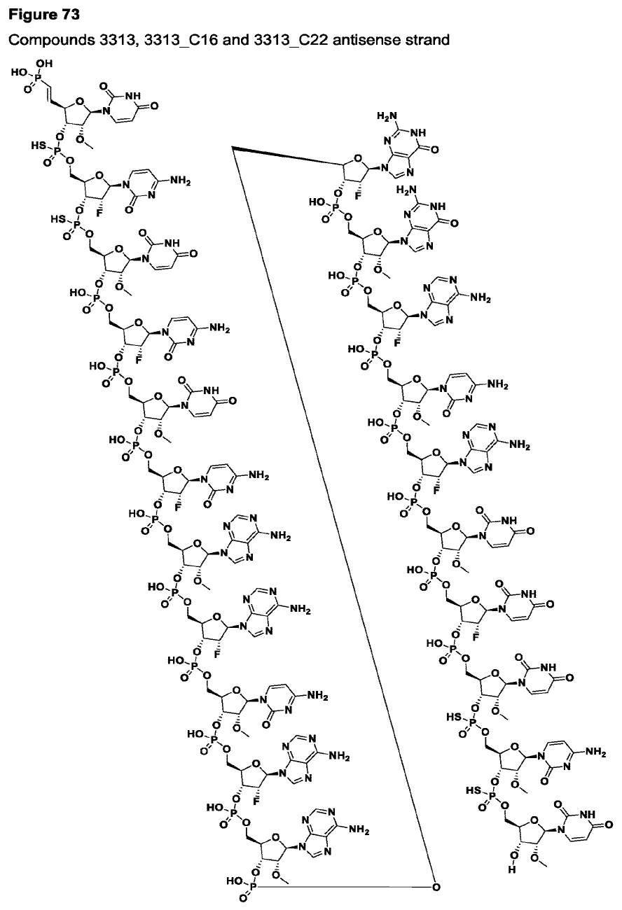

The invention provides a compound comprising the structure as shown in FIG. 73 (antisense strand of compounds 3313, 3313_C16 and 3313_C22).

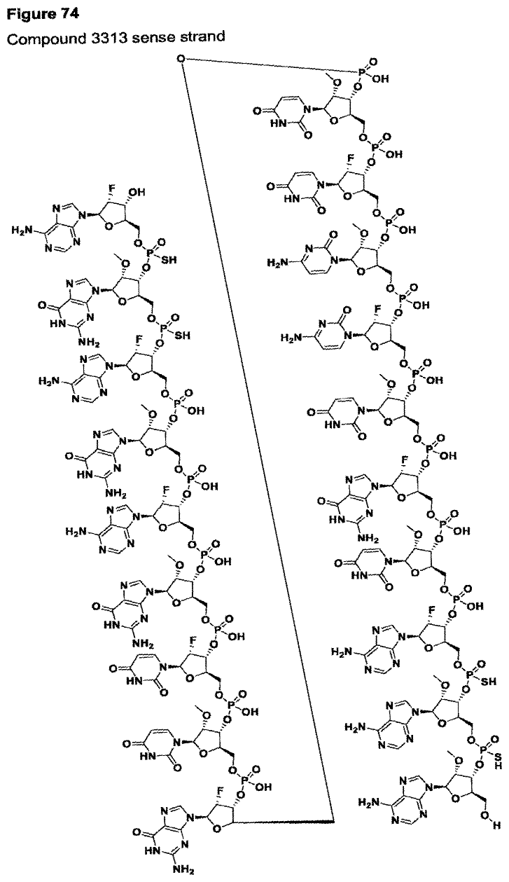

The invention provides a compound comprising the structure as shown in FIG. 74 (sense strand of compound 3313).

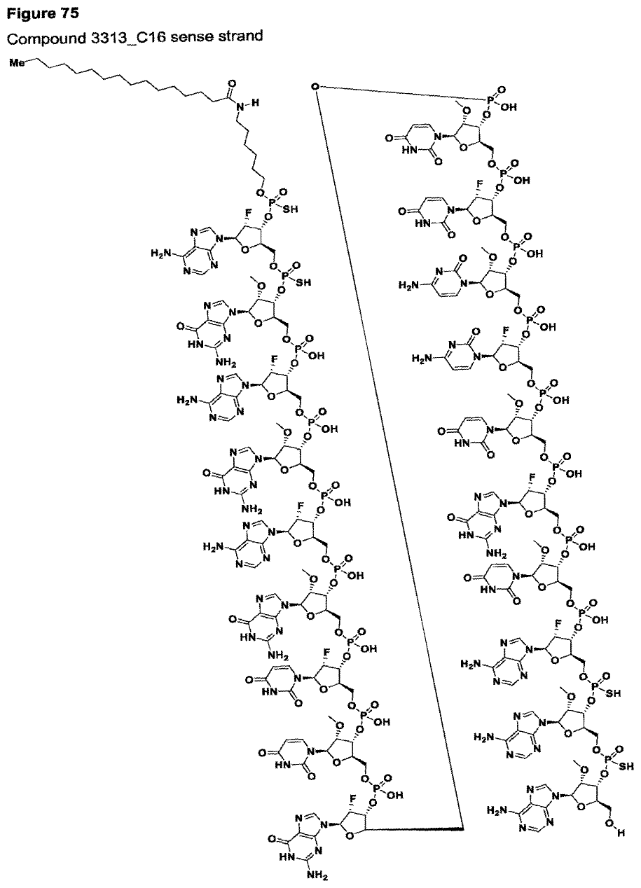

The invention provides a compound comprising the structure as shown in FIG. 75 (the sense strand of compound 3313_C16).

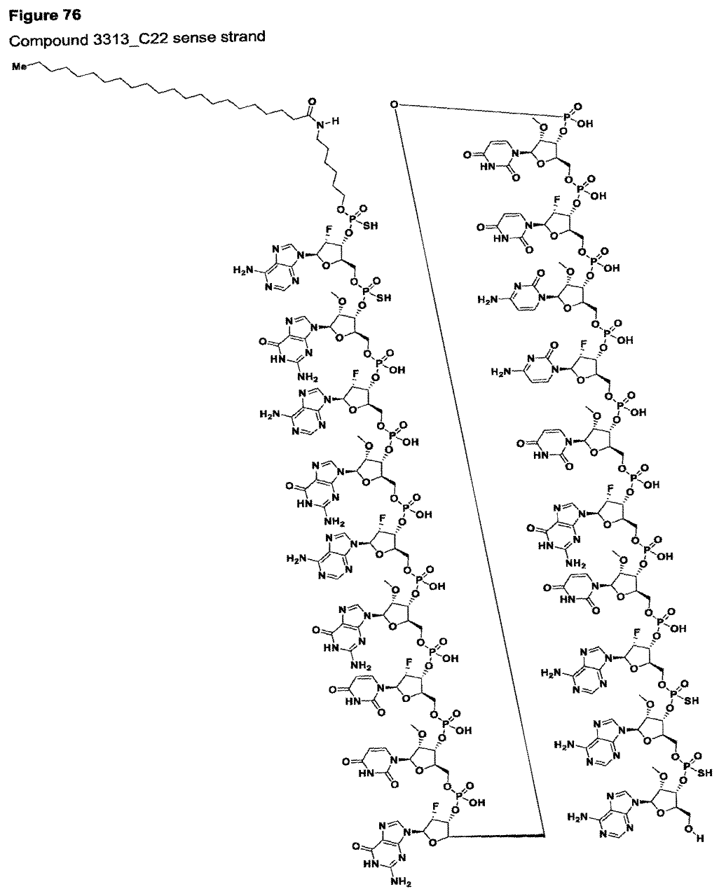

The invention provides a compound comprising the structure as shown in FIG. 76 (the sense strand of compound 3313_C22).

### Embodiments of the Invention

The invention is described in the following numbered paragraphs:

1. A compound comprising a double stranded ribonucleic acid (dsRNA) for reducing the expression of Janus kinase 1 (JAK1), the dsRNA comprising a sense strand and an antisense strand,
wherein the sense strand comprises a first contiguous nucleotide sequence of at least 15 nucleotides in length,
wherein the antisense strand comprises a second contiguous nucleotide sequence of at least 15 nucleotides in length which is complementary to a JAK1 nucleic acid sequence which comprises or consists of SEQ ID NO: 1 or a naturally occurring variant thereof, and
wherein the first contiguous nucleotide sequence and the second contiguous nucleotide sequence form a double stranded region of complementarity.
   2. The compound of paragraph 1, wherein the dsRNA is a siRNA.
   3. The compound of paragraph 1 or paragraph 2, wherein the second contiguous nucleotide sequence is complementary to a target sequence within the JAK1 nucleic acid sequence, wherein the target sequence is any one of the sequences of SEQ ID NOs 385-575, preferably any one of the sequences of SEQ ID NOs 385, 386, 405, 447, 456, 479, 498, 512, 517 and 530.
   4. The compound of paragraph 3, wherein the second contiguous nucleotide sequence is at least 80% complementary, at least 90% complementary, at least 95% complementary or fully complementary to the target sequence.
   5. The compound of any one of paragraphs 1-4, wherein the double stranded region of complementarity is 15, 16, 17, 18, 19, 20 or 21 nucleotides long, preferably 19 nucleotides long.
   6. The compound of any one of paragraphs 1-5, wherein the double stranded region of complementarity comprises one or more mismatches, such as one, two, three, four or five mismatches.
   7. The compound of any one of paragraphs 1-6, wherein the first contiguous nucleotide sequence and the second contiguous nucleotide sequence within the double stranded region of complementarity are at least 80% complementary, at least 90% complementary, at least 95% complementary or fully complementary.
   8. The compound of any one of paragraphs 1-7, wherein the second contiguous nucleotide sequence comprises a seed region, wherein the sequence of the seed region comprises or consists of any one of the sequences of SEQ ID NOs 576-766, preferably any one of the sequences of SEQ ID NOs 576, 577, 596, 638, 647, 670, 689, 703, 708 and 721.
   9. The compound of any one of paragraphs 1-8, wherein the second contiguous nucleotide sequence is 15, 16, 17, 18, 19, 20 or 21 nucleotides long, preferably 20 or 21 nucleotides long.
   10. The compound of any one of paragraphs 1-9, wherein the second contiguous nucleotide sequence corresponds to a portion of SEQ ID NO: 2 in which thymine (T) nucleobases are replaced with uracil (U) nucleobases.
   11. The compound of any one of paragraphs 1-10, wherein the second contiguous nucleotide sequence comprises or consists of a sequence having at least 80% identity to any one of the sequences of SEQ ID NOs 194-384, such as at least 85%, 90%, 91%, 92%, 93%, 94%, 95%, 96%, 97%, 98% or 99% identity to any one of the sequences of SEQ ID NOs 194-384.
   12. The compound of any one of paragraphs 1-11, wherein the second contiguous nucleotide sequence comprises or consists of any one of the sequences of SEQ ID NOS 194-384, preferably any one of the sequences of SEQ ID NOs 194, 195, 214, 256, 265, 288, 307, 321, 326 and 339.
   13. The compound of any one of paragraphs 1-12, wherein the sequence of the antisense strand consists of the second contiguous nucleotide sequence.
   14. The compound of any one of paragraphs 1-13, wherein the antisense strand comprises a uracil nucleotide, such as a Vinyl-phosphonate 2′-OMe uracil, located at the 5′ end of the antisense strand.
   15. The compound of any one of paragraphs 1-14, wherein the antisense strand is 19, 20, 21, 22, 23, 24, 25, 26 or 27 nucleotides long, preferably 21 nucleotides long.
   16. The compound of any one of paragraphs 1-15, wherein the first contiguous nucleotide sequence is 15, 16, 17, 18, 19, 20 or 21 nucleotides long, preferably 19 nucleotides long.
   17. The compound of any one of paragraphs 1-16, wherein the first contiguous nucleotide sequence comprises or consists of any one of the sequences of SEQ ID NOs 3-193, preferably any one of the sequences of SEQ ID NOs 3, 4, 21, 63, 72, 95, 114, 128, 133 and 146.
   18. The compound of any one of paragraphs 1-17, wherein the sequence of the sense strand consists of the first contiguous nucleotide sequence.
   19. The compound of any one of paragraphs 1-18, wherein the sense strand is 17, 18, 19, 20, 21, 22, 23, 24 or 25 nucleotides long, preferably 19 nucleotides long.
   20. The compound of any one of paragraphs 1-19, wherein the antisense strand and the sense strand form a duplex selected from the group consisting of duplex numbers 1-191 as shown in Table 1, preferably duplex numbers 1, 2, 21, 63, 72, 95, 114, 128, 133, and 146 as shown in Table 1.
   21. The compound of any one of paragraphs 1-20, wherein the dsRNA comprises at least one modified nucleotide.
   22. The compound of any one of paragraphs 1-21, wherein the antisense strand comprises at least one modified nucleotide.
   23. The compound of any one of paragraphs 1-22, wherein the sense strand comprises at least one modified nucleotide.
   24. The compound of any one of paragraphs 21-23, wherein the at least one modified nucleotide comprises a modified sugar moiety, preferably wherein the modified sugar moiety is independently selected from a bicyclic sugar moiety or a non-bicyclic sugar moiety, more preferably wherein the modified sugar moiety is a non-bicyclic sugar moiety.
   25. The compound of paragraph 24, wherein the non-bicyclic sugar moiety is independently selected from 2′-O-alkyl-RNA, 2′-O-methyl-RNA (2′OMe modified sugar), 2′-alkoxy-RNA, 2′-O-methoxyethyl-RNA, 2′-amino-DNA, 2′-fluoro-DNA (2′F modified sugar), arabino nucleic acid (ANA), 2′-fluoro-ANA, Glycol nucleic acid (GNA), and unlocked nucleic acid (UNA), preferably wherein the non-bicyclic sugar moiety is independently selected from a 2′F modified sugar and a 2′OMe modified sugar.
   26. The compound of paragraph 25, comprising one or more 2′F modified sugar and/or one or more 2′OMe modified sugar moiety.
   27. The compound of paragraph 25 or paragraph 26, wherein at least 50% of the sugar moieties are 2′OMe modified sugar moieties, preferably wherein 50-85% of the sugar moieties are 2′OMe modified sugar moieties, more preferably wherein 68-85% of the sugar moieties are 2′OMe modified sugar moieties.
   28. The compound of any one of paragraphs 25-27, wherein each sugar moiety in both the sense strand and antisense strand is independently selected from a 2′OMe modified sugar and a 2′F modified sugar.
   29. The compound of any one of paragraphs 1-28, wherein the dsRNA comprises at least one least one modified internucleotide linkage.
   30. The compound of any one of paragraphs 1-29, wherein the antisense strand comprises at least one modified internucleotide linkage.
   31. The compound of any one of paragraphs 1-30, wherein the sense strand comprises at least one modified internucleotide linkage.
   32. The compound of any one of paragraphs 29-31, wherein the modified internucleotide linkage is independently selected from a phosphorothioate internucleotide linkage, a diphosphorothioate internucleotide linkage and a boranophosphate internucleotide linkage.
   33. The compound of any one of paragraphs 1-32, wherein each internucleotide linkage of the dsRNA is either a phosphodiester internucleotide linkage or a phosphorothioate internucleotide linkage.
   34. The compound of any one of paragraphs 1-33, wherein the dsRNA comprises at least one modified nucleobase.
   35. The compound of paragraph 34, wherein the modified nucleobase is 5-methyl cytosine.
   36. The compound of any one of paragraphs 1-35, wherein the dsRNA is covalently attached to at least one conjugate moiety.
   37. The compound of paragraph 36, wherein a conjugate moiety is covalently attached to the sense strand; preferably covalently attached at the 3′-end of the sense strand and/or covalently attached at the 5′-end of the sense strand.
   38. The compound of paragraph 36 or paragraph 37, wherein a conjugate moiety is covalently attached to the antisense strand; preferably covalently attached at the 3′-end of the antisense strand and/or covalently attached at the 5′-end of the antisense strand.
   39. The compound of any one of paragraphs 36-38, wherein the conjugate moiety is covalently attached to the 3′-end of the sense strand.
   40. The compound of any one of paragraphs 36-39, wherein the conjugate moiety is independently selected from the group consisting of carbohydrates, fatty acids, cell surface receptor ligands, drug substances, hormones, lipophilic substances, polymers, proteins, peptides, toxins (e.g. bacterial toxins), vitamins, viral proteins (e.g. capsids) and combinations thereof.
   41. The compound of any one of paragraphs 36-40, wherein the conjugate moiety is a fatty acid, preferably a C16 fatty acid or a C22 fatty acid, more preferably a C22 fatty acid.
   42. The compound of paragraph 41, wherein the fatty acid is palmitic acid or behenic acid, preferably behenic acid.
   43. The compound of any one of paragraphs 36-42, wherein the dsRNA is covalently attached to the conjugate moiety via a linker.
   44. The compound of paragraph 43, wherein the linker is a cleavable linker.
   45. The compound of paragraph 44, wherein the cleavable linker comprises or consists of 1 to 3 linker nucleotides.
   46. The compound of paragraph 43, wherein the linker comprises or consists of a C2 to C36 amino alkyl linker, preferably wherein the linker comprises or consists of a C6 to C12 amino alkyl linker, most preferably wherein the linker comprises or consists of a C6 amino alkyl linker.
   47. The compound of any one of paragraphs 36-42, wherein the dsRNA is directly attached to the conjugate moiety.
   48. The compound of any one of paragraphs 1-47, wherein the compound is capable of decreasing the expression of JAK1 mRNA by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%, compared to a control.
   49. The compound of any one of paragraphs 1-48, wherein the compound is capable of decreasing the expression of JAK1 protein by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100%, compared to a control.
   50. The compound of paragraph 48 or paragraph 49, wherein the control is a cell that has not been exposed to the compound.
   51. A compound selected from compounds 614, 673, 724, 728, 753, 756, 818, 874, 875, 876, 877, 878, 883, 884, 1069, 1075, 1085, 1107, 1108, 1138, 1182, 1189, 1190, 1304, 1306, 1311, 1367, 1368, 1372, 1412, 1413, 1432, 1579, 1580, 1581, 1583, 1584, 1586, 1587, 1588, 1595, 1596, 1601, 1602, 1603, 1608, 1609, 1611, 1640, 1642, 1671, 1672, 1673, 1674, 1677, 1678, 1690, 1692, 1698, 1699, 1723, 1769, 1770, 1780, 1798, 1876, 1927, 1928, 1929, 1936, 1952, 1954, 1956, 1958, 1978, 2066, 2068, 2102, 2111, 2138, 2146, 2148, 2205, 2206, 2218, 2229, 2230, 2237, 2238, 2239, 2269, 2308, 2317, 2318, 2319, 2320, 2321, 2322, 2323, 2520, 2527, 2647, 2761, 2762, 2763, 2764, 2811, 2962, 2975, 2977, 3028, 3032, 3081, 3131, 3134, 3141, 3144, 3146, 3147, 3159, 3160, 3229, 3247, 3250, 3251, 3252, 3254, 3255, 3258, 3259, 3260, 3261, 3265, 3268, 3272, 3275, 3276, 3278, 3279, 3281, 3282, 3283, 3284, 3285, 3286, 3313, 3314, 3323, 3353, 3365, 3367, 3368, 3371, 3372, 3376, 3409, 3505, 3556, 3557, 3558, 3559, 3654, 3662, 3663, 3683, 3689, 3694, 3695, 3698, 3702, 3719, 3781, 3894, 4099, 4169, 4239, 4305, 4374, 4411, 4475, 4612, 4671, 4672, 4679, 4682, 4683, 4684, 4690, 4794, 4803, and 4807, as shown in Table 3, preferably a compound selected from compounds 614, 673, 1182, 1770, 1954, 2319, 3131, 3255, 3265, and 3313 as shown in Table 3.
   52. A compound selected from compounds 614, 673, 1182, 1770, 1954, 2319, 3131, 3255, 3265, 3313, 614_C16, 673_C16, 1182_C16, 1770_C16, 1954_C16, 2319_C16, 3131_C16, 3255_C16, 3265_C16, 3313_C16, 614_C22, 673_C22, 1182_C22, 1770_C22, 1954_C22, 2319_C22, 3131_C22, 3255_C22, 3265_C22 and 3313_C22.
   53. A compound selected from compounds 614, 673, 1182, 1770, 1954, 2319, 3131, 3255, 3265, 3313, 614_C16, 673_C16, 1182_C16, 1770_C16, 1954_C16, 2319_C16, 3131_C16, 3255_C16, 3265_C16, 3313_C16, 614_C22, 673_C22, 1182_C22, 1770_C22, 1954_C22, 2319_C22, 3131_C22, 3255_C22, 3265_C22 and 3313_C22 as shown in Table 4.
   54. A compound comprising or consisting of the structure (compound 614):

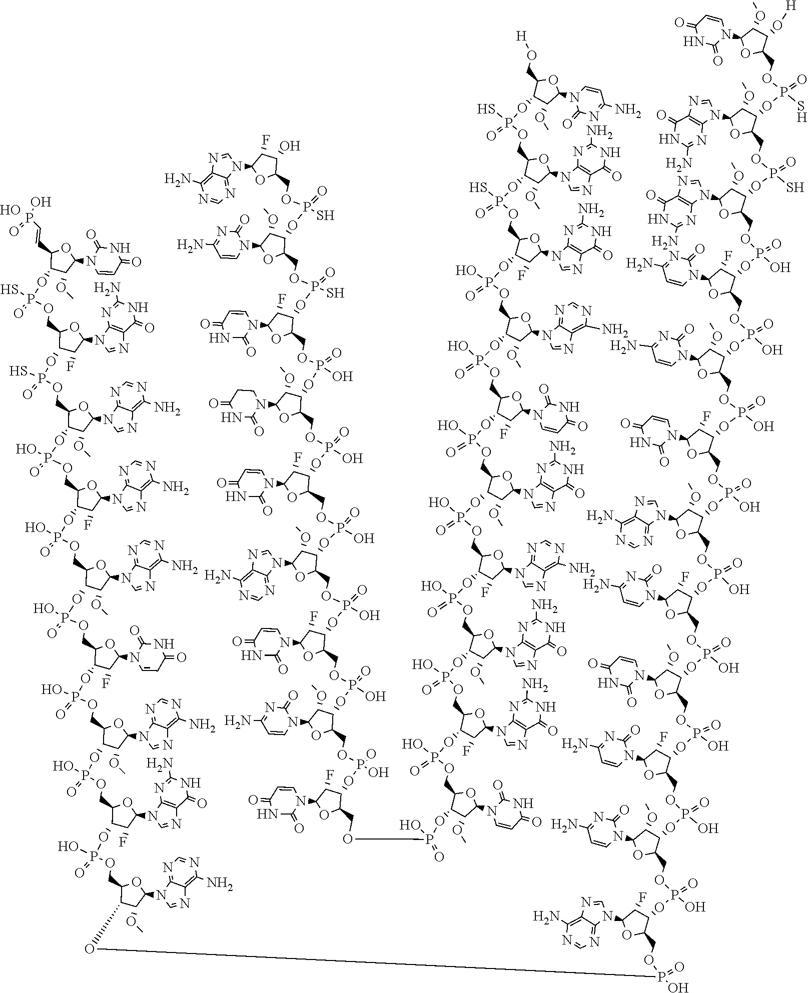

55. A compound comprising or consisting of the structure (compound 614_C16):

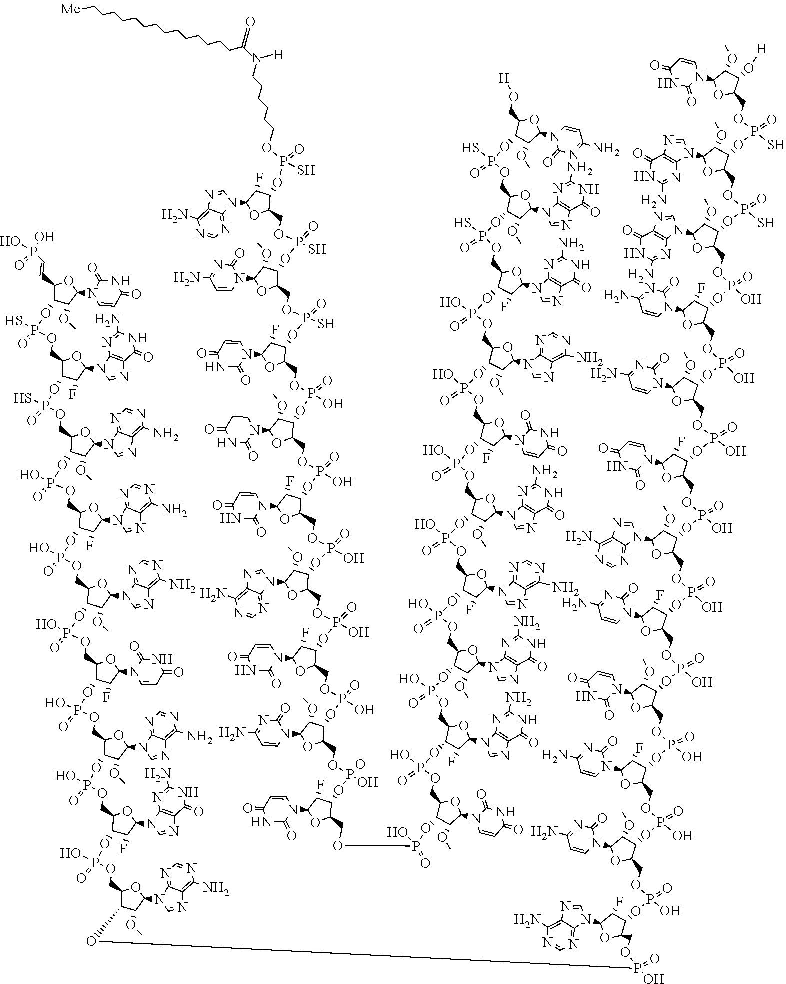

56. A compound comprising or consisting of the structure (compound 614_C22):

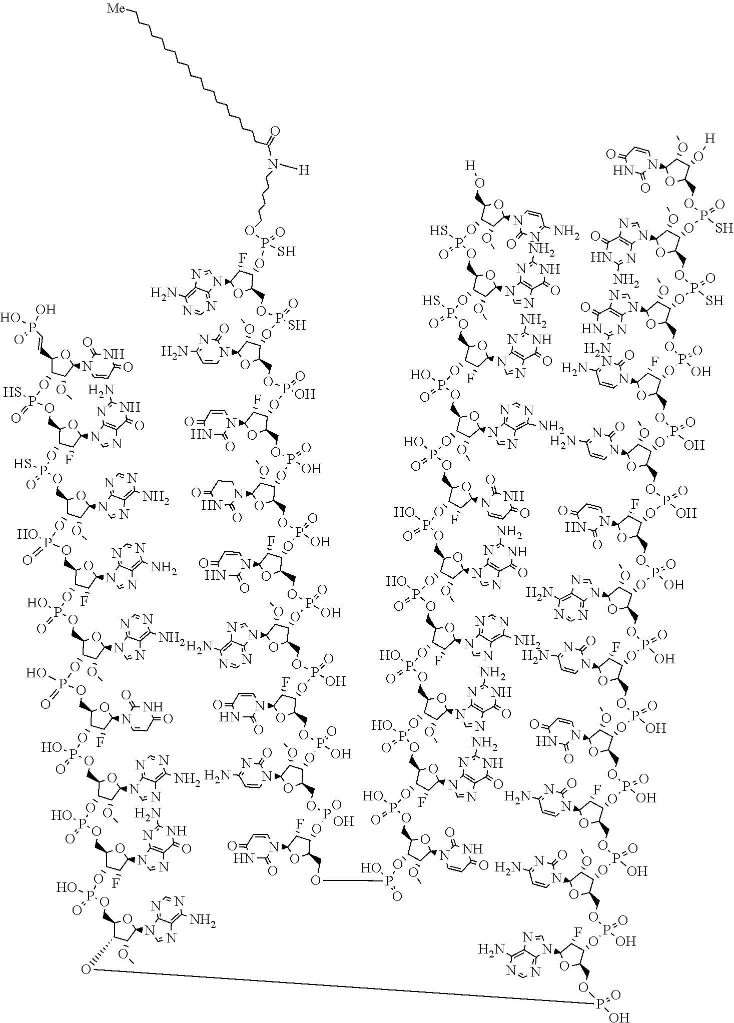

57. A compound comprising or consisting of the structure (compound 673):

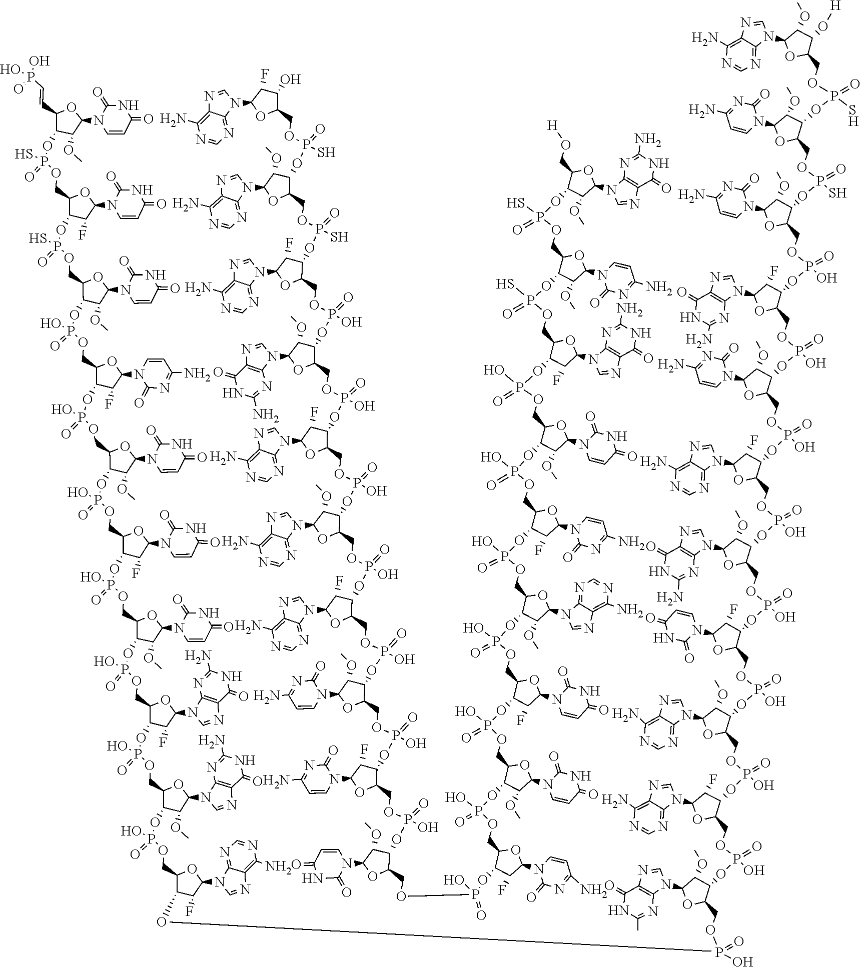

58. A compound comprising or consisting of the structure (compound 673_C16):

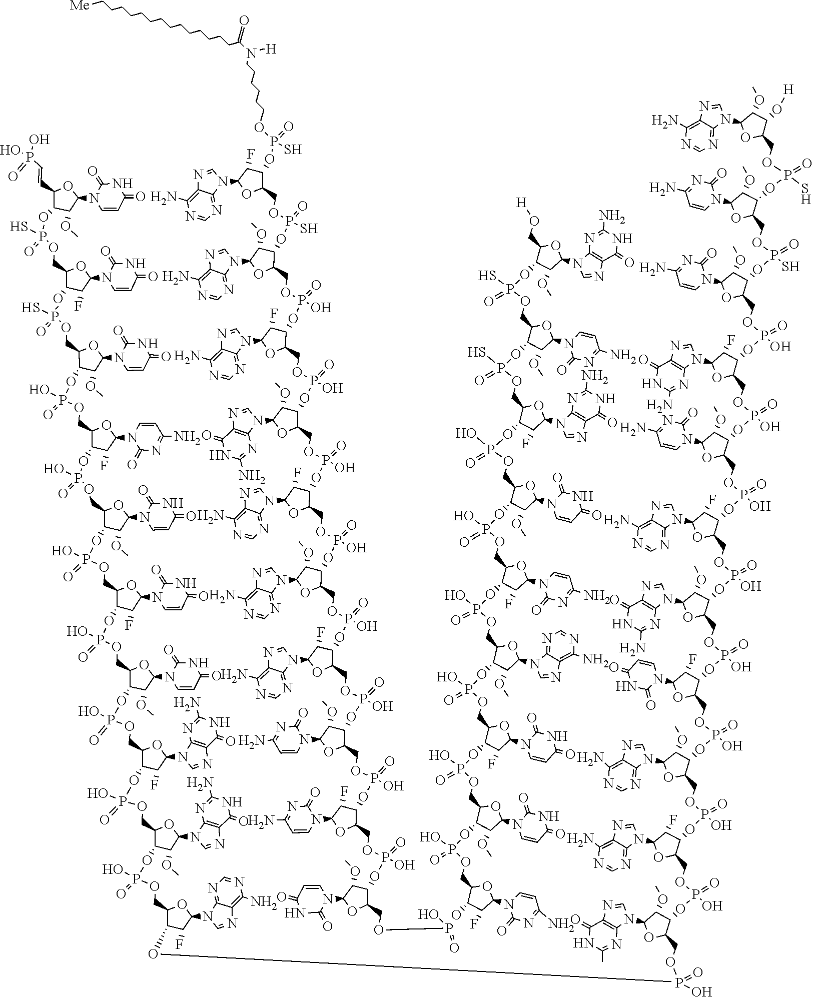

59. A compound comprising or consisting of the structure (compound 673_C22):

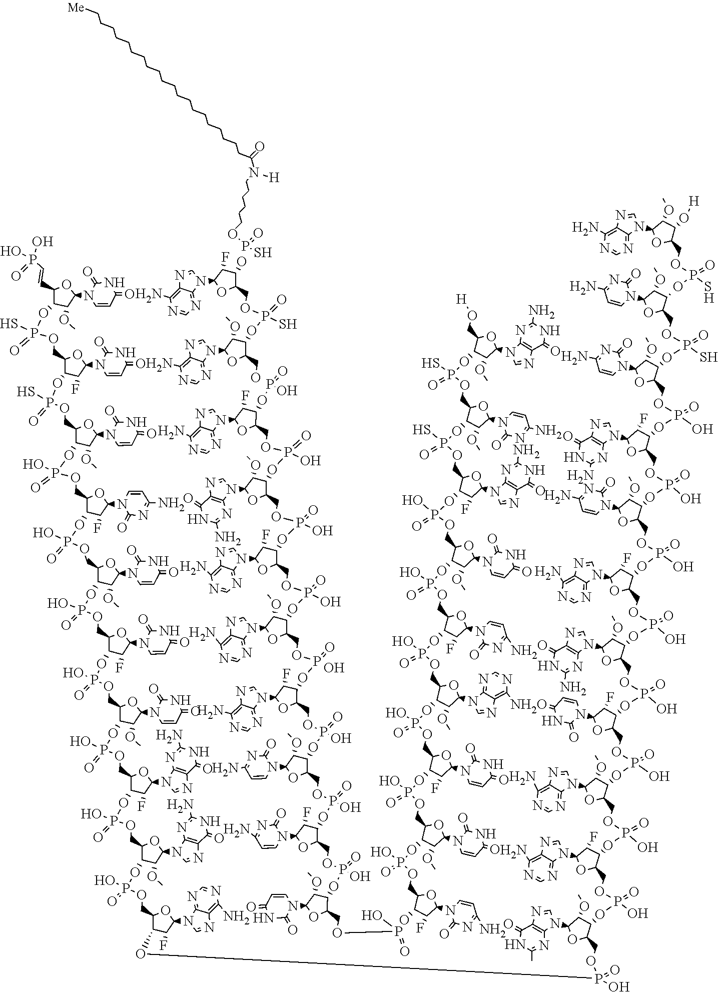

60. A compound comprising or consisting of the structure (compound 1182):

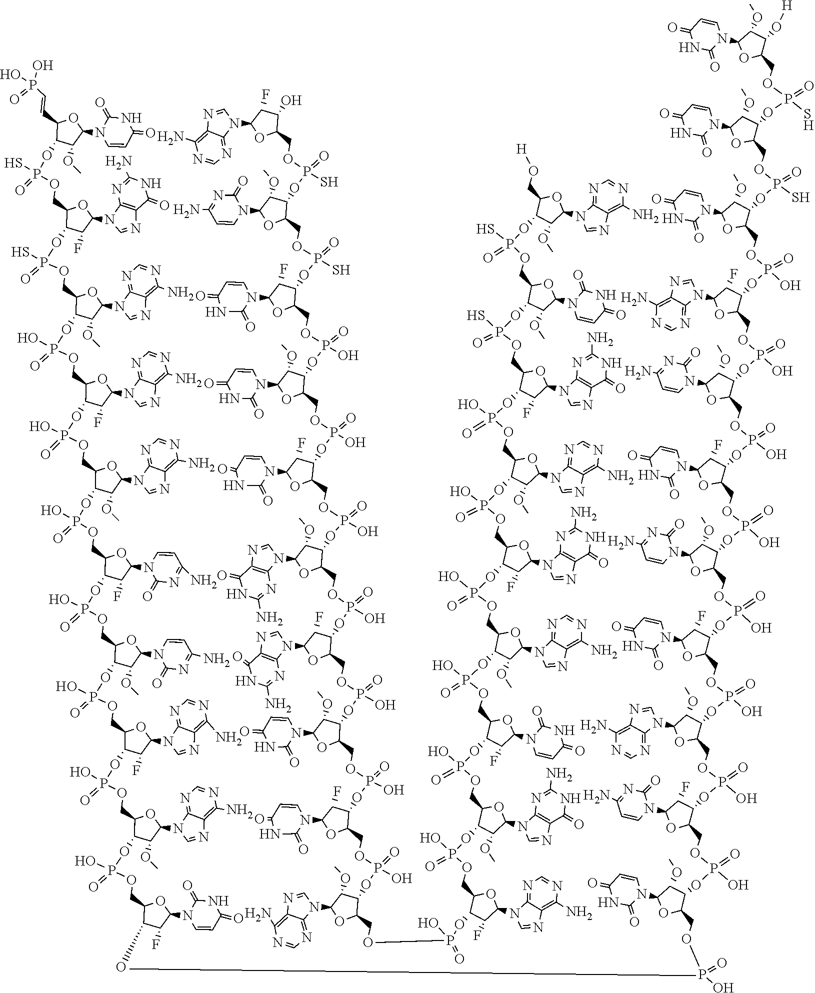

61. A compound comprising or consisting of the structure (compound 1182_C16):

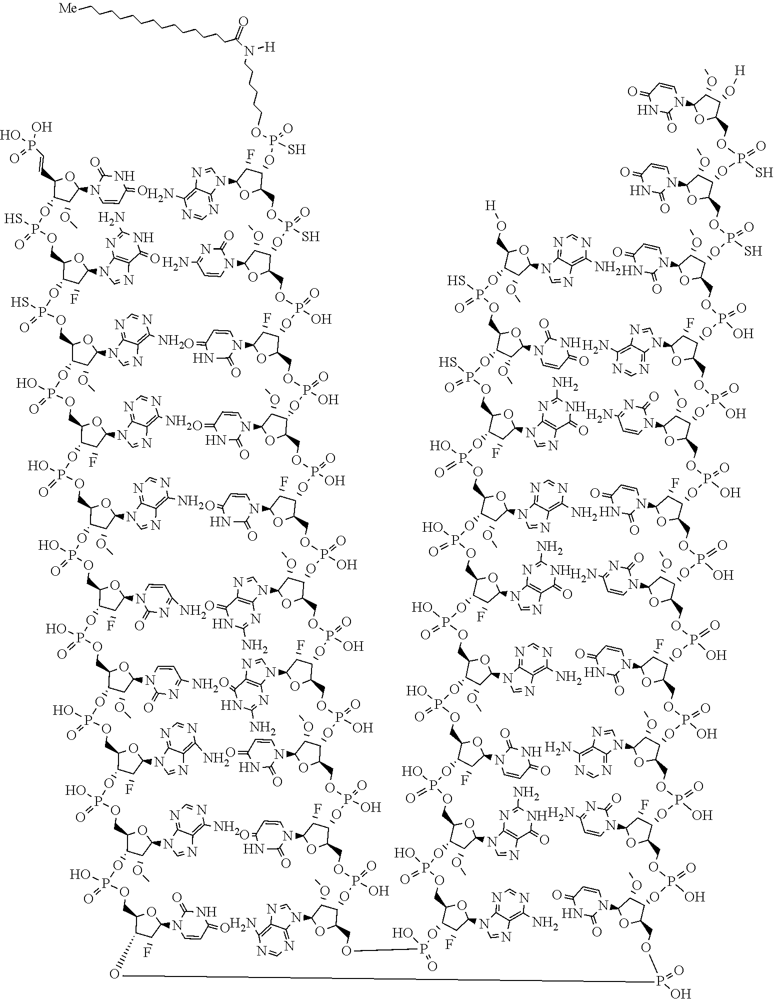

62. A compound comprising or consisting of the structure (compound 1182_C22):

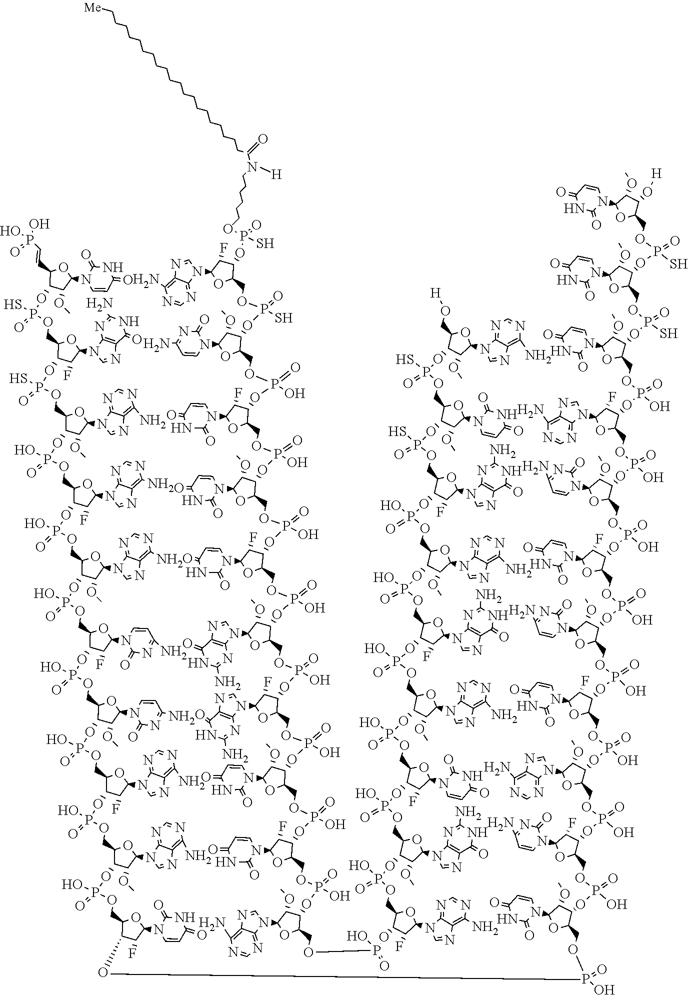

63. A compound comprising or consisting of the structure (compound 1770):

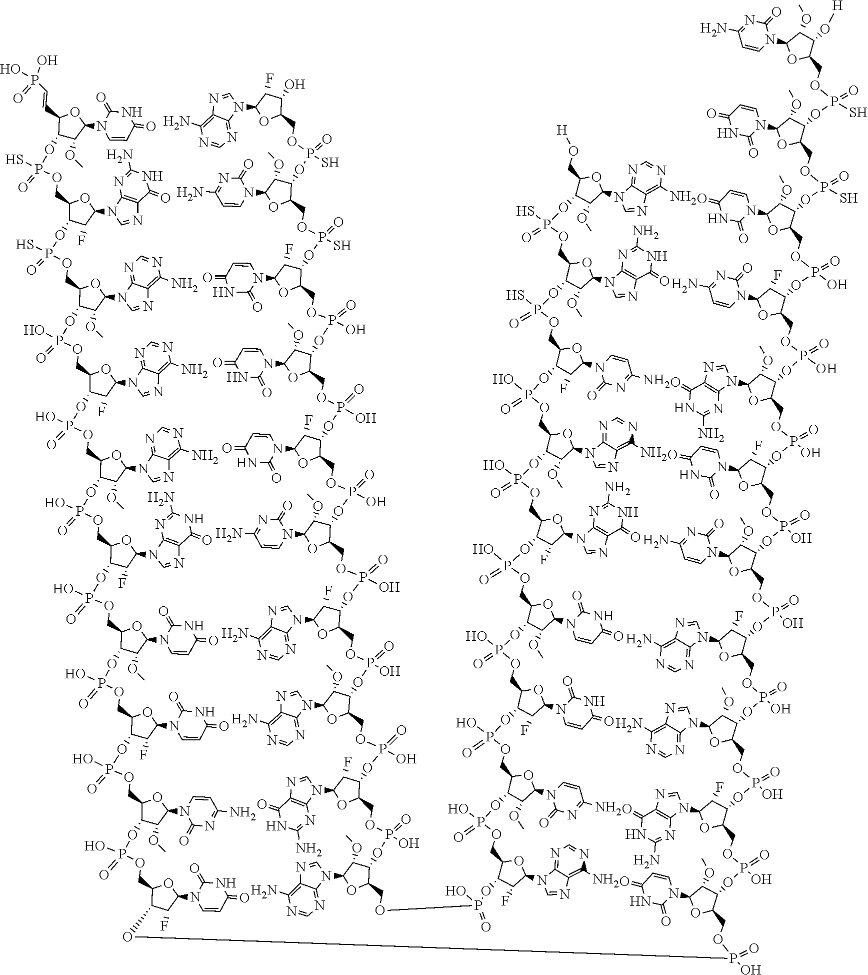

64. A compound comprising or consisting of the structure (compound 1770_C16):

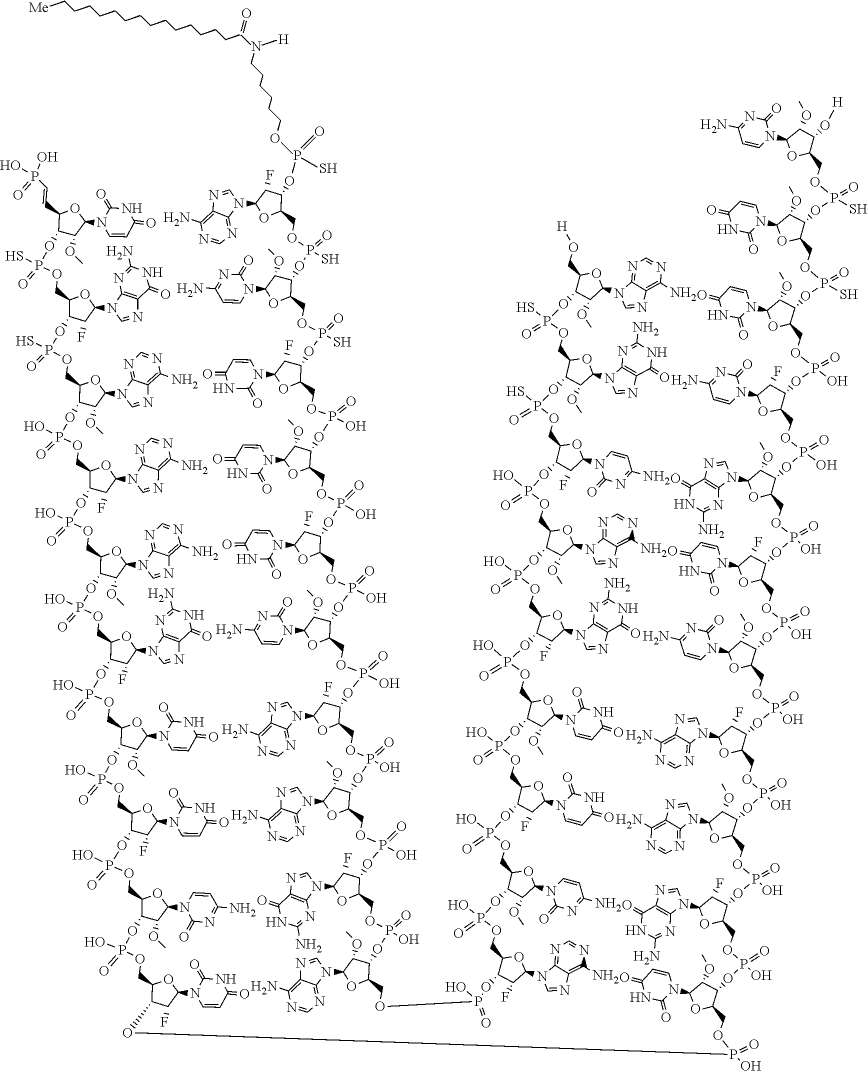

65. A compound comprising or consisting of the structure (compound 1770_C22):

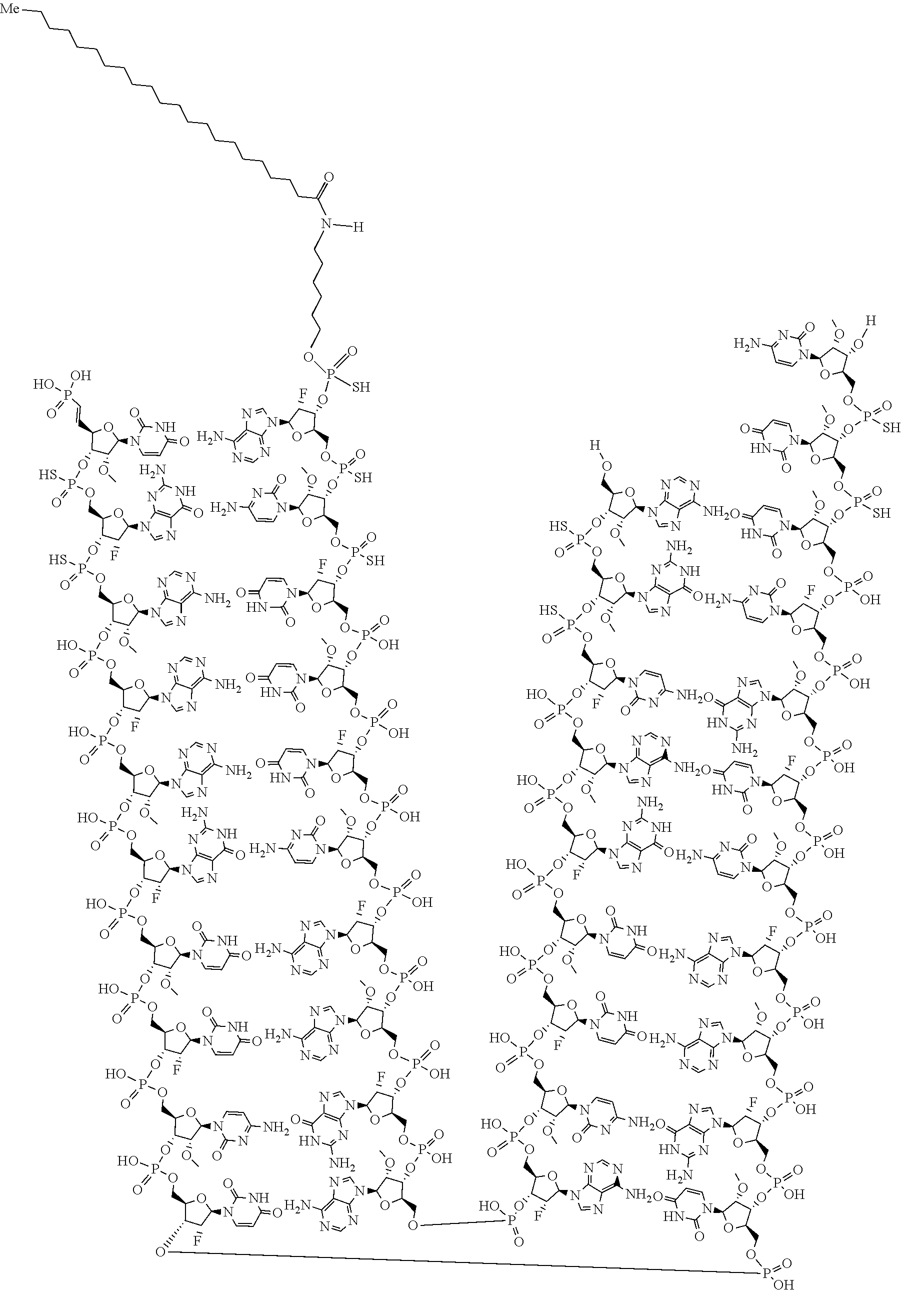

66. A compound comprising or consisting of the structure (compound 1954):

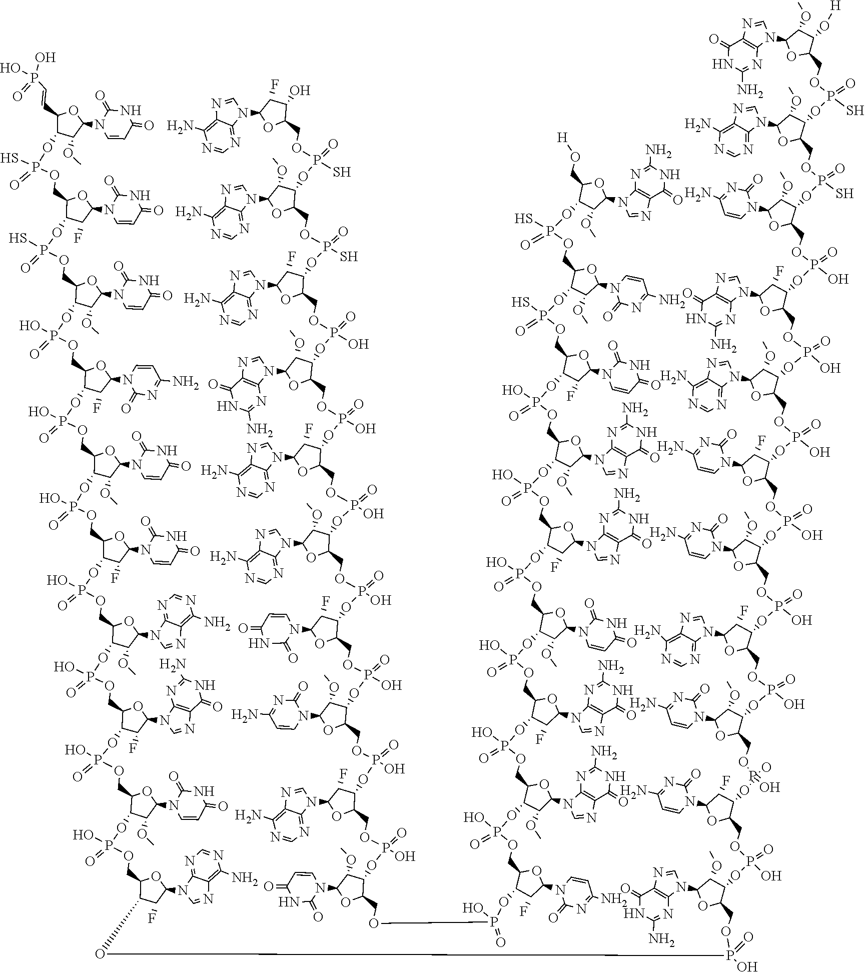

67. A compound comprising or consisting of the structure (compound 1954_C16):

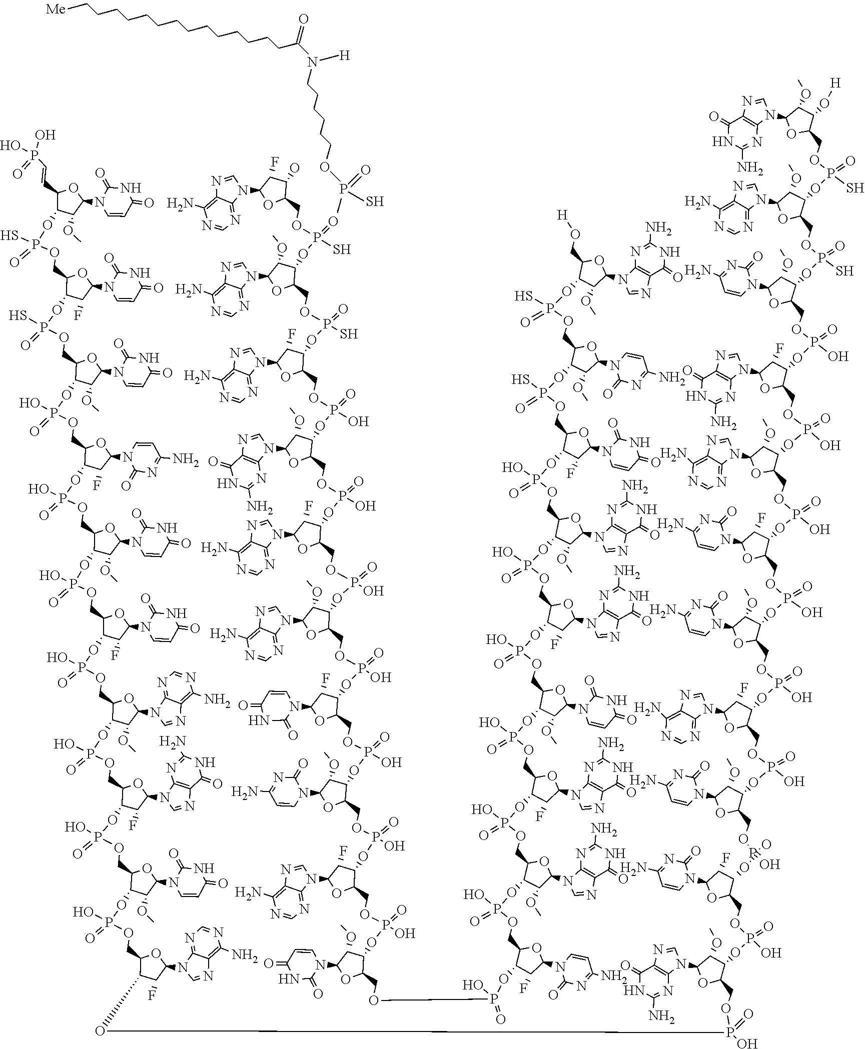

68. A compound comprising or consisting of the structure (compound 1954_C22):

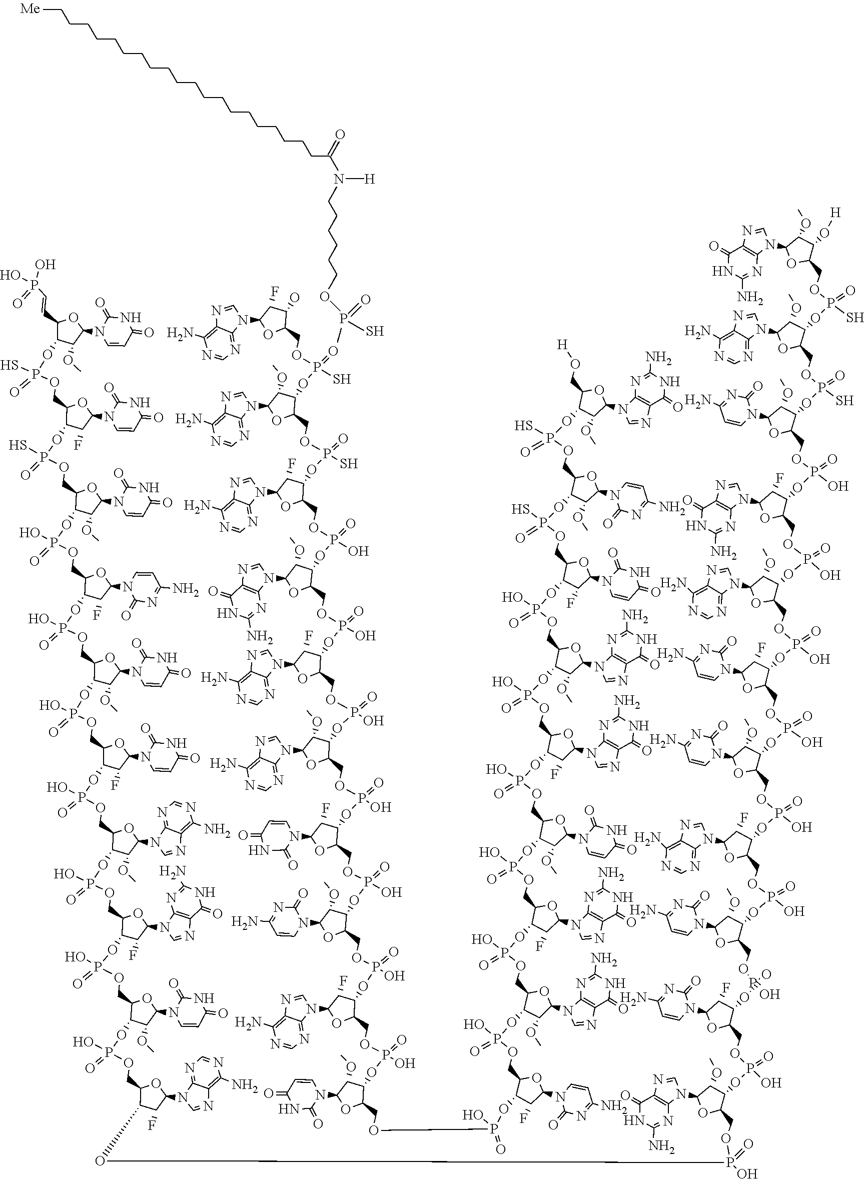

69. A compound comprising or consisting of the structure (compound 2319):

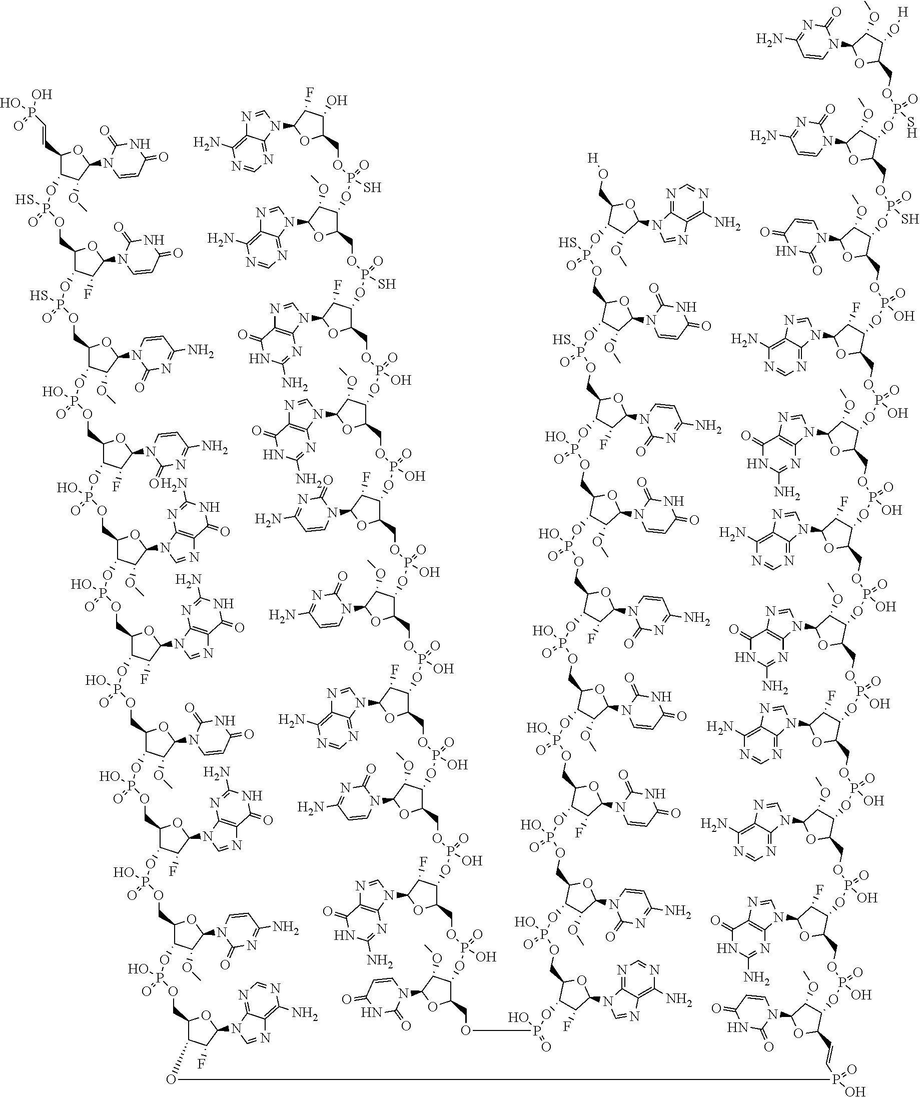

70. A compound comprising or consisting of the structure (compound 2319_C16):

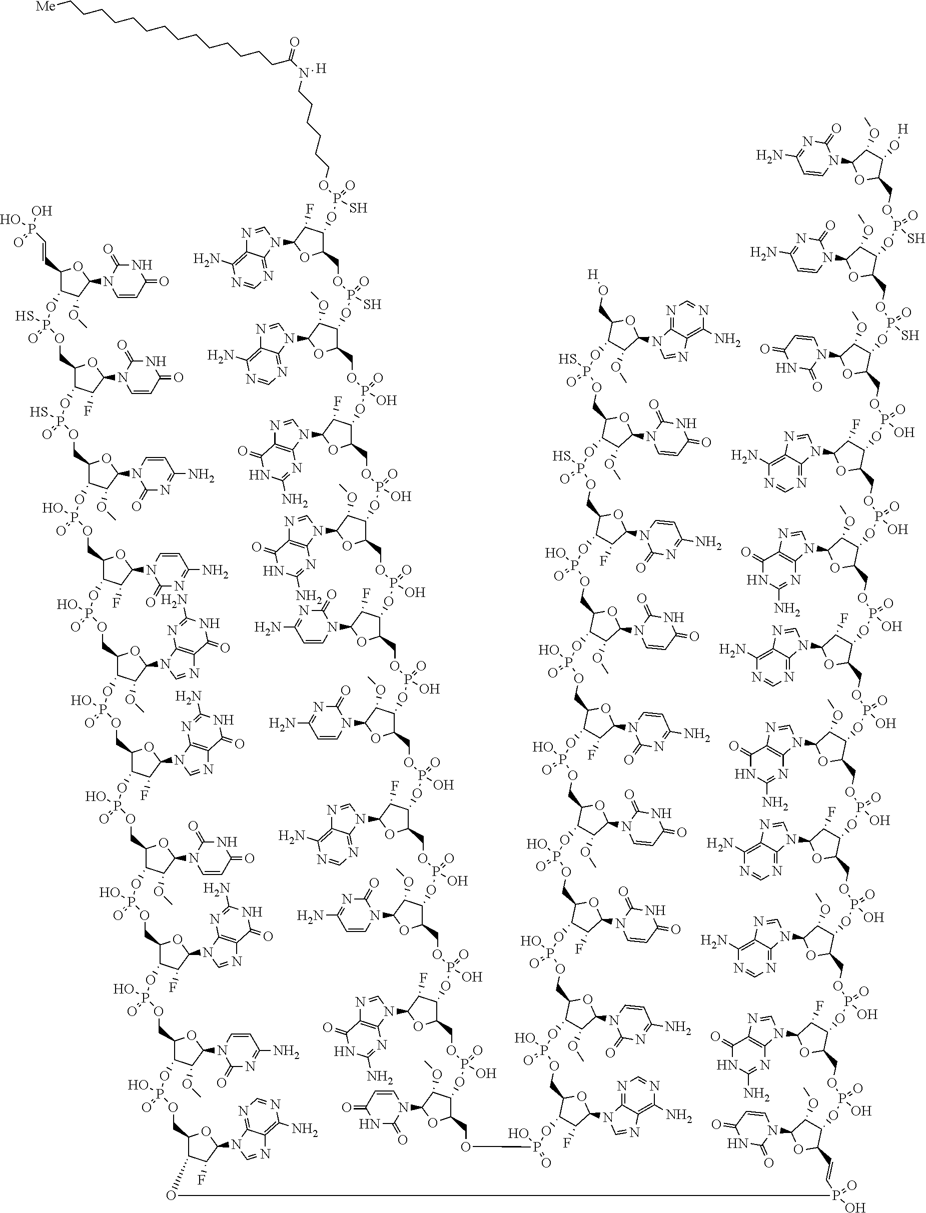

71. A compound comprising or consisting of the structure (compound 2319_C22):

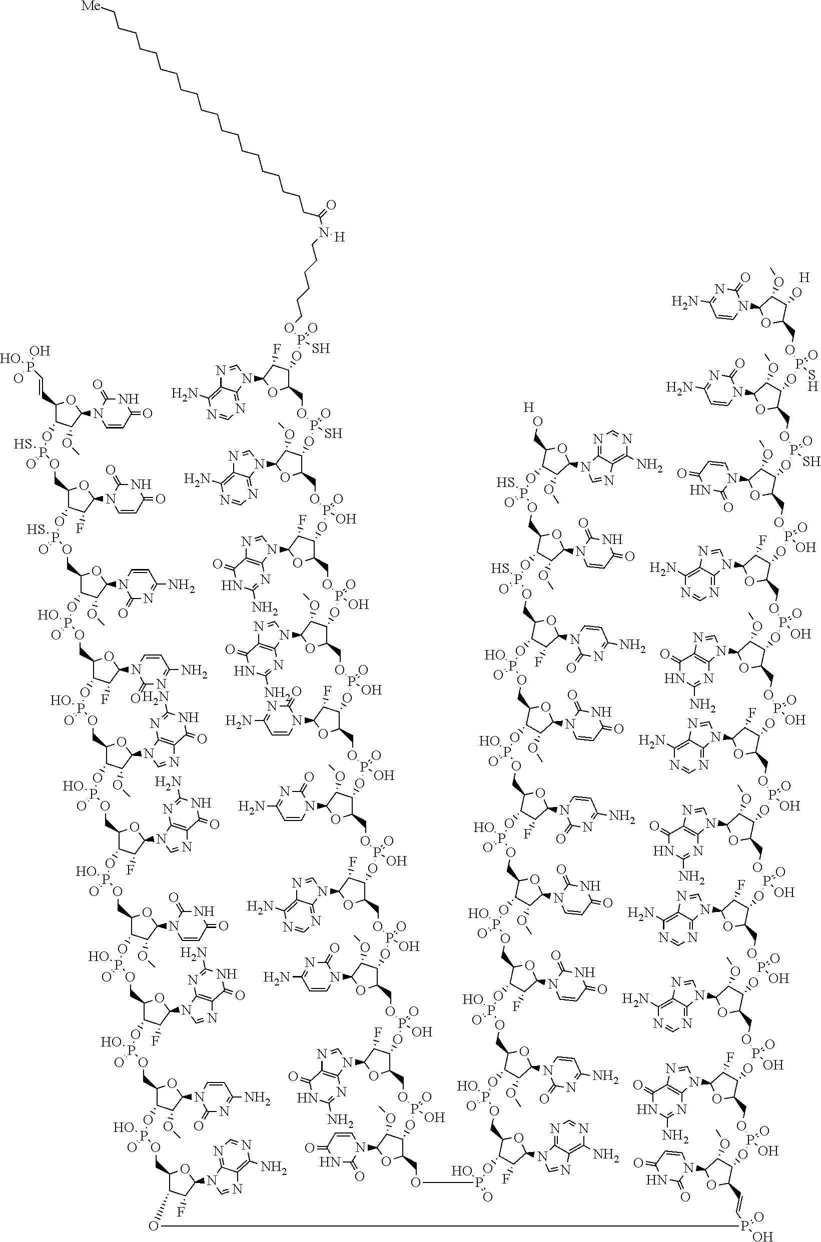

72. A compound comprising or consisting of the structure (compound 3131):

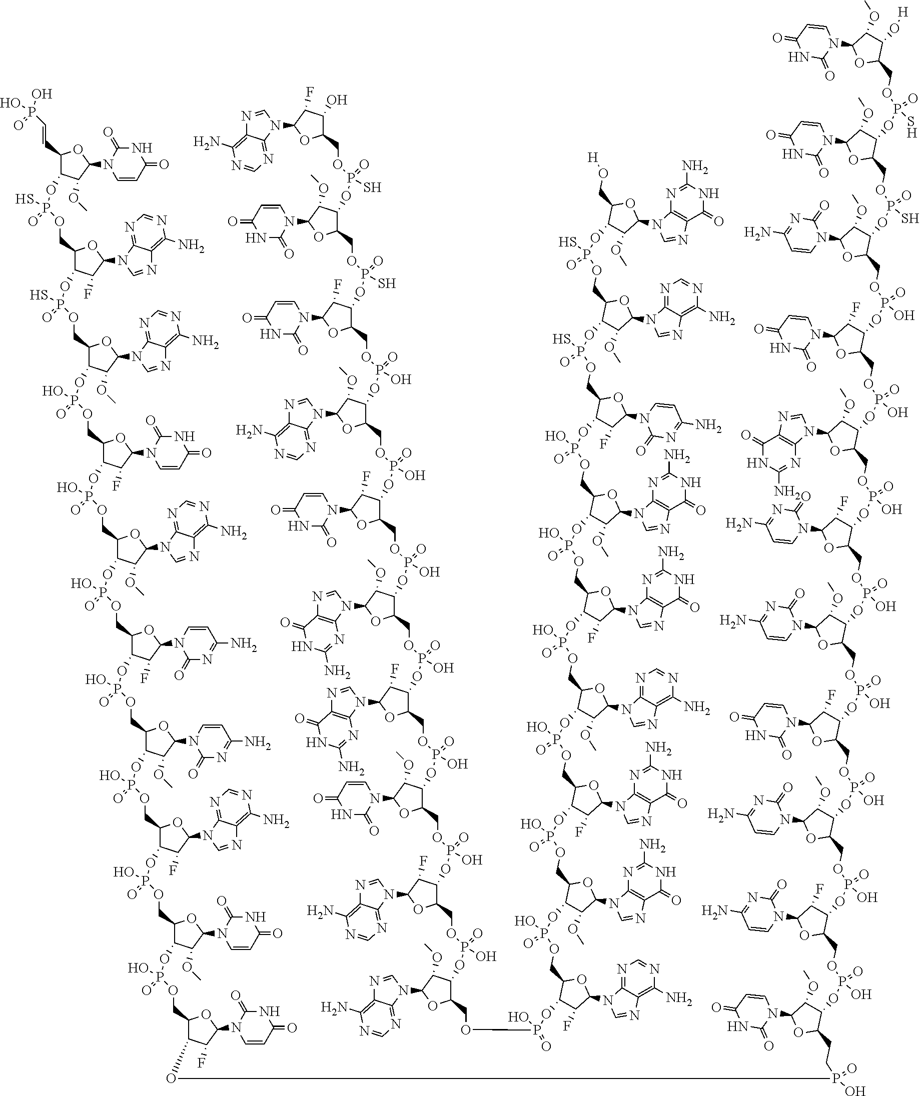

73. A compound comprising or consisting of the structure (compound 3131_C16):

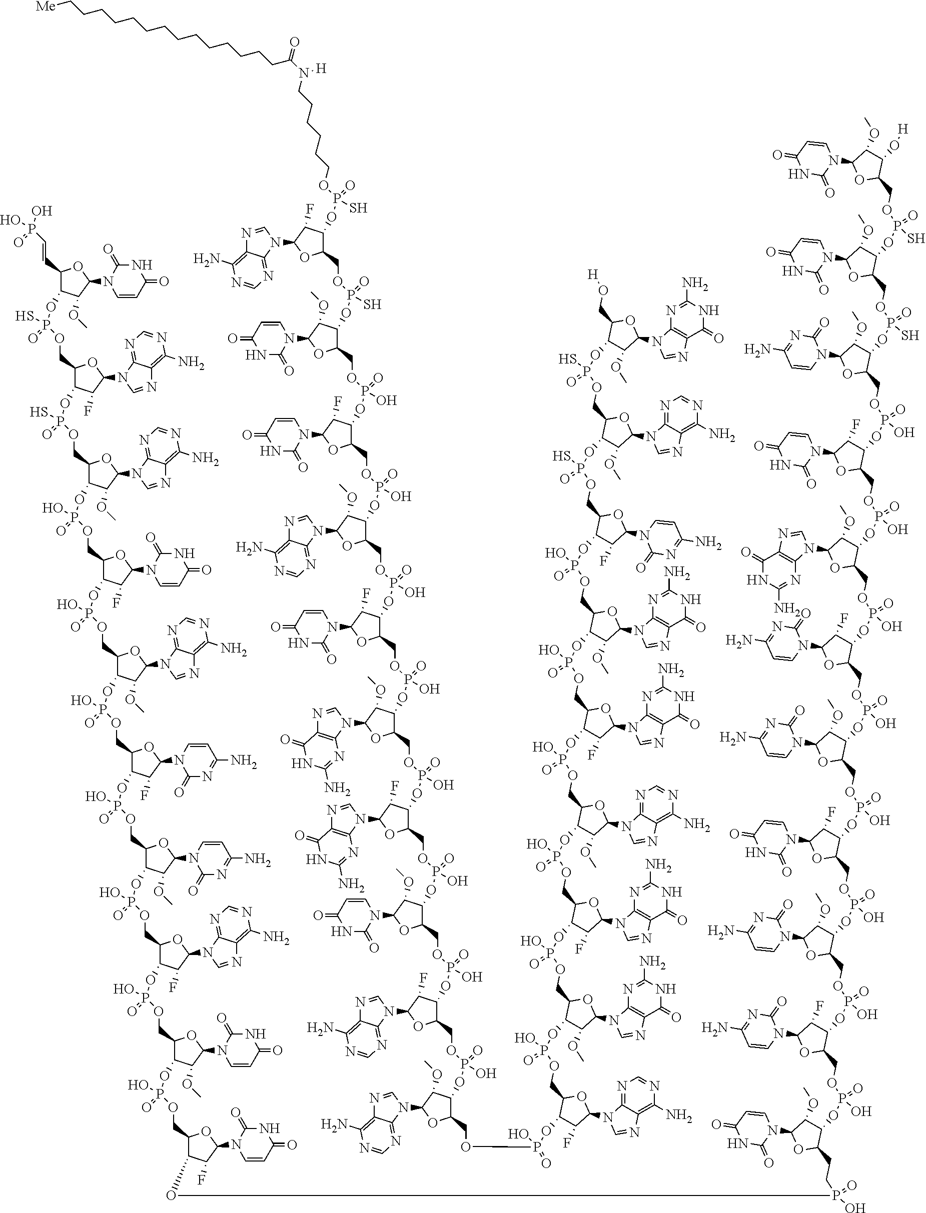

74. A compound comprising or consisting of the structure (compound 3131_C22):

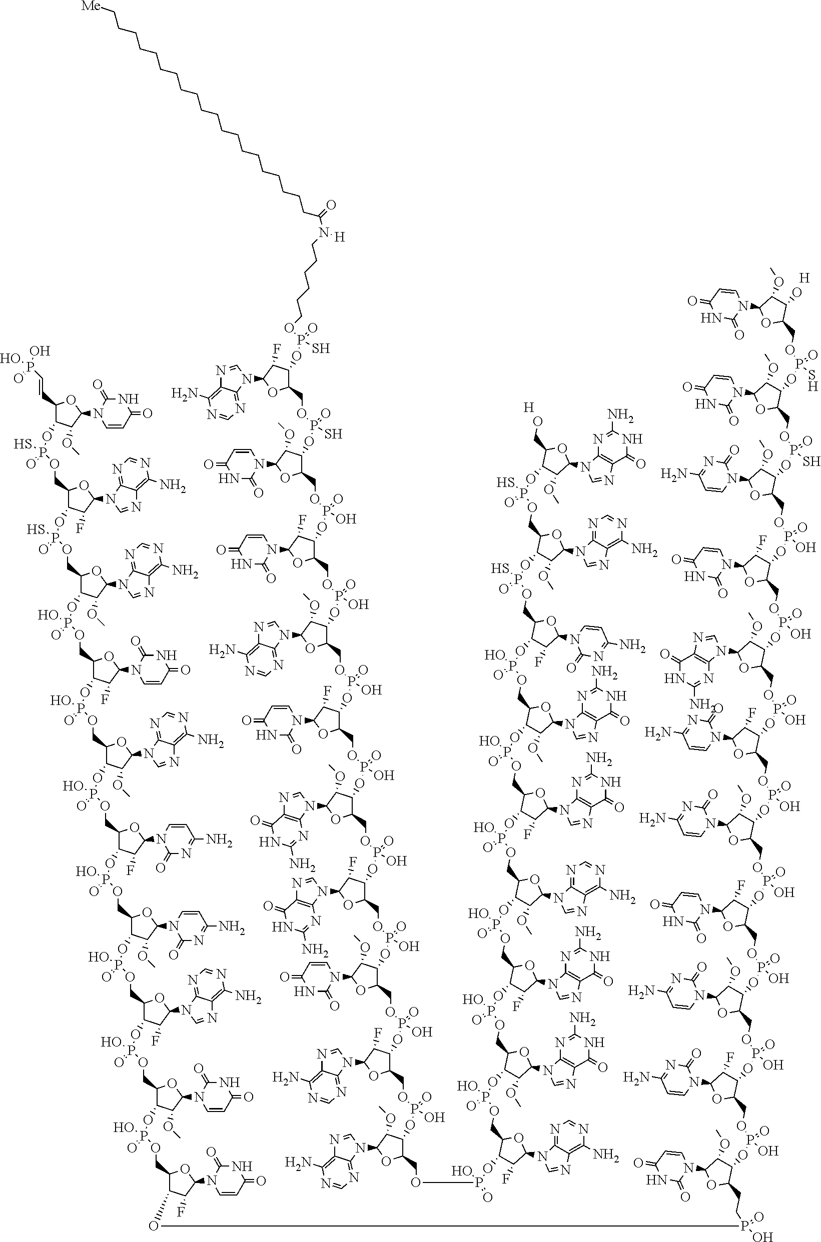

75. A compound comprising or consisting of the structure (compound 3255):

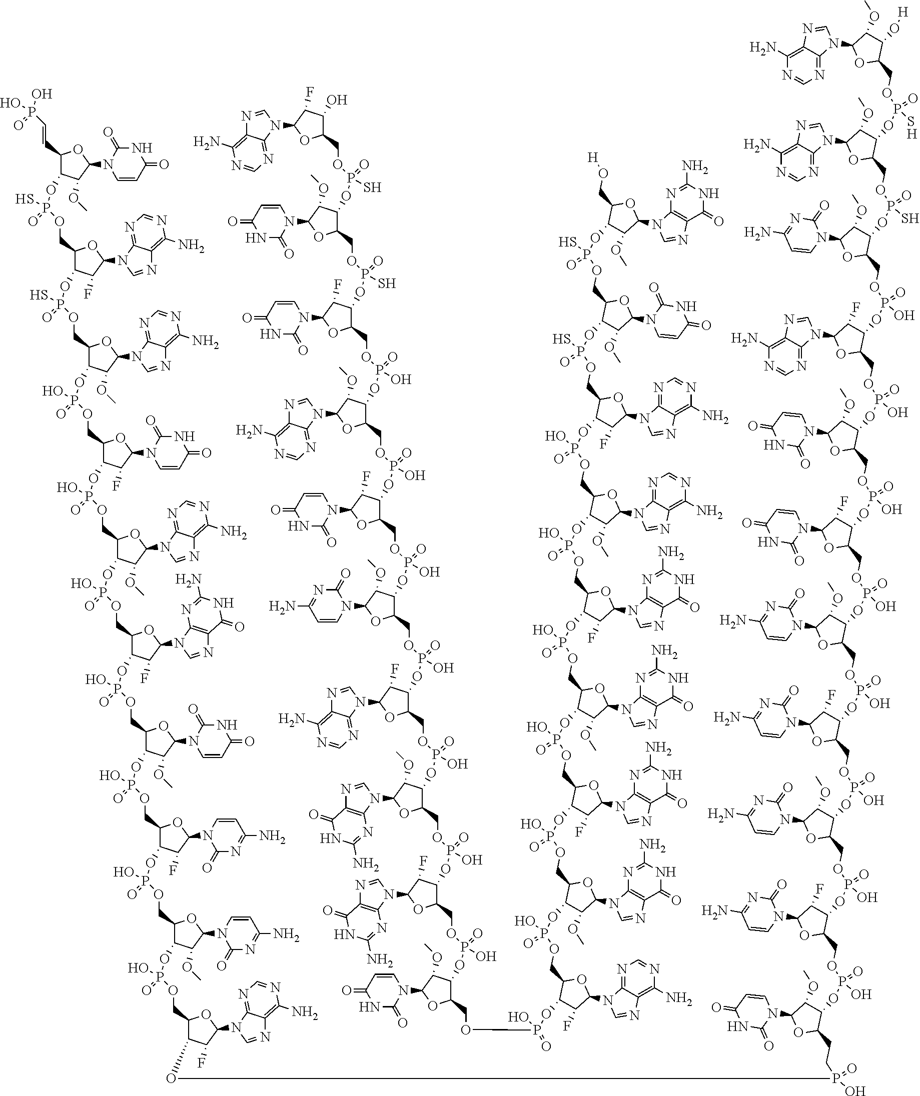

76. A compound comprising or consisting of the structure (compound 3255_C16):

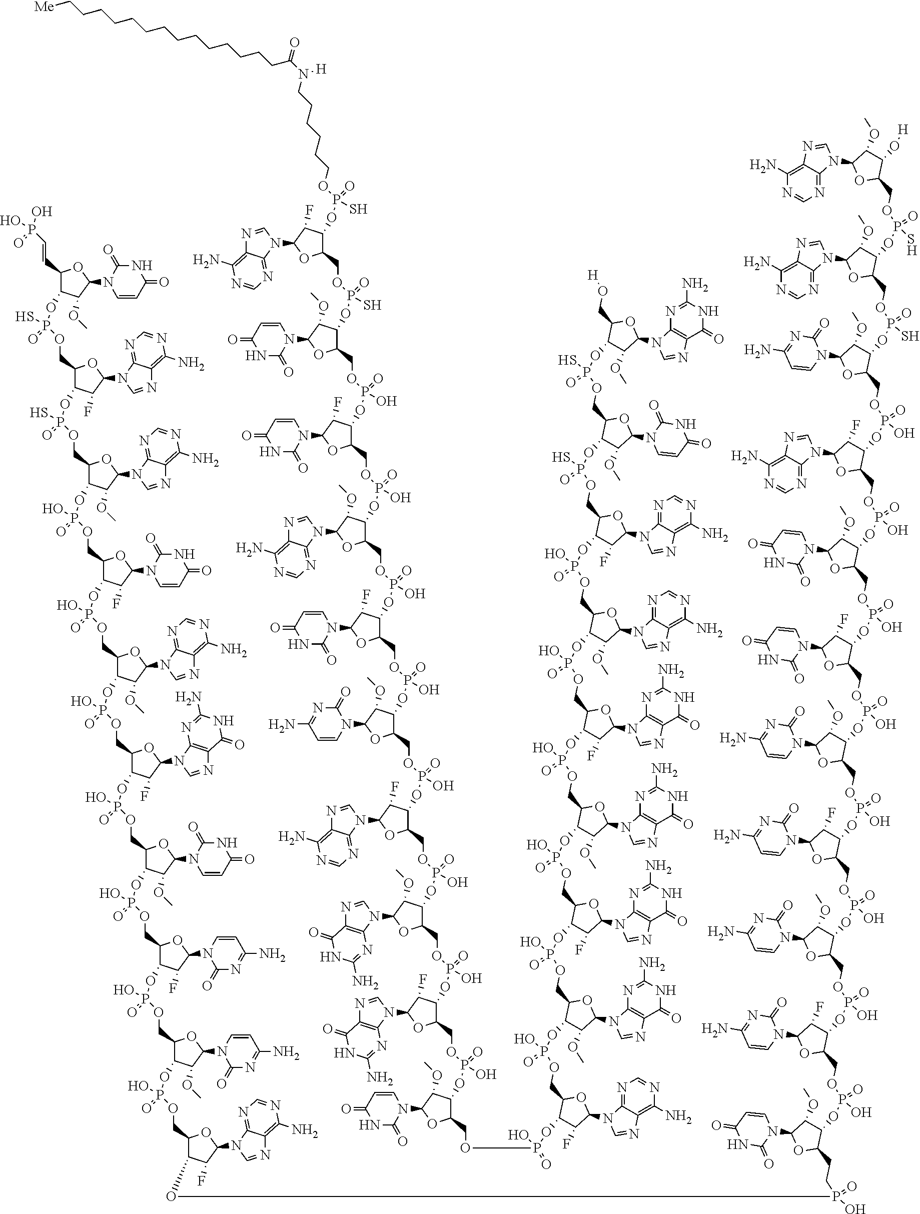

77. A compound comprising or consisting of the structure (compound 3255_C22):

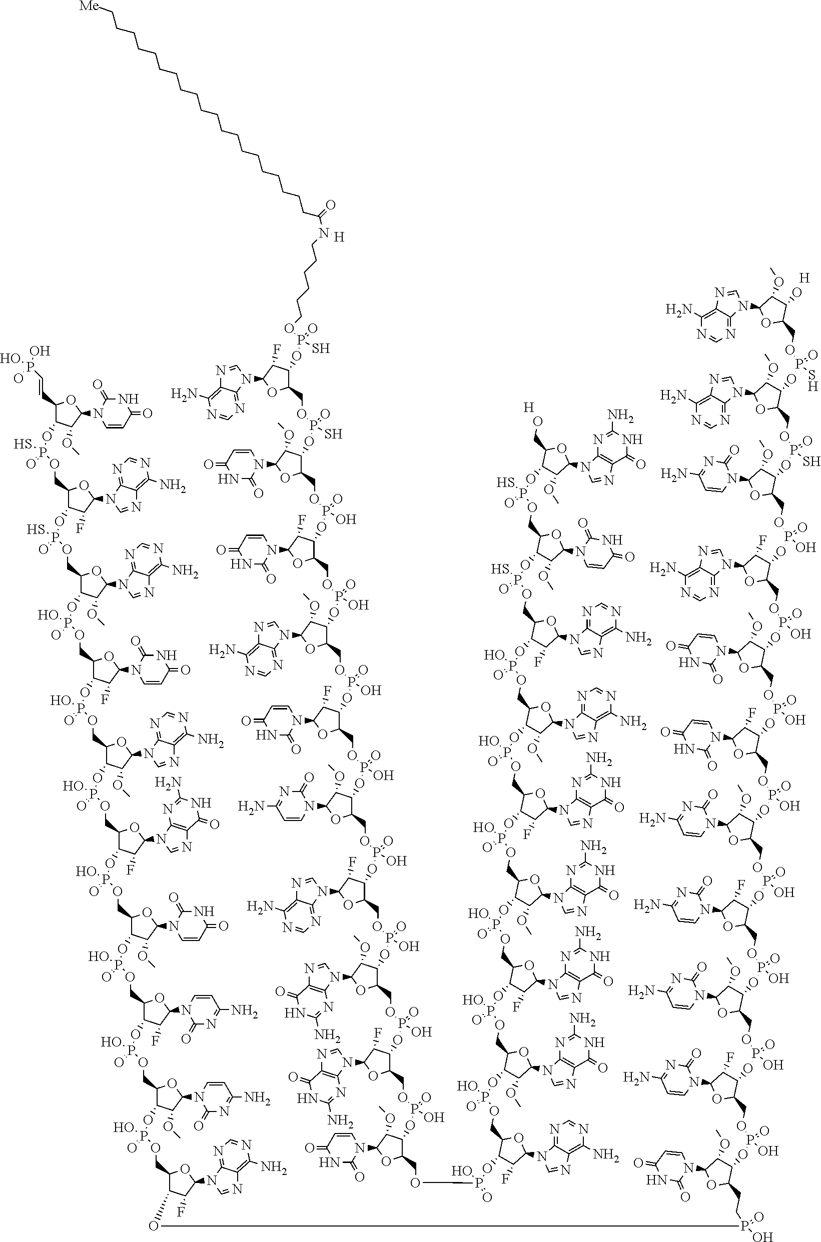

78. A compound comprising or consisting of the structure (compound 3265):

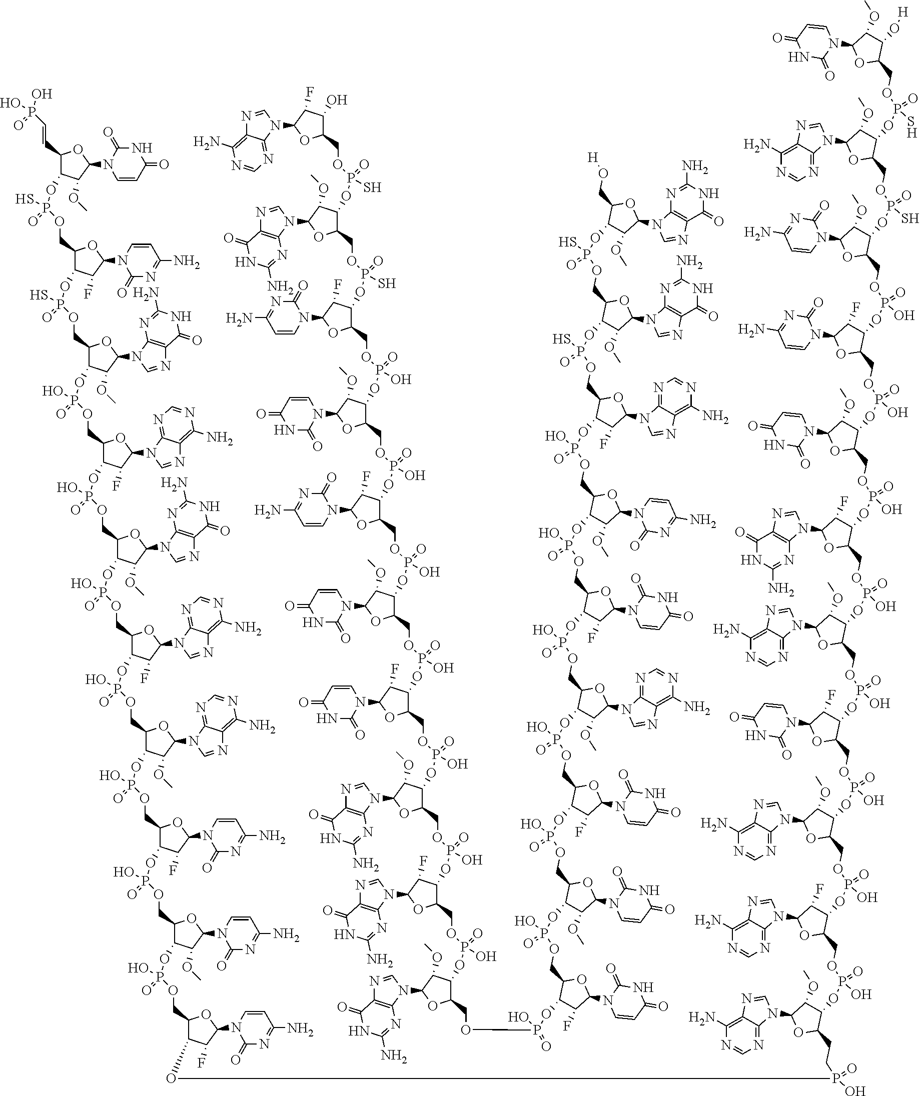

79. A compound comprising or consisting of the structure (compound 3265_C16):

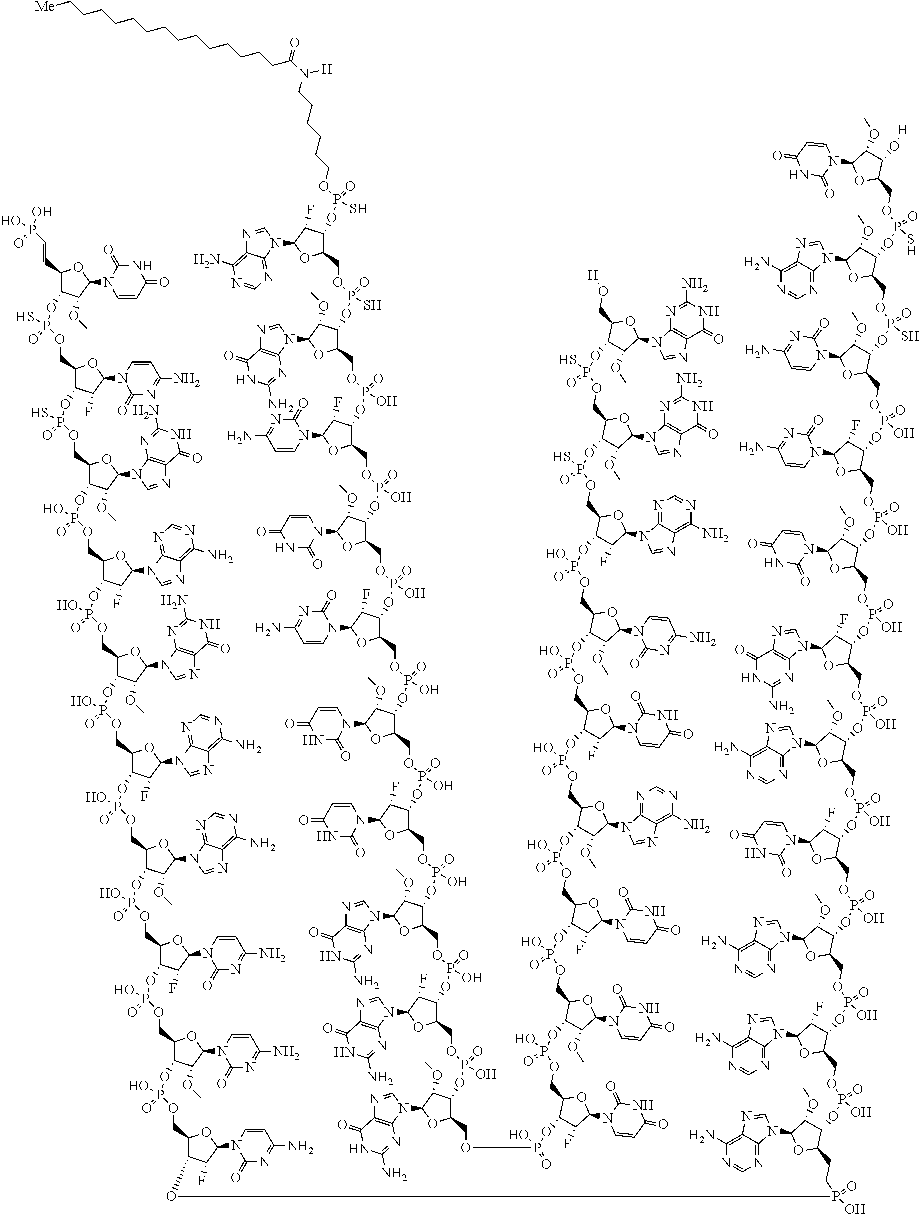

80. A compound comprising or consisting of the structure (compound 3265_C22):

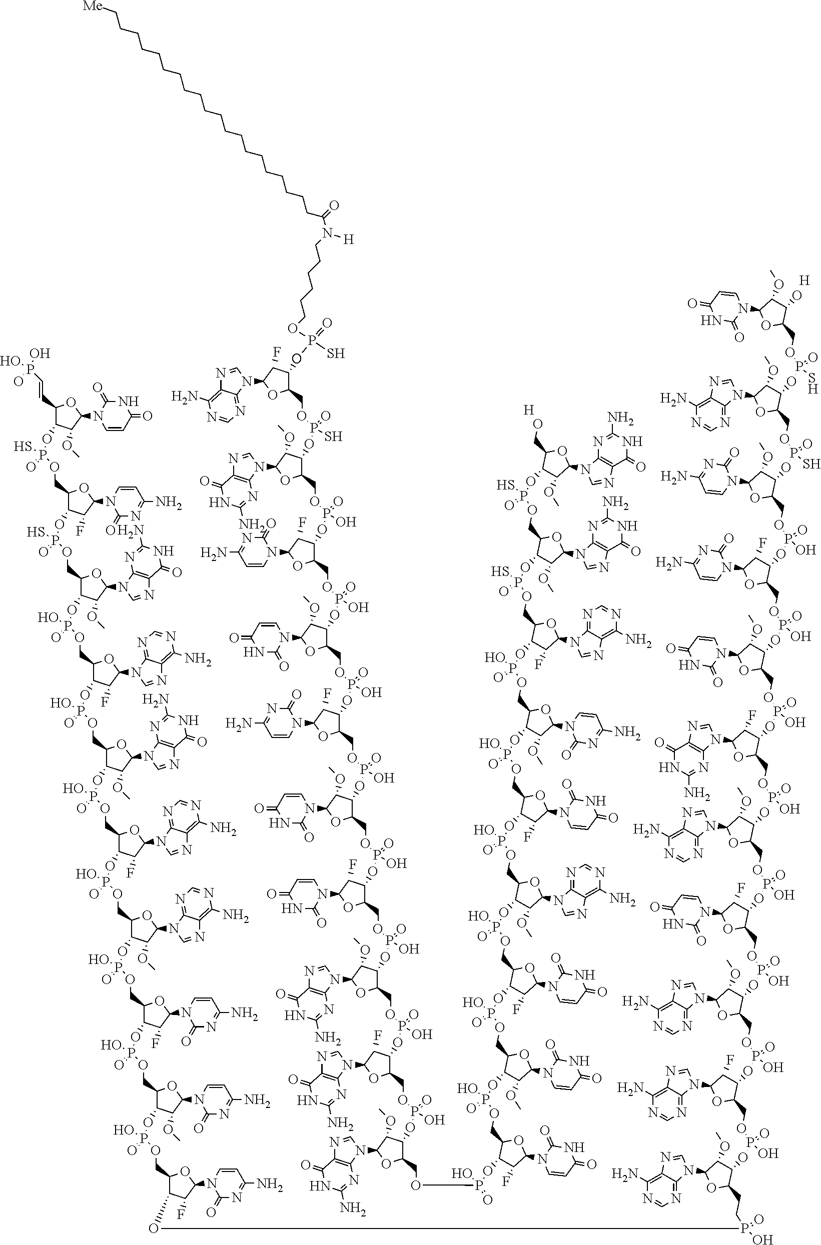

81. A compound comprising or consisting of the structure (compound 3313):

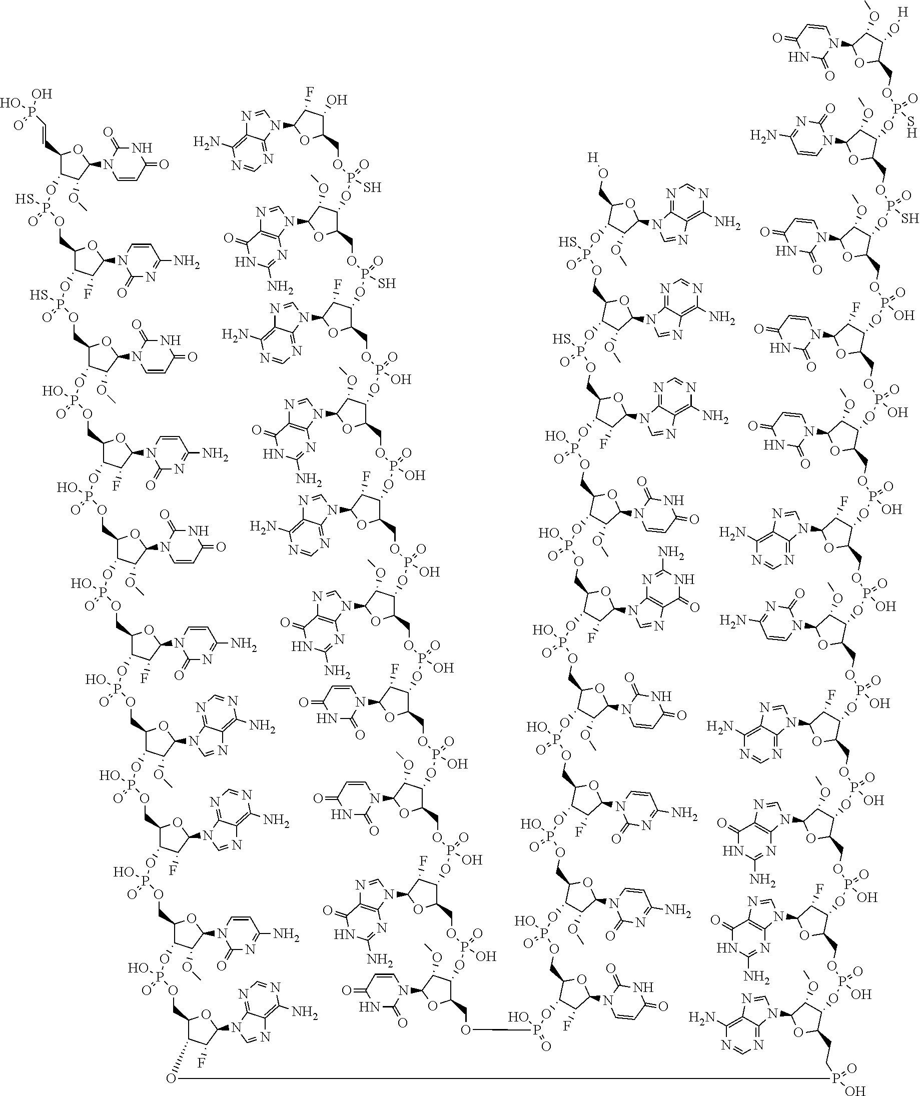

82. A compound comprising or consisting of the structure (compound 3313_C16):

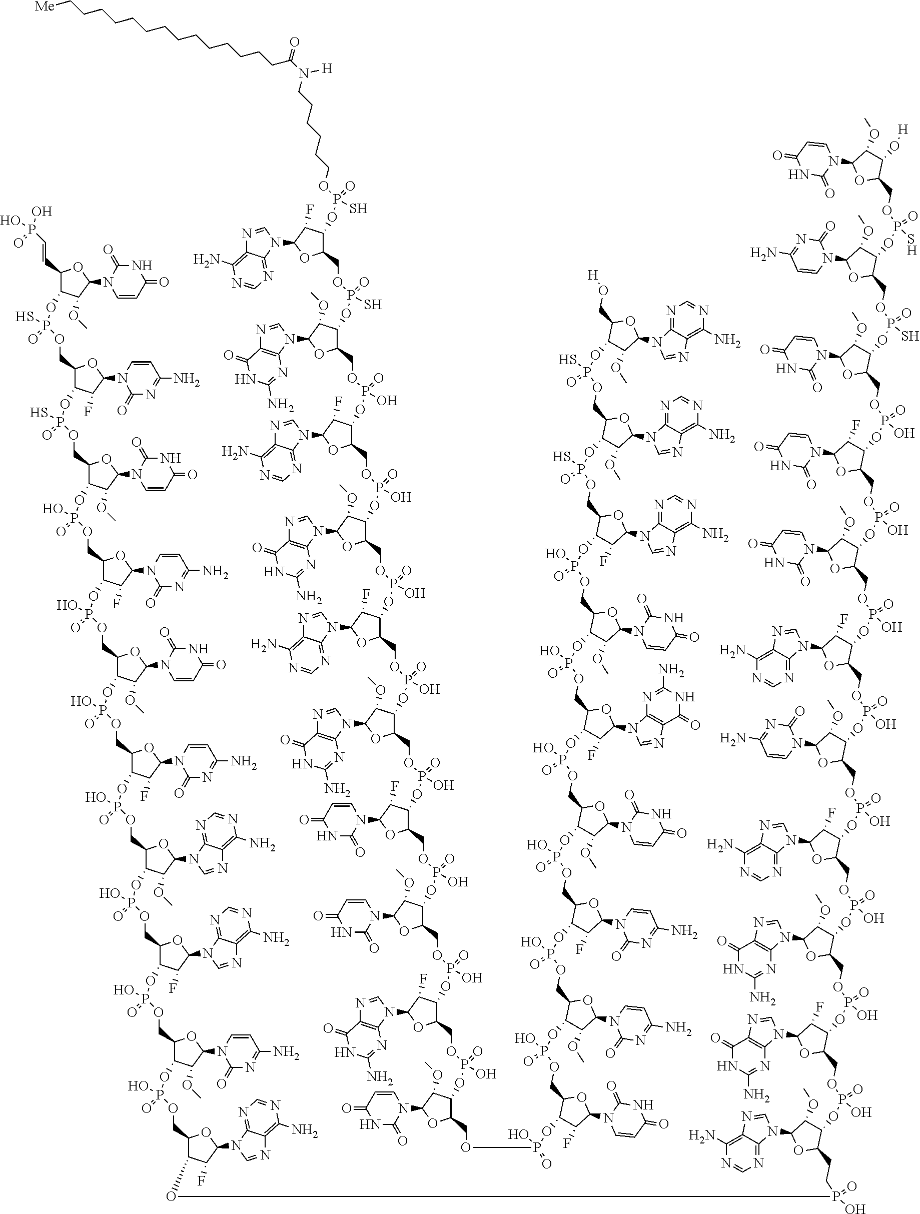

83. A compound comprising or consisting of the structure (compound 3313_C22):

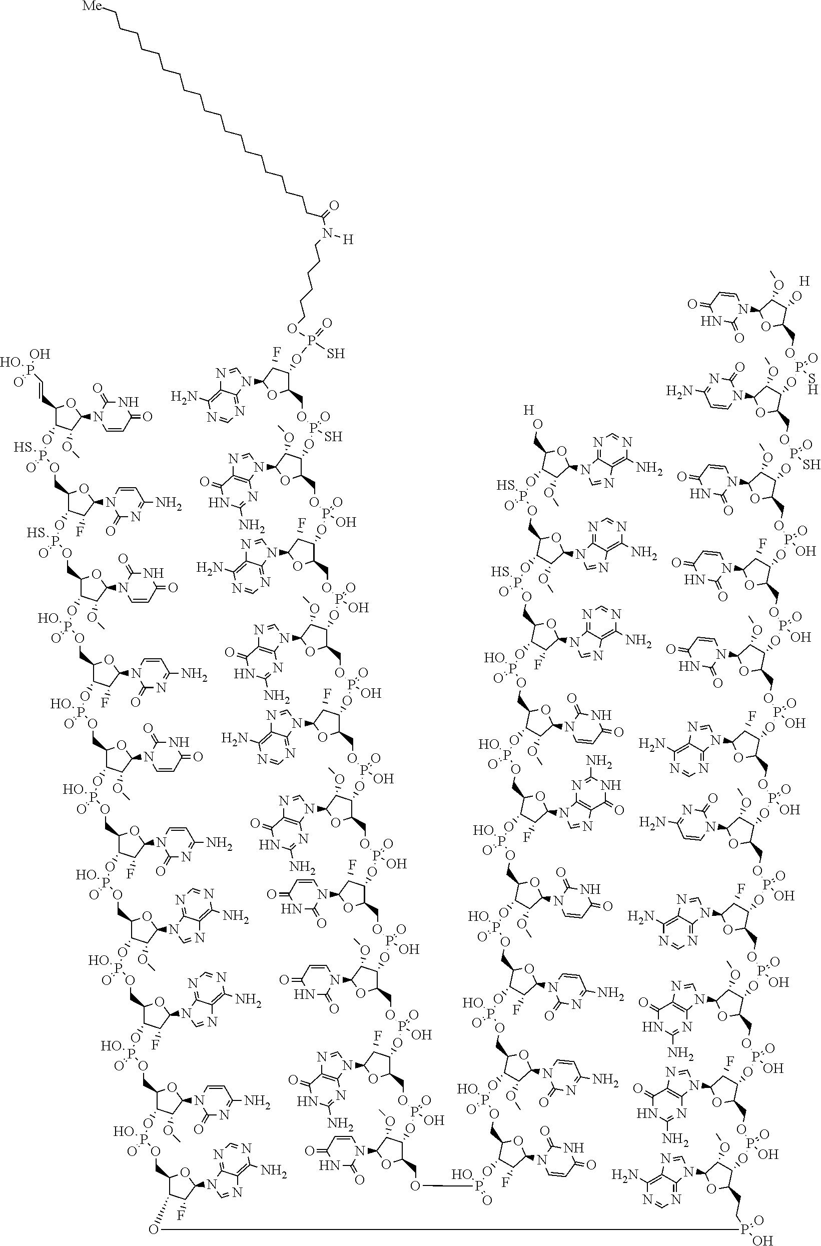

84. The compound of any one of paragraphs 1-83, wherein the compound is in the form of a pharmaceutically acceptable salt, preferably a sodium salt or a potassium salt.
85. The compound of any one of paragraphs 1-84, wherein the compound is encapsulated in a lipid-based delivery vehicle, covalently linked to or encapsulated in a dendrimer, or conjugated to an aptamer.
86. A pharmaceutical composition comprising the compound of any one of paragraphs 1-85, and a pharmaceutically acceptable diluent, solvent, carrier, salt and/or adjuvant; preferably an aqueous diluent or solvent; more preferably phosphate buffered saline.
87. The pharmaceutical composition of paragraph 86, wherein the pharmaceutical composition comprises one or more additional therapeutic agents, preferably a JAK1 inhibitor, more preferably a JAK1 antagonist therapeutic.
88. The pharmaceutical composition of paragraph 87, wherein the additional therapeutic agent is an anti-JAK1 antibody.
89. An in vivo or in vitro method for suppressing JAK1 expression in a target cell, the method comprising administering the compound of any one of paragraphs 1-85 or the pharmaceutical composition of any one of paragraphs 86-88, in an effective amount, to the cell.
90. The method of paragraph 89, wherein the cell is a mammalian cell, preferably a human cell.
91. The method of paragraph 89 or paragraph 90, wherein the expression of JAK1 mRNA is decreased by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95% or about 100% compared to a control.
92. The method of any one of paragraphs 89-91, wherein the expression of JAK1 protein is decreased by at least about 10%, at least about 15%, at least about 20%, at least about 25%, at least about 30%, at least about 35%, at least about 40%, at least about 45%, at least about 50%, at least 55%, at least about 60%, at least about 65%, at least about 70%, at least about 75%, at least about 80%, at least about 85%, at least about 90%, at least about 95% or about 100%, compared to a control.
93. The method of paragraph 91 or paragraph 92, wherein the control is a cell that has not been exposed to the compound.
94. The method of any one of paragraphs 89-93, wherein the method comprises administering one or more additional therapeutic agents, preferably a JAK1 inhibitor, more preferably a JAK1 antagonist therapeutic.
95. The method of paragraph 94, wherein the additional therapeutic agent is an anti-JAK1 antibody.
96. A method for treating or preventing a disease comprising administering a therapeutically or prophylactically effective amount of the compound of any one or paragraphs 1-85 or the pharmaceutical composition of any one of paragraphs 86-88 to a subject suffering from or susceptible to a disease.
97. The compound of any one of paragraphs 1-85 or the pharmaceutical composition of any one of paragraphs 86-88, for use in a method for treating or preventing a disease.
98. Use of the compound of any one of paragraphs 1-85 or the pharmaceutical composition of any one of paragraphs 86-88 for the preparation of a medicament for a method of treatment or prevention of a disease in a subject.
99. The method of paragraph 96, or the compound or pharmaceutical composition for use of paragraph 97, or the use of paragraph 98, wherein the method comprises administering one or more additional therapeutic agents, preferably a JAK1 inhibitor, preferably a JAK1 antagonist therapeutic.
100. The method, or the compound or pharmaceutical composition for use, or the use, of paragraph 99, wherein the additional therapeutic agent is an anti-JAK1 antibody.
101. The method, the compound or pharmaceutical composition for use, or the use of any one of paragraphs 96-100, wherein the disease is associated with increased expression of JAK1.
102. The method, the compound or pharmaceutical composition for use, or the use of any one of paragraphs 96-101, wherein the disease is selected from the group consisting of inflammatory bowel disease, organ transplant rejection, graft-versus-host disease, multiple sclerosis, rheumatoid arthritis (RA), juvenile idiopathic arthritis, psoriasis, dermatitis, diabetic nephropathy, systemic lupus erythematosus (SLE), dry eye disease, cancer, myelofibrosis, and asthma, preferably dry eye disease.
103. A kit comprising the compound of any one of paragraphs 1-85 and instructions for use.
104. The kit of paragraph 103, wherein the kit further comprises one or more additional therapeutic agents, preferably a JAK1 inhibitor, more preferably a JAK1 antagonist therapeutic.
105. The kit of paragraph 104, wherein the additional therapeutic agent is an anti-JAK1 antibody.

### EXAMPLES

### Example 1—Single Dose Screen of JAK1 siRNA in Cells

The ability of 191 JAK1 siRNAs (see Table 3) to reduce JAK1 mRNA in U-87 MG cells was tested.

Cells were supplied by the following source: U-87 MG (also known as HTB-14) from ATCC (American Type Culture Collections, Lot #: 999002999, passage 11).

For transfection of cells of with hsJAK1 targeting siRNAs (and PBS as control), cells were seeded at a density of 20,000 cells/well in regular 96-well collagene-coated plates. Transfection of cells with siRNAs was carried out using the commercially available transfection reagent LF2000 (Thermo, Lot #: 2357799) according to the manufacturer's instructions. The experiment for the entire set of siRNAs was done in U-87 MG cells at a final siRNA concentration of 2 nM.

Solutions with siRNA were made using PBS to 5000 nM. Furthermore, the solutions were diluted to 50 nM using a series of 10-fold dilutions with PBS to be used in transfection mix. 50 μL/well of Transfection mix was made using siRNA solution, Opti-MEM and LF2000 using the following mixing ratio:

25 μL/well of siRNA mix (siRNA solution+Opti-MEM):

siRNA mix was made from mixing 6 parts of siRNA stock solution and 19 parts of Opti-MEM.

50 nM siRNA stock solution was used to test siRNA in at 2 nM in the final well.

25 μL/well of LF2000 mix (LF2000+Opti-MEM):

LF2000 mix was made from mixing 2 parts of LF2000 with 98 parts of Opti-MEM.

siRNA mix and LF2000 mix were prepared separately and incubated at room temperature for 5 min.

siRNA mix and LF2000 mix were mixed 1:1 to give Transfection mix and incubated for 15 min at room temperature.

50 μL of Transfection mix was combined with 100 μL of cell suspension in 96 well plate, and incubated for 24 hours at 37° C./5% CO2 in humidified incubator.

For each siRNA and PBS as control, at least four wells were transfected in parallel, and individual data points were collected from each well. After 24 h of incubation with siRNA post-transfection the cells were lysed and relative mRNA expression of target and control genes was quantified using bDNA assay. The branched DNA (bDNA) assay was performed according to manufacturer's instructions (QuantiGene RNA Assays for Gene Expression Profiling, ThermoFischer Scientific).

For each well, the on-target mRNA levels were normalized to the hsGAPDH mRNA levels. The activity of any siRNA was expressed as percent hsJAK1 on-target mRNA concentration (normalized to hsGAPDH mRNA) in treated cells, relative to the mean hsJAK1 on-target mRNA concentration (normalized to hsGAPDH mRNA) across control wells. For analyzing the data, the mean ratio of hsJAK1/hsGAPDH with all negative control (PBS) treatments was artificially set to 100% and used for data normalization.

The results are shown in Table 5 below.

The results were used to select siRNAs for the next step: the top performing 47 siRNA showing best knockdown (less remaining JAK1 mRNA in %) of the entire set was selected for IC50 determination in Example 2.

All compounds have the following design (“parent design”):

Modification pattern of sense strand from 5′ to 3′:
5′-[2′OMe]-PS-[2′OMe]-PS-[2′F]-[2′OMe]-[2′F]-[2′OMe]-[2′F]-[2′OMe]-[2′F]-[2′OMe]-[2′F]-[2′OMe]-[2′F]-[2′OMe]-[2′F]-[2′OMe]-[2′F]-PS-[2′OMe]-PS-[2′F]-3′

Modification pattern of antisense strand from 3′ to 5′:
3′-[2′OMe]-PS-[2′OMe]-PS-[2′OMe]-[2′F]-[2′OMe]-[2′F]-[2′OMe]-[2′F]-[2′OMe]-[2′F]-[2′OMe]-[2′F]-[2′OMe]-[2′F]-[2′OMe]-[2′F]-[2′OMe]-[2′F]-[2′OMe]-PS-[2′F]-PS-[VP-2′-OMe]-5′

with [2′OMe]representing a 2′-OMe RNA,

[2′F] representing a 2′-F RNA,

[VP-2′-OMe] representing a Vinyl-phosphonate 2′-OMe RNA, and

PS representing phosphorothioate internucleotide linkages (in the absence of mention, the internucleotide linkages is a phosphodiester internucleotide linkage).

TABLE 5
Results from single dose screen of JAK1 siRNA in U-251 MG cells

| | 2 nM siRNA | |
|---|---|---|
| Compound | relative remaining | |
| # | JAK1 mRNA in % | s.d. |
| 614 | 10.07 | 0.27 |
| 673 | 11.34 | 0.83 |
| 724 | 90.62 | 4.30 |
| 728 | 33.51 | 2.87 |
| 753 | 33.69 | 1.49 |
| 756 | 14.83 | 1.06 |
| 818 | 48.22 | 1.28 |
| 874 | 92.12 | 1.11 |
| 875 | 95.26 | 2.41 |
| 876 | 38.62 | 1.73 |
| 877 | 103.61 | 3.67 |
| 878 | 72.24 | 0.81 |
| 883 | 17.21 | 1.26 |
| 884 | 57.88 | 2.77 |
| 1069 | 82.22 | 4.38 |
| 1075 | 73.00 | 1.26 |
| 1085 | 53.17 | 1.00 |
| 1107 | 11.85 | 0.27 |
| 1108 | 10.99 | 0.56 |
| 1138 | 10.66 | 0.37 |
| 1182 | 9.68 | 1.10 |
| 1189 | 8.97 | 0.52 |
| 1190 | 9.89 | 0.40 |
| 1304 | 12.00 | 0.85 |
| 1306 | 19.02 | 3.08 |
| 1311 | 21.76 | 0.55 |
| 1367 | 58.17 | 7.47 |
| 1368 | 27.79 | 1.98 |
| 1372 | 56.61 | 3.91 |
| 1412 | 12.42 | 2.18 |
| 1413 | 21.02 | 1.07 |
| 1432 | 41.34 | 3.11 |
| 1579 | 80.48 | 11.59 |
| 1580 | 88.07 | 9.15 |
| 1581 | 11.28 | 0.72 |
| 1583 | 85.58 | 4.89 |
| 1584 | 70.66 | 5.29 |
| 1586 | 99.92 | 8.21 |
| 1587 | 51.68 | 3.72 |
| 1588 | 41.40 | 3.63 |
| 1595 | 99.32 | 2.59 |
| 1596 | 49.38 | 1.67 |
| 1601 | 34.90 | 2.20 |
| 1602 | 27.45 | 3.14 |
| 1603 | 76.46 | 3.44 |
| 1608 | 89.62 | 4.38 |
| 1609 | 10.72 | 0.71 |
| 1611 | 81.69 | 6.35 |
| 1640 | 46.28 | 2.66 |
| 1642 | 36.37 | 2.00 |
| 1671 | 14.03 | 0.42 |
| 1672 | 63.81 | 2.35 |
| 1673 | 93.02 | 5.15 |
| 1674 | 23.83 | 7.12 |
| 1677 | 16.97 | 0.56 |
| 1678 | 17.19 | 2.52 |
| 1690 | 96.83 | 5.52 |
| 1692 | 13.03 | 1.03 |
| 1698 | 11.50 | 2.09 |
| 1699 | 23.08 | 1.21 |
| 1723 | 81.74 | 2.43 |
| 1769 | 26.87 | 1.48 |
| 1770 | 8.29 | 0.87 |
| 1780 | 25.38 | 1.31 |
| 1798 | 48.37 | 3.08 |
| 1876 | 21.73 | 9.73 |
| 1927 | 45.00 | 4.11 |
| 1928 | 81.50 | 4.78 |
| 1929 | 67.82 | 2.73 |
| 1936 | 13.23 | 1.19 |
| 1952 | 89.00 | 4.38 |
| 1954 | 10.42 | 1.58 |
| 1956 | 76.18 | 2.52 |
| 1958 | 27.20 | 1.65 |
| 1978 | 91.17 | 7.46 |
| 2066 | 17.74 | 10.01 |
| 2068 | 61.65 | 10.27 |
| 2102 | 72.37 | 1.28 |
| 2111 | 58.10 | 4.81 |
| 2138 | 23.98 | 2.34 |
| 2146 | 13.70 | 1.05 |
| 2148 | 19.93 | 0.93 |
| 2205 | 13.65 | 3.16 |
| 2206 | 84.55 | 3.63 |
| 2218 | 15.51 | 1.10 |
| 2229 | 37.81 | 2.85 |
| 2230 | 18.21 | 1.51 |
| 2237 | 74.31 | 7.92 |
| 2238 | 27.08 | 3.55 |
| 2239 | 88.42 | 8.60 |
| 2269 | 13.60 | 1.51 |
| 2308 | 91.99 | 8.11 |
| 2317 | 89.92 | 4.92 |
| 2318 | 87.73 | 7.56 |
| 2319 | 13.89 | 1.21 |
| 2320 | 95.53 | 4.34 |
| 2321 | 101.79 | 4.03 |
| 2322 | 69.11 | 2.32 |
| 2323 | 75.49 | 2.47 |
| 2520 | 24.73 | 3.69 |
| 2527 | 14.16 | 0.41 |
| 2647 | 84.43 | 1.81 |
| 2761 | 82.57 | 2.40 |
| 2762 | 15.23 | 4.46 |
| 2763 | 69.99 | 8.59 |
| 2764 | 68.60 | 2.77 |
| 2811 | 10.78 | 0.60 |
| 2962 | 81.74 | 4.58 |
| 2975 | 86.94 | 4.88 |
| 2977 | 87.23 | 4.19 |
| 3028 | 42.03 | 2.36 |
| 3032 | 20.03 | 1.64 |
| 3081 | 14.74 | 2.02 |
| 3131 | 8.61 | 0.94 |
| 3134 | 11.92 | 0.99 |
| 3141 | 11.91 | 3.58 |
| 3144 | 18.52 | 0.49 |
| 3146 | 28.73 | 1.86 |
| 3147 | 23.00 | 6.75 |
| 3159 | 37.56 | 0.94 |
| 3160 | 12.28 | 0.52 |
| 3229 | 17.96 | 1.03 |
| 3247 | 15.97 | 0.89 |
| 3250 | 37.09 | 2.72 |
| 3251 | 71.24 | 6.83 |
| 3252 | 60.86 | 3.34 |
| 3254 | 73.99 | 3.47 |
| 3255 | 14.00 | 0.11 |
| 3258 | 17.73 | 0.98 |
| 3259 | 11.21 | 1.32 |
| 3260 | 24.20 | 1.38 |
| 3261 | 28.26 | 2.07 |
| 3265 | 11.93 | 0.40 |
| 3268 | 14.17 | 0.93 |
| 3272 | 14.52 | 0.37 |
| 3275 | 21.69 | 3.69 |
| 3276 | 86.52 | 3.02 |
| 3278 | 14.40 | 1.30 |
| 3279 | 76.09 | 3.56 |
| 3281 | 89.83 | 3.30 |
| 3282 | 71.03 | 2.54 |
| 3283 | 52.87 | 1.33 |
| 3284 | 92.03 | 6.06 |
| 3285 | 93.43 | 5.11 |
| 3286 | 70.49 | 8.43 |
| 3313 | 18.41 | 0.64 |
| 3314 | 91.14 | 5.65 |
| 3323 | 18.86 | 0.79 |
| 3353 | 19.50 | 1.46 |
| 3365 | 18.47 | 2.41 |
| 3367 | 19.80 | 1.06 |
| 3368 | 13.64 | 1.01 |
| 3371 | 17.95 | 1.26 |
| 3372 | 22.45 | 0.64 |
| 3376 | 24.69 | 1.20 |
| 3409 | 76.13 | 3.61 |
| 3505 | 13.41 | 0.92 |
| 3556 | 95.32 | 4.96 |
| 3557 | 88.65 | 6.23 |
| 3558 | 22.45 | 1.21 |
| 3559 | 51.46 | 1.41 |
| 3654 | 11.52 | 0.98 |
| 3662 | 13.44 | 1.21 |
| 3663 | 12.86 | 1.20 |
| 3683 | 49.61 | 3.06 |
| 3689 | 55.38 | 4.29 |
| 3694 | 91.70 | 10.40 |
| 3695 | 43.35 | 10.12 |
| 3698 | 77.00 | 3.40 |
| 3702 | 28.57 | 2.30 |
| 3719 | 94.12 | 2.37 |
| 3781 | 19.70 | 3.76 |
| 3894 | 63.11 | 2.74 |
| 4099 | 18.33 | 1.51 |
| 4169 | 17.95 | 1.16 |
| 4239 | 21.39 | 6.66 |
| 4305 | 41.80 | 2.24 |
| 4374 | 90.56 | 1.57 |
| 4411 | 20.91 | 1.51 |
| 4475 | 24.88 | 0.93 |
| 4612 | 61.55 | 4.81 |
| 4671 | 49.37 | 1.67 |
| 4672 | 57.16 | 15.90 |
| 4679 | 37.36 | 8.94 |
| 4682 | 96.45 | 6.51 |
| 4683 | 75.49 | 2.99 |
| 4684 | 39.57 | 2.87 |
| 4690 | 99.23 | 11.17 |
| 4794 | 20.28 | 0.78 |
| 4803 | 61.80 | 1.80 |
| 4807 | 16.55 | 0.52 |

### Example 2—IC50 Values and Maximum Efficacy of Selected JAK1 siRNAs in Three Cell Lines

Amongst the 191 compounds tested in Example 1, 47 compounds were selected for further analysis due to their efficacious in vitro reduction of JAK1 mRNA and their favourable cross reactivity across e.g. human, cynomolgus, mouse and rabbit.

47 compounds were tested at 10 different concentrations from 24 nM and then four-fold dilutions. Based on the concentration response curves, the concentration reducing the remaining JAK1 mRNA to 50% (IC50) was determined in three cell lines:
human U-87 MG using hsJAK1 as gene of interest and hsGAPDH as normalization gene,
mouse mmJAK1 as gene of interest and mmGAPDH as normalization gene, and
rabbit SIRC using ocJAK1 as gene of interest and ocGAPDH as normalization gene.

Human U-87 MG:

Cells were supplied by the following source: U-87 MG (also known as HTB-14) from ATCC (American Type Culture Collections, Lot #: 999002999, passage 11).

Mouse Hepa1-6:

Cells were supplied by the following source: Hepa1-6 from ATCC (American Type Culture Collections, Lot #: 63048648, passage 14).

Rabbit SIRC:

Cells were supplied by the following source: SIRC from ATCC (American Type Culture Collections, Lot #: 70014309, passage 12).

siRNA was tested at 10 concentrations in each cell line. The highest concentration was 24 nM, going down in 9× four-fold dilution between each concentration. siRNA was diluted to 5 μM stock using PBS.

25 μL/well siRNA mix (siRNA stock+Opti-MEM):

The highest siRNA mix solutions were prepared using 5 M siRNA stock diluted with Opti-MEM in order to produce 24 nM. The following 9 concentrations with four-fold dilution between each concentration were made using 1 part of the previous concentration mixed together with 3 parts of Opti-MEM.

25 μL/well of LF2000 mix (LF2000+Opti-MEM):

LF2000 mix was made from mixing 2 parts of LF2000 with 98 parts of Opti-MEM.

siRNA mix and LF2000 mix were mixed and tested. The experimental setup and bDNA analysis of mRNAs of interest were as described in Example 1. All data were generated in quadruplicates for human and rabbit cell lines, and in duplicates for mouse cell line.

For each siRNA tested at 10 different concentrations, based on the concentration response curves (Excel add-in XLfit software tool), the concentration reducing the remaining JAK1 mRNA to 50% (IC50) and the maximum efficacy (Max inhibition) (%) was determined. The results are shown in Table 6 below.

TABLE 6
IC50 values and maximum efficacy of selected JAK1 siRNAs

| | Rabbit | | Mouse | | Human | |
|---|---|---|---|---|---|---|
| | | Max. | | Max. | | Max. |
| Compound # | | inhi- | | inhi- | | inhi- |
| (Start pos. in | IC50 | bition | IC50 | bition | IC50 | bition |
| NM_002227.4) | (nM) | (%) | (nM) | (%) | (nM) | (%) |
| 614 | 0.0011 | 91.2 | 8.1670 | 56.2 | 0.0007 | 91.0 |
| 673 | 0.0084 | 91.9 | 0.0270 | 85.4 | 0.0011 | 90.7 |
| 756 | 0.2070 | 86.7 | 0.8670 | 82.4 | 0.0062 | 88.3 |
| 883 | 0.0157 | 90.0 | 0.1680 | 82.8 | 0.0030 | 90.0 |
| 1107 | 0.0232 | 88.2 | 0.0330 | 88.8 | 0.0019 | 91.4 |
| 1108 | 1.9695 | 57.3 | 0.0090 | 92.4 | 0.0009 | 91.5 |
| 1182 | 0.0029 | 93.5 | | 0.7 | 0.0009 | 93.3 |
| 1304 | 0.0783 | 74.5 | 0.0270 | 87.2 | 0.0032 | 90.5 |
| 1306 | 0.0363 | 75.5 | 0.0700 | 68.3 | 0.0040 | 81.9 |
| 1412 | 0.0089 | 89.5 | 0.2330 | 82.1 | 0.0037 | 89.2 |
| 1609 | 0.0018 | 90.5 | 0.0410 | 87.0 | 0.0013 | 91.1 |
| 1671 | 0.0283 | 91.2 | 0.1160 | 87.8 | 0.0058 | 89.6 |
| 1677 | 0.0167 | 88.2 | 0.0540 | 86.7 | 0.0044 | 86.6 |
| 1678 | 0.0288 | 90.2 | 0.2670 | 80.8 | 0.0075 | 87.8 |
| 1692 | 0.0183 | 89.8 | 0.0810 | 87.3 | 0.0032 | 90.2 |
| 1698 | 0.0110 | 88.3 | 0.0320 | 84.6 | 0.0038 | 90.9 |
| 1770 | 0.0041 | 94.6 | 0.0170 | 90.8 | 0.0006 | 91.8 |
| 1936 | | 44.0 | 0.0610 | 86.7 | 0.0050 | 87.6 |
| 1954 | 0.0051 | 94.2 | | 5.8 | 0.0017 | 91.3 |
| 2146 | 0.0114 | 89.8 | 0.4980 | 73.0 | 0.0017 | 90.0 |
| 2205 | 0.0109 | 87.5 | 0.1740 | 81.0 | 0.0020 | 88.6 |
| 2218 | 0.0249 | 89.9 | 0.1370 | 84.0 | 0.0042 | 87.6 |
| 2269 | 0.0065 | 89.5 | 18.0110 | 66.9 | 0.0012 | 90.3 |
| 2319 | 0.0061 | 87.0 | 0.0180 | 86.8 | 0.0008 | 91.1 |
| 2762 | 0.0097 | 89.4 | 0.0470 | 86.0 | 0.0020 | 89.8 |
| 2811 | 0.0202 | 89.3 | 0.0570 | 83.8 | 0.0010 | 91.1 |
| 3081 | 0.1353 | 79.2 | | 39.3 | 0.0058 | 88.5 |
| 3131 | 0.0088 | 90.7 | 0.2400 | 85.0 | 0.0009 | 91.0 |
| 3134 | 0.0058 | 90.7 | | 6.0 | 0.0026 | 89.4 |
| 3141 | 0.0092 | 91.6 | | 47.6 | 0.0024 | 90.5 |
| 3144 | 0.0067 | 92.0 | 0.1140 | 82.6 | 0.0041 | 87.9 |
| 3160 | 0.0092 | 92.0 | 0.3030 | 84.8 | 0.0041 | 90.6 |
| 3247 | 0.0250 | 89.3 | 1.3260 | 75.5 | 0.0063 | 87.2 |
| 3255 | 0.0039 | 93.5 | 2.3880 | 60.3 | 0.0017 | 89.1 |
| 3258 | 0.0073 | 91.1 | | 44.3 | 0.0032 | 85.5 |
| 3259 | 0.0086 | 91.6 | 0.1310 | 85.6 | 0.0028 | 89.3 |
| 3265 | 0.0052 | 90.6 | 0.0340 | 88.1 | 0.0014 | 89.7 |
| 3313 | 0.0054 | 91.1 | 0.0240 | 86.4 | 0.0018 | 87.5 |
| 3323 | 0.0170 | 90.8 | 0.3000 | 85.2 | 0.0072 | 86.3 |
| 3365 | | −0.8 | 0.0560 | 85.6 | 0.0083 | 84.5 |
| 3367 | | −0.7 | 0.0710 | 85.8 | 0.0066 | 84.0 |
| 3368 | | 5.7 | 0.0269 | 86.0 | 0.0017 | 88.8 |
| 3371 | | 0.5 | 0.0240 | 87.4 | 0.0049 | 85.9 |
| 3654 | 0.0170 | 89.2 | 0.2630 | 83.7 | 0.0016 | 91.3 |
| 3662 | 0.0190 | 90.5 | | 39.8 | 0.0105 | 86.6 |
| 3663 | 0.0158 | 90.8 | | 26.1 | 0.0067 | 87.4 |
| 4807 | | 3.5 | 0.1840 | 70.7 | 0.0048 | 88.2 |

### Example 3—JAK1 mRNA Expression after Transfection of HCEC Cells with the Selected siRNAs Targeting JAK1 (Compound 614, 673, 1182, 1770, 1954, 2319, 3131, 3255, 3265, and 3313)

From Example 2 above, 10 compounds were selected (compounds 614, 673, 1182, 1770, 1954, 2319, 3131, 3255, 3265, and 3313) based on best potencies (IC50) in human HCEC and Rabbit SIRC1.

Primary Human Cornea Epithelial cells HCEC (PCS-700-010, ATCC) were plated in 96 well plates with 15000 cells per well in full growth medium (Corneal Epithelial Cell Basal Medium ATCC PCS-700-030 and Corneal Epithelial Cell Growth Kit ATCC PCS-700-040). The plated cells were transfected the day after with naked siRNA molecules corresponding to Compound #614, 673, 1182, 1770, 1954, 2319, 3131, 3255, 3265, and 3313, in PBS for final concentrations of 1 nM and three-fold dilutions for 7 doses using RNAiMax (ThermoFisher) according to manufacturer's protocol (ThermoFisher). After 1 day, cells were lysed using 350 μL Lysis buffer using the MagNA Pure 96 system according to manufacturer's instructions (Roche LifeScience) and extracted in 50 μL RNAse free water. One-step qPCR was done using qScript® One-Step qRT-PCR Kit, Low ROX™ according to manufacturer's protocol (QuantaBio).

The following TaqMan gene expression assays were used

JAK1 (FAM): Hs01026985_m1 (Catalog number: 4351372, TaqMan Thermofisher Scientific) and GAPDH (VIC): Hs02786624_g1 (Catalog number: 4448489, TaqMan Thermofisher Scientific).

JAK1 mRNA concentrations were quantified relative to the housekeeping gene GAPDH using QuantStudio™ Real-time PCR system Software (Applied Biosystem) and normalized to only PBS treated HCEC cells (PBS set to 100%)

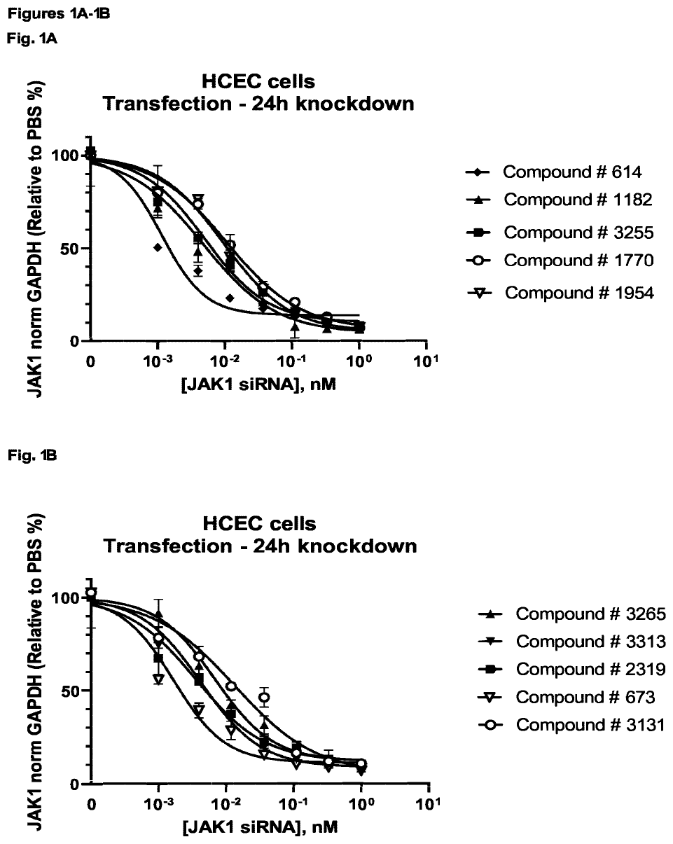

All the 10 siRNA tested, targeting JAK1, were very potent and efficacious with potencies less than 25 pM as shown in FIG. 1a (compounds 614, 1182, 3255, 1770 and 1954) and FIG. 1b (compounds 3265, 3313 2319, 673, 3131).

### Example 4—JAK1 mRNA Expression after Transfection of HCEC Cells Gymnosis for 5 Days of SIRC1 Cells with the C22 Conjugated siRNAs Targeting JAK1 (Compound 614_C22, 673_C22, 1182_C22, 1770_C22, 1954_C22, 2319_C22, 3131_C22, 3255_C22, 3265_C22, and 3313_C22)

Rabbit SIRC1 cells (Statens Seruminstitut Rabbit Cornea, ATCC, CCL-60) cells were plated in 96 well plates 5000 cells per well in full growth medium and treated with C22-conjugated siRNA molecules (C22-conjugated compound 614_C22, 673_C22, 1182_C22, 1770_C22, 1954_C22, 2319_C22, 3131_C22, 3255_C22, 3265_C22, and 3313_C22) in PBS for final concentrations of 30 UM and three-fold dilutions for 7 doses.

After 5 days, cells were lysed using 350 μL Lysis buffer using the MagNA Pure 96 system according to manufacturer's instructions (Roche LifeScience) and extracted in 50 μL RNAse free water. One-step qPCR was done using qScript® One-Step qRT-PCR Kit, Low ROX™) according to Manufacturer's protocol (QuantaBio).

For the qPCR, the following TaqMan gene expression assays were used: JAK1 (FAM): Oc06751244_m1 (Catalogue number: 4351372, TaqMan Thermofisher Scientific) and GAPDH (VIC): Oc03823402_g1 (Catalogue number: 4331182, TaqMan Thermofisher Scientific)

JAK1 mRNA concentrations were quantified relative to the housekeeping gene GAPDH using QuantStudio™ Real-time PCR system Software (Applied Biosystem) and normalized to only PBS treated SIRC1 cells (PBS set to 100%).

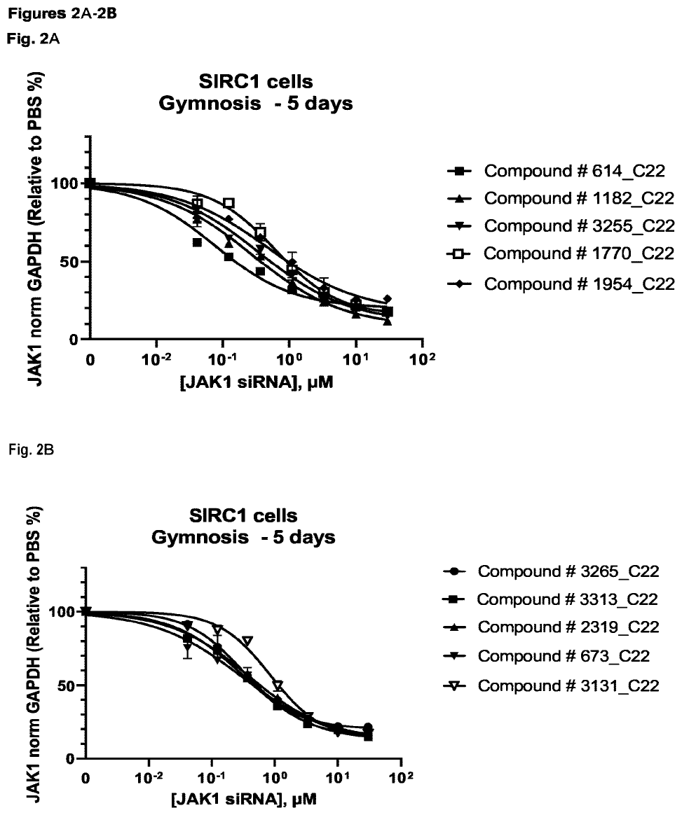

All the conjugated siRNA tested targeting JAK1 (compound #614_C22, 673_C22, 1182_C22, 1770_C22, 1954_C22, 2319_C22, 3131_C22, 3255_C22, 3265_C22, and 3313_C22) were potent and efficacious with potencies less than 1 μM as shown in FIG. 2a (compounds 614, 1182, 3255, 1770 and 1954) and FIG. 2b (compounds 3265, 3313 2319, 673, 3131).

### Example 5—JAK1 mRNA Expression after Gymnosis for 5 Days of HCEC Cells with the C22 Conjugated siRNAs Targeting JAK1 (Compound #614_C22, 673_C22, 1182_C22, 1770_C22, 1954_C22, 2319_C22, 3131_C22, 3255_C22, 3265_C22, and 3313_C22)

HCEC, primary Human Cornea Epithelial cells (PCS-700-010, ATCC) were plated in 96 well plates 5000 cells per well in full growth medium and treated the day after with C22—conjugated siRNA molecules (compound #614_C22, 673_C22, 1182_C22, 1770_C22, 1954_C22, 2319_C22, 3131_C22, 3255_C22, 3265_C22, and 3313_C22) in PBS for final concentrations of 30 UM and three-fold dilutions for 7 doses.

After 5 days cells were lysed using 350 μL Lysis buffer using the MagNA Pure 96 system according to manufacturer's instructions (Roche LifeScience) and extracted in 50 μL RNAse free water. One-step qPCR was done using qScript® One-Step qRT-PCR Kit, Low ROX™ according to manufacturer's protocol (QuantaBio).

The following TaqMan gene expression assays were used: JAK1 (FAM): Hs01026985_m1 (Catalogue number: 4351372, TaqMan Thermofisher Scientific) and GAPDH (VIC): Hs02786624_g1 (Catalog number: 4448489, TaqMan Thermofisher Scientific).

JAK1 mRNA concentrations were quantified relative to the housekeeping gene GAPDH using QuantStudio™ Real-time PCR system Software (Applied Biosystem) and normalized to only PBS treated HCEC cells (PBS set to 100%).

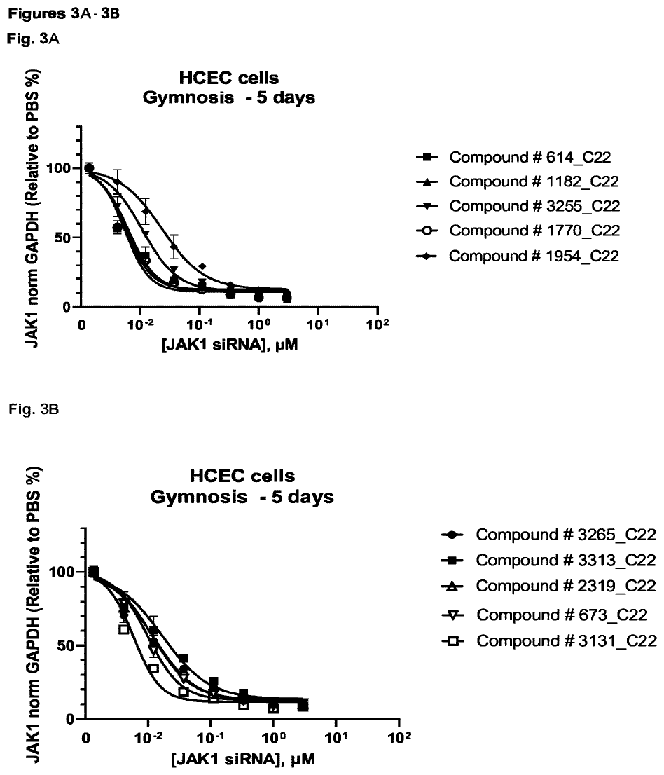

All the C22-conjugated siRNA targeting JAK1 (compound #614_C22, 673_C22, 1182_C22, 1770_C22, 1954_C22, 2319_C22, 3131_C22, 3255_C22, 3265_C22, and 3313_C22) were very potent with potencies less than 25 nM, as shown in FIGS. 3a and 3b.

Table 7 below recapitulates the results of Examples 3, 4 and 5.

TABLE 7
Potencies for compound # 614_C22, 673_C22, 1182_C22, 1770_C22,
1954_C22, 2319_C22, 3131_C22, 3255_C22, 3265_C22, and 3313_C22
in both SIRC1 and HCEC cells (see Examples 3, 4 and 5).

| | | | | IC50 HCEC | IC50 SIRC1 |
|---|---|---|---|---|---|
| | | | | human | Rabbit |
| | | Corresponding | IC50 HCEC | C22-siRNA | C22-siRNA |
| Compound | Corresponding | antisense | human (pM) | (nM)— | (μM)— |
| # | Duplex # | SEQ ID NO | Transfect | Gymnosis | Gymnosis |
| 614 | 1 | 194 | 5.1 | 5.7 | 0.077 |
| 673 | 2 | 195 | 7.6 | 9.9 | 0.30 |
| 1182 | 21 | 214 | 4.3 | 6.3 | 0.28 |
| 1770 | 63 | 256 | 3.6 | 5.5 | 0.61 |
| 1954 | 72 | 265 | 10.4 | 23 | 0.48 |
| 2319 | 95 | 288 | 5.8 | 12 | 0.38 |
| 3131 | 114 | 307 | 5.8 | 5.9 | 0.86 |
| 3255 | 128 | 321 | 8.6 | 11 | 0.36 |
| 3265 | 133 | 326 | 13.7 | 12 | 0.30 |
| 3313 | 146 | 339 | 20.5 | 17 | 0.32 |

### Example 6—In Vivo Efficacy of JAK1 siRNAs

The ability of ten C22-conjugated JAK1 siRNAs to reduce JAK1 mRNA in the eyes of rabbits was tested.

New Zealand white rabbits were dosed by topical administration in the eye, 3 times pr day with a least 4 h between for 5 days with 20 μl of a 25 μg/μL solution (500 μg pr dose) of C22-conjugated siRNA molecules (Compound #614_C22, 673_C22, 1182_C22, 1770_C22, 1954_C22, 2319_C22, 3131_C22, 3255_C22, 3265_C22, and 3313_C22) in PBS. Three days after last dosing, EYEPRIM (OPIA technologies) samples were taken from the bulbar conjunctiva (Saline n=4, siRNA treated groups n=8). Following sacrifice of the animal, bulbar conjunctiva will be exposed and an EYEPRIME membrane will be pressed against the inferior bulbar conjunctiva for 3 seconds. Then the membrane was removed from the EYEPRIM. While doing so, the membrane was held with forceps at the time of ejection to avoid the membrane falling/flying away. The membrane was snap-frozen into a 2 mL Eppendorf tube.

The EYEPRIM samples were homogenized using the TissueLyser II (Qiagen) in 500 μL MagnaPure Tissue Lysis buffer (Roche LifeScience) after adding a metal bead and mRNA was extracted from 350 μL Lysis buffer using the MagNA Pure 96 system according to manufacturer's instructions (Roche LifeScience) and extracted in 50 μL RNAse-free water. cDNA synthesis was performed with 4 μL input RNA using IScript Advanced cDNA Synthesis Kit for RT-qPCR (Bio-Rad) and 2 μL was used as input for digital droplet PCR using ddPCR supermix for probes (no dUTP) (Bio-Rad) according to manufacturer's protocol.

The following TaqMan gene expression assays were used: JAK1 (FAM): Oc06751244_m1 (Catalogue number: 4351372, TaqMan Thermofisher Scientific) and GAPDH (VIC): Oc03823402_g1 (Catalogue number: 4331182, TaqMan Thermofisher Scientific)

JAK1 mRNA concentrations were quantified relative to the housekeeping gene GAPDH using QuantaSoft Software (Bio-Rad) and normalized to PBS treated rabbits (PBS set to 1)

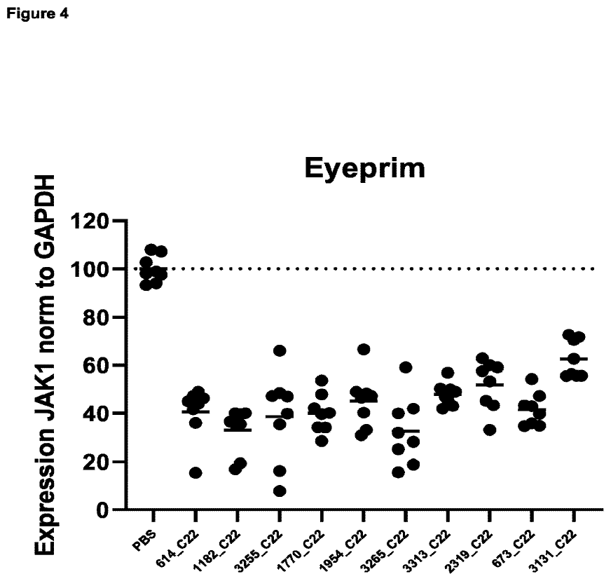

The results are show in FIG. 4:
67% knockdown for compound #1182_C22 and 3265_C22,
61% knockdown for compound #3255_C22,
60% knockdown for compound #614_C22, and 1170_C22, and
37% to 58% knockdown for compound #1954_C22; 3313_C22; 2319_C22; 673_C22; and 3131_C22 in the conjunctiva EYEPRIM samples.

### Example 7—In Vivo Efficacy of Compound #614_C22, and 1182_C22 in IFNg-Stimulated Eyes

New Zealand white rabbits were dosed by topical administration in the eye, 3 times pr day with a least 4 h between for 5 days with 20 μl of a 25 μg/μL solution (500 μg pr dose) of C22-conjugated siRNA molecules (Compound #614_C22, and 1182_C22).

siRNA in PBS solution were dosed in the left eye only, with only PBS in the right eye. Three days after last dosing, rabbit IFNg (Kingfisher Biotech) was dosed topically using 25 μL of 100 μg/mL (2.5 μg pr dose) in PBS three times with 2 h between each dose, in both eyes. 1 h after last IFNg dosing, EYEPRIM (OPIA technologies) samples were taken from the bulbar conjunctiva (Saline n=4, siRNA treated groups n=8). Following sacrifice of the animal, bulbar conjunctiva were exposed and an EYEPRIME membrane was pressed against the inferior bulbar conjunctiva for 3 seconds. Then the membrane was removed from the EYEPRIM. While doing so, the membrane was held with forceps at the time of ejection to avoid the membrane falling/flying away. The membrane was snap-frozen into a 2 mL Eppendorf tube.

The EYEPRIM samples were homogenized using a TissueLyser II (Qiagen) in 500 μL MagnaPure Tissue Lysis buffer (Roche LifeScience) after adding a metal bead, and mRNA was extracted from 350 μL Lysis buffer using the MagNA Pure 96 system according to manufacturer's instructions (Roche LifeScience) and extracted in 50 μL RNAse-free water. cDNA synthesis was performed with 4 μL input RNA using IScript Advanced cDNA Synthesis Kit for RT-qPCR (Bio-Rad) and 2 μL was used as input for digital droplet PCR using ddPCR supermix for probes (no dUTP) (Bio-Rad) according to manufacturer's protocol.

The following TaqMan gene expression assays were used:
JAK1 (FAM): Oc06751244_m1 (Catalog number: 4351372, TaqMan Thermofisher Scientific)
GAPDH (VIC): Oc03823402_g1 (Catalog number: 4331182, TaqMan Thermofisher Scientific)
CXCL10 (FAM): Oc06781609_g1 (Catalog number: 4351372, TaqMan Thermofisher Scientific)

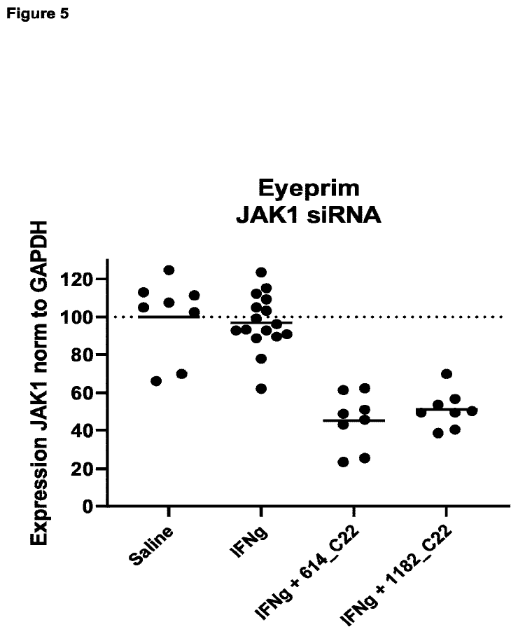

JAK1 mRNA concentrations were quantified relative to the housekeeping gene GAPDH using QuantaSoft Software (Bio-Rad) and normalized to PBS-treated rabbits (PBS set to 1) The results are show in FIG. 5. JAK1 knockdown was 55% knockdown (Compound #614_C22, and 1182_C22).

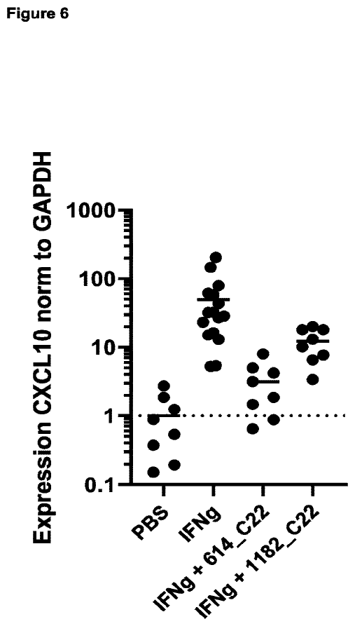

The results for INFg stimulation are shown in FIG. 6. INFg stimulation induced a 50-fold induction of the IFNg induced target CXCL10. Treatment with C22-conjugated siRNA (Compound #614_C22, and 1182_C22) substantially reduced the induction of CXCL10 by IFNg.

### Example 8—In Vivo Efficacy of JAK1 C16 siRNAs

The ability of JAK1 C16 siRNAs (i.e., twelve siRNA duplexes each conjugated to palmitic acid) to reduce JAK1 mRNA in the eyes of rabbits was tested.

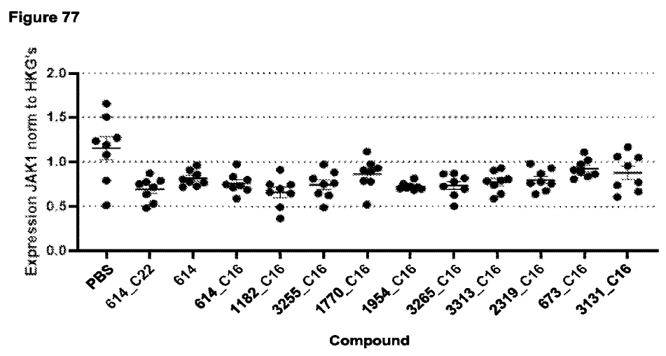

PBS as a negative control, ten C16 siRNAs and two control siRNAs (namely a C22 siRNA (compound 614_C22) and a naked siRNA (compound 614)) were each administered to rabbits (n=2/group, for a total of 26 rabbits) three times daily for five days. To evaluate the effect of the compounds on JAK1 expression, at termination of the animals on Day 7, samples of conjunctival cells (using Eyeprim sampling), cornea, palpebral conjunctiva, retina, kidney, and liver were taken from all animals and JAK1 mRNA levels were determined using qPCR. JAK1 expression was normalised to a combined set of housekeeping genes (“HKG”), namely HPRT1 (Hypoxanthin-Guanin-Phosphoribosyltransferase), PPIA, (peptidylprolyl isomerase A) and GAPDH (glyceraldehyde-3-phosphate dehydrogenase). The results are shown in FIG. 77.

All tested C16 siRNAs, namely compounds 614_C16, 673_C16, 1182_C16, 1770_C16, 1954_C16, 2319_C16, 3131_C16, 3255_C16, 3265_C16 and 3313_C16, effectively suppress JAK1 expression.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, biochemistry, cell biology or related fields are intended to be within the scope of the following claims.

### SEQUENCE LISTING

SEQ ID NO: 1—JAK1 mRNA
SEQ ID NO: 2—JAK1 cDNA (complementary to SEQ ID NO: 1)
SEQ ID NOs 3-193—Sense strand sequences
SEQ ID NOS 194-384—Antisense strand sequences
SEQ ID NOs 385-575—Target site sequences (21 or 20 nts)
SEQ ID NOS 576-766—Seed region sequences
SEQ ID NOS 767-957—Seed region target sequences
SEQ ID NOS 958-1148—Sense strands
SEQ ID NOS 1149-1339—Antisense strands

TABLE 8
Summary of compound numbers

| | | Sense | Antisense | | | Sense | Antisense |
|---|---|---|---|---|---|---|---|
| Compound # | | strand | strand | Target | Seed | strand | strand |
| (Start pos. in | Duplex | sequence | sequence | SEQ ID | SEQ | SEQ ID | SEQ ID |
| NM_002227.4) | # | SEQ ID NO | SEQ ID NO | NO | ID NO | NO | NO |
| 614 | 1 | 3 | 194 | 385 | 576 | 958 | 1149 |
| 673 | 2 | 4 | 195 | 386 | 577 | 959 | 1150 |
| 724 | 3 | 5 | 196 | 387 | 578 | 960 | 1151 |
| 728 | 4 | 6 | 197 | 388 | 579 | 961 | 1152 |
| 753 | 5 | 7 | 198 | 389 | 580 | 962 | 1153 |
| 756 | 6 | 8 | 199 | 390 | 581 | 963 | 1154 |
| 818 | 7 | 9 | 200 | 391 | 582 | 964 | 1155 |
| 874 | 8 | 10 | 201 | 392 | 583 | 965 | 1156 |
| 875 | 9 | 11 | 202 | 393 | 584 | 966 | 1157 |
| 876 | 10 | 12 | 203 | 394 | 585 | 967 | 1158 |
| 877 | 11 | 13 | 204 | 395 | 586 | 968 | 1159 |
| 878 | 12 | 14 | 205 | 396 | 587 | 969 | 1160 |
| 883 | 13 | 15 | 206 | 397 | 588 | 670 | 1161 |
| 884 | 14 | 16 | 207 | 398 | 589 | 971 | 1162 |
| 1069 | 15 | 17 | 208 | 399 | 590 | 972 | 1163 |
| 1075 | 16 | 18 | 209 | 400 | 591 | 973 | 1164 |
| 1085 | 17 | 19 | 210 | 401 | 592 | 974 | 1165 |
| 1107 | 18 | 20 | 211 | 402 | 593 | 975 | 1166 |
| 1108 | 19 | 21 | 212 | 403 | 594 | 976 | 1167 |
| 1138 | 20 | 22 | 213 | 404 | 595 | 977 | 1168 |
| 1182 | 21 | 23 | 214 | 405 | 596 | 978 | 1169 |
| 1189 | 22 | 24 | 215 | 406 | 597 | 979 | 1170 |
| 1190 | 23 | 25 | 216 | 407 | 598 | 980 | 1171 |
| 1304 | 24 | 26 | 217 | 408 | 599 | 981 | 1172 |
| 1306 | 25 | 27 | 218 | 409 | 600 | 982 | 1173 |
| 1311 | 26 | 28 | 219 | 410 | 601 | 983 | 1174 |
| 1367 | 27 | 29 | 220 | 411 | 602 | 984 | 1175 |
| 1368 | 28 | 30 | 221 | 412 | 603 | 985 | 1176 |
| 1372 | 29 | 31 | 222 | 413 | 604 | 986 | 1177 |
| 1412 | 30 | 32 | 223 | 414 | 605 | 987 | 1178 |
| 1413 | 31 | 33 | 224 | 415 | 606 | 988 | 1179 |
| 1432 | 32 | 34 | 225 | 416 | 607 | 989 | 1180 |
| 1579 | 33 | 35 | 226 | 417 | 608 | 990 | 1181 |
| 1580 | 34 | 36 | 227 | 418 | 609 | 991 | 1182 |
| 1581 | 35 | 37 | 228 | 419 | 610 | 992 | 1183 |
| 1583 | 36 | 38 | 229 | 420 | 611 | 993 | 1184 |
| 1584 | 37 | 39 | 230 | 421 | 612 | 994 | 1185 |
| 1586 | 38 | 40 | 231 | 422 | 613 | 995 | 1186 |
| 1587 | 39 | 41 | 232 | 423 | 614 | 996 | 1187 |
| 1588 | 40 | 42 | 233 | 424 | 615 | 997 | 1188 |
| 1595 | 41 | 43 | 234 | 425 | 616 | 998 | 1189 |
| 1596 | 42 | 44 | 235 | 426 | 617 | 999 | 1190 |
| 1601 | 43 | 45 | 236 | 427 | 618 | 1000 | 1191 |
| 1602 | 44 | 46 | 237 | 428 | 619 | 1001 | 1192 |
| 1603 | 45 | 47 | 238 | 429 | 620 | 1002 | 1193 |
| 1608 | 46 | 48 | 239 | 430 | 621 | 1003 | 1194 |
| 1609 | 47 | 49 | 240 | 431 | 622 | 1004 | 1195 |
| 1611 | 48 | 50 | 241 | 432 | 623 | 1005 | 1196 |
| 1640 | 49 | 51 | 242 | 433 | 624 | 1006 | 1197 |
| 1642 | 50 | 52 | 243 | 434 | 625 | 1007 | 1198 |
| 1671 | 51 | 53 | 244 | 435 | 626 | 1008 | 1199 |
| 1672 | 52 | 54 | 245 | 436 | 627 | 1009 | 1200 |
| 1673 | 53 | 55 | 246 | 437 | 628 | 1010 | 1201 |
| 1674 | 54 | 56 | 247 | 438 | 629 | 1011 | 1202 |
| 1677 | 55 | 57 | 248 | 439 | 630 | 1012 | 1203 |
| 1678 | 56 | 58 | 249 | 440 | 631 | 1013 | 1204 |
| 1690 | 57 | 59 | 250 | 441 | 632 | 1014 | 1205 |
| 1692 | 58 | 60 | 251 | 442 | 633 | 1015 | 1206 |
| 1698 | 59 | 61 | 252 | 443 | 634 | 1016 | 1207 |
| 1699 | 60 | 62 | 253 | 444 | 635 | 1017 | 1208 |
| 1723 | 61 | 63 | 254 | 445 | 636 | 1018 | 1209 |
| 1769 | 62 | 64 | 255 | 446 | 637 | 1019 | 1210 |
| 1770 | 63 | 65 | 256 | 447 | 638 | 1020 | 1211 |
| 1780 | 64 | 66 | 257 | 448 | 639 | 1021 | 1212 |
| 1798 | 65 | 67 | 258 | 449 | 640 | 1022 | 1213 |
| 1876 | 66 | 68 | 259 | 450 | 641 | 1023 | 1214 |
| 1927 | 67 | 69 | 260 | 451 | 642 | 1024 | 1215 |
| 1928 | 68 | 70 | 261 | 452 | 643 | 1025 | 1216 |
| 1929 | 69 | 71 | 262 | 453 | 644 | 1026 | 1217 |
| 1936 | 70 | 72 | 263 | 454 | 645 | 1027 | 1218 |
| 1952 | 71 | 73 | 264 | 455 | 646 | 1028 | 1219 |
| 1954 | 72 | 74 | 265 | 456 | 647 | 1029 | 1220 |
| 1956 | 73 | 75 | 266 | 457 | 648 | 1030 | 1221 |
| 1958 | 74 | 76 | 267 | 458 | 649 | 1031 | 1222 |
| 1978 | 75 | 77 | 268 | 459 | 650 | 1032 | 1223 |
| 2066 | 76 | 78 | 269 | 460 | 651 | 1033 | 1224 |
| 2068 | 77 | 79 | 270 | 461 | 652 | 1034 | 1225 |
| 2102 | 78 | 80 | 271 | 462 | 653 | 1035 | 1226 |
| 2111 | 79 | 81 | 272 | 463 | 654 | 1036 | 1227 |
| 2138 | 80 | 82 | 273 | 464 | 655 | 1037 | 1228 |
| 2146 | 81 | 83 | 274 | 465 | 656 | 1038 | 1229 |
| 2148 | 82 | 84 | 275 | 466 | 657 | 1039 | 1230 |
| 2205 | 83 | 85 | 276 | 467 | 658 | 1040 | 1231 |
| 2206 | 84 | 86 | 277 | 468 | 659 | 1041 | 1232 |
| 2218 | 85 | 87 | 278 | 469 | 660 | 1042 | 1233 |
| 2229 | 86 | 88 | 279 | 470 | 661 | 1043 | 1234 |
| 2230 | 87 | 89 | 280 | 471 | 662 | 1044 | 1235 |
| 2237 | 88 | 90 | 281 | 472 | 663 | 1045 | 1236 |
| 2238 | 89 | 91 | 282 | 473 | 664 | 1046 | 1237 |
| 2239 | 90 | 92 | 283 | 474 | 665 | 1047 | 1238 |
| 2269 | 91 | 93 | 284 | 475 | 666 | 1048 | 1239 |
| 2308 | 92 | 94 | 285 | 476 | 667 | 1049 | 1240 |
| 2317 | 93 | 95 | 286 | 477 | 668 | 1050 | 1241 |
| 2318 | 94 | 96 | 287 | 478 | 669 | 1051 | 1242 |
| 2319 | 95 | 97 | 288 | 479 | 670 | 1052 | 1243 |
| 2320 | 96 | 98 | 289 | 480 | 671 | 1053 | 1244 |
| 2321 | 97 | 99 | 290 | 481 | 672 | 1054 | 1245 |
| 2322 | 98 | 100 | 291 | 482 | 673 | 1055 | 1246 |
| 2323 | 99 | 101 | 292 | 483 | 674 | 1056 | 1247 |
| 2520 | 100 | 102 | 293 | 484 | 675 | 1057 | 1248 |
| 2527 | 101 | 103 | 294 | 485 | 676 | 1058 | 1249 |
| 2647 | 102 | 104 | 295 | 486 | 677 | 1059 | 1250 |
| 2761 | 103 | 105 | 296 | 487 | 678 | 1060 | 1251 |
| 2762 | 104 | 106 | 297 | 488 | 679 | 1061 | 1252 |
| 2763 | 105 | 107 | 298 | 489 | 680 | 1062 | 1253 |
| 2764 | 106 | 108 | 299 | 490 | 681 | 1063 | 1254 |
| 2811 | 107 | 109 | 300 | 491 | 682 | 1064 | 1255 |
| 2962 | 108 | 110 | 301 | 492 | 683 | 1065 | 1256 |
| 2975 | 109 | 111 | 302 | 493 | 684 | 1066 | 1257 |
| 2977 | 110 | 112 | 303 | 494 | 685 | 1067 | 1258 |
| 3028 | 111 | 113 | 304 | 495 | 686 | 1068 | 1259 |
| 3032 | 112 | 114 | 305 | 496 | 687 | 1069 | 1260 |
| 3081 | 113 | 115 | 306 | 497 | 688 | 1070 | 1261 |
| 3131 | 114 | 116 | 307 | 498 | 689 | 1071 | 1262 |
| 3134 | 115 | 117 | 308 | 499 | 690 | 1072 | 1263 |
| 3141 | 116 | 118 | 309 | 500 | 691 | 1073 | 1264 |
| 3144 | 117 | 119 | 310 | 501 | 692 | 1074 | 1265 |
| 3146 | 118 | 120 | 311 | 502 | 693 | 1075 | 1266 |
| 3147 | 119 | 121 | 312 | 503 | 694 | 1076 | 1267 |
| 3159 | 120 | 122 | 313 | 504 | 695 | 1077 | 1268 |
| 3160 | 121 | 123 | 314 | 505 | 696 | 1078 | 1269 |
| 3229 | 122 | 124 | 315 | 506 | 697 | 1079 | 1270 |
| 3247 | 123 | 125 | 316 | 507 | 698 | 1080 | 1271 |
| 3250 | 124 | 126 | 317 | 508 | 699 | 1081 | 1272 |
| 3251 | 125 | 127 | 318 | 509 | 700 | 1082 | 1273 |
| 3252 | 126 | 128 | 319 | 510 | 701 | 1083 | 1274 |
| 3254 | 127 | 129 | 320 | 511 | 702 | 1084 | 1275 |
| 3255 | 128 | 130 | 321 | 512 | 703 | 1085 | 1276 |
| 3258 | 129 | 131 | 322 | 513 | 704 | 1086 | 1277 |
| 3259 | 130 | 132 | 323 | 514 | 705 | 1087 | 1278 |
| 3260 | 131 | 133 | 324 | 515 | 706 | 1088 | 1279 |
| 3261 | 132 | 134 | 325 | 516 | 707 | 1089 | 1280 |
| 3265 | 133 | 135 | 326 | 517 | 708 | 1090 | 1281 |
| 3268 | 134 | 136 | 327 | 518 | 709 | 1091 | 1282 |
| 3272 | 135 | 137 | 328 | 519 | 710 | 1092 | 1283 |
| 3275 | 136 | 138 | 329 | 520 | 711 | 1093 | 1284 |
| 3276 | 137 | 139 | 330 | 521 | 712 | 1094 | 1285 |
| 3278 | 138 | 140 | 331 | 522 | 713 | 1095 | 1286 |
| 3279 | 139 | 141 | 332 | 523 | 714 | 1096 | 1287 |
| 3281 | 140 | 142 | 333 | 524 | 715 | 1097 | 1288 |
| 3282 | 141 | 143 | 334 | 525 | 716 | 1098 | 1289 |
| 3283 | 142 | 144 | 335 | 526 | 717 | 1099 | 1290 |
| 3284 | 143 | 145 | 336 | 527 | 718 | 1100 | 1291 |
| 3285 | 144 | 146 | 337 | 528 | 719 | 1101 | 1292 |
| 3286 | 145 | 147 | 338 | 529 | 720 | 1102 | 1293 |
| 3313 | 146 | 148 | 339 | 530 | 721 | 1103 | 1294 |
| 3314 | 147 | 149 | 340 | 531 | 722 | 1104 | 1295 |
| 3323 | 148 | 150 | 341 | 532 | 723 | 1105 | 1296 |
| 3353 | 149 | 151 | 342 | 533 | 724 | 1106 | 1297 |
| 3365 | 150 | 152 | 343 | 534 | 725 | 1107 | 1298 |
| 3367 | 151 | 153 | 344 | 535 | 726 | 1108 | 1299 |
| 3368 | 152 | 154 | 345 | 536 | 727 | 1109 | 1300 |
| 3371 | 153 | 155 | 346 | 537 | 728 | 1110 | 1301 |
| 3372 | 154 | 156 | 347 | 538 | 729 | 1111 | 1302 |
| 3376 | 155 | 157 | 348 | 539 | 730 | 1112 | 1303 |
| 3409 | 156 | 158 | 349 | 540 | 731 | 1113 | 1304 |
| 3505 | 157 | 159 | 350 | 541 | 732 | 1114 | 1305 |
| 3556 | 158 | 160 | 351 | 542 | 733 | 1115 | 1306 |
| 3557 | 159 | 161 | 352 | 543 | 734 | 1116 | 1307 |
| 3558 | 160 | 162 | 353 | 544 | 735 | 1117 | 1308 |
| 3559 | 161 | 163 | 354 | 545 | 736 | 1118 | 1309 |
| 3654 | 162 | 164 | 355 | 546 | 737 | 1119 | 1310 |
| 3662 | 163 | 165 | 356 | 547 | 738 | 1120 | 1311 |
| 3663 | 164 | 166 | 357 | 548 | 739 | 1121 | 1312 |
| 3683 | 165 | 167 | 358 | 549 | 740 | 1122 | 1313 |
| 3689 | 166 | 168 | 359 | 550 | 741 | 1123 | 1314 |
| 3694 | 167 | 169 | 360 | 551 | 742 | 1124 | 1315 |
| 3695 | 168 | 170 | 361 | 552 | 743 | 1125 | 1316 |
| 3698 | 169 | 171 | 362 | 553 | 744 | 1126 | 1317 |
| 3702 | 170 | 172 | 363 | 554 | 745 | 1127 | 1318 |
| 3719 | 171 | 173 | 364 | 555 | 746 | 1128 | 1319 |
| 3781 | 172 | 174 | 365 | 556 | 747 | 1129 | 1320 |
| 3894 | 173 | 175 | 366 | 557 | 748 | 1130 | 1321 |
| 4099 | 174 | 176 | 367 | 558 | 749 | 1131 | 1322 |
| 4169 | 175 | 177 | 368 | 559 | 750 | 1132 | 1323 |
| 4239 | 176 | 178 | 369 | 560 | 751 | 1133 | 1324 |
| 4305 | 177 | 179 | 370 | 561 | 752 | 1134 | 1325 |
| 4374 | 178 | 180 | 371 | 562 | 753 | 1135 | 1326 |
| 4411 | 179 | 181 | 372 | 563 | 754 | 1136 | 1327 |
| 4475 | 180 | 182 | 373 | 564 | 755 | 1137 | 1328 |
| 4612 | 181 | 183 | 374 | 565 | 756 | 1138 | 1329 |
| 4671 | 182 | 184 | 375 | 566 | 757 | 1139 | 1330 |
| 4672 | 183 | 185 | 376 | 567 | 758 | 1140 | 1331 |
| 4679 | 184 | 186 | 377 | 568 | 759 | 1141 | 1332 |
| 4682 | 185 | 187 | 378 | 569 | 760 | 1142 | 1333 |
| 4683 | 186 | 188 | 379 | 570 | 761 | 1143 | 1334 |
| 4684 | 187 | 189 | 380 | 571 | 762 | 1144 | 1335 |
| 4690 | 188 | 190 | 381 | 572 | 763 | 1145 | 1336 |
| 4794 | 189 | 191 | 382 | 573 | 764 | 1146 | 1337 |
| 4803 | 190 | 192 | 383 | 574 | 765 | 1147 | 1338 |
| 4807 | 191 | 193 | 384 | 575 | 766 | 1148 | 1339 |

1. A compound comprising a double stranded ribonucleic acid (dsRNA) for reducing the expression of Janus kinase 1 (JAK1), the dsRNA comprising a sense strand and an antisense strand,
wherein the sense strand comprises a first contiguous nucleotide sequence of at least 15 nucleotides in length,
wherein the antisense strand comprises a second contiguous nucleotide sequence of at least 15 nucleotides in length which is complementary to a JAK1 nucleic acid sequence which comprises or consists of SEQ ID NO: 1 or a naturally occurring variant thereof, and
wherein the first contiguous nucleotide sequence and the second contiguous nucleotide sequence form a double stranded region of complementarity.

2. The compound of claim 1, wherein the second contiguous nucleotide sequence is complementary to a target sequence within the JAK1 nucleic acid sequence, wherein the target sequence is any one of the sequences of SEQ ID NOs 385-575, preferably any one of the sequences of SEQ ID NOs 385, 386, 405, 447, 456, 479, 498, 512, 517 and 530.

3. The compound of claim 1, wherein the second contiguous nucleotide sequence comprises a seed region, wherein the sequence of the seed region comprises or consists of any one of the sequences of SEQ ID NOs 576-766.

4. The compound of claim 1, wherein the second contiguous nucleotide sequence comprises or consists of a sequence having at least 80% identity to any one of the sequences of SEQ ID NOs 194-384, such as at least 85%, 90%, 91%, 92%, 93%, 94%, 95%, 96%, 97%, 98% or 99% identity to any one of the sequences of SEQ ID NOs 194-384, preferably wherein the second contiguous nucleotide sequence comprises or consists of any one of the sequences of SEQ ID NOs 194-384.

5. The compound of claim 1, wherein the antisense strand comprises a uracil nucleotide, such as a Vinyl-phosphonate 2′-OMe uracil, located at the 5′ end of the antisense strand.

6. The compound of claim 1, wherein the first contiguous nucleotide sequence comprises or consists of any one of the sequences of SEQ ID NOs 3-193, preferably any one of the sequences of SEQ ID NOs 3, 4, 21, 63, 72, 95, 114, 128, 133 and 146.

7. The compound of claim 1, wherein the dsRNA comprises at least one modified nucleotide.

8. The compound of claim 7, wherein the non-bicyclic sugar moiety is independently selected from 2′-O-alkyl-RNA, 2′-O-methyl-RNA (2′OMe modified sugar), 2′-alkoxy-RNA, 2′-O-methoxyethyl-RNA, 2′-amino-DNA, 2′-fluoro-DNA (2′F modified sugar), arabino nucleic acid (ANA), 2′-fluoro-ANA, Glycol nucleic acid (GNA), and unlocked nucleic acid (UNA), preferably wherein the non-bicyclic sugar moiety is independently selected from a 2′F modified sugar and a 2′OMe modified sugar.

9. The compound of claim 1, wherein the dsRNA comprises at least one least one modified internucleotide linkage.

10. The compound of claim 1, wherein each internucleotide linkage of the dsRNA is either a phosphodiester internucleotide linkage or a phosphorothioate internucleotide linkage.

11. The compound of claim 1, wherein the dsRNA is covalently attached to at least one conjugate moiety.

12. The compound of claim 11, wherein the dsRNA is covalently attached to the conjugate moiety via a linker.

13. The compound of claim 1, wherein the compound is selected from compounds 614, 673, 724, 728, 753, 756, 818, 874, 875, 876, 877, 878, 883, 884, 1069, 1075, 1085, 1107, 1108, 1138, 1182, 1189, 1190, 1304, 1306, 1311, 1367, 1368, 1372, 1412, 1413, 1432, 1579, 1580, 1581, 1583, 1584, 1586, 1587, 1588, 1595, 1596, 1601, 1602, 1603, 1608, 1609, 1611, 1640, 1642, 1671, 1672, 1673, 1674, 1677, 1678, 1690, 1692, 1698, 1699, 1723, 1769, 1770, 1780, 1798, 1876, 1927, 1928, 1929, 1936, 1952, 1954, 1956, 1958, 1978, 2066, 2068, 2102, 2111, 2138, 2146, 2148, 2205, 2206, 2218, 2229, 2230, 2237, 2238, 2239, 2269, 2308, 2317, 2318, 2319, 2320, 2321, 2322, 2323, 2520, 2527, 2647, 2761, 2762, 2763, 2764, 2811, 2962, 2975, 2977, 3028, 3032, 3081, 3131, 3134, 3141, 3144, 3146, 3147, 3159, 3160, 3229, 3247, 3250, 3251, 3252, 3254, 3255, 3258, 3259, 3260, 3261, 3265, 3268, 3272, 3275, 3276, 3278, 3279, 3281, 3282, 3283, 3284, 3285, 3286, 3313, 3314, 3323, 3353, 3365, 3367, 3368, 3371, 3372, 3376, 3409, 3505, 3556, 3557, 3558, 3559, 3654, 3662, 3663, 3683, 3689, 3694, 3695, 3698, 3702, 3719, 3781, 3894, 4099, 4169, 4239, 4305, 4374, 4411, 4475, 4612, 4671, 4672, 4679, 4682, 4683, 4684, 4690, 4794, 4803, and 4807, as shown in Table 3, preferably a compound selected from compounds 614, 673, 1182, 1770, 1954, 2319, 3131, 3255, 3265, and 3313 as shown in Table 3.

14. The compound of claim 1, wherein the compound is selected from compounds 614, 673, 1182, 1770, 1954, 2319, 3131, 3255, 3265, 3313, 614_C16, 673_C16, 1182_C16, 1770_C16, 1954_C16, 2319_C16, 3131_C16, 3255_C16, 3265_C16, 3313_C16, 614_C22, 673_C22, 1182_C22, 1770_C22, 1954_C22, 2319_C22, 3131_C22, 3255_C22, 3265_C22 and 3313_C22.

15. The compound of claim 1, wherein the compound is in the form of a pharmaceutically acceptable salt, preferably a sodium salt or a potassium salt.

16. A pharmaceutical composition comprising the compound of claim 1, and a pharmaceutically acceptable diluent, solvent, carrier, salt and/or adjuvant, preferably an aqueous diluent or solvent; more preferably phosphate buffered saline.

17. An in vivo or in vitro method for suppressing JAK1 expression in a target cell, the method comprising administering the compound of claim 1, in an effective amount, to the target cell.

18. (canceled)

19. The method of claim 18, wherein the disease is selected from the group consisting of inflammatory bowel disease, organ transplant rejection, graft-versus-host disease, multiple sclerosis, rheumatoid arthritis (RA), juvenile idiopathic arthritis, psoriasis, dermatitis, diabetic nephropathy, systemic lupus erythematosus (SLE), dry eye disease, cancer, myelofibrosis, and asthma, preferably dry eye disease.

20. A kit comprising the compound of claim 1 and instructions for use.

21. An in vivo or in vitro method for suppressing JAK1 expression in a target cell, the method comprising administering the pharmaceutical composition of claim 16, in an effective amount, to the target cell.

22. A method for treating or preventing a disease comprising administering a therapeutically or prophylactically effective amount of the compound of claim 1 to a subject suffering from or susceptible to a disease.

23. A method for treating or preventing a disease comprising administering a therapeutically or prophylactically effective amount of the pharmaceutical composition of claim 16 to a subject suffering from or susceptible to a disease.
